(19) United States
(12) Patent Application Publication
(10) Pub. No.: US 2026/0347453 A1
(43) Pub. Date: Nov. 5, 2026

(54) ANTI-CSP ANTIBODIES

(71) Applicant: ATRECA, INC., San Carlos, CA (US)

(72) Inventors: Daniel Eric EMERLING, San Carlos, CA (US); Randal R. KETCHEM, Snohomish, WA (US); Shaun M. LIPPOW, San Carlos, CA (US); Wayne VOLKMUTH, San Carlos, CA (US); Katherine L. WILLIAMS, San Carlos, CA (US)

(21) Appl. No.: 19/443,385

(22) Filed: Jan. 8, 2026

Related U.S. Application Data
(63) Continuation of application No. 18/101,933, filed on Jan. 26, 2023, now Pat. No. 12,545,723.
Application No. 18/101,933 is a continuation of application No. 17/842,351, filed on Jun. 16, 2022, now Pat. No. 11,655,290.
(60) Provisional application No. 63/211,820, filed on Jun. 17, 2021.

(51) Int. Cl. C07K 16/20; A61K 39/00; A61P 33/06

(52) U.S. Cl. CPC C07K 16/205; A61P 33/06; A61K 2039/505; C07K 2317/33; C07K 2317/92

(57) ABSTRACT

The present disclosure provides anti-circumsporozoite (CSP) antibodies, compositions comprising such antibodies. Also disclosed are methods of producing the disclosed antibodies and methods of treating or preventing malaria using the same.

### CROSS-REFERENCE TO RELATED APPLICATIONS

This application is a continuation of U.S. patent application Ser. No. 18/101,933, filed on Jan. 26, 2023, which is a continuation of U.S. patent application Ser. No. 17/842,351, filed Jun. 16, 2022, which claims priority to U.S. Provisional Application No. 63/211,820, filed Jun. 17, 2021, the content of each of the aforementioned applications is incorporated by reference in its entirety for all purposes.

### SEQUENCE LISTING

This application contains a Sequence Listing which has been submitted in xml format via EFS-Web and is hereby incorporated by reference in its entirety. Said XML copy, created on Jan. 25, 2023, is named 087996.0108.xml and is 490,821 bytes in size.

### FIELD

The present disclosure relates to compositions for treating or preventing malaria, and to antibodies conferring protection against infection by malarial parasites such as Plasmodium falciparum by insect vector transmission. The present disclosure also relates to methods for treating, preventing, or diagnosing Plasmodium infection in a mammal.

### BACKGROUND

Malaria causes a large burden of morbidity and mortality, especially in the developing world. The causative agent of malaria is a protozoal parasite, which is transmitted by mosquitoes. Several infectious Plasmodium species cause malaria, the deadliest of which is Plasmodium falciparum. Others include P. vivax, P. ovale, and P. malariae. A first-generation vaccine (RTS,S) has been developed using portions of the malaria protein CSP, including part of the NANP repeats. CSP-based vaccines have consistently shown 30-50% efficacy in the prevention of erythrocytic-stage infection. This level of efficacy is not sufficient for eradication and new pre-erythrocytic treatments will need superior efficacy. Despite the existence of other anti-malarial products such as mefloquinine, doxycycline, and atovaquone/proguanil, there is a need for new antimalarials options for cases that are resistant to existing antimalarial drugs.

### SUMMARY

The present disclosure provides to antibodies targeting Plasmodium falciparum. In certain non-limiting embodiments, the antibody is a recombinant anti-circumsporozoite (CSP) antibody. In certain embodiments, the recombinant antibody comprises a light chain variable region (VL) and a heavy chain variable region (VH), wherein the light chain variable region comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and the heavy chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the VL comprises the amino acid sequence set forth in SEQ ID NO: 163; the amino acid sequence set forth in SEQ ID NO: 23; the amino acid sequence set forth in SEQ ID NO: 33; the amino acid sequence set forth in SEQ ID NO: 43; the amino acid sequence set forth in SEQ ID NO: 53; the amino acid sequence set forth in SEQ ID NO: 63; the amino acid sequence set forth in SEQ ID NO: 73; the amino acid sequence set forth in SEQ ID NO: 83; the amino acid sequence set forth in SEQ ID NO: 93; the amino acid sequence set forth in SEQ ID NO: 103; the amino acid sequence set forth in SEQ ID NO: 113; the amino acid sequence set forth in SEQ ID NO: 123; the amino acid sequence set forth in SEQ ID NO: 133; the amino acid sequence set forth in SEQ ID NO: 143; the amino acid sequence set forth in SEQ ID NO: 153; or the amino acid sequence set forth in SEQ ID NO: 173. In certain embodiments, the VH comprises the amino acid sequence set forth in SEQ ID NO: 164; the amino acid sequence set forth in SEQ ID NO: 24; the amino acid sequence set forth in SEQ ID NO: 34; the amino acid sequence set forth in SEQ ID NO: 44; the amino acid sequence set forth in SEQ ID NO: 54; the amino acid sequence set forth in SEQ ID NO: 64; the amino acid sequence set forth in SEQ ID NO: 74; the amino acid sequence set forth in SEQ ID NO: 84; the amino acid sequence set forth in SEQ ID NO: 94; the amino acid sequence set forth in SEQ ID NO: 104; the amino acid sequence set forth in SEQ ID NO: 114; the amino acid sequence set forth in SEQ ID NO: 124; the amino acid sequence set forth in SEQ ID NO: 134; the amino acid sequence set forth in SEQ ID NO: 144; the amino acid sequence set forth in SEQ ID NO: 154; or the amino acid sequence set forth in SEQ ID NO: 174.

In certain embodiments, the VL comprises the amino acid sequence set forth in SEQ ID NO: 163, and the VH comprises the amino acid sequence set forth in SEQ ID NO: 164; the VL comprises the amino acid sequence set forth in SEQ ID NO: 33, and the VH comprises the amino acid sequence set forth in SEQ ID NO: 34; the VL comprises the amino acid sequence set forth in SEQ ID NO: 43, and the VH comprises the amino acid sequence set forth in SEQ ID NO: 44; the VL comprises the amino acid sequence set forth in SEQ ID NO: 53, and the VH comprises the amino acid sequence set forth in SEQ ID NO: 54; the VL comprises the amino acid sequence set forth in SEQ ID NO: 63, and the VH comprises the amino acid sequence set forth in SEQ ID NO: 64; the VL comprises the amino acid sequence set forth in SEQ ID NO: 73, and the VH comprises the amino acid sequence set forth in SEQ ID NO: 74; the VL comprises the amino acid sequence set forth in SEQ ID NO: 83, and the VH comprises the amino acid sequence set forth in SEQ ID NO: 84; the VL comprises the amino acid sequence set forth in SEQ ID NO: 93, and the VH comprises the amino acid sequence set forth in SEQ ID NO: 94; the VL comprises the amino acid sequence set forth in SEQ ID NO: 103, and the VH comprises the amino acid sequence set forth in SEQ ID NO: 104; the VL comprises the amino acid sequence set forth in SEQ ID NO: 113, and the VH comprises the amino acid sequence set forth in SEQ ID NO: 114; the VL comprises the amino acid sequence set forth in SEQ ID NO: 123, and the VH comprises the amino acid sequence set forth in SEQ ID NO: 124; the VL comprises the amino acid sequence set forth in SEQ ID NO: 133, and the VH comprises the amino acid sequence set forth in SEQ ID NO: 134; the VL comprises the amino acid sequence set forth in SEQ ID NO: 143, and the VH comprises the amino acid sequence set forth in SEQ ID NO: 144; the VL comprises the amino acid sequence set forth in SEQ ID NO: 153, and the VH comprises the amino acid sequence set forth in SEQ ID NO: 154; or the VL comprises the amino acid sequence set forth in SEQ ID NO: 173, and the VH comprises the amino acid sequence set forth in SEQ ID NO: 174.

In certain embodiments, the VL comprises the amino acid sequence set forth in SEQ ID NO: 63, and the VH comprises the amino acid sequence set forth in SEQ ID NO: 64. In certain embodiments, the VL comprises the amino acid sequence set forth in SEQ ID NO: 133, and the VH comprises the amino acid sequence set forth in SEQ ID NO: 134. In certain embodiments, the VL comprises the amino acid sequence set forth in SEQ ID NO: 163, and the VH comprises the amino acid sequence set forth in SEQ ID NO: 164.

In certain embodiments, the recombinant antibody comprises a light chain (LC) and a heavy chain (HC). In certain embodiments, the LC comprises the amino acid sequence set forth in SEQ ID NO: 27; the amino acid sequence set forth in SEQ ID NO: 37; the amino acid sequence set forth in SEQ ID NO: 47; the amino acid sequence set forth in SEQ ID NO: 57; the amino acid sequence set forth in SEQ ID NO: 67; the amino acid sequence set forth in SEQ ID NO: 77; the amino acid sequence set forth in SEQ ID NO: 87; the amino acid sequence set forth in SEQ ID NO: 97; the amino acid sequence set forth in SEQ ID NO: 107; the amino acid sequence set forth in SEQ ID NO: 117; the amino acid sequence set forth in SEQ ID NO: 127; the amino acid sequence set forth in SEQ ID NO: 137; the amino acid sequence set forth in SEQ ID NO: 147; the amino acid sequence set forth in SEQ ID NO: 157; the amino acid sequence set forth in SEQ ID NO: 167; or the amino acid sequence set forth in SEQ ID NO: 177. In certain embodiments, the HC comprises the amino acid sequence set forth in SEQ ID NO: 28 or SEQ ID NO: 29; the amino acid sequence set forth in SEQ ID NO: 38 or SEQ ID NO: 39; the amino acid sequence set forth in SEQ ID NO: 48 or SEQ ID NO: 49; the amino acid sequence set forth in SEQ ID NO: 58 or SEQ ID NO: 59; the amino acid sequence set forth in SEQ ID NO: 68 or SEQ ID NO: 69; the amino acid sequence set forth in SEQ ID NO: 78 or SEQ ID NO: 79; the amino acid sequence set forth in SEQ ID NO: 88 or SEQ ID NO: 89; the amino acid sequence set forth in SEQ ID NO: 98 or SEQ ID NO: 99; the amino acid sequence set forth in SEQ ID NO: 108 or SEQ ID NO: 109; the amino acid sequence set forth in SEQ ID NO: 118 or SEQ ID NO: 119; the amino acid sequence set forth in SEQ ID NO: 128 or SEQ ID NO: 129; the amino acid sequence set forth in SEQ ID NO: 138 or SEQ ID NO: 139; the amino acid sequence set forth in SEQ ID NO: 148 or SEQ ID NO: 149; the amino acid sequence set forth in SEQ ID NO: 158 or SEQ ID NO: 159; the amino acid sequence set forth in SEQ ID NO: 168 or SEQ ID NO: 169; or the amino acid sequence set forth in SEQ ID NO: 178 or SEQ ID NO: 179.

In certain embodiments, the LC comprises the amino acid sequence set forth in SEQ ID NO: 27, and the HC comprises the amino acid sequence set forth in SEQ ID NO: 28 or SEQ ID NO: 29; the LC comprises the amino acid sequence set forth in SEQ ID NO: 37, and the HC comprises the amino acid sequence set forth in SEQ ID NO: 38 or SEQ ID NO: 39; the LC comprises the amino acid sequence set forth in SEQ ID NO: 47, and the HC comprises the amino acid sequence set forth in SEQ ID NO: 48 or SEQ ID NO: 49; the LC comprises the amino acid sequence set forth in SEQ ID NO: 57, and the HC comprises the amino acid sequence set forth in SEQ ID NO: 58 or SEQ ID NO: 59; the LC comprises the amino acid sequence set forth in SEQ ID NO: 67, and the HC comprises the amino acid sequence set forth in SEQ ID NO: 68 or SEQ ID NO: 69; the LC comprises the amino acid sequence set forth in SEQ ID NO: 77, and the HC comprises the amino acid sequence set forth in SEQ ID NO: 78 or SEQ ID NO: 79; the LC comprises the amino acid sequence set forth in SEQ ID NO: 87, and the HC comprises the amino acid sequence set forth in SEQ ID NO: 88 or SEQ ID NO: 89; the LC comprises the amino acid sequence set forth in SEQ ID NO: 97, and the HC comprises the amino acid sequence set forth in SEQ ID NO: 98 or SEQ ID NO: 99; the LC comprises the amino acid sequence set forth in SEQ ID NO: 107, and the HC comprises the amino acid sequence set forth in SEQ ID NO: 108 or SEQ ID NO: 109; the LC comprises the amino acid sequence set forth in SEQ ID NO: 117, and the HC comprises the amino acid sequence set forth in SEQ ID NO: 118 or SEQ ID NO: 119; the LC comprises the amino acid sequence set forth in SEQ ID NO: 127, and the HC comprises the amino acid sequence set forth in SEQ ID NO: 128 or SEQ ID NO: 129; the LC comprises the amino acid sequence set forth in SEQ ID NO: 137, and the HC comprises the amino acid sequence set forth in SEQ ID NO: 138 or SEQ ID NO: 139; the LC comprises the amino acid sequence set forth in SEQ ID NO: 147, and the HC comprises the amino acid sequence set forth in SEQ ID NO: 148 or SEQ ID NO: 149; the LC comprises the amino acid sequence set forth in SEQ ID NO: 157, and the HC comprises the amino acid sequence set forth in SEQ ID NO: 158 or SEQ ID NO: 159; the LC comprises the amino acid sequence set forth in SEQ ID NO: 167, and the HC comprises the amino acid sequence set forth in SEQ ID NO: 168 or SEQ ID NO: 169; or the LC comprises the amino acid sequence set forth in SEQ ID NO: 177, and the HC comprises the amino acid sequence set forth in SEQ ID NO: 178 or SEQ ID NO: 179.

In certain embodiments, the LC comprises the amino acid sequence set forth in SEQ ID NO: 67, and the HC comprises the amino acid sequence set forth in SEQ ID NO: 69. In certain embodiments, the LC comprises the amino acid sequence set forth in SEQ ID NO: 137, and the HC comprises the amino acid sequence set forth in SEQ ID NO: 139. In certain embodiments, the LC comprises the amino acid sequence set forth in SEQ ID NO: 167, and the HC comprises the amino acid sequence set forth in SEQ ID NO: 169.

In certain embodiments, the recombinant antibody comprises a light chain variable region (VL) and a heavy chain variable region (VH), wherein the VL comprises the amino acid sequence set forth in SEQ ID NO: 13. In certain embodiments, the VL comprises at least one amino acid substitution. In certain embodiments, the at least one amino acid substitution is at position 1 and/or at position 44. In certain embodiments, the amino acid substitution at position 1 is E1Q. In certain embodiments, the amino acid substitution at position 44 is R44T. In certain embodiments, the VH comprises the amino acid sequence set forth in SEQ ID NO: 14. In certain embodiments, the VH comprises at least one amino acid substitution. In certain embodiments, the at least one amino acid substitution is at position 21, position 23, position 88, position 98, or a combination thereof. In certain embodiments, the amino acid substitution at position 1 is E1Q. In certain embodiments, the amino acid substitution at position 44 is R44T. In certain embodiments, the amino acid substitution at position 21 is P21S. In certain embodiments, the amino acid substitution at position 23 is T23A. In certain embodiments, the amino acid substitution at position 80 is I80T. In certain embodiments, the amino acid substitution at position 90 is T90A.

In certain embodiments, the recombinant antibody comprises a heavy chain (HC) comprising the amino acid sequence set forth in SEQ ID NO: 18. In certain embodiments, the HC comprises at least one amino acid substitution. In certain embodiments, the at least one amino acid substitution is at position 438 and/or or at position 444. In certain embodiments, the amino acid substitution at position 438 is M438L. In certain embodiments, the amino acid substitution at position 444 is N444S.

In certain non-limiting embodiments, the present disclosure also provides a recombinant anti-circumsporozoite (CSP) antibody comprising a light chain variable region (VL) and a heavy chain variable region (VH), wherein the VL comprises the amino acid sequence set forth in SEQ ID NO: 163 and the VH comprises the amino acid sequence set forth in SEQ ID NO: 164. In certain embodiments, the recombinant antibody comprises a light chain (LC) and a heavy chain (HC), wherein the LC comprises the amino acid sequence set forth in SEQ ID NO: 167 and the HC comprises the amino acid sequence set forth in SEQ ID NO: 169.

In certain non-limiting embodiments, the present disclosure provides a recombinant anti-circumsporozoite (CSP) antibody comprising a light chain variable region (VL) and a heavy chain variable region (VH), wherein the VL comprises the amino acid sequence set forth in SEQ ID NO: 63 and the VH comprises the amino acid sequence set forth in SEQ ID NO: 64. In certain embodiments, the recombinant antibody comprises a light chain (LC) and a heavy chain (HC), wherein the LC comprises the amino acid sequence set forth in SEQ ID NO: 67 and the HC comprises the amino acid sequence set forth in SEQ ID NO: 69.

In certain non-limiting embodiments, the present disclosure further provides a recombinant anti-circumsporozoite (CSP) antibody comprising a light chain variable region (VL) and a heavy chain variable region (VH), wherein the VL comprises the amino acid sequence set forth in SEQ ID NO: 133 and the VH comprises the amino acid sequence set forth in SEQ ID NO: 134. In certain embodiments, the recombinant antibody comprises a light chain (LC) and a heavy chain (HC), wherein the LC comprises the amino acid sequence set forth in SEQ ID NO: 137 and the HC comprises the amino acid sequence set forth in SEQ ID NO: 139.

In certain non-limiting embodiments, the present disclosure provides a recombinant anti-circumsporozoite (CSP) antibody comprising a light chain variable region (VL) and a heavy chain variable region (VH), wherein the light chain variable region comprises a CDR1 comprising the amino acid sequence set forth in SEQ ID NO: 183, a CDR2 comprising the amino acid sequence set forth in SEQ ID NO: 184, and a CDR3 comprising the amino acid sequence set forth in SEQ ID NO: 185; and the heavy chain variable region comprises a CDR1 comprising the amino acid sequence set forth in SEQ ID NO: 186, a CDR2 comprising the amino acid sequence set forth in SEQ ID NO: 187, and a CDR3 comprising the amino acid sequence set forth in SEQ ID NO: 188. In certain embodiments, the VL comprises the amino acid sequence set forth in SEQ ID NO: 195, and the VH comprises the amino acid sequence set forth in SEQ ID NO: 196. In certain embodiments, the antibody comprises comprising a LC and a HC. In certain embodiments, the LC comprises the amino acid sequence set forth in SEQ ID NO: 199, and the HC comprises the amino acid sequence set forth in SEQ ID NO: 200 or SEQ ID NO: 201.

In certain non-limiting embodiments, the present disclosure provides a recombinant anti-circumsporozoite (CSP) antibody comprising a light chain variable region (VL) and a heavy chain variable region (VH), wherein the light chain variable region comprises a CDR1 comprising the amino acid sequence set forth in SEQ ID NO: 205, a CDR2 comprising the amino acid sequence set forth in SEQ ID NO: 206, and a CDR3 comprising the amino acid sequence set forth in SEQ ID NO: 207; and the heavy chain variable region comprises a CDR1 comprising the amino acid sequence set forth in SEQ ID NO: 208, a CDR2 comprising the amino acid sequence set forth in SEQ ID NO: 209, and a CDR3 comprising the amino acid sequence set forth in SEQ ID NO: 210.

In certain embodiments, the amino acid sequence set forth in SEQ ID NO: 227; the amino acid sequence set forth in SEQ ID NO: 237; the amino acid sequence set forth in SEQ ID NO: 247; the amino acid sequence set forth in SEQ ID NO: 257; or the amino acid sequence set forth in SEQ ID NO: 267. In certain embodiments, the VH comprises the amino acid sequence set forth in SEQ ID NO: 228; the amino acid sequence set forth in SEQ ID NO: 238; the amino acid sequence set forth in SEQ ID NO: 248; the amino acid sequence set forth in SEQ ID NO: 258; or the amino acid sequence set forth in SEQ ID NO: 268.

In certain embodiments, the VL comprises the amino acid sequence set forth in SEQ ID NO: 227, and the VH comprises the amino acid sequence set forth in SEQ ID NO: 228; the VL comprises the amino acid sequence set forth in SEQ ID NO: 237, and the VH comprises the amino acid sequence set forth in SEQ ID NO: 238; the VL comprises the amino acid sequence set forth in SEQ ID NO: 247, and the VH comprises the amino acid sequence set forth in SEQ ID NO: 248; the VL comprises the amino acid sequence set forth in SEQ ID NO: 257, and the VH comprises the amino acid sequence set forth in SEQ ID NO: 258; or the VL comprises the amino acid sequence set forth in SEQ ID NO: 267, and the VH comprises the amino acid sequence set forth in SEQ ID NO: 268.

In certain embodiments, the recombinant antibody comprises a LC and a HC. In certain embodiments, the LC comprises the amino acid sequence set forth in SEQ ID NO: 231; the amino acid sequence set forth in SEQ ID NO: 241; the amino acid sequence set forth in SEQ ID NO: 251; the amino acid sequence set forth in SEQ ID NO: 261; or the amino acid sequence set forth in SEQ ID NO: 271. In certain embodiments, the HC comprises the amino acid sequence set forth in SEQ ID NO: 232 or SEQ ID NO: 233; the amino acid sequence set forth in SEQ ID NO: 242 or SEQ ID NO: 243; the amino acid sequence set forth in SEQ ID NO: 252 or SEQ ID NO: 253; the amino acid sequence set forth in SEQ ID NO: 262 or SEQ ID NO: 263; or the amino acid sequence set forth in SEQ ID NO: 272 or SEQ ID NO: 273.

In certain embodiments, the LC comprises the amino acid sequence set forth in SEQ ID NO: 231, and the HC comprises the amino acid sequence set forth in SEQ ID NO: 232 or SEQ ID NO: 233; the LC comprises the amino acid sequence set forth in SEQ ID NO: 241, and the HC comprises the amino acid sequence set forth in SEQ ID NO: 242 or SEQ ID NO: 243; the LC comprises the amino acid sequence set forth in SEQ ID NO: 251, and the HC comprises the amino acid sequence set forth in SEQ ID NO: 252 or SEQ ID NO: 253; the LC comprises the amino acid sequence set forth in SEQ ID NO: 261, and the HC comprises the amino acid sequence set forth in SEQ ID NO: 262 or SEQ ID NO: 263; or the LC comprises the amino acid sequence set forth in SEQ ID NO: 271, and the HC comprises the amino acid sequence set forth in SEQ ID NO: 272 or SEQ ID NO: 273.

In certain embodiments, the VH comprises the amino acid sequence set forth in SEQ ID NO: 218. In certain embodiments, the VH comprises at least one amino acid substitution. In certain embodiments, the at least one amino acid substitution is at position 40, position 69, position 80, position 85, position 120, or a combination thereof. In certain embodiments, the amino acid substitution at position 40 is T40A. In certain embodiments, the amino acid substitution at position 69 is I69T. In certain embodiments, the amino acid substitution at position 80 is S80Y. In certain embodiments, the amino acid substitution at position 85 is G85S. In certain embodiments, the amino acid substitution at position 120 is I120T. In certain embodiments, the HC comprises the amino acid sequence set forth in SEQ ID NO: 222. In certain embodiments, the HC comprises at least one amino acid substitution. In certain embodiments, the at least one amino acid substitution is at position 434 and/or at position 440. In certain embodiments, the amino acid substitution at position 434 is M434L. In certain embodiments, the amino acid substitution at position 440 is N440S.

In certain embodiments, the recombinant antibody exhibits at least 20% reduction in parasite liver load as compared to a reference antibody. In certain embodiments, the recombinant antibody exhibits at least 20% increase in survival rate as compared to a reference antibody. In certain embodiments, the recombinant antibody exhibits increased conformational stability as compared to a reference antibody. In certain embodiments, the recombinant antibody exhibits increased colloidal stability as compared to a reference antibody. In certain embodiments, the reference antibody is AB-000317. In certain embodiments, the reference antibody is AB-000224. In certain embodiments, the reference antibody is AB-007088.

In certain embodiments, the recombinant antibody binds to a NANP repeat region. In certain embodiments, the recombinant antibody binds to a polypeptide comprising or consisting of the amino acid sequence set forth in SEQ ID NO: 280.

In certain embodiments, the recombinant antibody comprises at least one modification relative to the native AB-000224 variable heavy chain amino acid sequence set forth in SEQ ID NO: 14. In certain embodiments, the recombinant antibody comprises at least one modification relative to the native AB-000224 variable light chain amino acid sequence set forth in SEQ ID NO: 13. In certain embodiments, the recombinant antibody comprises at least one modification relative to the native AB-000224 variable heavy chain amino acid sequence set forth in SEQ ID NO: 14 and at least one modification relative to the native AB-000224 variable light chain amino acid sequence set forth in SEQ ID NO: 13.

In certain embodiments, the recombinant antibody comprises at least one modification relative to the native AB-007088 variable heavy chain amino acid sequence set forth in SEQ ID NO: 218. In certain embodiments, the recombinant antibody comprises at least one modification relative to the native AB-007088 variable light chain amino acid sequence set forth in SEQ ID NO: 217. In certain embodiments, the recombinant antibody comprises at least one modification relative to the native AB-007088 variable heavy chain amino acid sequence set forth in SEQ ID NO: 218 and at least one modification relative to the native AB-000224 variable light chain amino acid sequence set forth in SEQ ID NO: 217.

In certain non-limiting embodiments, the present disclosure provides a polynucleotide encoding an antibody disclosed herein. In certain non-limiting embodiments, the present disclosure provides an expression vector comprising the polynucleotide disclosed herein. In certain non-limiting embodiments, the present disclosure provides a host cell comprising the expression vector or the polynucleotide disclosed herein.

In certain non-limiting embodiments, the present disclosure provides a composition comprising the antibody disclosed herein. In certain embodiments, the composition further comprises a pharmaceutically acceptable carrier.

In certain non-limiting embodiments, the present disclosure provides a method of preventing and/or treating malaria in a subject in need thereof, comprising administering an effective amount of the antibody disclosed herein or of the composition disclosed herein. In certain embodiments, the subject is a pediatric patient.

In certain non-limiting embodiments, the present disclosure provides the antibodies or compositions disclosed herein for use in the prevention and/or treatment of malaria in a subject in need thereof. Additionally, in certain non-limiting embodiments, the present disclosure provides the antibodies or compositions disclosed herein for the manufacture of a medicament for the prevention and/or treatment of malaria in a subject in need thereof. Furthermore, the present disclosure provides use of the antibodies or compositions disclosed herein for the prevention and/or treatment and/or prevention of malaria in a subject in need thereof. In certain embodiments, the subject is a pediatric patient.

### BRIEF DESCRIPTION OF THE DRAWINGS

FIG. 1A illustrates parasite liver load following administration of experimental anti-CSP antibodies, AB-000224 and AB-007088. Parasite liver burden load was measured by bioluminescence (photons/sec) generated from the fluorescent sporozoites (y-axis). The individual points indicate the total amount of bioluminescence measured in a single mouse and by extension, the sporozoite liver burden. AB-001245 is a non-malaria-specific antibody that was used as a negative control. AB-000317 is a positive control. FIG. 1B illustrates the associated human IgG serum concentrations determined by ELISA obtained 15 hours following antibody administration. The individual points indicate the total amount of human IgG in a single mouse. AB-001245 is a non-malaria-specific antibody that was used as a negative control. AB-000317 is a positive control.

FIGS. 2A and 2C illustrate the survival rate of mice following administration of anti-CSP antibody AB-000224 and exposure to mosquitoes infected with chimeric P. berghei expressing P. falciparum CSP protein in two experiments. FIG. 2B shows the associated human IgG serum concentrations determined by ELISA obtained 15 hours following antibody administration in the experiment shown in FIG. 2A. AB-001245 is a non-malaria-specific antibody that was used as a negative control. AB-000317 is a positive control.

FIGS. 3A and 3C illustrate the survival rate of mice following administration of anti-CSP antibody AB-007088 and exposure to mosquitoes infected with chimeric P. berghei expressing P. falciparum CSP protein in two experiments. FIG. 3B shows the associated human IgG serum concentrations determined by ELISA obtained 15 hours following antibody administration in the experiment shown in FIG. 3A. AB-001245 is a non-malaria-specific antibody that was used as a negative control. AB-000317 is a positive control.

FIGS. 4A-4B illustrate sequences for the AB-000224 antibody. FIG. 4A shows the AB-000224 Lambda Light Chain sequence (SEQ ID NO: 286). FIG. 4B shows the AB-000224 IgG1 Heavy Chain sequence (SEQ ID NO: 287). Framework and CDR regions are designated using the ASN system.

FIGS. 5A-5B illustrate sequences for the AB-007088 antibody. FIG. 5A shows the AB-007088 Lambda Light Chain sequence (SEQ ID NO: 288). FIG. 5B shows the AB-007088 IgG1 Heavy Chain sequence (SEQ ID NO: 289). Framework and CDR regions are designated using the ASN system.

FIG. 6 shows the ASN numbering system for the light chain of AB-000224.

FIG. 7 shows the ASN numbering system for the heavy chain of AB-000224.

FIG. 8 shows the ASN numbering system for the light chain of AB-007088.

FIG. 9 shows the ASN numbering system for the heavy chain of AB-007088.

FIGS. 10A-10F illustrate parasite liver load following administration of experimental anti-CSP antibodies variants disclosed herein. Results are expressed as percentage inhibition, where naïve infected were considered as 100%. FIGS. 10G-10I show concentration of human antibodies that were circulating in the mice at the time of infection as determined by ELISA for the assays shown in FIGS. 10D-F, respectively. AB-001245 is a non-malaria-specific antibody that was used as a negative control. AB-000317 is a positive control.

FIGS. 11A-11C illustrate the survival rate of mice following administration of anti-CSP antibody variants disclosed herein and exposure to mosquitoes infected with chimeric P. berghei expressing P. falciparum CSP protein. FIGS. 11D-F show concentration of human antibodies that were circulating in the mice at the time of infection as determined by ELISA for the assays shown in FIGS. 11A-C, respectively. AB-001245 is a non-malaria-specific antibody that was used as a negative control. AB-000317 is a positive control.

FIG. 12A illustrates the biophysical characterization of AB-000224 and variants thereof. FIG. 12B shows the ranking of the AB-000224 and variants thereof based on all data obtained during biophysical characterization. FIG. 12C shows the ranking of the AB-000224 and variants thereof excluding polyreactivity and including titer.

FIG. 13A illustrates the biophysical characterization of AB-007088 and variants thereof. FIG. 13B shows the ranking of the AB-007088 and variants thereof based on all data obtained during biophysical characterization. FIG. 13C shows the ranking of the AB-007088 and variants thereof excluding polyreactivity and including titer.

### DETAILED DESCRIPTION

The present disclosure is based, at least in part, on the finding of several anti-CSP antibody variants. The present disclosure surprisingly shows that the disclosed anti-CSP antibodies and variants thereof have superior properties as compared to previously disclosed antibodies.

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

As used in this specification and the appended claims, the singular forms “a,” “an” and “the” include plural referents unless the context dictates otherwise. Thus, for example, reference to “a protein” or an “antibody” includes a plurality of proteins or antibodies, respectively; reference to “a cell” includes mixtures of cells and the like.

As used herein, the term “about” or “approximately” refers to the usual error range for the respective value readily known to the skilled person in this technical field, for example, ±20%, ±10%, or ±5%, are within the intended meaning of the recited value.

As used herein, the term “antibody” means an isolated or recombinant binding agent that comprises the necessary variable region sequences to specifically bind an antigenic epitope. Therefore, an “antibody” as used herein is any form of antibody or fragment thereof that exhibits the desired biological activity, e.g., binding the specific target antigen. Thus, it is used in the broadest sense and specifically covers a monoclonal antibody (including full-length monoclonal antibodies), human antibodies, chimeric antibodies, nanobodies, diabodies, multispecific antibodies (e.g., bispecific antibodies), and antibody fragments including but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab′)2, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

As used herein, “recombinant antibody” refers to an antibody wherein the exact amino acid sequence of the antibody is not naturally found in a given organism (e.g., an antibody from a mammal). In certain embodiments, this term can refer to an antibody including one or more amino acid residues that are not found in a naturally occurring antibody. In certain embodiments, a recombinant antibody can have a CDR including an amino acid residue that is not found in a naturally occurring antibody (e.g., an antibody from a mammal). In another exemplary embodiment, a recombinant antibody can have a framework (FR) including an amino acid residue that is not found in a naturally occurring antibody (e.g., an antibody from a mammal). In certain embodiments, a recombinant antibody can have a constant region including an amino acid residue that is not found in a naturally occurring antibody (e.g., an antibody from a mammal). In certain embodiments, a recombinant antibody is variant of a naturally occurring antibody (e.g., AB-000224) including at least one modification, e.g., substitution, relative to the native variable heavy chain amino acid sequence or variable light chain amino acid sequence. For example, but without any limitation, a recombinant antibody can be an anti-CSP antibody AB-000224 disclosed herein comprising at least one modification, e.g., substitution, relative to the native AB-000224 variable heavy chain amino acid sequence (SEQ ID NO: 14) or variable light chain amino acid sequence (SEQ ID NO: 13) described herein. A recombinant antibody has improved developability, e.g., decreased heterogeneity, increased yield, increased stability, improved net charges to improve pharmacokinetics, and/or reduced immunogenicity.

As used herein, the terms, “anti-CSP antibody” and “CSP antibody” are used synonymously and refer to an antibody that binds to Plasmodium falciparum circumsporozoite (CSP) antigen.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

As used herein, “V-region” refers to an antibody variable region domain comprising the segments of Framework 1, CDR1, Framework 2, CDR2, Framework 3, CDR3, and Framework 4. The heavy chain V-region, VH, is a consequence of rearrangement of a V-gene (HV), a D-gene (HD), and a J-gene (HJ), in what is known as V (D) J recombination during B-cell differentiation. The light chain V-region, VL, is a consequence of the rearrangement of a V-gene (LV) and a J-gene. In certain embodiments, the terms “VH” and “heavy chain variable” refer to the heavy chain V-region of an antibody. In certain embodiments, the terms “VL” and “light chain variable” refer to the light chain V-region of an antibody.

As used herein, “complementarity-determining region (CDR)” refers to the three hypervariable regions (HVRs) in each chain that interrupt the four “framework” regions established by the light and heavy chain variable regions. The CDRs are the primary contributors to binding to an epitope of an antigen. The CDRs of each chain are referred to as CDR1, CDR2, and CDR3 numbered sequentially starting from the N-terminus, and are also identified by the chain in which the particular CDR is located. Thus, a VH CDR3 (HCDR3) is located in the variable domain of the heavy chain of the antibody in which it is found, whereas a VL CDR3 (LCDR3) is the CDR3 from the variable domain of the light chain of the antibody in which it is found. The term “CDR” is used interchangeably with “HVR” when referring to CDR sequences.

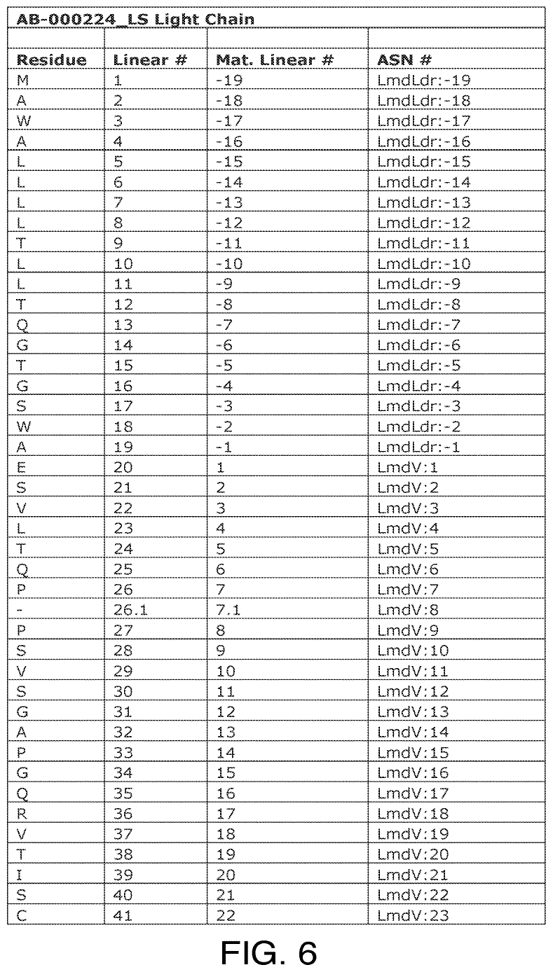

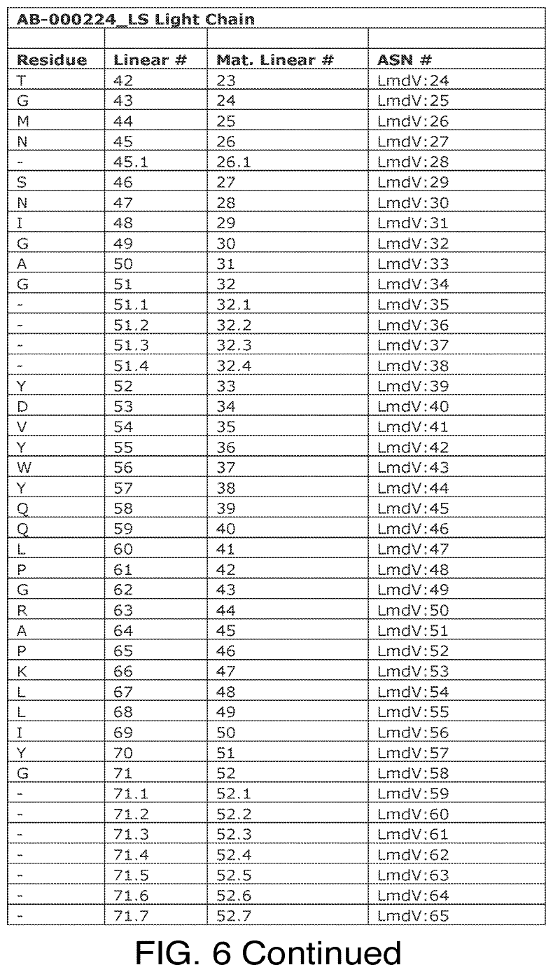

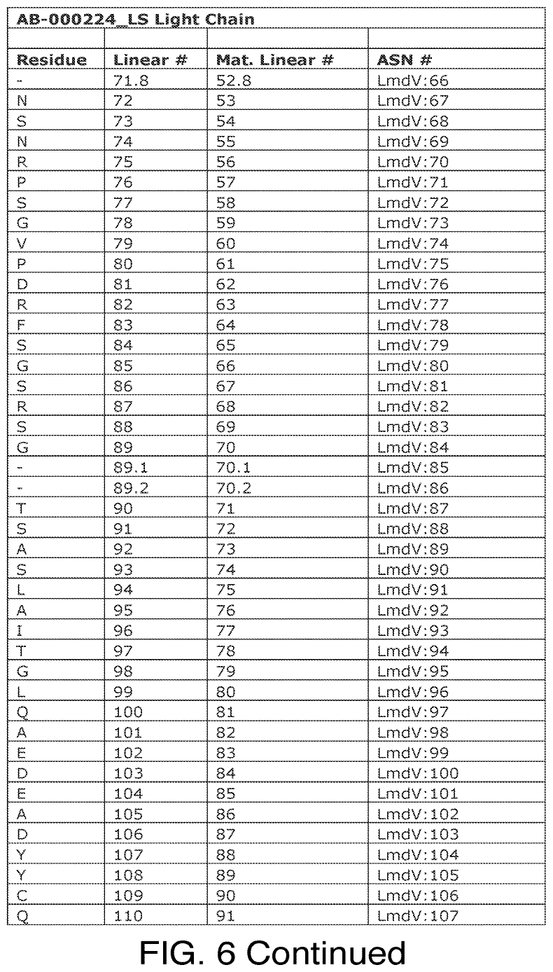

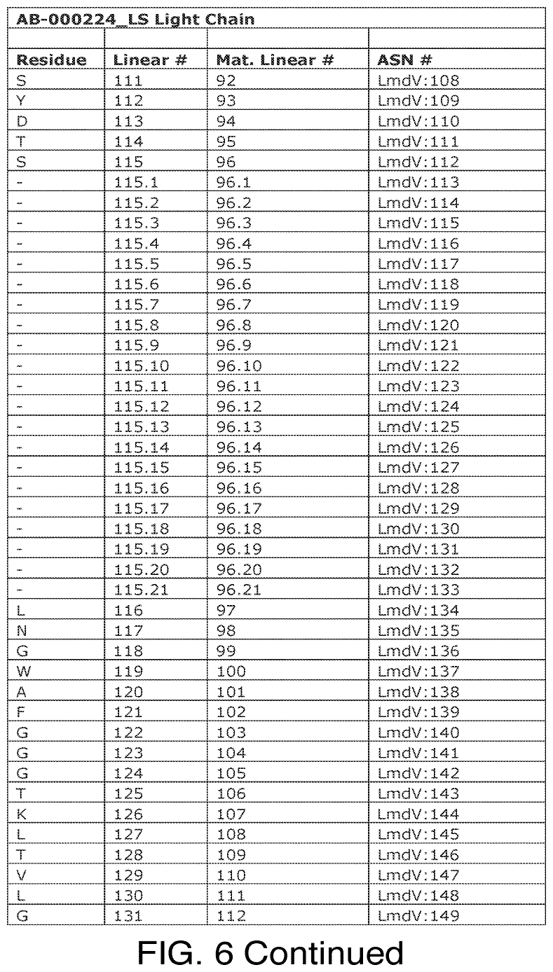

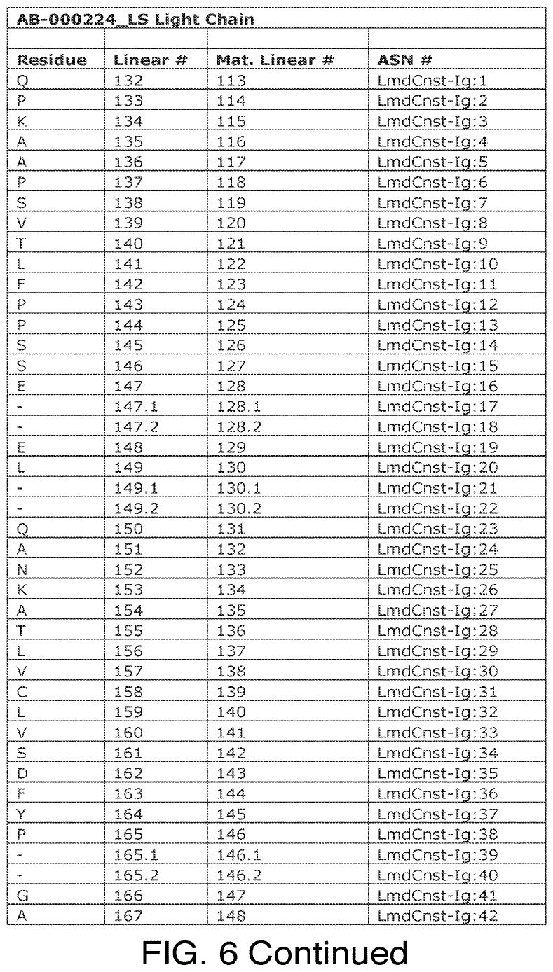

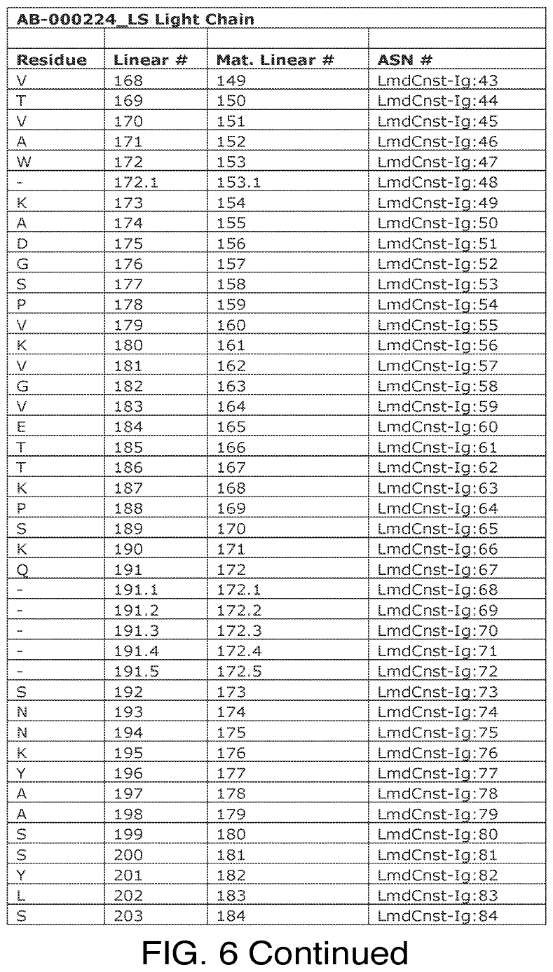

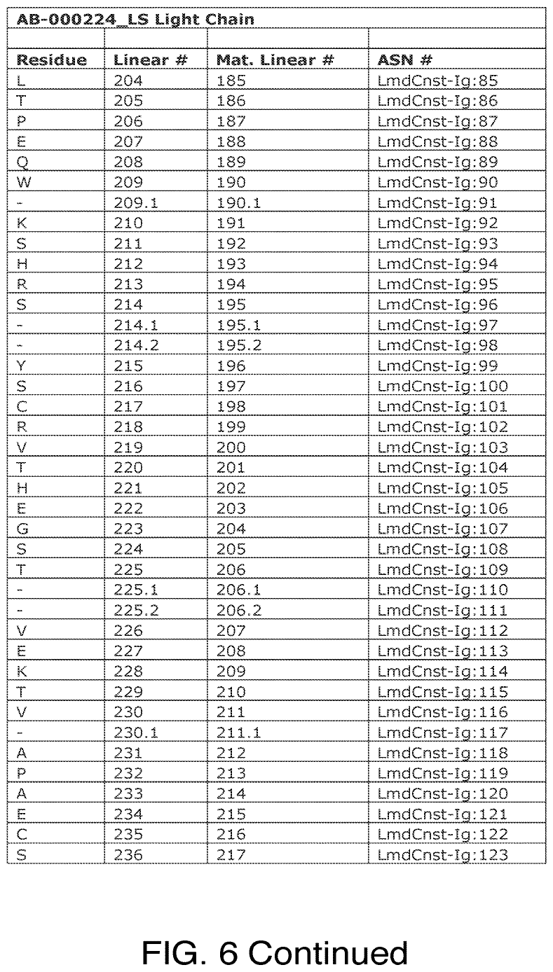

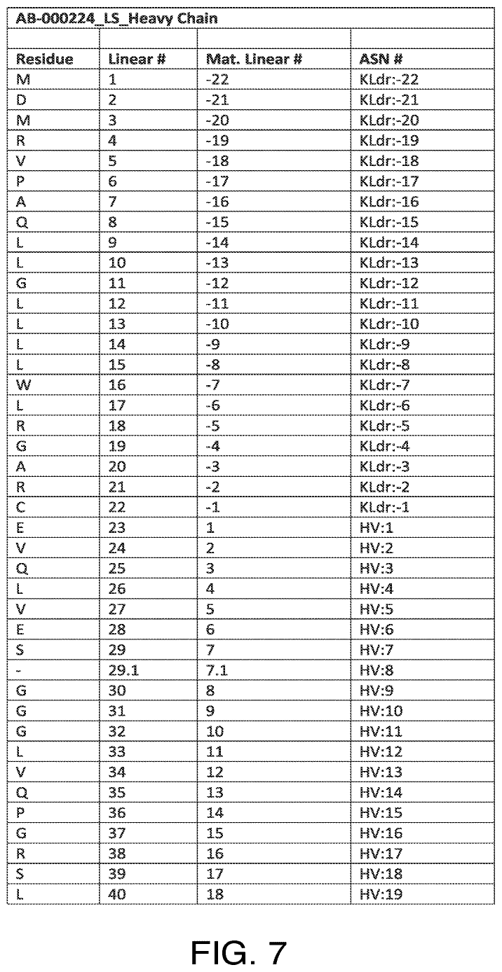

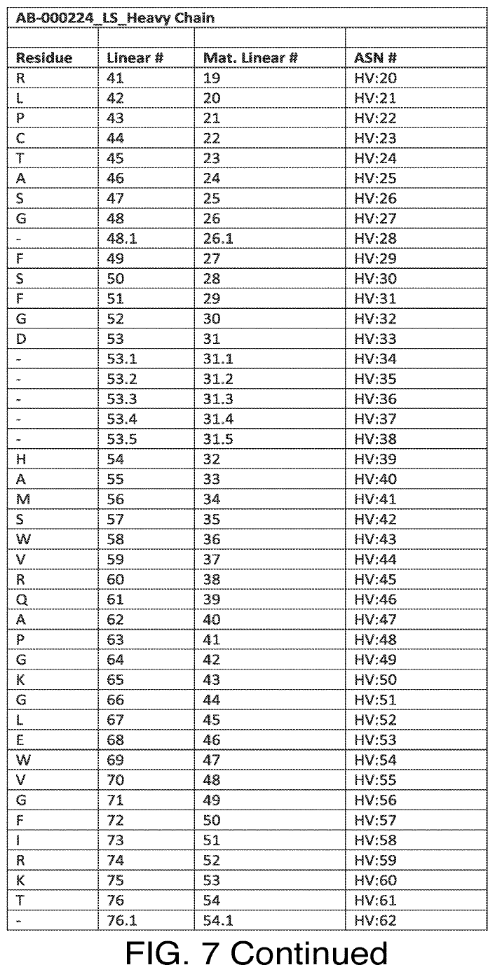

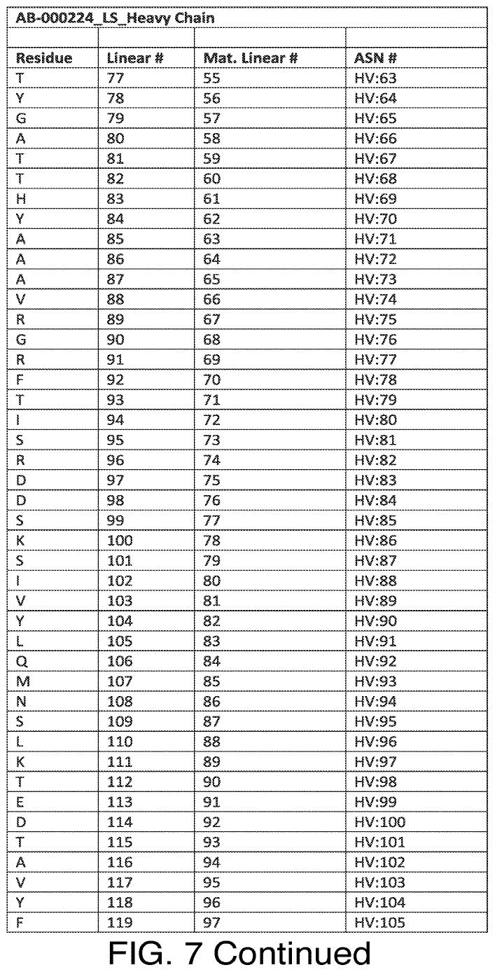

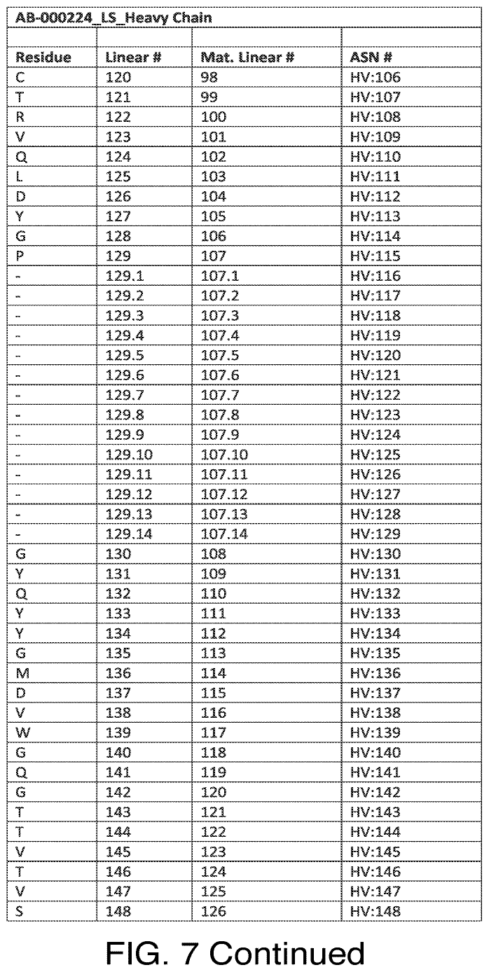

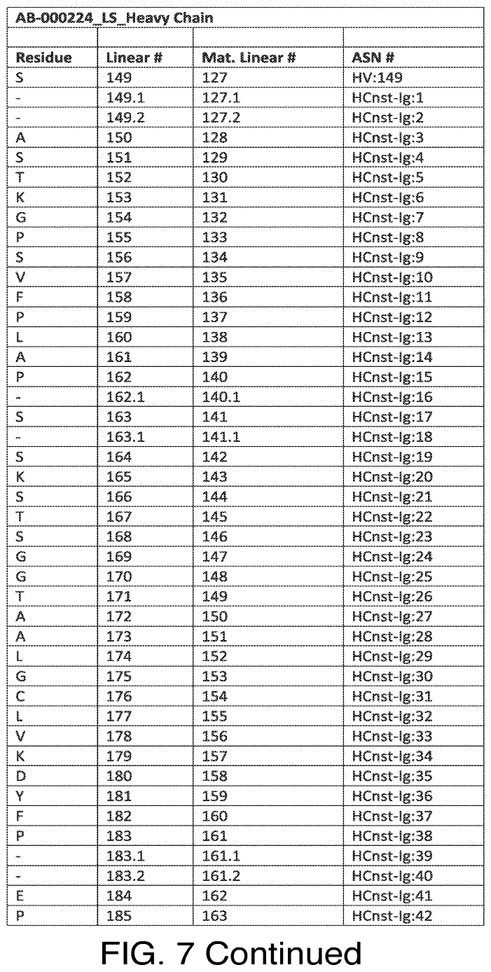

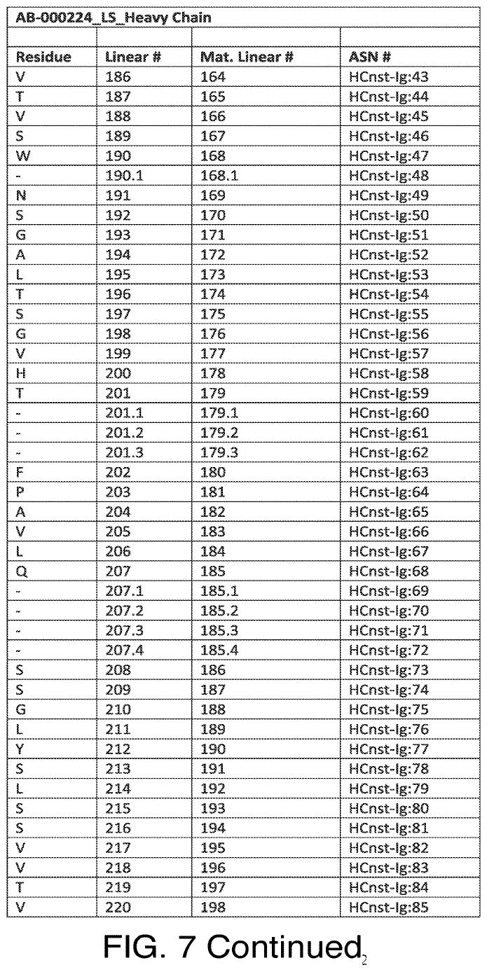

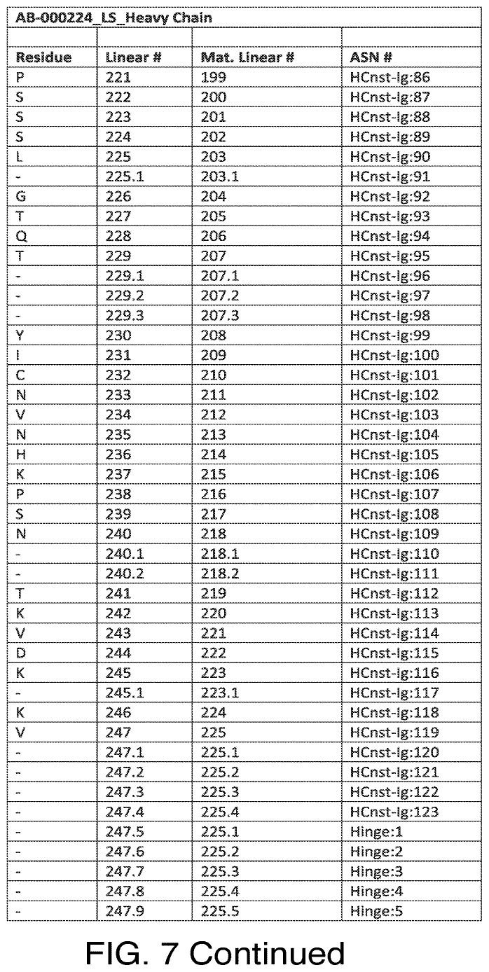

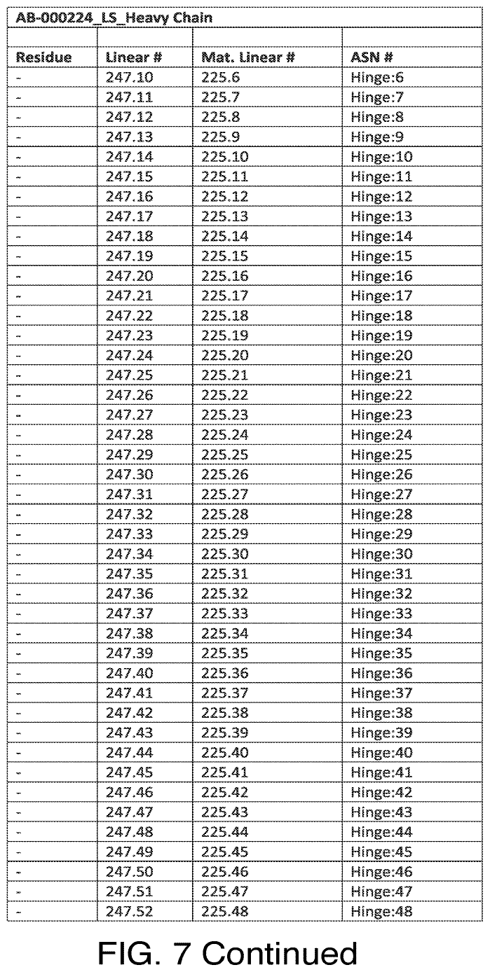

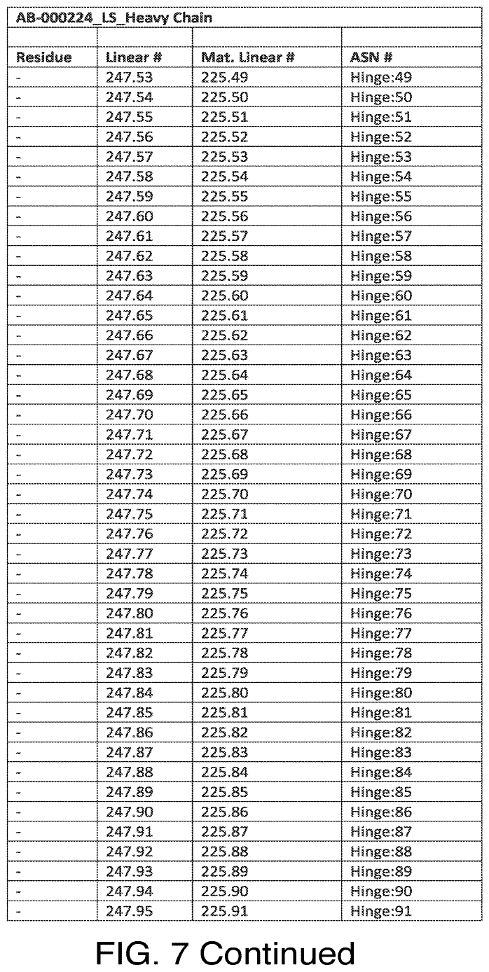

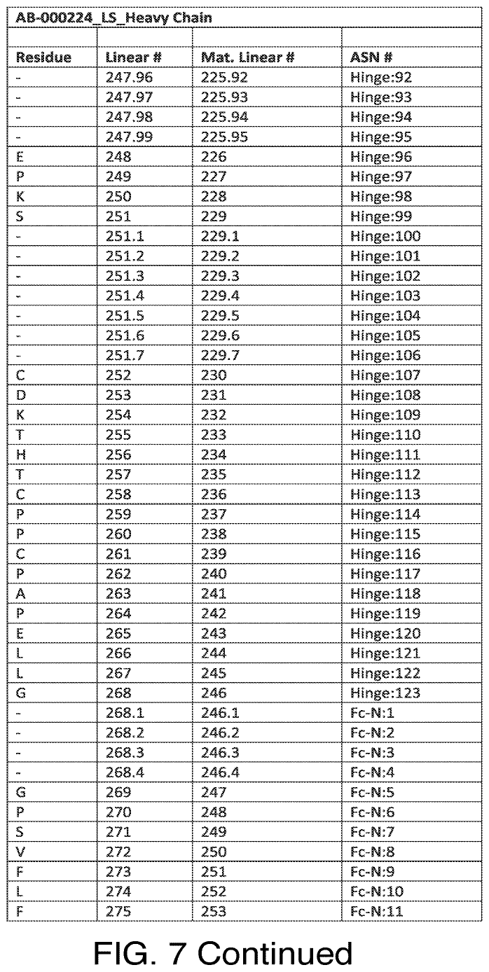

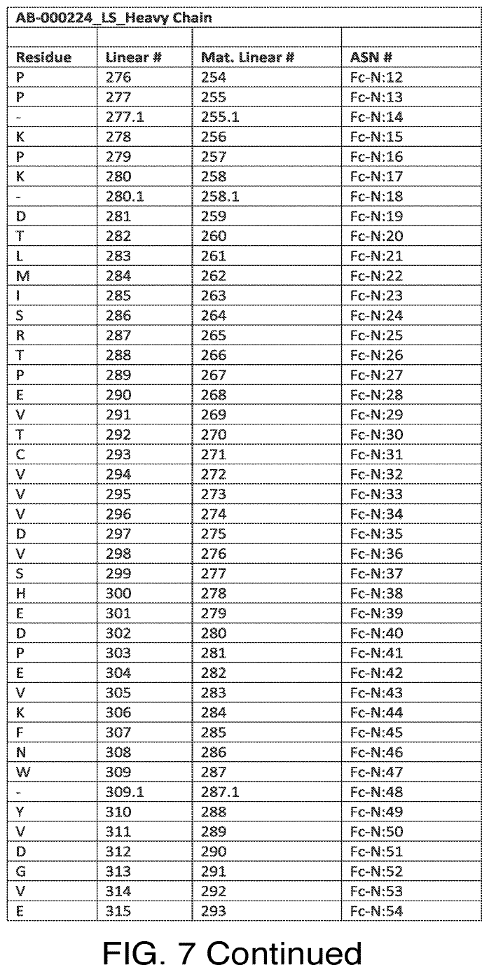

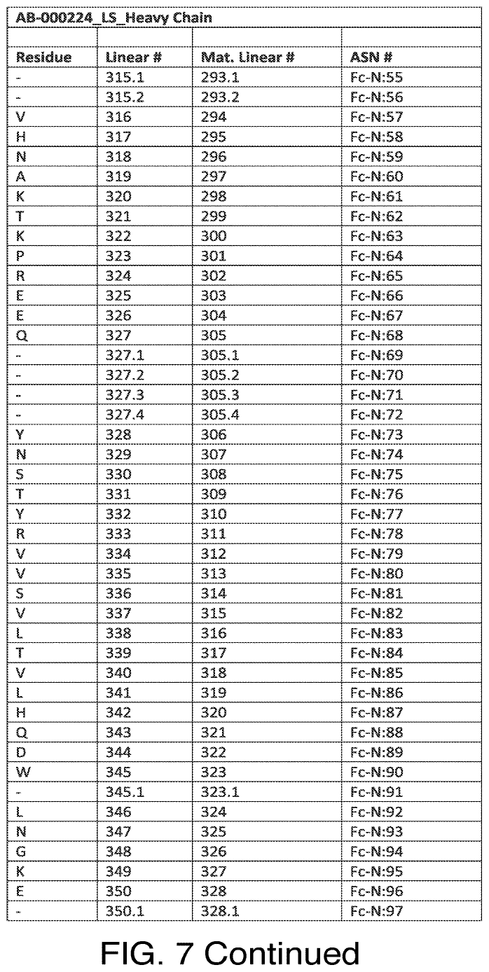

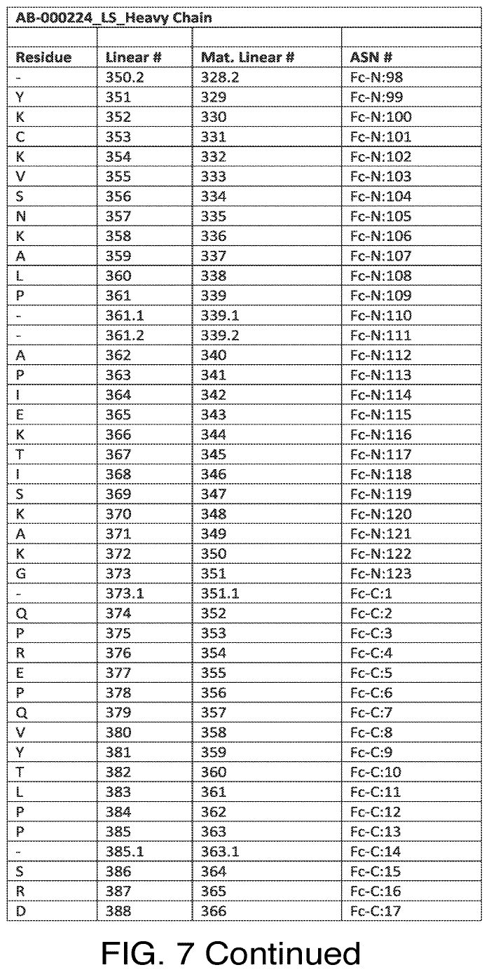

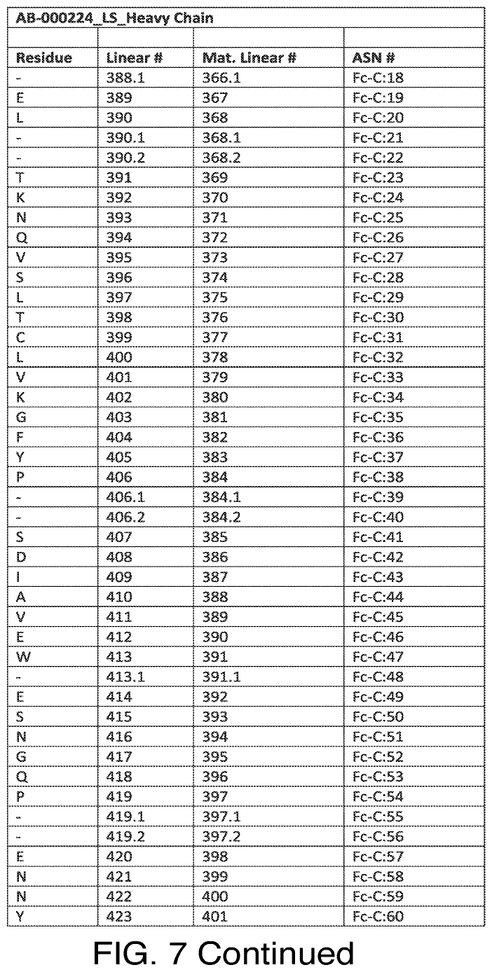

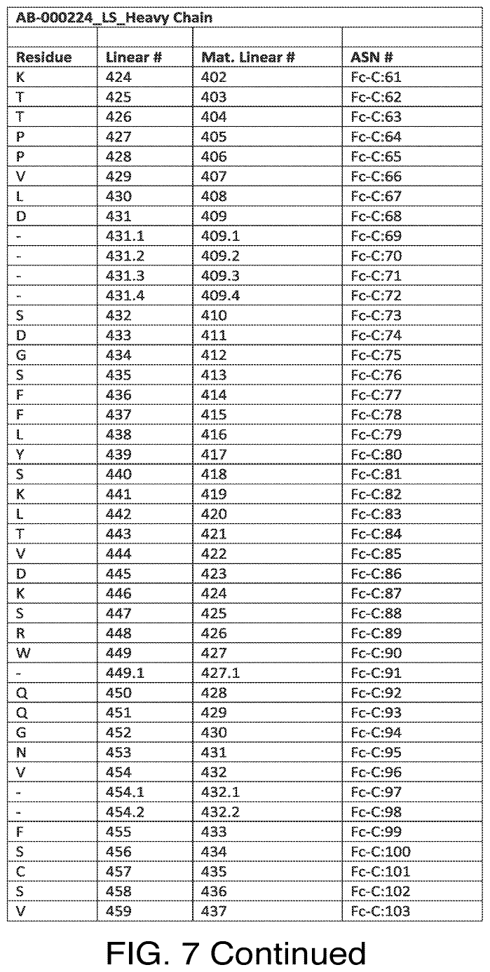

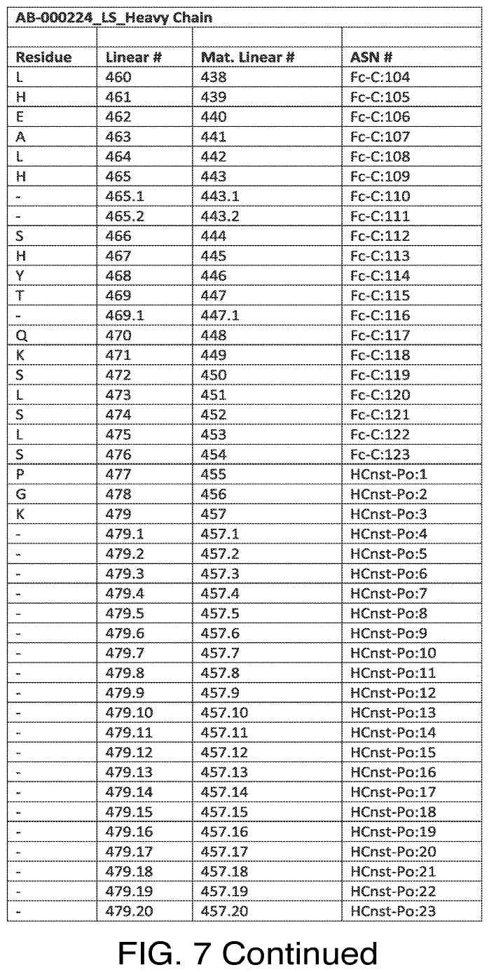

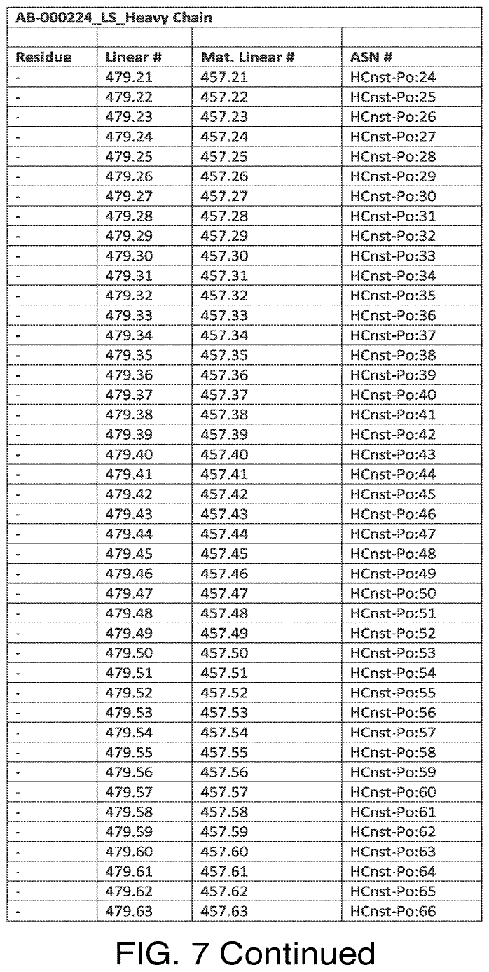

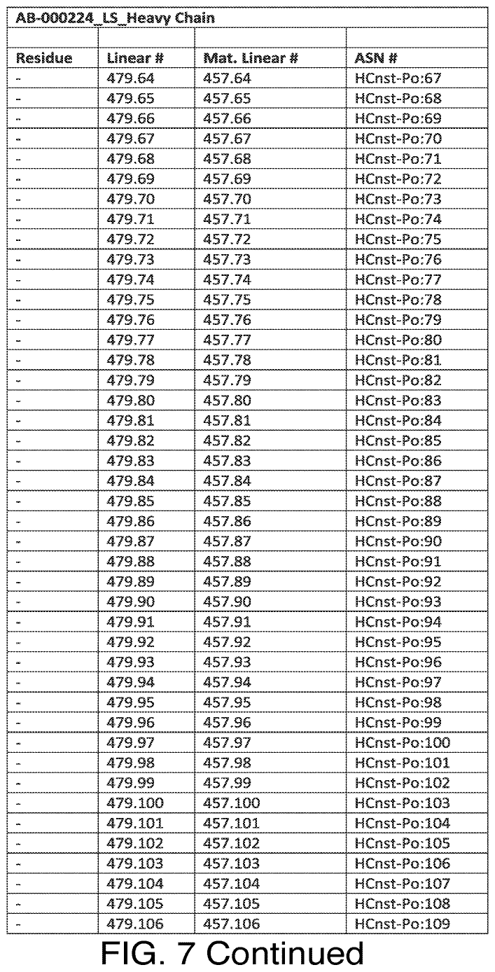

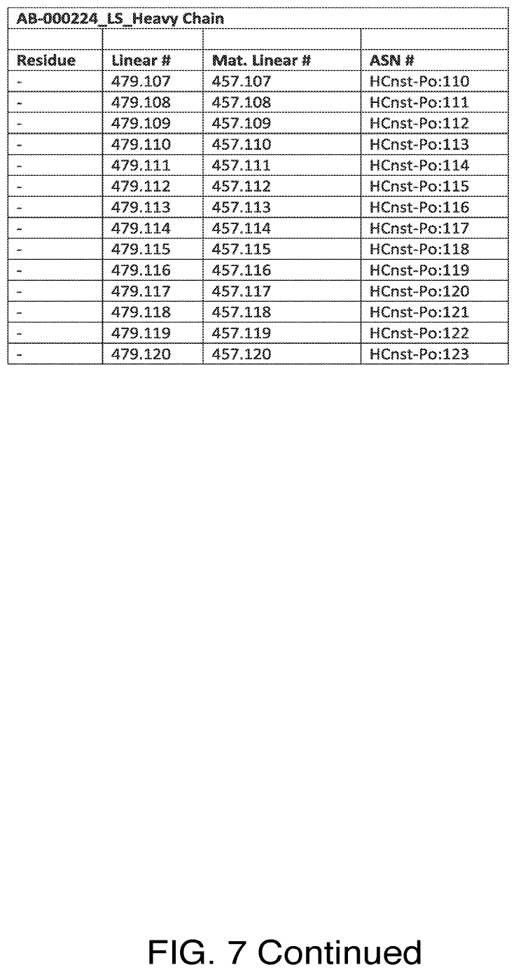

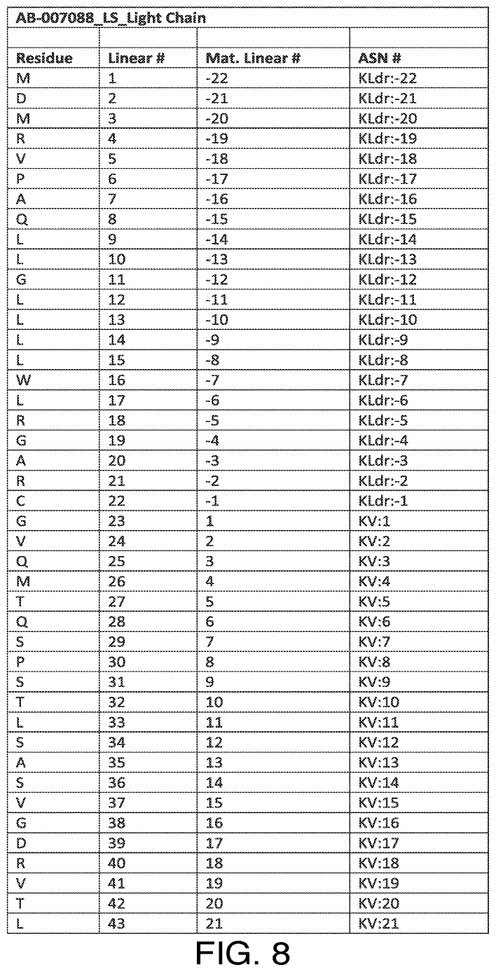

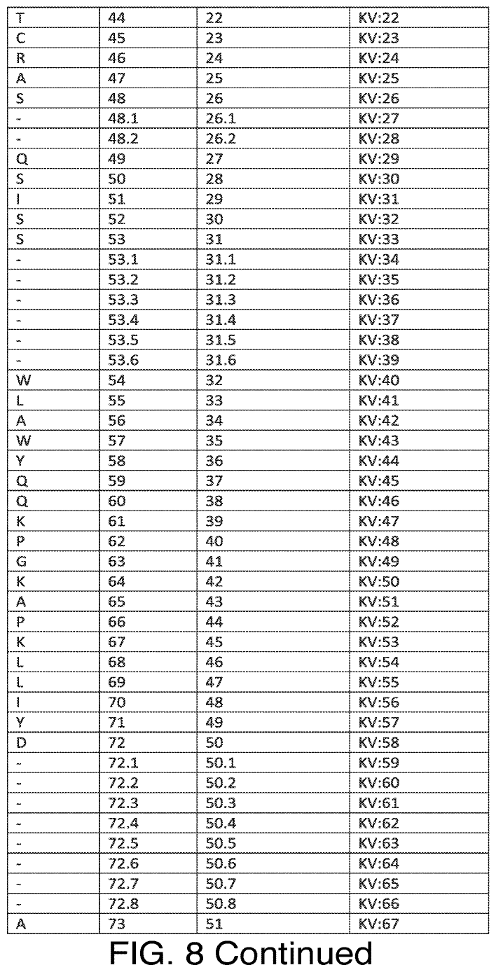

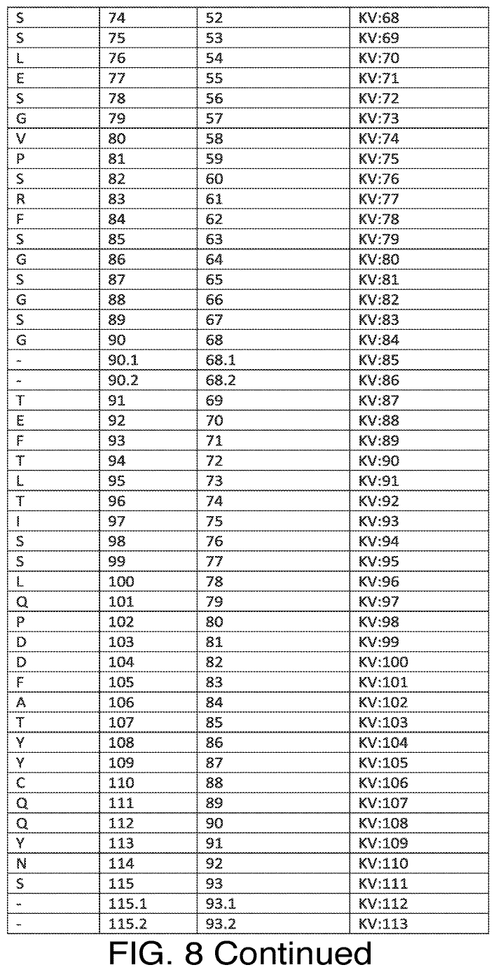

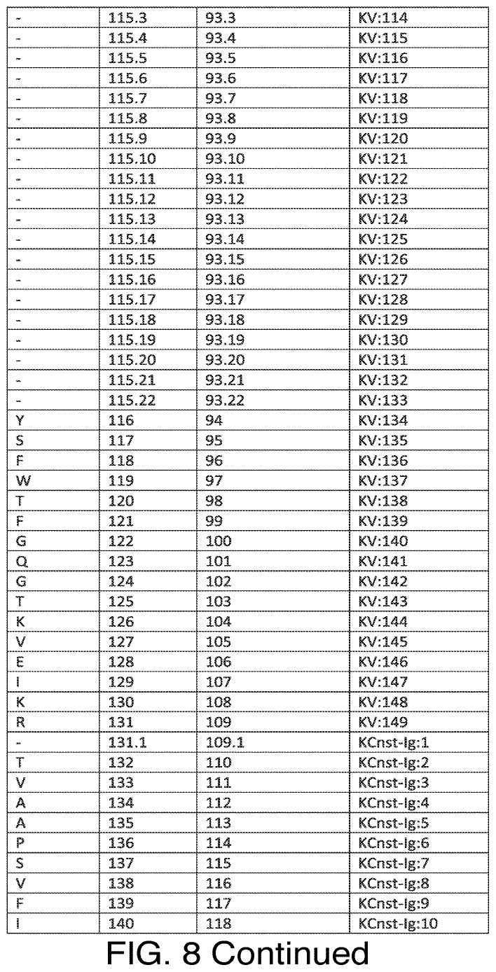

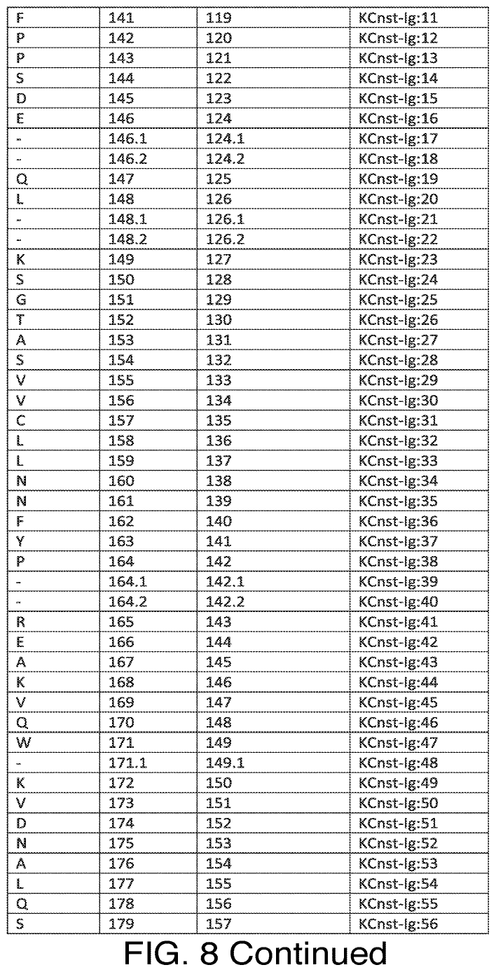

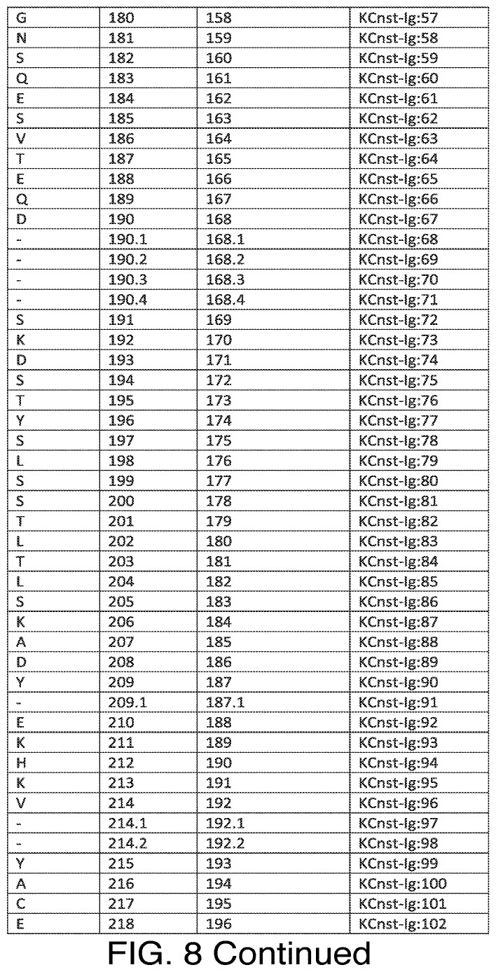

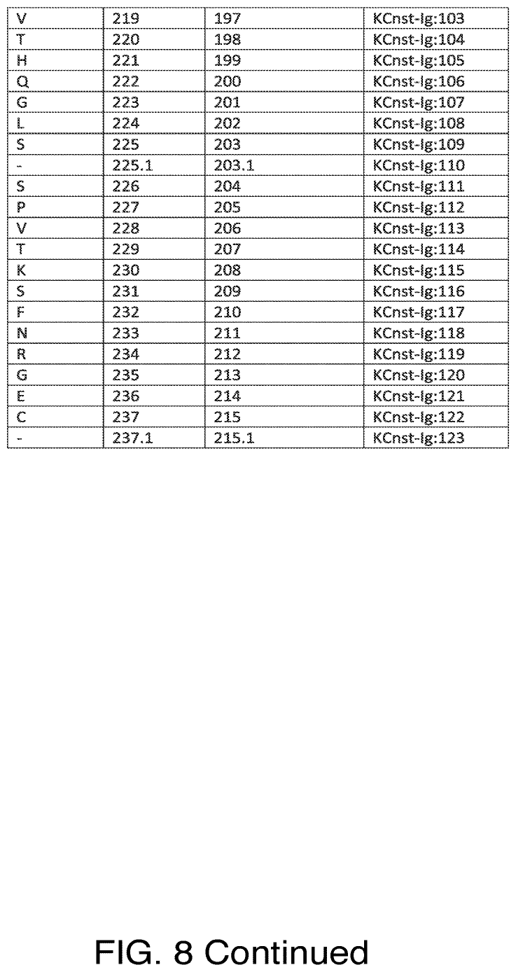

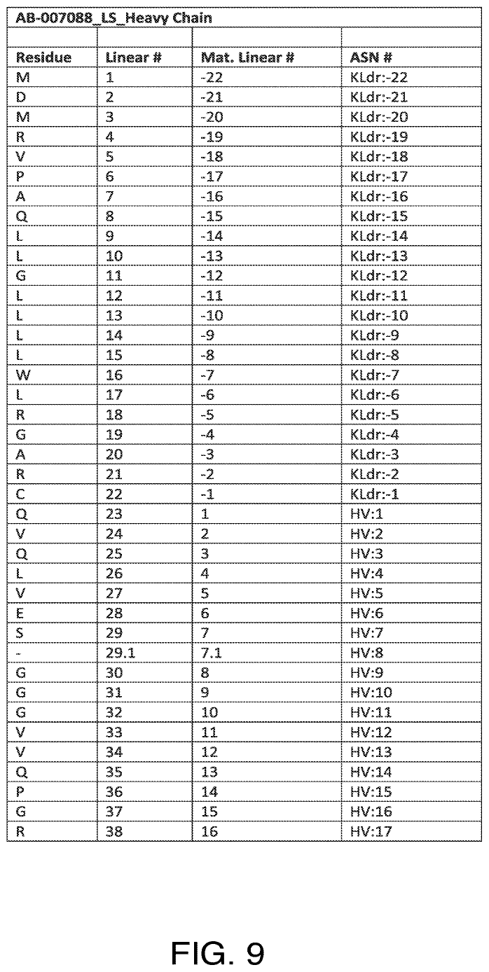

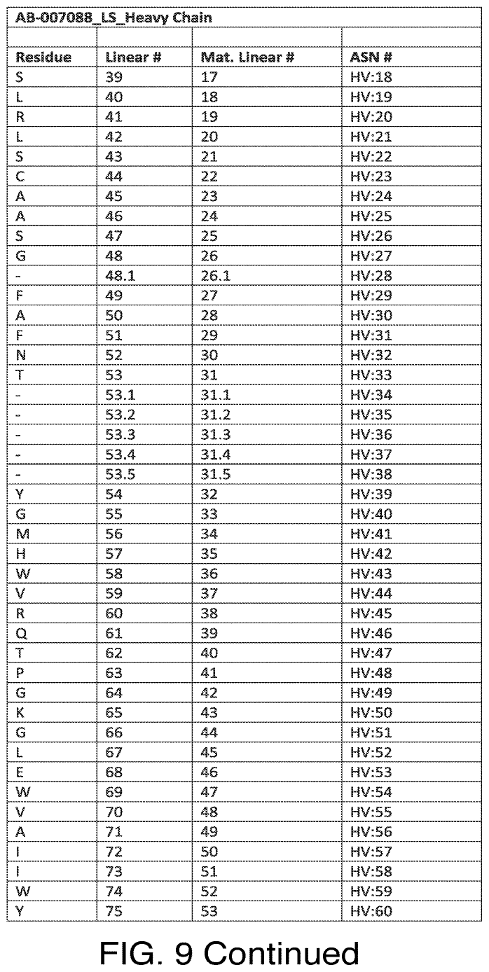

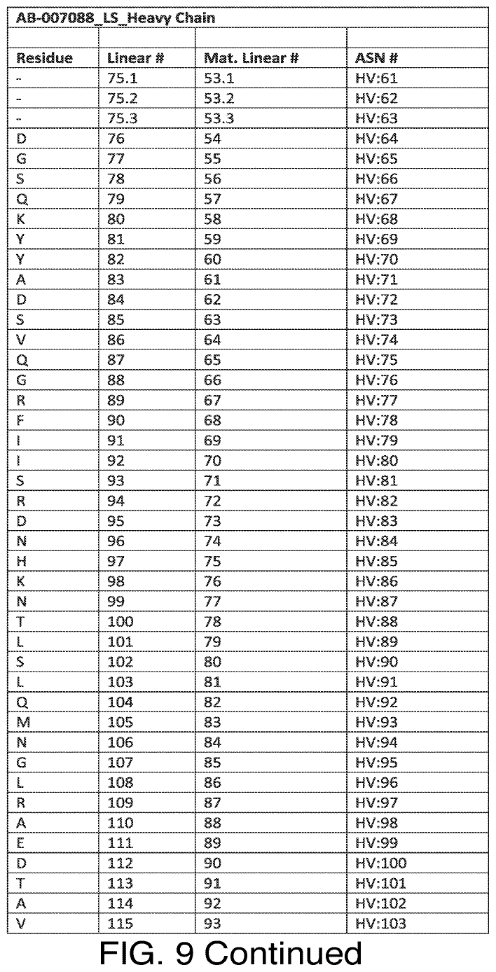

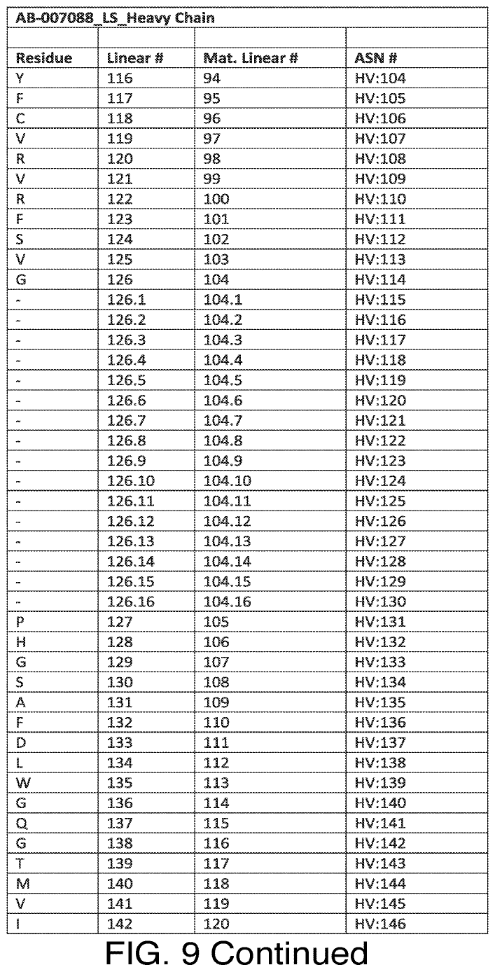

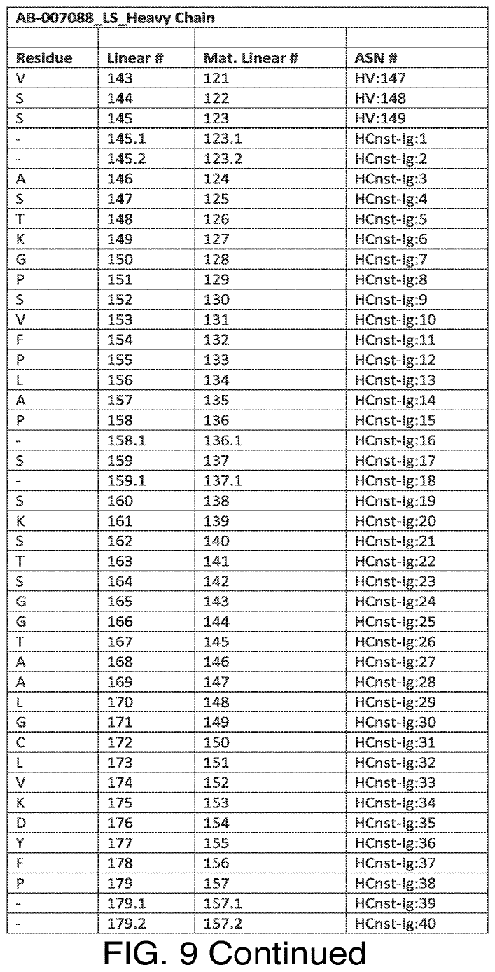

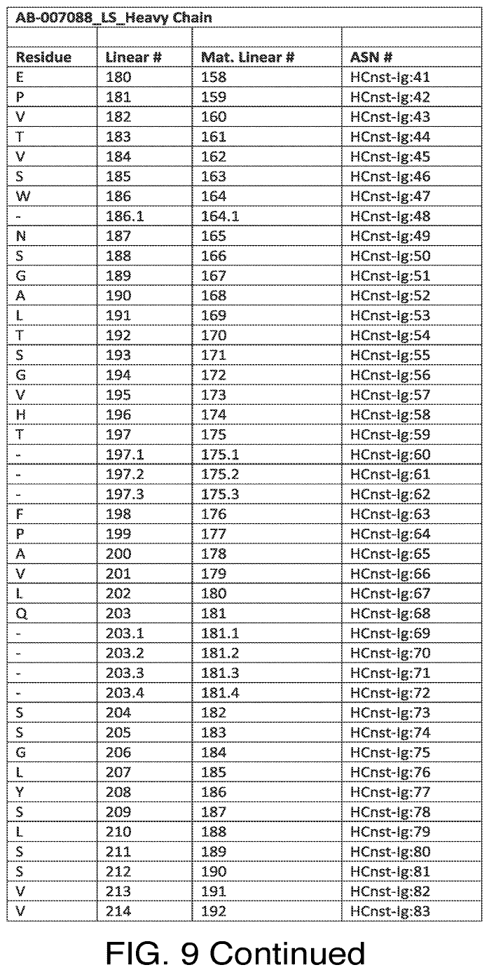

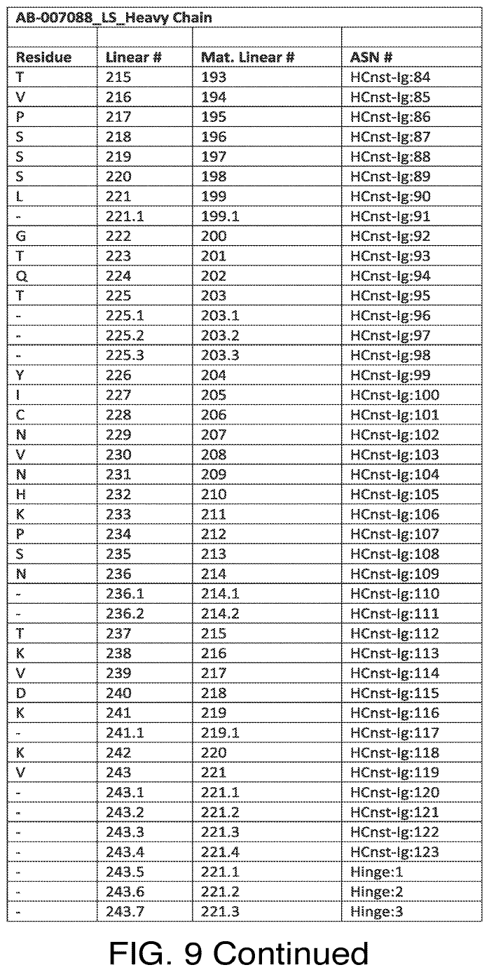

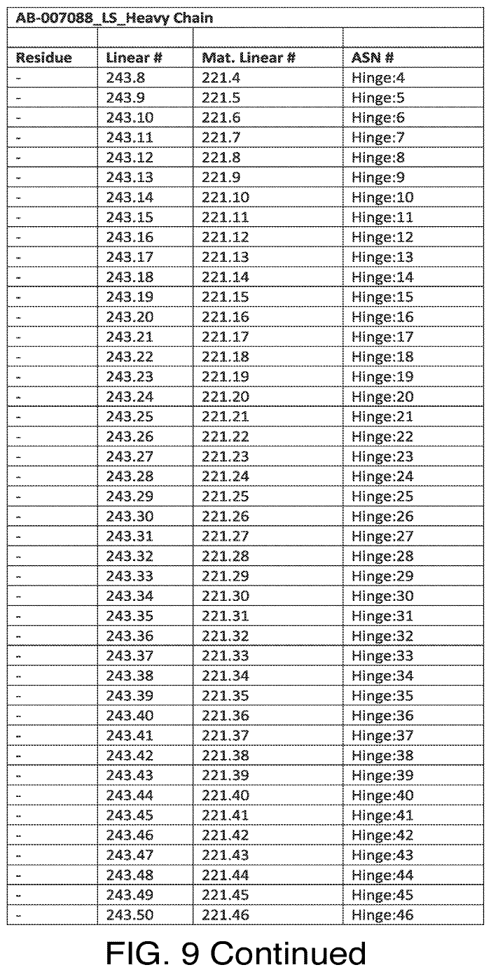

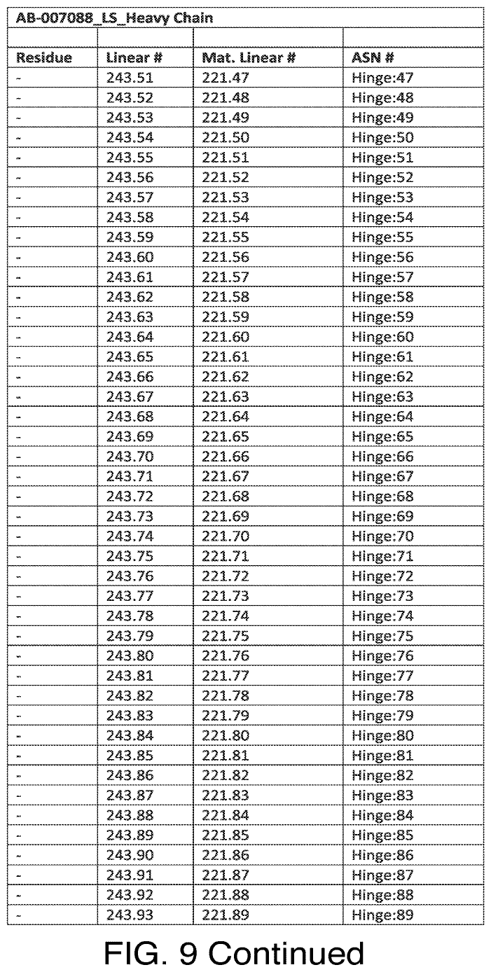

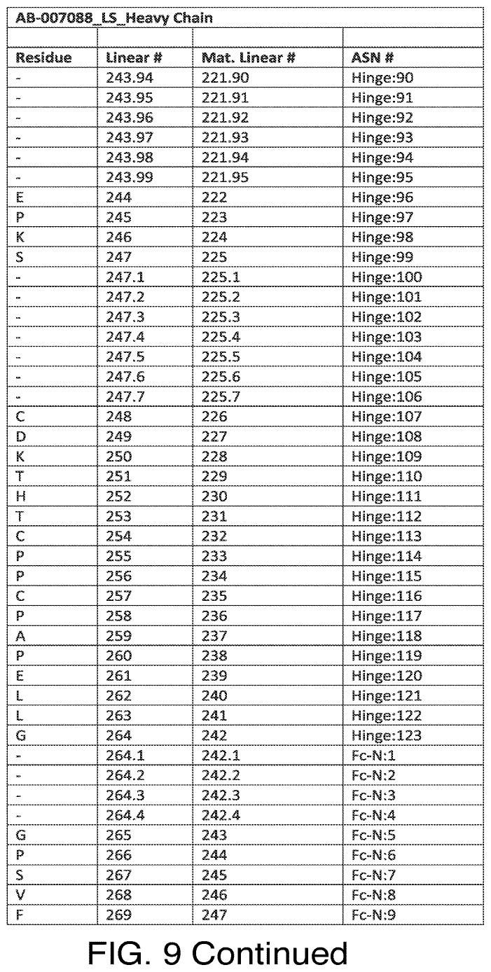

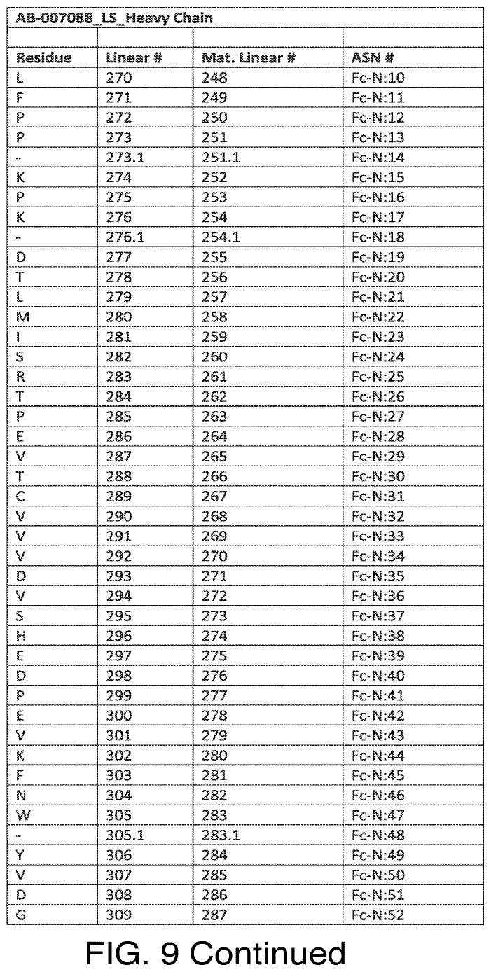

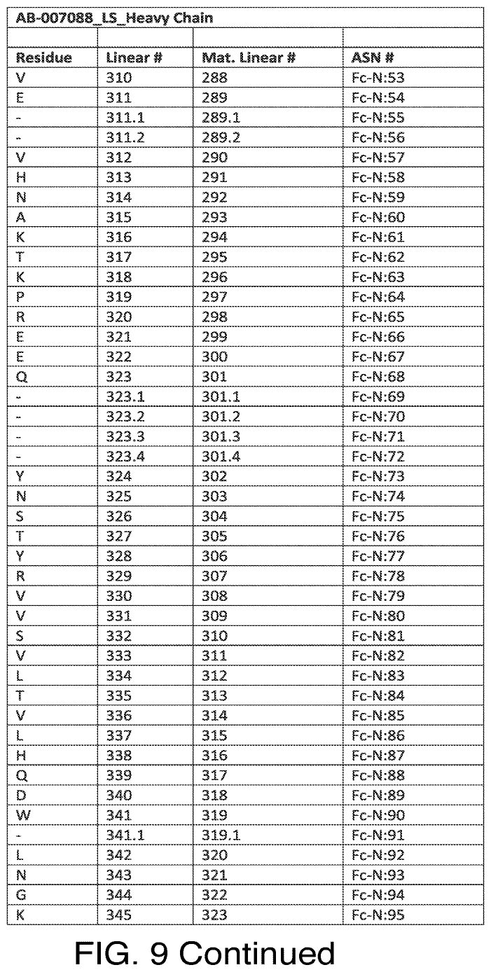

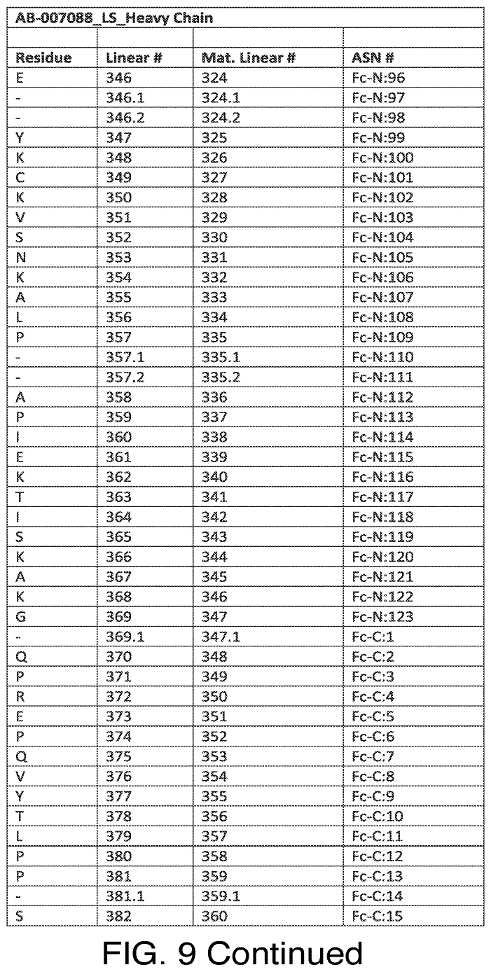

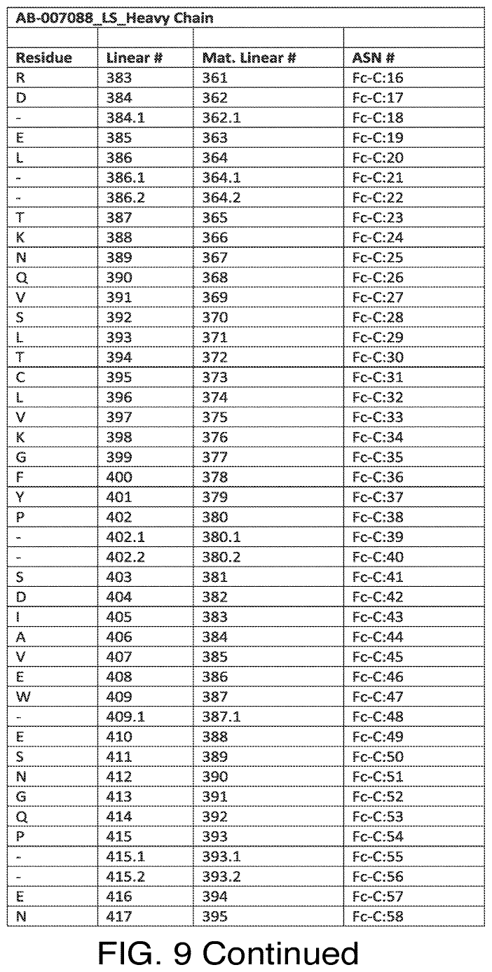

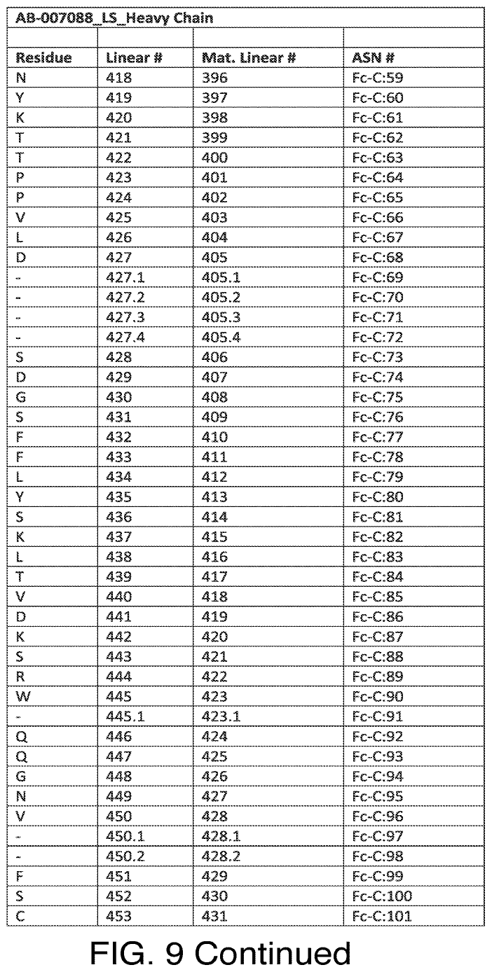

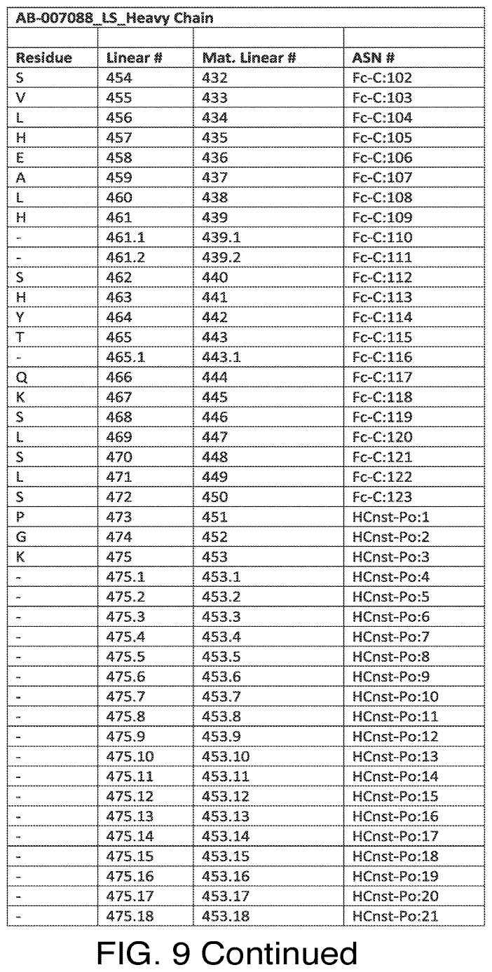

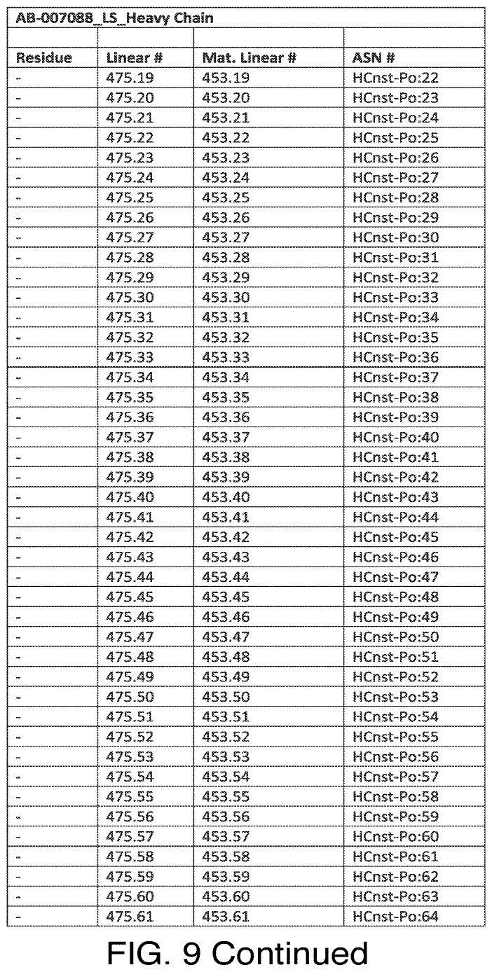

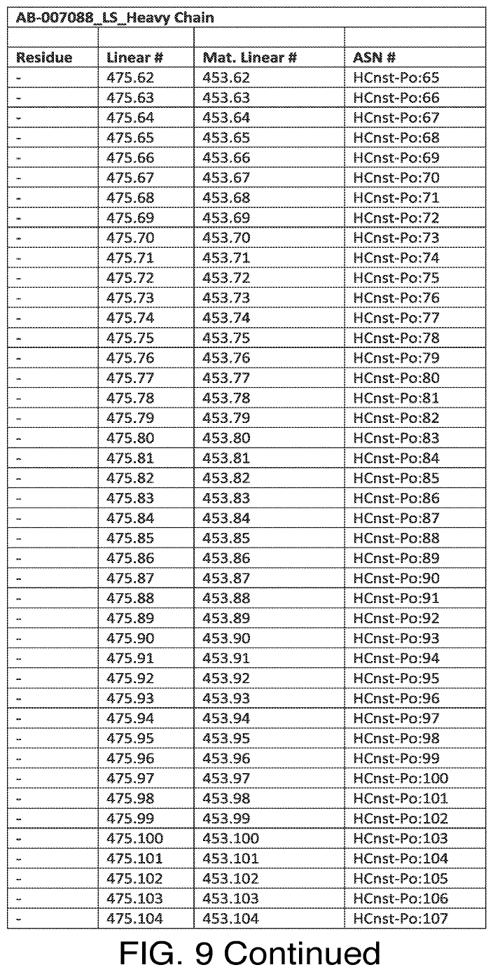

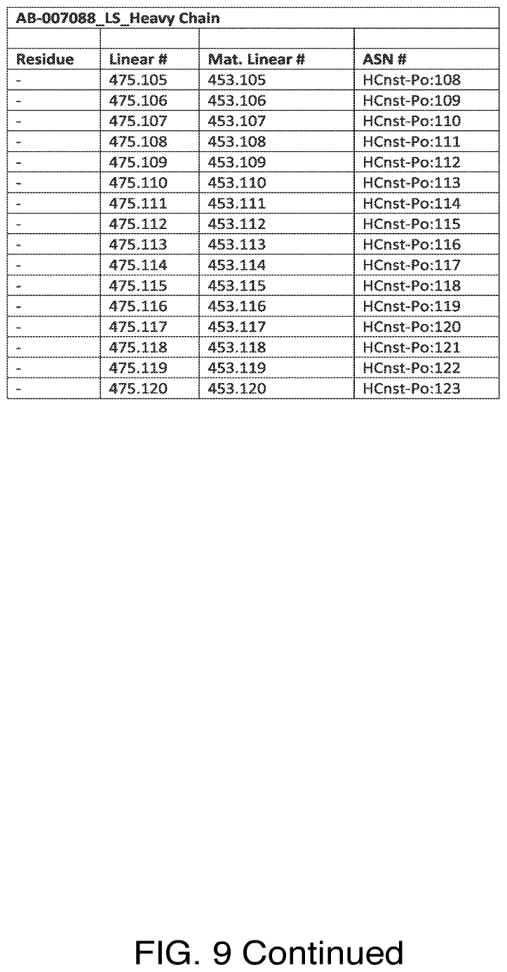

The amino acid sequences of the CDRs and framework regions can be determined using various definitions in the art, e.g., Kabat, Chothia, international ImMunoGeneTics database (IMGT), and AbM (see, e.g., Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, Structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996). Reference to CDRs as determined by Kabat numbering is based, for example, on Kabat et al., Sequences of Proteins of Immunological Interest, 5th Ed. Public Health Service, National Institute of Health, Bethesda, MD (1991)). Chothia CDRs are determined as defined by Chothia (see, e.g., Chothia and Lesk J. Mol. Biol. 196:901-917 (1987)). In certain embodiments, the amino acid sequences of the CDRs and framework regions are numbered using the Antibody Structural Numbering (ASN) system. Antibody Structural Numbering (ASN) is a numbering system developed based on the AHo numbering system (Honegger & Plückthun, J. Mol. Biol. 309:657-670 (2001)) defined by Annemarie Honegger for the variable region, but extended to include constant domains. FIGS. 6-7 illustrates ASN numbering for AB-000224-LS light and heavy chain, respectively. FIGS. 8-9 illustrates ASN numbering for AB-007088-LS light and heavy chain, respectively.

An “Fc region” refers to the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cy2 and Cy3 and the hinge between Cy1 and Cy. It is understood in the art that the boundaries of the Fc region may vary, however, the human IgG heavy chain Fc region is usually defined to comprise residues C226 or P230 to its carboxyl-terminus, using the numbering according to the EU index as in Kabat et al. (1991, NIH Publication 91-3242, National Technical Information Service, Springfield, Va.). The term “Fc region” may refer to this region in isolation or this region in the context of an antibody or antibody fragment. “Fc region” includes naturally occurring allelic variants of the Fc region as well as modifications that modulate effector function. Fc regions also include variants that don't result in alterations to biological function. For example, one or more amino acids can be deleted from the N-terminus or C-terminus of the Fc region of an immunoglobulin without substantial loss of biological function. Such variants can be selected according to general rules known in the art to have minimal effect on activity (see, e.g., Bowie, et al., Science 247:306-1310, 1990). For example, for IgG4 antibodies, a single amino acid substitution (S228P according to Kabat numbering; designated IgG4Pro) may be introduced to abolish the heterogeneity observed in recombinant IgG4 antibodies (see, e.g., Angal, et al., Mol Immunol 30:105-108, 1993). In certain embodiments, the Fc region includes substitutions that improve pharmacokinetics properties of an antibody, e.g., increased serum half-life. Non-limiting examples of substitutions of the Fc region can be found in U.S. Pat. No. 8,088,376, the content of which is incorporated by reference in its entirety.

The term “equilibrium dissociation constant” abbreviated (KD), refers to the dissociation rate constant (kd, time⁻¹) divided by the association rate constant (ka, time⁻¹ M⁻¹). Equilibrium dissociation constants can be measured using any method. Thus, in certain embodiments, the antibodies of the present disclosure have a KD of less than about 50 nM, typically less than about 25 nM, or less than 10 nM, e.g., less than about 5 nM, or than about 1 nM and often less than about 10 nM as determined by surface plasmon resonance analysis using a biosensor system such as a Biacore® system performed at 37° C. In certain embodiments, an antibody of the present disclosure has a KD of less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸ M, less than 5×10⁻⁹M, less than 10⁻⁹M, less than 5×10⁻¹⁰M, less than 10⁻¹⁰M, less than 5×10⁻¹¹M, less than 10⁻¹¹M, less than 5×10⁻¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁵M or lower as measured as a bivalent antibody. As used herein, an “improved” KD refers to a lower KD. In certain embodiments, an antibody of the present disclosure has a KD of less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸ M, less than 5×10⁻⁹M, less than 10⁻⁹M, less than 5×10⁻¹⁰M, less than 10⁻¹⁰M, less than 5×10⁻¹¹M, less than 10⁻¹¹M, less than 5×10⁻¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁵M or lower as measured as a monovalent antibody, such as a monovalent Fab. In certain embodiments, an anti-CSP antibody of the present disclosure has KD less than 100 μM, e.g., or less than 75 μM, e.g., in the range of 1 to 100 μM, when measured by surface plasmon resonance analysis using a biosensor system such as a Biacore® system performed at 37° C. In certain embodiments, an anti-CSP antibody of the present disclosure has KD of greater than 100 μM, e.g., in the range of 100-1000 μM or 500-1000 μM when measured by surface plasmon resonance analysis using a biosensor system such as a Biacore® system performed at 37° C.

The term “monovalent molecule” as used herein refers to a molecule that has one antigen-binding site, e.g., a Fab or scFv.

The term “bivalent molecule” as used herein refers to a molecule that has two antigen-binding sites. In certain embodiments, a bivalent molecule of the present invention is a bivalent antibody or a bivalent fragment thereof. In certain embodiments, a bivalent molecule of the present invention is a bivalent antibody. In certain embodiments, a bivalent molecule of the present invention is an IgG. In certain embodiments, monoclonal antibodies have a bivalent basic structure. IgG and IgE have only one bivalent unit, while IgA and IgM consist of multiple bivalent units (2 and 5, respectively) and thus have higher valencies. This bivalency increases the avidity of antibodies for antigens.

The terms “monovalent binding” or “monovalently binds to” as used herein refer to the binding of one antigen-binding site to its antigen.

The terms “bivalent binding” or “bivalently binds to” as used herein refer to the binding of both antigen-binding sites of a bivalent molecule to its antigen. In certain embodiments, both antigen-binding sites of a bivalent molecule share the same antigen specificity.

The term “valency” as used herein refers to the number of different binding sites of an antibody for an antigen. A monovalent antibody includes one binding site for an antigen. A bivalent antibody (e.g., a bivalent IgG antibody) includes two binding sites for the same antigen.

The term “affinity” as used herein refers to either the single or combined strength of one or both arms of an antibody (e.g., an IgG antibody) binding to either a simple or complex antigen-expressing one or more epitopes. As defined here, the term “affinity” does not imply a specific number of valencies between the two binding partners.

The phrase “specifically (or selectively) binds” to an antigen or target or “specifically (or selectively) immunoreactive with,” when referring to a protein or peptide, refers to a binding reaction whereby the antibody binds to the antigen or target of interest with an affinity that can be distinguished from non-specific interactions occurring between two proteins.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, e.g., the length of the two sequences, when compared and aligned for maximum correspondence over a comparison window or designated region. Alignment for purposes of determining percent amino acid sequence identity can be performed in various methods, including, without any limitation, BLAST, BLAST-2, ALIGN, or Megalign (DNASTAR) software. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). In certain embodiments, BLAST 2.0 can be used with the default parameters to determine percent sequence identity.

A “substitution,” as used herein, denotes the replacement of one or more amino acids or nucleotides by different amino acids or nucleotides, respectively.

A “conservative” substitution as used herein refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe, Tyr and Trp; (iv) nitrogen ring amino acids His and Trp; (v) aliphatic hydrophobic amino acids Ala, Val, Leu and Ile, (vi) hydrophobic sulfur-containing amino acids Met and Cys, which are not as hydrophobic as Val, Leu, and Ile, (vii) small polar uncharged amino acids Ser, Thr, Asp, and Asn (viii) small hydrophobic or neutral amino acids Gly, Ala, and Pro; (ix) amide-comprising amino acids Asn and Gln; and (xi) beta-branched amino acids Thr, Val, and Ile. Reference to the charge of an amino acid refers to the charge at pH 6-7.

As used herein, the terms “nucleic acid” and “polynucleotide” are used interchangeably and as used herein refer to both sense and anti-sense strands of RNA, cDNA, genomic DNA, and synthetic forms and mixed polymers of the above. In certain embodiments, a polynucleotide refers to a polyribonucleotide, polydeoxynucleotide or a modified form of either type of nucleotide, and combinations thereof. The terms also include, but are not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitutions of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular, and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or fragment thereof” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector,” as used herein, refers to a recombinant construct in which a nucleic acid sequence of interest is inserted into the vector. Certain vectors can direct the expression of nucleic acids to which they are operatively linked. Such vectors are referred to herein as “expression vectors”.

The terms “host cell,” “host cell line,” and “host cell culture” are used interchangeably and refer to cells into which exogenous nucleic acid has been introduced, including the progeny of such cells. A host cell can be a recombinant host cell and includes the primary transformed cell and progeny derived therefrom without regard to the number of passages.

A polypeptide “variant,” as the term is used herein, is a polypeptide that typically differs from a polypeptide specifically disclosed herein in one or more substitutions, deletions, additions, and/or insertions. In the present invention, a “variant” with reference to the sequences described in the “Anti-CSP Antibody Variants” section refers to an engineered sequence, rather than a naturally occurring sequence.

The term “comparable,” in the context of describing the strength of binding of two antibodies to the same target, refers to two dissociation constant (KD) values calculated from two binding reactions that are within three (3) fold from each other. In certain embodiments, the ratio between the first KD (the KD of the binding reaction between the first antibody and the target) and the second KD (the KD of the binding reaction between the second antibody and the target) is within the range of 1:3 or 3:1, endpoints exclusive. A lower KD value denotes stronger binding. For example, without any limitation, an antibody variant that has stronger binding as compared to AB-000224 binds to the target with a KD that is at least ⅓ of the KD measured against the same target for AB-000224.

### Anti-CSP Antibodies

The present disclosure provides anti-CSP antibodies AB-000224 and AB-007088 and variants thereof. AB-000224 and AB-007088 were discovered in antibody repertoires generated by Immune Repertoire Capture® (IRC®) technology from plasmablast B cells isolated from two donors enrolled in a Phase 2a study evaluating the efficacy of the RTS,S vaccine in preventing malaria infection. The IRC® technology and its use in antibody discovery is well known and disclosed in, e.g., WO 2012148497A2, the entire content of which is herein incorporated by reference. The RTS,S vaccine is a pseudo-viral particle vaccine that combines the hepatitis B surface antigen and the central repeat and C-terminal regions of the Plasmodium falciparum (P. falciparum) circumsporozoite protein (CSP). RTS,S consists of two polypeptides; RTS is a single polypeptide chain corresponding to amino acids 207 to 395 of P. falciparum (3D7) that is fused to HBsAg and S is a polypeptide of 226 amino acids that corresponds to HBsAg. Stoute, et. al., N Engl J Med; 336:86-91 (1997); RTS,S Clinical Trials Partnership, PLoS Med. 11(7):e1001685, (2014), WO1993/10152.

CSP is composed of an N-terminal domain containing a heparan sulfate binding site for hepatocyte adhesion, a central repeat region, and a structured C-terminal α-thrombospondin repeat (αTSR) that is followed by a GPI anchor, which attaches CSP to the sporozoite membrane. The central repeat region of CSP is highly immunogenic, and in all P. falciparum strains with a CSP sequence available, the repeat region is composed of 1 NPDP repeat, 3-5 NVDP repeats, and 35-41 NANP repeats (e.g., a total of Jan. 4, 2038 of NPDP/NVDP/NANP motifs are present in the P. falciparum 3D7 strain). The repeat region begins with the junctional NPDP sequence, typically followed by three alternations of NANP and NVDP sequences, and continues with the remaining NANP repeats, with most P. falciparum strains having one NVDP interspersed in the middle of the long NANP repeat region. Pholcharee, T. et al., J. Mol. Bio. 432:1048-1063 (2020).

In certain embodiments, the anti-CSP antibodies disclosed herein bind to the central repeat region of P. falciparum CSP. In certain embodiments, the antibodies disclosed herein bind to P. falciparum CSP protein in the repeat and/or junctional regions that contain NPNA, NPDP, and/or NVDP motifs. In certain embodiments, the anti-CSP antibodies disclosed herein bind to the NANP repeat region of P. falciparum CSP. In certain embodiments, the anti-CSP antibodies disclosed herein bind to a polypeptide comprising the amino acid sequence of (NPNA) 3 (SEQ ID NO: 280).

In certain embodiments, the present disclosure provides anti-CSP antibody variants of AB-000224. In certain embodiments, the present disclosure provides anti-CSP antibody variants of AB-007088. In certain embodiments, the variants exhibit protective effects in vivo, e.g., as shown by a reduction in parasite number in a mouse model of malaria infection.

In certain embodiments, the anti-CSP variants disclosed herein maintain the binding specificity, activity and stability and/or manufacturing properties of the parental antibody. In certain embodiments, the anti-CSP variants disclosed herein generated have improved developability, e.g., as identified through various in vitro assays, such as aggregation assessment by HPLC or UPLC, hydrophobic interaction chromatography (HIC), polyspecificity assays (e.g., baculovirus particle binding), self-interaction nanoparticle spectroscopy (SINS), or mass spec analysis after incubation in an accelerated degradation condition such as high temperature, low pH, high pH, or oxidative H₂O₂. Mutations are successful if the activity is maintained (or enhanced) while removing or reducing the severity of the liability.

Antibody liabilities are further described in Table 1 below:

TABLE 1
Description of potential development liabilities

| | | | |
|---|---|---|---|
| Free cysteine¹ | Yield, heterogeneity, | sequence | High |
| | stability, activity | comprises an | |
| | | odd number of | |
| | | cysteines | |
| N-linked | Yield, | N(—P)(S, T)² | High |
| glycosylation | heterogeneity, | | |
| | activity | | |
| Abnormal net charge | Platform fit, PK | Sharma 2014³ | High |
| Patches of | Stability, PK | Sharma 2014 | High |
| hydrophobicity | | | |
| Patches of same | Stability, PK | N/A (based on | Medium |
| charge | | structure) | |
| Proteolysis | Stability, PK | (K, R)(K, R)⁴ | Medium |
| Proteolysis | Stability, PK | DP | Medium |
| Asparagine | Heterogeneity, | NG; | Medium; |
| deamidation | stability, | N(A, N, S, T)⁵ | Low |
| | activity | | |
| Aspartate | Heterogeneity, | DG; | Medium; |
| isomerization | stability, | D(A, D, S, T)⁶ | Low |
| | activity | | |
| Lysine glycation | Heterogeneity, | K | Low |
| | stability, | | |
| | activity | | |
| Methionine oxidation | Heterogeneity, | M | Low |
| | stability, | | |
| | activity | | |
| Tryptophan oxidation | Heterogeneity, | W | Low |
| | stability, | | |
| | activity | | |

¹“Free cysteine” refers to a cysteine that does not form a disulfide bond with another cysteine and thus is left “free” as thiols. The presence of free cysteines in the antibody can be a potential development liability. Typically, an odd net number of cysteines in the protein shows a likelihood there is a free cysteine.
²The N-linked glycosylation site is N-X-S/T, where X is any residue other than proline.
³Sharma et al., Proc. Natl. Acad. Sci. USA 111: 18601-18606, 2014.
⁴This motif consists of a K or R, followed by a K or R. Stated differently, the motif can be KK, KR, RK, or RR.
⁵The dipeptide NG poses a medium risk of development liability. The dipeptides NA, NN, NS, and NT pose a low risk of development liability. N may also exhibit low risk of liability for other successor residues, e.g., D, H, or P. Stated differently, dipeptide ND, NH, or NP poses a low risk of development liability.
⁶Similarly to the above, the dipeptide DG poses a medium risk of development liability. The dipeptides DA, DD, DS, and DT pose a low risk of development liability. D may also exhibit low risk of development liability for other successor residues, e.g., N, H, or P.

Another goal for engineering variants is to reduce the risk of clinical immunogenicity. For example, reducing the generation of anti-drug antibodies against the therapeutic antibody. In certain embodiments, the anti-CSP antibody variants have reduced immunogenicity as compared to the parental antibody.

The factors that drive clinical immunogenicity can be classified into two groups. First are factors that are intrinsic to the drug, such as sequence, post-translational modifications, aggregates, degradation products, and contaminants. Second are factors related to how the drug is used, such as dose level, dose frequency, route of administration, patient immune status, and patient HLA type.

One approach to engineering a variant to be as much like self as possible is to identify a close germline sequence and mutate as many mismatched positions (also known as “germline deviations”) to the germline residue type as possible. This approach applies for germline genes IGHV, IGHJ, IGKV, IGKJ, IGLV, and IGLJ, and accounts for all of the variable heavy (V11) and variable light (VL) regions except for part of H-CDR3. Germline gene IGHD codes for part of the H-CDR3 region but typically exhibits too much variation in how it is recombined with IGHV and IGHJ (e.g., forward or reverse orientation, any of three translation frames, and 5′ and 3′ modifications and non-templated additions) to present a “self” sequence template from a population perspective.

Each germline gene can present as different alleles in the population. The least immunogenic drug candidate, in terms of minimizing the percent of patients with an immunogenic response, would likely be one that matches an allele commonly found in the patient population. Single nucleotide polymorphism (SNP) data from the human genome can be used to approximate the frequency of alleles in the population.

Another approach to engineering a lead for reduced immunogenicity risk is to use in silico predictions of immunogenicity, such as the prediction of T cell epitopes, or use in vitro assays of immunogenicity, such as ex vivo human T cell activation. For example, services such as those offered by Lonza, United Kingdom, are available that employ platforms for prediction of HLA binding and in vitro assessment to further identify potential epitopes.

In certain embodiments, antibody variants are additionally designed to enhance the efficacy of the antibody. Design parameters for this aspect focused on CDRs, e.g., CDR3. Positions to be mutated were identified based on structural analysis of antibody-antigen co-crystals (Oyen et al., Proc. Natl. Acad Sci. USA 114:E10438-E10445, 2017) and based on sequence information of other antibodies from the same lineage as AB-000224 or AB-007088.

### 1. Approaches to Mutation Design

Development liabilities can be removed or reduced by one or more mutations. Mutations are designed to preserve antibody structure and function while removing or reducing development liabilities and to improve function. In certain embodiments, mutations to chemically similar residues were identified to maintain size, shape, charge, and/or polarity. Non-limiting examples of mutations are described in Table 2 below:

TABLE 2

| | | | |
|---|---|---|---|
| Free cysteine | Odd #C | High | C(A, S) |
| N-linked glycosylation | N(—P)(S, T) | High | N-*(Q, D, S, A); |
| | | | (S, T)-*(A, N) |
| Proteolytic cleavage | (K, R)(K, R) | Medium | K, R-*(Q, S, A) |
| Proteolytic cleavage | DP | Medium | D(E, S, A) |
| Asparagine deamidation | NG; | Medium; | N-*(Q, S, A); |
| | N(A, N, S, T)* | Low | G-*(A, S) |
| Aspartate isomerization | DG; | Medium; | D-*(E, S, A); |
| | D(A, D, S, T)* | Low | G-*(A, S) |
| Lysine glycation | K | Low | K(R, Q, S, A) |
| Methionine oxidation | M | Low | M-*(Q, L, S, A) |
| Tryptophan oxidation | W | Low | W-*(Y, F) |
| Proteolytic cleavage | (K, R)(K, R) | Medium | K, R-*(Q, S, A) |

### 2. Anti-CSP Antibody Variants of AB-000224

In certain embodiments, a variant of an anti-CSP antibody AB-000224 disclosed herein comprises modifications compared to AB-000224 that provide improved pharmacokinetic properties, increased serum stability, stronger binding, and/or improved in vivo protective effects compared to AB-000224. In certain embodiments, a variant of an anti-CSP antibody AB-000224 disclosed herein exhibits reduced immunogenicity and/or increased manufacturability as compared to AB-000224. In certain embodiments, a variant of an anti-CSP antibody AB-000224 disclosed herein has at least one modification, e.g., substitution, relative to the native AB-000224 variable heavy chain amino acid sequence (SEQ ID NO: 14) or variable light chain amino acid sequence (SEQ ID NO: 13) described herein, and has improved developability, e.g., decreased heterogeneity, increased yield, increased stability, improved net charges to improve pharmacokinetics, and/or reduced immunogenicity. In certain embodiments, a VH region or a VL region of such a variant of an anti-CSP antibody AB-000224 disclosed herein has at least two, three, four, five, or six, or more modifications, e.g., substitutions. In certain embodiments, a variant of the an anti-CSP antibody AB-000224 disclosed herein has a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications, e.g. substitutions, including both variable regions, compared to AB-000224.

In certain embodiments, a variant of an anti-CSP antibody AB-000224 disclosed herein exhibits increased serum half-life as compared to AB-000224. In certain embodiments, a variant of an anti-CSP antibody AB-000224 disclosed herein has at least one modification, e.g., substitution, relative to the native AB-000224 Fc region of the heavy chain sequence described herein, and has improved pharmacokinetics properties, e.g., half-life. In certain embodiments, an Fc region of the heavy chain of such a variant of an anti-CSP antibody AB-000224 disclosed herein has at least two, three, four, five, or six, or more modifications, e.g., substitutions. In certain embodiments, a variant of an anti-CSP antibody AB-000224 disclosed herein has a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications, e.g. substitutions, including both heavy and light chains, compared to AB-000224. In certain non-limiting embodiments, an Fc region of the heavy chain of a variant of an anti-CSP antibody AB-000224 disclosed herein can include an isoleucine at position 250, a tyrosine at position 252, an isoleucine at position 259, a glutamine at position 307, a phenylalanine at position 308, a leucine at position 319, a leucine at position 428, a histidine at position 434, a phenylalanine at position 434, an alanine at position 434, a serine at position 434, a methionine at position 434, or a combination thereof, wherein the numbering is defined by EU index as in Kabat. In certain embodiments, an Fc region of the heavy chain of a variant of an anti-CSP antibody AB-000224 disclosed herein includes a leucine at position 428 and a serine at position 434, wherein the numbering is defined by EU index as in Kabat.

The light and heavy chain CDRs of AB-000224 as defined by Kabat numbering system are shown in Table 3 below:

TABLE 3
AB-000224 CDR sequences (Kabat)

| | CDR1 | CDR2 | CDR3 |
|---|---|---|---|
| VL region | TGMNSNIGAGYDVY | GNSNRPS | QSYDTSLNGWA |
| | (SEQ ID NO: 1) | (SEQ ID NO: 2) | (SEQ ID NO: 3) |
| VH region | DHAMS | FIRKTTYGATTHYAAAVRG | VQLDYGPGYQYYGMDV |
| | (SEQ ID NO: 4) | (SEQ ID NO: 5) | (SEQ ID NO: 6) |

The light and heavy chain CDRs of AB-000224 as defined by ASN numbering system are shown in Table 4 below:

TABLE 4
AB-000224 CDR sequences (ASN)

| | CDR1 | CDR2 | CDR3 |
|---|---|---|---|
| VL region | TGMNSNIGAGYDVY | GNSNRPS | QSYDTSLNGWA |
| | (SEQ ID NO: 7) | (SEQ ID NO: 8) | (SEQ ID NO: 9) |
| VH region | DHAMS | FIRKTTYGATTHYAAAVRG | VQLDYGPGYQYYGMDV |
| | (SEQ ID NO: 10) | (SEQ ID NO: 11) | (SEQ ID NO: 12) |

The heavy chain variable region (VH) and light chain variable region (VL) sequences and heavy and light chain sequences of AB-000224 are shown in Table 5 below:

TABLE 5
AB-000224 variable region and full chain sequences

| | |
|---|---|
| VL region | ESVLTQPPSVSGAPGQRVTISCTGMNSNIGAGYDVYWYQQLPGRAPKLLIYGNSNRP |
| | SGVPDRFSGSRSGTSASLAITGLQAEDEADYYCQSYDTSLNGWAFGGGTKLTVLG |
| | (SEQ ID NO: 13) |
| VH region | EVQLVESGGGLVQPGRSLRLPCTASGFSFGDHAMSWVRQAPGKGLEWVGFIRKTTYG |
| | ATTHYAAAVRGRFTISRDDSKSIVYLQMNSLKTEDTAVYFCTRVQLDYGPGYQYYGM |
| | DVWGQGTTVTVSS (SEQ ID NO: 14) |
| DNA for VL region | GAGTCTGTGCTGACGCAGCCGCCCTCAGTGTCTGGGGCCCCAGGGCAGAGGGTCACC |
| | ATCTCCTGCACTGGGATGAACTCCAACATCGGGGCAGGTTATGATGTATACTGGTAC |
| | CAACAACTTCCAGGAAGAGCCCCCAAACTCCTCATCTATGGTAACAGCAATCGGCCC |
| | TCAGGGGTCCCTGACCGATTCTCTGGCTCCAGGTCTGGCACCTCAGCCTCCCTGGCC |
| | ATCACTGGGCTCCAGGCTGAGGATGAGGCTGATTATTACTGCCAGTCCTATGACACC |
| | AGCCTGAATGGTTGGGCTTTCGGCGGAGGGACCAAGTTGACCGTCCTAGGC [SEQ |
| | ID NO: 15] |
| DNA for VH region | GAGGTGCAGCTGGTGGAGTCTGGGGGAGGCTTGGTACAGCCAGGGCGGTCCCTGAGA |
| | CTCCCCTGTACAGCCTCTGGGTTTAGTTTTGGTGATCATGCTATGAGCTGGGTCCGC |
| | CAGGCTCCAGGGAAGGGGCTGGAGTGGGTAGGTTTCATTAGAAAGACAACTTATGGT |
| | GCGACAACACACTACGCCGCGGCTGTGAGAGGCAGATTCACCATCTCGCGAGATGAT |
| | TCTAAAAGCATTGTCTATCTGCAAATGAACAGCCTGAAAACCGAGGACACAGCCGTG |
| | TATTTCTGTACTAGAGTGCAGCTTGACTATGGCCCGGGATACCAGTACTACGGTATG |
| | GACGTCTGGGGCCAAGGGACCACGGTCACCGTCTCCTCA [SEQ ID NO: 16] |
| Light Chain | ESVLTQPPSVSGAPGQRVTISCTGMNSNIGAGYDVYWYQQLPGRAPKLLIYGNSNRP |
| | SGVPDRFSGSRSGTSASLAITGLQAEDEADYYCQSYDTSLNGWAFGGGTKLTVLGQP |
| | KAAPSVTLFPPSSEELQANKATLVCLVSDFYPGAVTVAWKADGSPVKVGVETTKPSK |
| | QSNNKYAASSYLSLTPEQWKSHRSYSCRVTHEGSTVEKTVAPAECS |
| | [SEQ ID NO: 17] |
| Heavy Chain version 1 | EVQLVESGGGLVQPGRSLRLPCTASGFSFGDHAMSWVRQAPGKGLEWVGFIRKTTYG |
| | ATTHYAAAVRGRFTISRDDSKSIVYLQMNSLKTEDTAVYFCTRVQLDYGPGYQYYGM |
| | DVWGQGTTVTVSSASTKGPSVFPLAPSSKSTSGGTAALGCLVKDYFPEPVTVSWNSG |
| | ALTSGVHTFPAVLQSSGLYSLSSWTVPSSSLGTQTYICNVNHKPSNTKVDKKVEPK |
| | SCDKTHTCPPCPAPELLGGPSVFLFPPKPKDTLMISRTPEVTCVVVDVSHEDPEVKF |
| | NWYVDGVEVHNAKTKPREEQYNSTYRWSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK [SEQ ID NO: 18] |
| Heavy Chain version 2 | EVQLVESGGGLVQPGRSLRLPCTASGFSFGDHAMSWVRQAPGKGLEWVGFIRKTTYG |
| | ATTHYAAAVRGRFTISRDDSKSIVYLQMNSLKTEDTAVYFCTRVQLDYGPGYQYYGM |
| | DVWGQGTTVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSHEDPEVKE |
| | NWYVDGVEVHNAKTKPREEQYNSTYRVVSVLTVLHQDWLNGKEYKCKVSNKALPAPI |
| | EKTISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVLHEALHSHYTQKSLSLSPG |
| | K [SEQ ID NO: 19] |
| DNA for Light Chain | GAGTCTGTGCTGACGCAGCCGCCCTCAGTGTCTGGGGCCCCAGGGCAGAGGGTCACC |
| | ATCTCCTGCACTGGGATGAACTCCAACATCGGGGCAGGTTATGATGTATACTGGTAC |
| | CAACAACTTCCAGGAAGAGCCCCCAAACTCCTCATCTATGGTAACAGCAATCGGCCC |
| | TCAGGGGTCCCTGACCGATTCTCTGGCTCCAGGTCTGGCACCTCAGCCTCCCTGGCC |
| | ATCACTGGGCTCCAGGCTGAGGATGAGGCTGATTATTACTGCCAGTCCTATGACACC |
| | AGCCTGAATGGTTGGGCTTTCGGCGGAGGGACCAAGTTGACCGTCCTAGGCCAGCCC |
| | AAGGCTGCCCCCTCGGTCACTCTGTTCCCACCCTCCTCTGAGGAGCTTCAAGCCAAC |
| | AAGGCCACACTGGTGTGTCTCGTAAGTGACTTCTACCCGGGAGCCGTGACAGTGGCC |
| | TGGAAGGCAGATGGCAGCCCCGTCAAGGTGGGAGTGGAGACCACCAAACCCTCCAAA |
| | CAAAGCAACAACAAGTATGCGGCCAGCAGCTACCTGAGCCTGACGCCCGAGCAGTGG |
| | AAGTCCCACAGAAGCTACAGCTGCCGGGTCACGCATGAAGGGAGCACCGTGGAGAAG |
| | ACAGTGGCCCCTGCAGAATGCTCT [SEQ ID NO: 20] |
| DNA for Heavy Chain | GAGGTGCAGCTGGTGGAGTCTGGGGGAGGCTTGG |
| version 1 | TACAGCCAGGGCGGTCCCTGAGACTCCCCTGTACAGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGCATTGTCTATCTGCAAATGAACAGCCTGAAAACCGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA |
| | [SEQ ID NO: 21] |
| DNA for Heavy Chain | GAGGTGCAGCTGGTGGAGTCTGGGGGAGGCTTGG |
| version 2 | TACAGCCAGGGCGGTCCCTGAGACTCCCCTGTACAGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGCATTGTCTATCTGCAAATGAACAGCCTGAAAACCGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 22] |

In certain embodiments, a variant of an anti-CSP antibody includes one, two, or three CDRs of a VL sequence of Table 5. In certain embodiments, a variant of an anti-CSP antibody includes at least one mutation, e.g., substitution, and no more than 10, 20, 30, 40, or 50 mutations in the VL amino acid sequence set forth in SEQ ID NO: 13. In certain embodiments, the VL of the variant of an anti-CSP antibody includes at least one, at least two, or at least three mutations, e.g., substitutions, in the CDR1 amino acid sequence set forth in SEQ ID NO: 1. In certain embodiments, the VL of the variant of an anti-CSP antibody includes at least one, at least two, or at least three mutations, e.g., substitutions, in the CDR2 amino acid sequence set forth in SEQ ID NO: 2. In certain embodiments, the VL of the variant of an anti-CSP antibody includes at least one, at least two, or at least three mutations, e.g., substitutions, in the CDR3 amino acid sequence set forth in SEQ ID NO: 3. In certain embodiments, the VL of the variant of an anti-CSP antibody includes at least one, at least two, or at least three mutations, e.g., substitutions, in the CDR1 amino acid sequence set forth in SEQ ID NO: 7. In certain embodiments, the VL of the variant of an anti-CSP antibody includes at least one, at least two, or at least three mutations, e.g., substitutions, in the CDR2 amino acid sequence set forth in SEQ ID NO: 8. In certain embodiments, the VL of the variant of an anti-CSP antibody includes at least one, at least two, or at least three mutations, e.g., substitutions, in the CDR3 amino acid sequence set forth in SEQ ID NO: 9. In certain embodiments, the mutation is a conservative substitution. In certain embodiments, the VL of the variant of an anti-CSP antibody includes at least one, at least two, or at least three mutations, e.g., substitutions, in the framework of the amino acid sequence set forth in SEQ ID NO: 13. An exemplary nucleic acid sequence of SEQ ID NO: 13 is set forth in SEQ ID NO: 15. In certain embodiments, the VL of the variant of an anti-CSP antibody includes a substitution at position 1 of the SEQ ID NO: 13. In certain embodiments, the substitution is E1Q. In certain embodiments, the VL of the variant of an anti-CSP antibody includes a substitution at position 4 of the SEQ ID NO: 13. In certain embodiments, the substitution is R44T.

In certain embodiments, a variant of an anti-CSP antibody includes one, two, or three CDRs of a VH sequence of Table 5. In certain embodiments, a variant of an anti-CSP antibody includes at least one mutation, e.g., substitutions, and no more than 10, 20, 30, 40, or 50 mutations in the VH amino acid sequence set forth in SEQ ID NO: 14. In certain embodiments, the VH of the variant of an anti-CSP antibody includes at least one, at least two, or at least three mutations, e.g., substitutions, in the CDR1 amino acid sequence set forth in SEQ ID NO: 4. In certain embodiments, the VH of the variant of an anti-CSP antibody includes at least one, at least two, or at least three mutations, e.g., substitutions, in the CDR2 amino acid sequence set forth in SEQ ID NO: 5. In certain embodiments, the VH of the variant of an anti-CSP antibody includes at least one, at least two, or at least three mutations, e.g., substitutions, in the CDR3 amino acid sequence set forth in SEQ ID NO: 6. In certain embodiments, the VH of the variant of an anti-CSP antibody includes at least one, at least two, or at least three mutations, e.g., substitutions, in the CDR1 amino acid sequence set forth in SEQ ID NO: 10. In certain embodiments, the VH of the variant of an anti-CSP antibody includes at least one, at least two, or at least three mutations, e.g., substitutions, in the CDR2 amino acid sequence set forth in SEQ ID NO: 11. In certain embodiments, the VH of the variant of an anti-CSP antibody includes at least one, at least two, or at least three mutations, e.g., substitutions, in the CDR3 amino acid sequence set forth in SEQ ID NO: 12. In certain embodiments, the mutation is a conservative substitution. In certain embodiments, the VH of the variant of an anti-CSP antibody includes a CDR2 having a substitution at position 12 of the SEQ ID NO: 5. In certain embodiments, the substitution is H12K. In certain embodiments, the VH of the variant of an anti-CSP antibody includes a CDR2 having a substitution at position 18 of the SEQ ID NO: 5. In certain embodiments, the substitution is R18K. In certain embodiments, the mutation is a conservative substitution. In certain embodiments, the VH of the variant of an anti-CSP antibody includes a CDR2 having a substitution at position 12 of the SEQ ID NO: 11. In certain embodiments, the substitution is H12K. In certain embodiments, the VH of the variant of an anti-CSP antibody includes a CDR2 having a substitution at position 18 of the SEQ ID NO: 11. In certain embodiments, the substitution is R18K. In certain embodiments, the VH of the variant of an anti-CSP antibody includes at least one, at least two, at least three mutations, or at least four mutations, e.g., substitutions, in the framework of the amino acid sequence set forth in SEQ ID NO: 14. An exemplary nucleic acid sequence of SEQ ID NO: 14 is set forth in SEQ ID NO: 16. In certain embodiments, the VH of the variant of an anti-CSP antibody includes a substitution at position 21 of the SEQ ID NO: 14. In certain embodiments, the substitution is P21S. In certain embodiments, the VH of the variant of an anti-CSP antibody includes a substitution at position 23 of the SEQ ID NO: 14. In certain embodiments, the substitution is T23A. In certain embodiments, the VH of the variant of an anti-CSP antibody includes a substitution at position 80 of the SEQ ID NO: 14. In certain embodiments, the substitution is I80T. In certain embodiments, the VH of the variant of an anti-CSP antibody includes a substitution at position 90 of the SEQ ID NO: 14. In certain embodiments, the substitution is T90A. In certain embodiments, the VH of the variant of an anti-CSP antibody includes a substitution at position 99 of the SEQ ID NO: 14. In certain embodiments, the substitution is T99A.

In certain embodiments, a variant of an anti-CSP antibody includes at least one mutation, e.g., substitution, and no more than 10, 20, 30, 40, or 50 mutations in the Fc region of the heavy chain amino acid sequence set forth in SEQ ID NO: 18. In certain embodiments, the heavy chain of the variant of an anti-CSP antibody has an amino acid sequence set forth in SEQ ID NO: 18. An exemplary nucleic acid sequence of SEQ ID NO: 18 is set forth in SEQ ID NO: 21. In certain embodiments, the heavy chain of the variant of an anti-CSP antibody includes a substitution at position 438 of the SEQ ID NO: 18. In certain embodiments, the substitution is M438L. In certain embodiments, the heavy chain of the variant of an anti-CSP antibody includes a substitution at position 444 of the SEQ ID NO: 18. In certain embodiments, the substitution is N444S. In certain embodiments, the heavy chain of the variant of an anti-CSP antibody includes a substitution at position 438 of the SEQ ID NO: 18 and a substitution at position 444 of the SEQ ID NO: 18. In certain embodiments, the substitutions are M438L and N444S. In certain embodiments, the heavy chain of the variant of an anti-CSP antibody has an amino acid sequence set forth in SEQ ID NO: 19. An exemplary nucleic acid sequence of SEQ ID NO: 19 is set forth in SEQ ID NO: 22.

In certain embodiments, the light chain of the anti-CSP antibody AB-000224 and variants thereof comprises a signal peptide. In certain embodiments, the signal peptide is an IGLV2-8 signal peptide. In certain embodiments, the signal peptide has an amino acid sequence set forth in SEQ ID NO: 277. In certain embodiments, the heavy chain of the anti-CSP antibody AB-000224 and variants thereof comprises a signal peptide. In certain embodiments, the signal peptide is an IGKV1-39 signal peptide. In certain embodiments, the signal peptide has an amino acid sequence set forth in SEQ ID NO: 278. SEQ ID NO: 277 and SEQ ID NO: 278 are provided below:

[SEQ ID NO: 277]
MAWALLLLTLLTQGTGSWA
[SEQ ID NO: 278]
MDMRVPAQLLGLLLLWLRGARC

In certain embodiments, the anti-CSP antibody variant comprises a light chain variable region comprising a CDR1, a CDR2, and a CDR3 and having the amino acid sequence set forth in SEQ ID NO: 23, as shown in Table 6. In certain embodiments, the anti-CSP antibody variant comprises a heavy chain variable region comprising a CDR1, a CDR2, and a CDR3 and having the amino acid sequence set forth in SEQ ID NO: 24, as shown in Table 6. In certain embodiments, the anti-CSP antibody variant comprises a light chain having the amino acid sequence set forth in SEQ ID NO: 27, as shown in Table 6. In certain embodiments, the anti-CSP antibody variant comprises a heavy chain having the amino acid sequence set forth in SEQ ID NO: 28, as shown in Table 6. In certain embodiments, the anti-CSP antibody variant comprises a heavy chain having the amino acid sequence set forth in SEQ ID NO: 29, as shown in Table 6. Exemplary nucleic acid sequences of SEQ ID NOS: 23, 24, 27, 28, and 29 are provided in Table 6 below.

TABLE 6
Antibody ID: AB-000224.001

| | KABAT | ASN |
|---|---|---|
| CDR1-VL | TGMNSNIGAGYDVY [SEQ ID | TGMNSNIGAGYDVY [SEQ ID NO: 7] |
| | NO: 1] | |
| CDR2-VL | GNSNRPS [SEQ ID NO: 2] | GNSNRPS [SEQ ID NO: 8] |
| CDR3-VL | QSYDTSLNGWA [SEQ ID NO: | QSYDTSLNGWA [SEQ ID NO: 9] |
| | 3] | |
| CDR1-VH | DHAMS [SEQ ID NO: 4] | DHAMS [SEQ ID NO: 10] |
| CDR2-VH | FIRKTTYGATTHYAAAVRG [SEQ | FIRKTTYGATTHYAAAVRG [SEQ ID |
| | ID NO: 5] | NO: 11] |
| CDR3-VH | VQLDYGPGYQYYGMDV [SEQ ID | VQLDYGPGYQYYGMDV [SEQ ID NO: |
| | NO: 6] | 12] |

| | |
|---|---|
| VL | QSVLTQPPSVSGAPGQRVTISCTGMNSNIGAGYDVYWYQQLPGTAPKLLIYGNSNRPS |
| | GVPDRFSGSRSGTSASLAITGLQAEDEADYYCQSYDTSLNGWAFGGGTKLTVLG |
| | [SEQ ID NO: 23] |
| VH | EVQLVESGGGLVQPGRSLRLSCTASGFSFGDHAMSWVRQAPGKGLEWVGFIRKTTYGA |
| | TTHYAAAVRGRFTISRDDSKSIVYLQMNSLKTEDTAVYFCTRVQLDYGPGYQYYGMDV |
| | WGQGTTVTVSS [SEQ ID NO: 24] |
| DNA for | CAGTCTGTGCTGACGCAGCCGCCCTCAGTGTCTGGGGCCCCAGGGCAGAGGGTCACCA |
| VL | TCTCCTGCACTGGGATGAACTCCAACATCGGGGCAGGTTATGATGTATACTGGTACCA |
| | ACAACTTCCAGGAACTGCCCCCAAACTCCTCATCTATGGTAACAGCAATCGGCCCTCA |
| | GGGGTCCCTGACCGATTCTCTGGCTCCAGGTCTGGCACCTCAGCCTCCCTGGCCATCA |
| | CTGGGCTCCAG |
| | GCTGAGGATGAGGCTGATTATTACTGCCAGTCCTATGACACCAGCCTGAATGGTTGGG |
| | CTTTCGGCGGAGGGACCAAGTTGACCGTCCTAGGC [SEQ ID NO: 25] |
| DNA for | GAGGTGCAGCTGGTGGAGTCTGGGGGAGGCTTGGTACAGCCAGGGCGGTCCCTGAGAC |
| VH | TCAGCTGTACAGCCTCTGGGTTTAGTTTTGGTGATCATGCTATGAGCTGGGTCCGCCA |
| | GGCTCCAGGGAAGGGGCTGGAGTGGGTAGGTTTCATTAGAAAGACAACTTATGGTGCG |
| | ACAACACACTACGCCGCGGCTGTGAGAGGCAGATTCACCATCTCGCGAGATGATTCTA |
| | AAAGCATTGTCTATCTGCAAATGAACAGCCTGAAAACCGAGGACACAGCCGTGTATTT |
| | CTGTACTAGAGTGCAGCTTGACTATGGCCCGGGATACCAGTACTACGGTATGGACGTC |
| | TGGGGCCAAGGGACCACGGTCACCGTCTCCTCA [SEQ ID NO: 26] |
| Light Chain | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS [SEQ ID NO: 27] |
| Heavy | EVQLVESGGGLVQPGRSLRLSCTASGFSFGDHAMSWVRQAPGKGLEWVGFIRKTTYGA |
| Chain | TTHYAAAVRGRFTISRDDSKSIVYLQMNSLKTEDTAVYFCTRVQLDYGPGYQYYGMDV |
| version 1 | WGQGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISK |
| | AKGQPREPQVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPGK [SEQ |
| | ID NO: 28] |
| Heavy | EVQLVESGGGLVQPGRSLRLSCTASGFSFGDHAMSWVRQAPGKGLEWVGFIRKTTYGA |
| Chain | TTHYAAAVRGRFTISRDDSKSIVYLQMNSLKTEDTAVYFCTRVQLDYGPGYQYYGMDV |
| version 2 | WGQGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISK |
| | AKGQPREPQVYTLPPSRDELTKNQVSLTCLVKGFYPSDIAVEWESNGQPENNYKTTPP |
| | VLDSDGSFFLYSKLTVDKSRWQQGNVFSCSVLHEALHSHYTQKSLSLSPGK [SEQ |
| | ID NO: 29] |
| DNA for | CAGTCTGTGCTGACGCAGCCGCCCTCAGTGTCTGGGGCCCCAGGGCAGAGGGTCACCA |
| Light Chain | TCTCCTGCACTGGGATGAACTCCAACATCGGGGCAGGTTATGATGTATACTGGTACCA |
| | ACAACTTCCAGGAACTGCCCCCAAACTCCTCATCTATGGTAACAGCAATCGGCCCTCA |
| | GGGGTCCCTGACCGATTCTCTGGCTCCAGGTCTGGCACCTCAGCCTCCCTGGCCATCA |
| | CTGGGCTCCAGGCTGAGGATGAGGCTGATTATTACTGCCAGTCCTATGACACCAGCCT |
| | GAATGGTTGGGCTTTCGGCGGAGGGACCAAGTTGACCGTCCTAGGCCAGCCCAAGGCT |
| | GCCCCCTCGGTCACTCTGTTCCCACCCTCCTCTGAGGAGCTTCAAGCCAACAAGGCCA |
| | CACTGGTGTGTCTCGTAAGTGACTTCTACCCGGGAGCCGTGACAGTGGCCTGGAAGGC |
| | AGATGGCAGCCCCGTCAAGGTGGGAGTGGAGACCACCAAACCCTCCAAACAAAGCAAC |
| | AACAAGTATGCGGCCAGCAGCTACCTGAGCCTGACGCCCGAGCAGTGGAAGTCCCACA |
| | GAAGCTACAGCTGCCGGGTCACGCATGAAGGGAGCACCGTGGAGAAGACAGTGGCCCC |
| | TGCAGAATGCTCT [SEQ ID NO: 30] |
| DNA for | GAGGTGCAGCTGGTGGAGTCTGGGGGAGGCTTGGTACAGCCAGGGCGGTCCCTGAGAC |
| Heavy | TCAGCTGTACAGCCTCTGGGTTTAGTTTTGGTGATCATGCTATGAGCTGGGTCCGCCA |
| Chain | GGCTCCAGGGAAGGGGCTGGAGTGGGTAGGTTTCATTAGAAAGACAACTTATGGTGCG |
| version 1 | ACAACACACTACGCCGCGGCTGTGAGAGGCAGATTCACCATCTCGCGAGATGATTCTA |
| | AAAGCATTGTCTATCTGCAAATGAACAGCCTGAAAACCGAGGACACAGCCGTGTATTT |
| | CTGTACTAGAGTGCAGCTTGACTATGGCCCGGGATACCAGTACTACGGTATGGACGTC |
| | TGGGGCCAAGGGACCACGGTCACCGTCTCCTCAGCCTCCACCAAGGGCCCATCGGTCT |
| | TCCCCCTGGCACCCTCCTCCAAGAGCACCTCTGGGGGCACAGCAGCCCTGGGCTGCCT |
| | GGTCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGCA |
| | GCGTGGTGACCGTGCCCTCCAGCAGCTTGGGCACCCAGACCTACATCTGCAACGTGAA |
| | TCACAAGCCCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTCAGTCTTCC |
| | TCTTCCCCCCAAAACCCAAGGACACCCTCATGATCTCCCGGACCCCTGAGGTCACATG |
| | CGTGGTGGTGGACGTGAGCCACGAAGACCCTGAGGTCAAGTTCAACTGGTACGTGGAC |
| | GGCGTGGAGGTGCATAATGCCAAGACAAAGCCGCGGGAGGAGCAGTACAACAGCACGT |
| | ACCGTGTGGTCAGCGTCCTCACCGTCCTGCACCAGGACTGGCTGAATGGCAAGGAGTA |
| | CAAGTGCAAGGTCTCCAACAAAGCCCTCCCAGCCCCCATCGAGAAAACCATCTCCAAA |
| | GCCAAAGGGCAGCCCCGAGAACCACAGGTGTACACCCTGCCCCCATCCCGGGATGAGC |
| | TGACCAAGAACCAGGTCAGCCTGACCTGCCTGGTCAAAGGCTTCTATCCCAGCGACAT |
| | CGCCGTGGAGTGGGAGAGCAATGGGCAGCCGGAGAACAACTACAAGACCACGCCTCCC |
| | GTGCTGGACTCCGACGGCTCCTTCTTCCTCTACAGCAAGCTCACCGTGGACAAGAGCA |
| | GGTGGCAGCAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 31] |
| DNA for | GAGGTGCAGCTGGTGGAGTCTGGGGGAGGCTTGGTACAGCCAGGGCGGTCCCTGAGAC |
| Heavy | TCAGCTGTACAGCCTCTGGGTTTAGTTTTGGTGATCATGCTATGAGCTGGGTCCGCCA |
| Chain | GGCTCCAGGGAAGGGGCTGGAGTGGGTAGGTTTCATTAGAAAGACAACTTATGGTGCG |
| version 2 | ACAACACACTACGCCGCGGCTGTGAGAGGCAGATTCACCATCTCGCGAGATGATTCTA |
| | AAAGCATTGTCTATCTGCAAATGAACAGCCTGAAAACCGAGGACACAGCCGTGTATTT |
| | CTGTACTAGAGTGCAGCTTGACTATGGCCCGGGATACCAGTACTACGGTATGGACGTC |
| | TGGGGCCAAGGGACCACGGTCACCGTCTCCTCAGCCTCCACCAAGGGCCCATCGGTCT |
| | TCCCCCTGGCACCCTCCTCCAAGAGCACCTCTGGGGGCACAGCAGCCCTGGGCTGCCT |
| | GGTCAAGGACTACTTCCCCGAACCGGTGACGGTGTCGTGGAACTCAGGCGCCCTGACC |
| | AGCGGCGTGCACACCTTCCCGGCTGTCCTACAGTCCTCAGGACTCTACTCCCTCAGCA |
| | GCGTGGTGACCGTGCCCTCCAGCAGCTTGGGCACCCAGACCTACATCTGCAACGTGAA |
| | TCACAAGCCCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTCAGTCTTCC |
| | TCTTCCCCCCAAAACCCAAGGACACCCTCATGATCTCCCGGACCCCTGAGGTCACATG |
| | CGTGGTGGTGGACGTGAGCCACGAAGACCCTGAGGTCAAGTTCAACTGGTACGTGGAC |
| | GGCGTGGAGGTGCATAATGCCAAGACAAAGCCGCGGGAGGAGCAGTACAACAGCACGT |
| | ACCGTGTGGTCAGCGTCCTCACCGTCCTGCACCAGGACTGGCTGAATGGCAAGGAGTA |
| | CAAGTGCAAGGTCTCCAACAAAGCCCTCCCAGCCCCCATCGAGAAAACCATCTCCAAA |
| | GCCAAAGGGCAGCCCCGAGAACCACAGGTGTACACCCTGCCCCCATCCCGGGATGAGC |
| | TGACCAAGAACCAGGTCAGCCTGACCTGCCTGGTCAAAGGCTTCTATCCCAGCGACAT |
| | CGCCGTGGAGTGGGAGAGCAATGGGCAGCCGGAGAACAACTACAAGACCACGCCTCCC |
| | GTGCTGGACTCCGACGGCTCCTTCTTCCTCTACAGCAAGCTCACCGTGGACAAGAGCA |
| | GGTGGCAGCAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 32] |

In certain embodiments, the anti-CSP antibody variant comprises a light chain variable region comprising a CDR1, a CDR2, and a CDR3 and having the amino acid sequence set forth in SEQ ID NO: 33, as shown in Table 7. In certain embodiments, the anti-CSP antibody variant comprises a heavy chain variable region comprising a CDR1, a CDR2, and a CDR3 and having the amino acid sequence set forth in SEQ ID NO: 34, as shown in Table 75. In certain embodiments, the anti-CSP antibody variant comprises a light chain having the amino acid sequence set forth in SEQ ID NO: 37, as shown in Table 7. In certain embodiments, the anti-CSP antibody variant comprises a heavy chain having the amino acid sequence set forth in SEQ ID NO: 38, as shown in Table 7. In certain embodiments, the anti-CSP antibody variant comprises a heavy chain having the amino acid sequence set forth in SEQ ID NO: 39, as shown in Table 7. Exemplary nucleic acid sequences of SEQ ID NOS: 33, 34, 37, 38, and 39 are provided in Table 7 below.

TABLE 7
Antibody ID: AB-000224.002

| | KABAT | ASN |
|---|---|---|
| CDR1- | TGMNSNIGAGYDVY | TGMNSNIGAGYDVY |
| VL | [SEQ ID NO: 1] | [SEQ ID NO: 7] |
| CDR2- | GNSNRPS | GNSNRPS |
| VL | [SEQ ID NO: 2] | [SEQ ID NO: 8] |
| CDR3- | QSYDTSLNGWA | QSYDTSLNGWA |
| VL | [SEQ ID NO: 3] | [SEQ ID NO: 9] |
| CDR1- | DHAMS | DHAMS |
| VH | [SEQ ID NO: 4] | [SEQ ID NO: 10] |
| CDR2- | FIRKTTYGATTHYAAAVRG | FIRKTTYGATTHYAAAVRG |
| VH | [SEQ ID NO: 5] | [SEQ ID NO: 11] |
| CDR3- | VQLDYGPGYQYYGMDV | VQLDYGPGYQYYGMDV |
| VH | [SEQ ID NO: 6] | [SEQ ID NO: 12] |

| | |
|---|---|
| VL | QSVLTQPPSVSGAPGQRVTISCTGMNSNIGAGYDVYWYQQLPGTAPKLLIYGNSNRPSGVP |
| | DRFSGSRSGTSASLAITGLQAEDEADYYCQSYDTSLNGWAFGGGTKLTVLG [SEQ ID |
| | NO: 33] |
| VH | EVQLVESGGGLVQPGRSLRLSCAASGFSFGDHAMSWVRQAPGKGLEWVGFIRKTTYGATTH |
| | YAAAVRGRFTISRDDSKSIVYLQMNSLKTEDTAVYFCTRVQLDYGPGYQYYGMDVWGQGTT |
| | VTVSS [SEQ ID NO: 34] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC [SEQ ID NO: 35] |
| DNA | GAGGTGCAGCTGGTGGAGTCTGGGGGAGGCTTGG |
| for VH | TACAGCCAGGGCGGTCCCTGAGACTCAGCTGTGCGGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGCATTGTCTATCTGCAAATGAACAGCCTGAAAACCGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 36] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 37] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | SIVYLQMNSLKTEDTAVYFCTRVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK [SEQ ID NO: 38] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | SIVYLQMNSLKTEDTAVYFCTRVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK [SEQ ID NO: 39] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT [SEQ ID NO: 40] |
| DNA | GAGGTGCAGCTGGTGGAGTCTGGGGGAGGCTTGGTACAGCCAGGGCGGTCCCTGAGACTCA |
| for | GCTGTGCGGCCTCTGGGTTTAGTTTTGGTGATCATGCTATGAGCTGGGTCCGCCAGGCTCC |
| Heavy | AGGGAAGGGGCTGGAGTGGGTAGGTTTCATTAGAAAGACAACTTATGGTGCGACAACACAC |
| Chain | TACGCCGCGGCTGTGAGAGGCAGATTCACCATCTCGCGAGATGATTCTAAAAGCATTGTCT |
| version | ATCTGCAAATGAACAGCCTGAAAACCGAGGACACAGCCGTGTATTTCTGTACTAGAGTGCA |
| 1 | GCTTGACTATGGCCCGGGATACCAGTACTACGGTATGGACGTCTGGGGCCAAGGGACCACG |
| | GTCACCGTCTCCTCAGCCTCCACCAAGGGCCCATCGGTCTTCCCCCTGGCACCCTCCTCCA |
| | AGAGCACCTCTGGGGGCACAGCAGCCCTGGGCTGCCTGGTCAAGGACTACTTCCCCGAACC |
| | GGTGACGGTGTCGTGGAACTCAGGCGCCCTGACCAGCGGCGTGCACACCTTCCCGGCTGTC |
| | CTACAGTCCTCAGGACTCTACTCCCTCAGCAGCGTGGTGACCGTGCCCTCCAGCAGCTTGG |
| | GCACCCAGACCTACATCTGCAACGTGAATCACAAGCCCAGCAACACCAAGGTGGACAAGAA |
| | AGTTGAGCCCAAATCTTGTGACAAAACTCACACATGCCCACCGTGCCCAGCACCTGAACTC |
| | CTGGGGGGACCGTCAGTCTTCCTCTTCCCCCCAAAACCCAAGGACACCCTCATGATCTCCC |
| | GGACCCCTGAGGTCACATGCGTGGTGGTGGACGTGAGCCACGAAGACCCTGAGGTCAAGTT |
| | CAACTGGTACGTGGACGGCGTGGAGGTGCATAATGCCAAGACAAAGCCGCGGGAGGAGCAG |
| | TACAACAGCACGTACCGTGTGGTCAGCGTCCTCACCGTCCTGCACCAGGACTGGCTGAATG |
| | GCAAGGAGTACAAGTGCAAGGTCTCCAACAAAGCCCTCCCAGCCCCCATCGAGAAAACCAT |
| | CTCCAAAGCCAAAGGGCAGCCCCGAGAACCACAGGTGTACACCCTGCCCCCATCCCGGGAT |
| | GAGCTGACCAAGAACCAGGTCAGCCTGACCTGCCTGGTCAAAGGCTTCTATCCCAGCGACA |
| | TCGCCGTGGAGTGGGAGAGCAATGGGCAGCCGGAGAACAACTACAAGACCACGCCTCCCGT |
| | GCTGGACTCCGACGGCTCCTTCTTCCTCTACAGCAAGCTCACCGTGGACAAGAGCAGGTGG |
| | CAGCAGGGGAACGTCTTCTCATGCTCCGTGATGCATGAGGCTCTGCACAACCACTACACAC |
| | AGAAGAGCCTCTCCCTGTCTCCGGGTAAA [SEQ ID NO: 41] |
| DNA | GAGGTGCAGCTGGTGGAGTCTGGGGGAGGCTTGGTACAGCCAGGGCGGTCCCTGAGACTCA |
| for | GCTGTGCGGCCTCTGGGTTTAGTTTTGGTGATCATGCTATGAGCTGGGTCCGCCAGGCTCC |
| Heavy | AGGGAAGGGGCTGGAGTGGGTAGGTTTCATTAGAAAGACAACTTATGGTGCGACAACACAC |
| Chain | TACGCCGCGGCTGTGAGAGGCAGATTCACCATCTCGCGAGATGATTCTAAAAGCATTGTCT |
| version | ATCTGCAAATGAACAGCCTGAAAACCGAGGACACAGCCGTGTATTTCTGTACTAGAGTGCA |
| 1 | GCTTGACTATGGCCCGGGATACCAGTACTACGGTATGGACGTCTGGGGCCAAGGGACCACG |
| | GTCACCGTCTCCTCAGCCTCCACCAAGGGCCCATCGGTCTTCCCCCTGGCACCCTCCTCCA |
| | AGAGCACCTCTGGGGGCACAGCAGCCCTGGGCTGCCTGGTCAAGGACTACTTCCCCGAACC |
| | GGTGACGGTGTCGTGGAACTCAGGCGCCCTGACCAGCGGCGTGCACACCTTCCCGGCTGTC |
| | CTACAGTCCTCAGGACTCTACTCCCTCAGCAGCGTGGTGACCGTGCCCTCCAGCAGCTTGG |
| | GCACCCAGACCTACATCTGCAACGTGAATCACAAGCCCAGCAACACCAAGGTGGACAAGAA |
| | AGTTGAGCCCAAATCTTGTGACAAAACTCACACATGCCCACCGTGCCCAGCACCTGAACTC |
| | CTGGGGGGACCGTCAGTCTTCCTCTTCCCCCCAAAACCCAAGGACACCCTCATGATCTCCC |
| | GGACCCCTGAGGTCACATGCGTGGTGGTGGACGTGAGCCACGAAGACCCTGAGGTCAAGTT |
| | CAACTGGTACGTGGACGGCGTGGAGGTGCATAATGCCAAGACAAAGCCGCGGGAGGAGCAG |
| | TACAACAGCACGTACCGTGTGGTCAGCGTCCTCACCGTCCTGCACCAGGACTGGCTGAATG |
| | GCAAGGAGTACAAGTGCAAGGTCTCCAACAAAGCCCTCCCAGCCCCCATCGAGAAAACCAT |
| | CTCCAAAGCCAAAGGGCAGCCCCGAGAACCACAGGTGTACACCCTGCCCCCATCCCGGGAT |
| | GAGCTGACCAAGAACCAGGTCAGCCTGACCTGCCTGGTCAAAGGCTTCTATCCCAGCGACA |
| | TCGCCGTGGAGTGGGAGAGCAATGGGCAGCCGGAGAACAACTACAAGACCACGCCTCCCGT |
| | GCTGGACTCCGACGGCTCCTTCTTCCTCTACAGCAAGCTCACCGTGGACAAGAGCAGGTGG |
| | CAGCAGGGGAACGTCTTCTCATGCTCCGTGCTGCATGAGGCTCTGCACTCCCACTACACAC |
| | AGAAGAGCCTCTCCCTGTCTCCGGGTAAA [SEQ ID NO: 42] |

In certain embodiments, the anti-CSP antibody variant comprises a light chain variable region comprising a CDR1, a CDR2, and a CDR3 and having the amino acid sequence set forth in SEQ ID NO: 43, as shown in Table 8. In certain embodiments, the anti-CSP antibody variant comprises a heavy chain variable region comprising a CDR1, a CDR2, and a CDR3 and having the amino acid sequence set forth in SEQ ID NO: 44, as shown in Table 8. In certain embodiments, the anti-CSP antibody variant comprises a light chain having the amino acid sequence set forth in SEQ ID NO: 47, as shown in Table 8. In certain embodiments, the anti-CSP antibody variant comprises a heavy chain having the amino acid sequence set forth in SEQ ID NO: 48, as shown in Table 8. In certain embodiments, the anti-CSP antibody variant comprises a heavy chain having the amino acid sequence set forth in SEQ ID NO: 49, as shown in Table 8. Exemplary nucleic acid sequences of SEQ ID NOS: 43, 44, 47, 48, and 49 are provided in Table 8 below.

TABLE 8
Antibody ID: AB-000224.003

| | KABAT | ASN |
|---|---|---|
| CDR1- | TGMNSNIGAGYDVY [SEQ ID NO: | TGMNSNIGAGYDVY [SEQ ID NO: 7] |
| VL | 1] | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG [SEQ ID | FIRKTTYGATTHYAAAVRG [SEQ ID NO: |
| VH | NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG [SEQ ID NO: 43] |
| VH | EVQLVESGGGLVQPGRSLRLSCTASGFS |
| | FGDHAMSWVRQAPGKGLEWVGFIRKTTYGATTHYAAAVRGRFTISRDDSK |
| | STVYLQMNSLKTEDTAVYFCTRVQLDYGPGYQYYGMDVWGQGTTVTVSS [SEQ ID NO: |
| | 44] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC [SEQ ID NO: 45] |
| DNA | GAGGTGCAGCTGGTGGAGTCTGGGGGAGGCTTGG |
| for VH | TACAGCCAGGGCGGTCCCTGAGACTCAGCTGTACAGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGCACTGTCTATCTGCAAATGAACAGCCTGAAAACCGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCA [SEQ ID NO: |
| | 46] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS [SEQ ID NO: 47] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | STVYLQMNSLKTEDTAVYFCTRVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRWSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK [SEQ ID NO: 48] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | STVYLQMNSLKTEDTAVYFCTRVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK [SEQ ID NO: 49] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT [SEQ ID NO: 50] |
| DNA | GAGGTGCAGCTGGTGGAGTCTGGGGGAGGCTTGGTACAGCCAGGGCGGTCCCTGAGACTCA |
| for | GCTGTACAGCCTCTGGGTTTAGTTTTGGTGATCATGCTATGAGCTGGGTCCGCCAGGCTCC |
| Heavy | AGGGAAGGGGCTGGAGTGGGTAGGTTTCATTAGAAAGACAACTTATGGTGCGACAACACAC |
| Chain | TACGCCGCGGCTGTGAGAGGCAGATTCACCATCTCGCGAGATGATTCTAAAAGCACTGTCT |
| version | ATCTGCAAATGAACAGCCTGAAAACCGAGGACACAGCCGTGTATTTCTGTACTAGAGTGCA |
| 1 | GCTTGACTATGGCCCGGGATACCAGTACTACGGTATGGACGTCTGGGGCCAAGGGACCACG |
| | GTCACCGTCTCCTCAGCCTCCACCAAGGGCCCATCGGTCTTCCCCCTGGCACCCTCCTCCA |
| | AGAGCACCTCTGGGGGCACAGCAGCCCTGGGCTGCCTGGTCAAGGACTACTTCCCCGAACC |
| | GGTGACGGTGTCGTGGAACTCAGGCGCCCTGACCAGCGGCGTGCACACCTTCCCGGCTGTC |
| | CTACAGTCCTCAGGACTCTACTCCCTCAGCAGCGTGGTGACCGTGCCCTCCAGCAGCTTGG |
| | GCACCCAGACCTACATCTGCAACGTGAATCACAAGCCCAGCAACACCAAGGTGGACAAGAA |
| | AGTTGAGCCCAAATCTTGTGACAAAACTCACACATGCCCACCGTGCCCAGCACCTGAACTC |
| | CTGGGGGGACCGTCAGTCTTCCTCTTCCCCCCAAAACCCAAGGACACCCTCATGATCTCCC |
| | GGACCCCTGAGGTCACATGCGTGGTGGTGGACGTGAGCCACGAAGACCCTGAGGTCAAGTT |
| | CAACTGGTACGTGGACGGCGTGGAGGTGCATAATGCCAAGACAAAGCCGCGGGAGGAGCAG |
| | TACAACAGCACGTACCGTGTGGTCAGCGTCCTCACCGTCCTGCACCAGGACTGGCTGAATG |
| | GCAAGGAGTACAAGTGCAAGGTCTCCAACAAAGCCCTCCCAGCCCCCATCGAGAAAACCAT |
| | CTCCAAAGCCAAAGGGCAGCCCCGAGAACCACAGGTGTACACCCTGCCCCCATCCCGGGAT |
| | GAGCTGACCAAGAACCAGGTCAGCCTGACCTGCCTGGTCAAAGGCTTCTATCCCAGCGACA |
| | TCGCCGTGGAGTGGGAGAGCAATGGGCAGCCGGAGAACAACTACAAGACCACGCCTCCCGT |
| | GCTGGACTCCGACGGCTCCTTCTTCCTCTACAGCAAGCTCACCGTGGACAAGAGCAGGTGG |
| | CAGCAGGGGAACGTCTTCTCATGCTCCGTGATGCATGAGGCTCTGCACAACCACTACACAC |
| | AGAAGAGCCTCTCCCTGTCTCCGGGTAAA [SEQ ID NO: 51] |
| DNA | GAGGTGCAGCTGGTGGAGTCTGGGGGAGGCTTGGTACAGCCAGGGCGGTCCCTGAGACTCA |
| for | GCTGTACAGCCTCTGGGTTTAGTTTTGGTGATCATGCTATGAGCTGGGTCCGCCAGGCTCC |
| Heavy | AGGGAAGGGGCTGGAGTGGGTAGGTTTCATTAGAAAGACAACTTATGGTGCGACAACACAC |
| Chain | TACGCCGCGGCTGTGAGAGGCAGATTCACCATCTCGCGAGATGATTCTAAAAGCACTGTCT |
| version | ATCTGCAAATGAACAGCCTGAAAACCGAGGACACAGCCGTGTATTTCTGTACTAGAGTGCA |
| 2 | GCTTGACTATGGCCCGGGATACCAGTACTACGGTATGGACGTCTGGGGCCAAGGGACCACG |
| | GTCACCGTCTCCTCAGCCTCCACCAAGGGCCCATCGGTCTTCCCCCTGGCACCCTCCTCCA |
| | AGAGCACCTCTGGGGGCACAGCAGCCCTGGGCTGCCTGGTCAAGGACTACTTCCCCGAACC |
| | GGTGACGGTGTCGTGGAACTCAGGCGCCCTGACCAGCGGCGTGCACACCTTCCCGGCTGTC |
| | CTACAGTCCTCAGGACTCTACTCCCTCAGCAGCGTGGTGACCGTGCCCTCCAGCAGCTTGG |
| | GCACCCAGACCTACATCTGCAACGTGAATCACAAGCCCAGCAACACCAAGGTGGACAAGAA |
| | AGTTGAGCCCAAATCTTGTGACAAAACTCACACATGCCCACCGTGCCCAGCACCTGAACTC |
| | CTGGGGGGACCGTCAGTCTTCCTCTTCCCCCCAAAACCCAAGGACACCCTCATGATCTCCC |
| | GGACCCCTGAGGTCACATGCGTGGTGGTGGACGTGAGCCACGAAGACCCTGAGGTCAAGTT |
| | CAACTGGTACGTGGACGGCGTGGAGGTGCATAATGCCAAGACAAAGCCGCGGGAGGAGCAG |
| | TACAACAGCACGTACCGTGTGGTCAGCGTCCTCACCGTCCTGCACCAGGACTGGCTGAATG |
| | GCAAGGAGTACAAGTGCAAGGTCTCCAACAAAGCCCTCCCAGCCCCCATCGAGAAAACCAT |
| | CTCCAAAGCCAAAGGGCAGCCCCGAGAACCACAGGTGTACACCCTGCCCCCATCCCGGGAT |
| | GAGCTGACCAAGAACCAGGTCAGCCTGACCTGCCTGGTCAAAGGCTTCTATCCCAGCGACA |
| | TCGCCGTGGAGTGGGAGAGCAATGGGCAGCCGGAGAACAACTACAAGACCACGCCTCCCGT |
| | GCTGGACTCCGACGGCTCCTTCTTCCTCTACAGCAAGCTCACCGTGGACAAGAGCAGGTGG |
| | CAGCAGGGGAACGTCTTCTCATGCTCCGTGCTGCATGAGGCTCTGCACTCCCACTACACAC |
| | AGAAGAGCCTCTCCCTGTCTCCGGGTAAA [SEQ ID NO: 52] |

In certain embodiments, the anti-CSP antibody variant comprises a light chain variable region comprising a CDR1, a CDR2, and a CDR3 and having the amino acid sequence set forth in SEQ ID NO: 53, as shown in Table 9. In certain embodiments, the anti-CSP antibody variant comprises a heavy chain variable region comprising a CDR1, a CDR2, and a CDR3 and having the amino acid sequence set forth in SEQ ID NO: 55, as shown in Table 9. In certain embodiments, the anti-CSP antibody variant comprises a light chain having the amino acid sequence set forth in SEQ ID NO: 57, as shown in Table 9. In certain embodiments, the anti-CSP antibody variant comprises a heavy chain having the amino acid sequence set forth in SEQ ID NO: 58, as shown in Table 9. In certain embodiments, the anti-CSP antibody variant comprises a heavy chain having the amino acid sequence set forth in SEQ ID NO: 59, as shown in Table 9. Exemplary nucleic acid sequences of SEQ ID NOS: 53, 54, 57, 58, and 59 are provided in Table 9 below.

TABLE 9
Antibody ID: AB-000224.004

| | KABAT | ASN |
|---|---|---|
| CDR1- | TGMNSNIGAGYDVY [SEQ ID NO: | TGMNSNIGAGYDVY [SEQ ID NO: 7] |
| VL | 1] | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG [SEQ ID | FIRKTTYGATTHYAAAVRG [SEQ ID NO: |
| VH | NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGAGYDVYWYQQLPGTAPKLLIYGNSNRPSGVP |
| | DRFSGSRSGTSASLAITGLQAEDEADYYCQSYDTSLNGWAFGGGTKLTVLG [SEQ ID |
| | NO: 53] |
| VH | EVQLVESGGGLVQPGRSLRLSCTASGFSFGDHAMSWVRQAPGKGLEWVGFIRKTTYGATTH |
| | YAAAVRGRFTISRDDSKSIVYLQMNSLKAEDTAVYFCTRVQLDYGPGYQYYGMDVWGQGTT |
| | VTVSS [SEQ ID NO: 54] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC [SEQ ID NO: 55] |
| DNA | GAGGTGCAGCTGGTGGAGTCTGGGGGAGGCTTGG |
| for VH | TACAGCCAGGGCGGTCCCTGAGACTCAGCTGTACAGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGOATTGTOTATCTGCAAATGAACAGCCTGAAAGCAGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCA [SEQ ID NO: |
| | 56] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS [SEQ ID NO: 57] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | SIVYLQMNSLKAEDTAVYFCTRVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK [SEQ ID NO: 58] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | SIVYLQMNSLKAEDTAVYFCTRVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK [SEQ ID NO: 59] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT [SEQ ID NO: 60] |
| DNA | GAGGTGCAGCTGGTGGAGTCTGGGGGAGGCTTGG |
| for | TACAGCCAGGGCGGTCCCTGAGACTCAGCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGCATTGTCTATCTGCAAATGAACAGCCTGAAAGCAGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 61] |
| DNA | GAGGTGCAGCTGGTGGAGTCTGGGGGAGGCTTGG |
| for | TACAGCCAGGGCGGTCCCTGAGACTCAGCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 2 | AGCATTGTCTATGTGCAAATGAACAGCCTGAAAGCAGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 62] |

In certain embodiments, the anti-CSP antibody variant comprises a light chain variable region comprising a CDR1, a CDR2, and a CDR3 and having the amino acid sequence set forth in SEQ ID NO: 63, as shown in Table 10. In certain embodiments, the anti-CSP antibody variant comprises a heavy chain variable region comprising a CDR1, a CDR2, and a CDR3 and having the amino acid sequence set forth in SEQ ID NO: 64, as shown in Table 10. In certain embodiments, the anti-CSP antibody variant comprises a light chain having the amino acid sequence set forth in SEQ ID NO: 67, as shown in Table 10. In certain embodiments, the anti-CSP antibody variant comprises a heavy chain having the amino acid sequence set forth in SEQ ID NO: 68, as shown in Table 10. In certain embodiments, the anti-CSP antibody variant comprises a heavy chain having the amino acid sequence set forth in SEQ ID NO: 69, as shown in Table 10. Exemplary nucleic acid sequences of SEQ ID NOS: 63, 64, 67, 68, and 69 are provided in Table 10 below.

TABLE 10
Antibody ID: AB-000224.005

| | KABAT | ASN |
|---|---|---|
| CDR1- | TGMNSNIGAGYDVY [SEQ ID NO: | TGMNSNIGAGYDVY [SEQ ID NO: 7] |
| VL | 1] | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG [SEQ ID | FIRKTTYGATTHYAAAVRG [SEQ ID NO: |
| VH | NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG |
| | [SEQ ID NO: 63] |
| VH | EVQLVESGGGLVQPGRSLRLSCTASGFS |
| | FGDHAMSWVRQAPGKGLEWVGFIRKTTYGATTHYAAAVRGRFTISRDDSK |
| | STVYLQMNSLKTEDTAVYFCARVQLDYGPGYQYYGMDVWGQGTTVTVSS |
| | [SEQ ID NO: 64] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC |
| | [SEQ ID NO: 65] |
| DNA | GAGGTGCAGCTGGTGGAGTCTGGGGGAGGCTTGG |
| for VH | TACAGCCAGGGCGGTCCCTGAGACTCAGCTGTACAGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGCATTGTCTATCTGCAAATGAACAGCCTGAAAACCGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 66] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 67] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | SIVYLQMNSLKTEDTAVYFCARVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 68] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | SIVYLQMNSLKTEDTAVYFCARVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK [SEQ ID NO: 69] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 70] |
| DNA | GAGGTGCAGCTGGTGGAGTCTGGGGGAGGCTTGG |
| for | TACAGCCAGGGCGGTCCCTGAGACTCAGCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGCATTGTCTATCTGCAAATGAACAGCCTGAAAACCGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 71] |
| DNA | GAGGTGCAGCTGGTGGAGTCTGGGGGAGGCTTGG |
| for | TACAGCCAGGGCGGTCCCTGAGACTCAGCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 2 | AGCATTGTCTATCTGCAAATGAACAGCCTGAAAACCGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 72] |

In certain embodiments, the anti-CSP antibody variant comprises a light chain variable region comprising a CDR1, a CDR2, and a CDR3 and having the amino acid sequence set forth in SEQ ID NO: 73, as shown in Table 11. In certain embodiments, the anti-CSP antibody variant comprises a heavy chain variable region comprising a CDR1, a CDR2, and a CDR3 and having the amino acid sequence set forth in SEQ ID NO: 74, as shown in Table 11. In certain embodiments, the anti-CSP antibody variant comprises a light chain having the amino acid sequence set forth in SEQ ID NO: 77, as shown in Table 11. In certain embodiments, the anti-CSP antibody variant comprises a heavy chain having the amino acid sequence set forth in SEQ ID NO: 78, as shown in Table 11. In certain embodiments, the anti-CSP antibody variant comprises a heavy chain having the amino acid sequence set forth in SEQ ID NO: 79, as shown in Table 11. Exemplary nucleic acid sequences of SEQ ID NOS: 73, 74, 77, 78, and 79 are provided in Table 11 below.

TABLE 11
Antibody ID: AB-000224.006

| | KABAT | ASN |
|---|---|---|
| CDR1- | TGMNSNIGAGYDVY [SEQ ID NO: | TGMNSNIGAGYDVY [SEQ ID NO: 7] |
| VL | 1] | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG [SEQ ID | FIRKTTYGATTHYAAAVRG [SEQ ID NO: |
| VH | NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG |
| | [SEQ ID NO: 73] |
| VH | EVQLVESGGGLVQPGRSLRLSCAASGFS |
| | FGDHAMSWVRQAPGKGLEWVGFIRKTTYGATTHYAAAVRGRFTISRDDSK |
| | STVYLQMNSLKTEDTAVYFCTRVQLDYGPGYQYYGMDVWGQGTTVTVSS |
| | [SEQ ID NO: 74] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC |
| | [SEQ ID NO: 75] |
| DNA | GAGGTGCAGCTGGTGGAGTCTGGGGGAGGCTTGG |
| for VH | TACAGCCAGGGCGGTCCCTGAGACTCAGCTGTGCGGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGCACTGTCTATCTGCAAATGAACAGCCTGAAAACCGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 76] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 77] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | STVYLQMNSLKTEDTAVYFCTRVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 78] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | STVYLQMNSLKTEDTAVYFCTRVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK |
| | [SEQ ID NO: 79] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 80] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGCACTGTCTATCTGCAAATGAACAGCCTGAAAACCGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 81] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 2 | AGCACTGTCTATCTGCAAATGAACAGCCTGAAAACCGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 82] |

In certain embodiments, the anti-CSP antibody variant comprises a light chain variable region comprising a CDR1, a CDR2, and a CDR3 and having the amino acid sequence set forth in SEQ ID NO: 83, as shown in Table 12. In certain embodiments, the anti-CSP antibody variant comprises a heavy chain variable region comprising a CDR1, a CDR2, and a CDR3 and having the amino acid sequence set forth in SEQ ID NO: 84, as shown in Table 12. In certain embodiments, the anti-CSP antibody variant comprises a light chain having the amino acid sequence set forth in SEQ ID NO: 87, as shown in Table 12. In certain embodiments, the anti-CSP antibody variant comprises a heavy chain having the amino acid sequence set forth in SEQ ID NO: 88, as shown in Table 12. In certain embodiments, the anti-CSP antibody variant comprises a heavy chain having the amino acid sequence set forth in SEQ ID NO: 89, as shown in Table 12. Exemplary nucleic acid sequences of SEQ ID NOS: 83, 84, 87, 88, and 89 are provided in Table 12 below.

TABLE 12
Antibody ID: AB-000224.007

| | KABAT | ASN |
|---|---|---|
| CDR1- | TGMNSNIGAGYDVY [SEQ ID NO: | TGMNSNIGAGYDVY [SEQ ID NO: 7] |
| VL | 1] | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG [SEQ ID | FIRKTTYGATTHYAAAVRG [SEQ ID NO: |
| VH | NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG |
| | [SEQ ID NO: 83] |
| VH | EVQLVESGGGLVQPGRSLRLSCAASGFS |
| | FGDHAMSWVRQAPGKGLEWVGFIRKTTYGATTHYAAAVRGRFTISRDDSK |
| | SIVYLQMNSLKAEDTAVYFCTRVQLDYGPGYQYYGMDVWGQGTTVTVSS |
| | [SEQ ID NO: 84] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC |
| | [SEQ ID NO: 85] |
| DNA | GAGGTGCAGCTGGTGGAGTCTGGGGGAGGCTTGG |
| for VH | TACAGCCAGGGCGGTCCCTGAGACTCAGCTGTGCGGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGOATTGTOTATCTGCAAATGAACAGCCTGAAAGCAGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 86] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 87] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | SIVYLQMNSLKAEDTAVYFCTRVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 88] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | SIVYLQMNSLKAEDTAVYFCTRVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK [SEQ ID NO: 89] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 90] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGGATTGTCTATGTGCAAATGAACAGCCTGAAAGCAGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 91] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 2 | AGCATTGTCTATCTGCAAATGAACAGCCTGAAAGCAGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 92] |

In certain embodiments, the anti-CSP antibody variant comprises a light chain variable region comprising a CDR1, a CDR2, and a CDR3 and having the amino acid sequence set forth in SEQ ID NO: 93, as shown in Table 13. In certain embodiments, the anti-CSP antibody variant comprises a heavy chain variable region comprising a CDR1, a CDR2, and a CDR3 and having the amino acid sequence set forth in SEQ ID NO: 94, as shown in Table 13. In certain embodiments, the anti-CSP antibody variant comprises a light chain having the amino acid sequence set forth in SEQ ID NO: 97, as shown in Table 13. In certain embodiments, the anti-CSP antibody variant comprises a heavy chain having the amino acid sequence set forth in SEQ ID NO: 98, as shown in Table 13. In certain embodiments, the anti-CSP antibody variant comprises a heavy chain having the amino acid sequence set forth in SEQ ID NO: 99, as shown in Table 13. Exemplary nucleic acid sequences of SEQ ID NOS: 93, 94, 97, 98, and 99 are provided in Table 13 below.

TABLE 13
Antibody ID: AB-000224.008

| | KABAT | ASN |
|---|---|---|
| CDR1- | TGMNSNIGAGYDVY [SEQ ID NO: | TGMNSNIGAGYDVY [SEQ ID NO: 7] |
| VL | 1] | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG [SEQ ID | FIRKTTYGATTHYAAAVRG [SEQ ID NO: |
| VH | NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG |
| | [SEQ ID NO: 93] |
| VH | EVQLVESGGGLVQPGRSLRLSCAASGFS |
| | FGDHAMSWVRQAPGKGLEWVGFIRKTTYGATTHYAAAVRGRFTISRDDSK |
| | SIVYLQMNSLKTEDTAVYFCARVQLDYGPGYQYYGMDVWGQGTTVTVSS |
| | [SEQ ID NO: 94] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC |
| | [SEQ ID NO: 95] |
| DNA | GAGGTGCAGCTGGTGGAGTCTGGGGGAGGCTTGG |
| for VH | TACAGCCAGGGCGGTCCCTGAGACTCAGCTGTGCGGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGCATTGTCTATCTGCAAATGAACAGCCTGAAAACCGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 96] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 97] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | SIVYLQMNSLKTEDTAVYFCARVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 98] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | SIVYLQMNSLKTEDTAVYFCARVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK [SEQ ID NO: 99] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 100] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGCATTGTCTATCTGCAAATGAACAGCCTGAAAACCGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 101] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 2 | AGCATTGTCTATCTGCAAATGAACAGCCTGAAAACCGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 102] |

In certain embodiments, the anti-CSP antibody variant comprises a light chain variable region comprising a CDR1, a CDR2, and a CDR3 and having the amino acid sequence set forth in SEQ ID NO: 103, as shown in Table 14. In certain embodiments, the anti-CSP antibody variant comprises a heavy chain variable region comprising a CDR1, a CDR2, and a CDR3 and having the amino acid sequence set forth in SEQ ID NO: 104, as shown in Table 14. In certain embodiments, the anti-CSP antibody variant comprises a light chain having the amino acid sequence set forth in SEQ ID NO: 107, as shown in Table 14. In certain embodiments, the anti-CSP antibody variant comprises a heavy chain having the amino acid sequence set forth in SEQ ID NO: 108, as shown in Table 14. In certain embodiments, the anti-CSP antibody variant comprises a heavy chain having the amino acid sequence set forth in SEQ ID NO: 109, as shown in Table 14. Exemplary nucleic acid sequences of SEQ ID NOS: 103, 104, 107, 108, and 109 are provided in Table 14 below.

TABLE 14
Antibody ID: AB-000224.009

| | KABAT | ASN |
|---|---|---|
| CDR1- | TGMNSNIGAGYDVY [SEQ ID NO: 1] | TGMNSNIGAGYDVY [SEQ ID NO: 7] |
| VL | | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG | FIRKTTYGATTHYAAAVRG [SEQ ID NO: |
| VH | [SEQ ID NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG |
| | [SEQ ID NO: 103] |
| VH | EVQLVESGGGLVQPGRSLRLSCTASGFS |
| | FGDHAMSWVRQAPGKGLEWVGFIRKTTYGATTHYAAAVRGRFTISRDDSK |
| | STVYLQMNSLKAEDTAVYFCTRVQLDYGPGYQYYGMDVWGQGTTVTVSS |
| | [SEQ ID NO: 104] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC |
| | [SEQ ID NO: 105] |
| DNA | GAGGTGCAGCTGGTGGAGTCTGGGGGAGGCTTGG |
| for VH | TACAGCCAGGGCGGTCCCTGAGACTCAGCTGTACAGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGCACTGTCTATCTGCAAATGAACAGCCTGAAAGCAGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 106] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 107] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | STVYLQMNSLKAEDTAVYFCTRVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 108] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | STVYLQMNSLKAEDTAVYFCTRVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK [SEQ ID NO: 109] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 110] |
| DNA | GAGGTGCAGCTGGTGGAGTCTGGGGGAGGCTTGG |
| for | TACAGCCAGGGCGGTCCCTGAGACTCAGCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGCACTGTCTATCTGCAAATGAACAGCCTGAAAGCAGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 111] |
| DNA | GAGGTGCAGCTGGTGGAGTCTGGGGGAGGCTTGG |
| for | TACAGCCAGGGCGGTCCCTGAGACTCAGCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 2 | AGCACTGTCTATCTGCAAATGAACAGCCTGAAAGCAGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 112] |

In certain embodiments, the anti-CSP antibody variant comprises a light chain variable region comprising a CDR1, a CDR2, and a CDR3 and having the amino acid sequence set forth in SEQ ID NO: 113, as shown in Table 15. In certain embodiments, the anti-CSP antibody variant comprises a heavy chain variable region comprising a CDR1, a CDR2, and a CDR3 and having the amino acid sequence set forth in SEQ ID NO: 114, as shown in Table 15. In certain embodiments, the anti-CSP antibody variant comprises a light chain having the amino acid sequence set forth in SEQ ID NO: 117, as shown in Table 15. In certain embodiments, the anti-CSP antibody variant comprises a heavy chain having the amino acid sequence set forth in SEQ ID NO: 118, as shown in Table 15. In certain embodiments, the anti-CSP antibody variant comprises a heavy chain having the amino acid sequence set forth in SEQ ID NO: 119, as shown in Table 15. Exemplary nucleic acid sequences of SEQ ID NOS: 113, 114, 117, 118, and 119 are provided in Table 15 below.

TABLE 15
Antibody ID: AB-000224.010

| | KABAT | ASN |
|---|---|---|
| CDR1- | TGMNSNIGAGYDVY [SEQ ID NO: 1] | TGMNSNIGAGYDVY [SEQ ID NO: 7] |
| VL | | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG [SEQ ID | FIRKTTYGATTHYAAAVRG [SEQ ID NO: |
| VH | NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG |
| | [SEQ ID NO: 113] |
| VH | EVQLVESGGGLVQPGRSLRLSCTASGFS |
| | FGDHAMSWVRQAPGKGLEWVGFIRKTTYGATTHYAAAVRGRFTISRDDSK |
| | STVYLQMNSLKTEDTAVYFCARVQLDYGPGYQYYGMDVWGQGTTVTVSS |
| | [SEQ ID NO: 114] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC |
| | [SEQ ID NO: 115] |
| DNA | GAGGTGCAGCTGGTGGAGTCTGGGGGAGGCTTGG |
| for VH | TACAGCCAGGGCGGTCCCTGAGACTCAGCTGTACAGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGCACTGTCTATCTGCAAATGAACAGCCTGAAAACCGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 116] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 117] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | STVYLQMNSLKTEDTAVYFCARVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 118] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | STVYLQMNSLKTEDTAVYFCARVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK [SEQ ID NO: 119] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 120] |
| DNA | GAGGTGCAGCTGGTGGAGTCTGGGGGAGGCTTGG |
| for | TACAGCCAGGGCGGTCCCTGAGACTCAGCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGCACTGTCTATCTGCAAATGAACAGCCTGAAAACCGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 121] |
| DNA | GAGGTGCAGCTGGTGGAGTCTGGGGGAGGCTTGG |
| for | TACAGCCAGGGCGGTCCCTGAGACTCAGCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 2 | AGCACTGTCTATCTGCAAATGAACAGCCTGAAAACCGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 122] |

In certain embodiments, the anti-CSP antibody variant comprises a light chain variable region comprising a CDR1, a CDR2, and a CDR3 and having the amino acid sequence set forth in SEQ ID NO: 123, as shown in Table 16. In certain embodiments, the anti-CSP antibody variant comprises a heavy chain variable region comprising a CDR1, a CDR2, and a CDR3 and having the amino acid sequence set forth in SEQ ID NO: 124, as shown in Table 16. In certain embodiments, the anti-CSP antibody variant comprises a light chain having the amino acid sequence set forth in SEQ ID NO: 127, as shown in Table 16. In certain embodiments, the anti-CSP antibody variant comprises a heavy chain having the amino acid sequence set forth in SEQ ID NO: 128, as shown in Table 16. In certain embodiments, the anti-CSP antibody variant comprises a heavy chain having the amino acid sequence set forth in SEQ ID NO: 129, as shown in Table 16. Exemplary nucleic acid sequences of SEQ ID NOS: 123, 124, 127, 128, and 129 are provided in Table 16 below.

TABLE 16
Antibody ID: AB-000224.011

| | KABAT | ASN |
|---|---|---|
| CDR1- | TGMNSNIGAGYDVY [SEQ ID NO: 1] | TGMNSNIGAGYDVY [SEQ ID NO: 7] |
| VL | | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG [SEQ ID | FIRKTTYGATTHYAAAVRG [SEQ ID NO: |
| VH | NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG |
| | [SEQ ID NO: 123] |
| VH | EVQLVESGGGLVQPGRSLRLSCTASGFS |
| | FGDHAMSWVRQAPGKGLEWVGFIRKTTYGATTHYAAAVRGRFTISRDDSK |
| | SIVYLQMNSLKAEDTAVYFCARVQLDYGPGYQYYGMDVWGQGTTVTVSS |
| | [SEQ ID NO: 124] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC |
| | [SEQ ID NO: 125] |
| DNA | GAGGTGCAGCTGGTGGAGTCTGGGGGAGGCTTGG |
| for VH | TACAGCCAGGGCGGTCCCTGAGACTCAGCTGTACAGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGCATTGTCTATCTGCAAATGAACAGCCTGAAAGCAGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 126] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 127] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | SIVYLQMNSLKAEDTAVYFCARVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 128] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | SIVYLQMNSLKAEDTAVYFCARVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK |
| | [SEQ ID NO: 129] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 130] |
| DNA | GAGGTGCAGCTGGTGGAGTCTGGGGGAGGCTTGG |
| for | TACAGCCAGGGCGGTCCCTGAGACTCAGCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGCATTGTCTATCTGCAAATGAACAGCCTGAAAGCAGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 131] |
| DNA | GAGGTGCAGCTGGTGGAGTCTGGGGGAGGCTTGG |
| for | TACAGCCAGGGCGGTCCCTGAGACTCAGCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 2 | AGGATTGTCTATGTGCAAATGAACAGCCTGAAAGCAGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA |
| | [SEQ ID NO: 132] |

In certain embodiments, the anti-CSP antibody variant comprises a light chain variable region comprising a CDR1, a CDR2, and a CDR3 and having the amino acid sequence set forth in SEQ ID NO: 133, as shown in Table 17. In certain embodiments, the anti-CSP antibody variant comprises a heavy chain variable region comprising a CDR1, a CDR2, and a CDR3 and having the amino acid sequence set forth in SEQ ID NO: 134, as shown in Table 17. In certain embodiments, the anti-CSP antibody variant comprises a light chain having the amino acid sequence set forth in SEQ ID NO: 137, as shown in Table 17. In certain embodiments, the anti-CSP antibody variant comprises a heavy chain having the amino acid sequence set forth in SEQ ID NO: 138, as shown in Table 17. In certain embodiments, the anti-CSP antibody variant comprises a heavy chain having the amino acid sequence set forth in SEQ ID NO: 139, as shown in Table 17. Exemplary nucleic acid sequences of SEQ ID NOS: 133, 134, 137, 138, and 139 are provided in Table 17 below.

TABLE 17
Antibody ID: AB-000224.012

| | KABAT | ASN |
|---|---|---|
| CDR1- | TGMNSNIGAGYDVY [SEQ ID NO: 1] | TGMNSNIGAGYDVY [SEQ ID NO: 7] |
| VL | | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG [SEQ ID | FIRKTTYGATTHYAAAVRG [SEQ ID NO: |
| VH | NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG |
| | [SEQ ID NO: 133] |
| VH | EVQLVESGGGLVQPGRSLRLSCAASGFS |
| | FGDHAMSWVRQAPGKGLEWVGFIRKTTYGATTHYAAAVRGRFTISRDDSK |
| | STVYLQMNSLKAEDTAVYFCTRVQLDYGPGYQYYGMDVWGQGTTVTVSS |
| | [SEQ ID NO: 134] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC |
| | [SEQ ID NO: 135] |
| DNA | GAGGTGCAGCTGGTGGAGTCTGGGGGAGGCTTGG |
| for VH | TACAGCCAGGGCGGTCCCTGAGACTCAGCTGTGCGGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGOACTGTOTATCTGCAAATGAACAGCCTGAAAGCAGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 136] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 137] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | STVYLQMNSLKAEDTAVYFCTRVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 138] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | STVYLQMNSLKAEDTAVYFCTRVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK |
| | [SEQ ID NO: 139] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 140] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGCACTGTCTATCTGCAAATGAACAGCCTGAAAGCAGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 141] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 2 | AGGACTGTCTATCTGCAAATGAACAGCCTGAAAGCAGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 142] |

In certain embodiments, the anti-CSP antibody variant comprises a light chain variable region comprising a CDR1, a CDR2, and a CDR3 and having the amino acid sequence set forth in SEQ ID NO: 143, as shown in Table 18. In certain embodiments, the anti-CSP antibody variant comprises a heavy chain variable region comprising a CDR1, a CDR2, and a CDR3 and having the amino acid sequence set forth in SEQ ID NO: 144, as shown in Table 18. In certain embodiments, the anti-CSP antibody variant comprises a light chain having the amino acid sequence set forth in SEQ ID NO: 147, as shown in Table 18. In certain embodiments, the anti-CSP antibody variant comprises a heavy chain having the amino acid sequence set forth in SEQ ID NO: 148, as shown in Table 18. In certain embodiments, the anti-CSP antibody variant comprises a heavy chain having the amino acid sequence set forth in SEQ ID NO: 149, as shown in Table 18. Exemplary nucleic acid sequences of SEQ ID NOS: 143, 144, 147, 148, and 149 are provided in Table 18 below.

TABLE 18
Antibody ID: AB-000224.013

| | KABAT | ASN |
|---|---|---|
| CDR1- | TGMNSNIGAGYDVY [SEQ ID NO: 1] | TGMNSNIGAGYDVY [SEQ ID NO: 7] |
| VL | | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG [SEQ ID | FIRKTTYGATTHYAAAVRG [SEQ ID NO: |
| VH | NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG |
| | [SEQ ID NO: 143] |
| VH | EVQLVESGGGLVQPGRSLRLSCAASGFS |
| | FGDHAMSWVRQAPGKGLEWVGFIRKTTYGATTHYAAAVRGRFTISRDDSK |
| | STVYLQMNSLKTEDTAVYFCARVQLDYGPGYQYYGMDVWGQGTTVTVSS |
| | [SEQ ID NO: 144] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC |
| | [SEQ ID NO: 145] |
| DNA | GAGGTGCAGCTGGTGGAGTCTGGGGGAGGCTTGG |
| for VH | TACAGCCAGGGCGGTCCCTGAGACTCAGCTGTGCGGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGCACTGTCTATCTGCAAATGAACAGCCTGAAAACCGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 146] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 147] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | STVYLQMNSLKTEDTAVYFCARVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 148] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | STVYLQMNSLKTEDTAVYFCARVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK [SEQ ID NO: 149] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 150] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGCACTGTCTATCTGCAAATGAACAGCCTGAAAACCGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA |
| | [SEQ ID NO: 151] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 2 | AGCACTGTCTATCTGCAAATGAACAGCCTGAAAACCGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 152] |

In certain embodiments, the anti-CSP antibody variant comprises a light chain variable region comprising a CDR1, a CDR2, and a CDR3 and having the amino acid sequence set forth in SEQ ID NO: 153, as shown in Table 19. In certain embodiments, the anti-CSP antibody variant comprises a heavy chain variable region comprising a CDR1, a CDR2, and a CDR3 and having the amino acid sequence set forth in SEQ ID NO: 154, as shown in Table 19. In certain embodiments, the anti-CSP antibody variant comprises a light chain having the amino acid sequence set forth in SEQ ID NO: 157, as shown in Table 19. In certain embodiments, the anti-CSP antibody variant comprises a heavy chain having the amino acid sequence set forth in SEQ ID NO: 158, as shown in Table 19. In certain embodiments, the anti-CSP antibody variant comprises a heavy chain having the amino acid sequence set forth in SEQ ID NO: 159, as shown in Table 19. Exemplary nucleic acid sequences of SEQ ID NOS: 153, 154, 157, 158, and 159 are provided in Table 19 below.

TABLE 19
Antibody ID: AB-000224.014

| | KABAT | ASN |
|---|---|---|
| CDR1- | TGMNSNIGAGYDVY [SEQ ID NO: 1] | TGMNSNIGAGYDVY [SEQ ID NO: 7] |
| VL | | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG [SEQ ID | FIRKTTYGATTHYAAAVRG [SEQ ID NO: |
| VH | NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG |
| | [SEQ ID NO: 153] |
| VH | EVQLVESGGGLVQPGRSLRLSCAASGFS |
| | FGDHAMSWVRQAPGKGLEWVGFIRKTTYGATTHYAAAVRGRFTISRDDSK |
| | SIVYLQMNSLKAEDTAVYFCARVQLDYGPGYQYYGMDVWGQGTTVTVSS |
| | [SEQ ID NO: 154] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC |
| | [SEQ ID NO: 155] |
| DNA | GAGGTGCAGCTGGTGGAGTCTGGGGGAGGCTTGG |
| for VH | TACAGCCAGGGCGGTCCCTGAGACTCAGCTGTGCGGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGOATTGTOTATCTGCAAATGAACAGCCTGAAAGCAGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 156] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 157] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | SIVYLQMNSLKAEDTAVYFCARVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 158] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | SIVYLQMNSLKAEDTAVYFCARVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK [SEQ ID NO: 159] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 160] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGCATTGTCTATCTGCAAATGAACAGCCTGAAAGCAGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 161] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 2 | AGGATTGTCTATGTGCAAATGAACAGCCTGAAAGCAGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 162] |

In certain embodiments, the anti-CSP antibody variant comprises a light chain variable region comprising a CDR1, a CDR2, and a CDR3 and having the amino acid sequence set forth in SEQ ID NO: 163, as shown in Table 20. In certain embodiments, the anti-CSP antibody variant comprises a heavy chain variable region comprising a CDR1, a CDR2, and a CDR3 and having the amino acid sequence set forth in SEQ ID NO: 164, as shown in Table 20. In certain embodiments, the anti-CSP antibody variant comprises a light chain having the amino acid sequence set forth in SEQ ID NO: 167, as shown in Table 20. In certain embodiments, the anti-CSP antibody variant comprises a heavy chain having the amino acid sequence set forth in SEQ ID NO: 168, as shown in Table 20. In certain embodiments, the anti-CSP antibody variant comprises a heavy chain having the amino acid sequence set forth in SEQ ID NO: 169, as shown in Table 20. Exemplary nucleic acid sequences of SEQ ID NOS: 163, 164, 167, 168, and 169 are provided in Table 20 below.

TABLE 20
Antibody ID: AB-000224.015

| | KABAT | ASN |
|---|---|---|
| CDR1- | TGMNSNIGAGYDVY [SEQ ID NO: | TGMNSNIGAGYDVY [SEQ ID NO: 7] |
| VL | 1] | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG | FIRKTTYGATTHYAAAVRG |
| VH | [SEQ ID NO: 5] | [SEQ ID NO: 11] |
| CDR3- | VQLDYGPGYQYYGMDV | VQLDYGPGYQYYGMDV |
| VH | [SEQ ID NO: 6] | [SEQ ID NO: 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG |
| | [SEQ ID NO: 163] |
| VH | EVQLVESGGGLVQPGRSLRLSCTASGFS |
| | FGDHAMSWVRQAPGKGLEWVGFIRKTTYGATTHYAAAVRGRFTISRDDSK |
| | STVYLQMNSLKAEDTAVYFCARVQLDYGPGYQYYGMDVWGQGTTVTVSS |
| | [SEQ ID NO: 164] |
| DNA for | CAGTCTGTGCTGACGCAGCCGCCCTCAGTGTCTGGGGCCCCAG |
| VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC |
| | [SEQ ID NO: 165] |
| DNA for | GAGGTGCAGCTGGTGGAGTCTGGGGGAGGCTTGG |
| VH | TACAGCCAGGGCGGTCCCTGAGACTCAGCTGTACAGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGCACTGTCTATCTGCAAATGAACAGCCTGAAAGCAGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 166] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 167] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | STVYLQMNSLKAEDTAVYFCARVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 168] |
| Heavy | EVQLVESGGGLVQPGRSLRLSCTASGFS |
| Chain | FGDHAMSWVRQAPGKGLEWVGFIRKTTYGATTHYAAAVRGRFTISRDDSK |
| version | STVYLQMNSLKAEDTAVYFCARVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTOKSLSLSPGK[SEQ ID NO: 169] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 170] |
| DNA | GAGGTGCAGCTGGTGGAGTCTGGGGGAGGCTTGG |
| for | TACAGCCAGGGCGGTCCCTGAGACTCAGCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGCACTGTCTATCTGCAAATGAACAGCCTGAAAGCAGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 171] |
| | GAGGTGCAGCTGGTGGAGTCTGGGGGAGGCTTGG |
| DNA | TACAGCCAGGGCGGTCCCTGAGACTCAGCTGTACAGCCTCTGGGTTTAGT |
| for | TTTGGTGATCATGCTATGAGCTGGGTCCGCCAGGCTCCAGGGAAGGGGCT |
| Heavy | GGAGTGGGTAGGTTTCATTAGAAAGACAACTTATGGTGCGACAACACACT |
| Chain | ACGCCGCGGCTGTGAGAGGCAGATTCACCATCTCGCGAGATGATTCTAAA |
| version | AGCACTGTCTATCTGCAAATGAACAGCCTGAAAGCAGAGGACACAGCCGT |
| 2 | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 172] |

In certain embodiments, the anti-CSP antibody variant comprises a light chain variable region comprising a CDR1, a CDR2, and a CDR3 and having the amino acid sequence set forth in SEQ ID NO: 173, as shown in Table 21. In certain embodiments, the anti-CSP antibody variant comprises a heavy chain variable region comprising a CDR1, a CDR2, and a CDR3 and having the amino acid sequence set forth in SEQ ID NO: 174, as shown in Table 21. In certain embodiments, the anti-CSP antibody variant comprises a light chain having the amino acid sequence set forth in SEQ ID NO: 177, as shown in Table 21. In certain embodiments, the anti-CSP antibody variant comprises a heavy chain having the amino acid sequence set forth in SEQ ID NO: 178, as shown in Table 21. In certain embodiments, the anti-CSP antibody variant comprises a heavy chain having the amino acid sequence set forth in SEQ ID NO: 179, as shown in Table 21. Exemplary nucleic acid sequences of SEQ ID NOS: 173, 174, 177, 178, and 179 are provided in Table 21 below.

TABLE 21
Antibody ID: AB-000224.016

| | KABAT | ASN |
|---|---|---|
| CDR1- | TGMNSNIGAGYDVY [SEQ ID NO: 1] | TGMNSNIGAGYDVY [SEQ ID NO: 7] |
| VL | | |
| CDR2- | GNSNRPS [SEQ ID NO: 2] | GNSNRPS [SEQ ID NO: 8] |
| VL | | |
| CDR3- | QSYDTSLNGWA [SEQ ID NO: 3] | QSYDTSLNGWA [SEQ ID NO: 9] |
| VL | | |
| CDR1- | DHAMS [SEQ ID NO: 4] | DHAMS [SEQ ID NO: 10] |
| VH | | |
| CDR2- | FIRKTTYGATTHYAAAVRG [SEQ ID | FIRKTTYGATTHYAAAVRG [SEQ ID NO: |
| VH | NO: 5] | 11] |
| CDR3- | VQLDYGPGYQYYGMDV [SEQ ID NO: | VQLDYGPGYQYYGMDV [SEQ ID NO: |
| VH | 6] | 12] |

| | |
|---|---|
| VL | QSVLTQPPSVSGAPGQRVTISCTGMNSNIGA |
| | GYDVYWYQQLPGTAPKLLIYGNSNRPSGVPDRFSGSRSGTSASLAITGLQ |
| | AEDEADYYCQSYDTSLNGWAFGGGTKLTVLG |
| | [SEQ ID NO: 173] |
| VH | EVQLVESGGGLVQPGRSLRLSCAASGFS |
| | FGDHAMSWVRQAPGKGLEWVGFIRKTTYGATTHYAAAVRGRFTISRDDSK |
| | STVYLQMNSLKAEDTAVYFCARVQLDYGPGYQYYGMDVWGQGTTVTVSS |
| | [SEQ ID NO: 174] |
| DNA | CAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAACTGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGC |
| | [SEQ ID NO: 175] |
| DNA | GAGGTGCAGCTGGTGGAGTCTGGGGGAGGCTTGG |
| for VH | TACAGCCAGGGCGGTCCCTGAGACTCAGCTGTGCGGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACACACT |
| | ACGCCGCGGCTGTGAGAGGCAGATTCACCATCTCGCGAGATGATTCTAAA |
| | AGOACTGTOTATCTGCAAATGAACAGCCTGAAAGCAGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 176] |
| Light | QSVLTQPPSVSGAPGQRVTISCTGMNSNIGA |
| Chain | GYDVYWYQQLPGTAPKLLIYGNSNRPSGVPDRFSGSRSGTSASLAITGLQ |
| | AEDEADYYCQSYDTSLNGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 177] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | STVYLQMNSLKAEDTAVYFCARVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 178] |
| Heavy | EVQLVESGGGLVQPGRSLRLSCAASGFS |
| Chain | FGDHAMSWVRQAPGKGLEWVGFIRKTTYGATTHYAAAVRGRFTISRDDSK |
| version | STVYLQMNSLKAEDTAVYFCARVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK [SEQ ID NO: 179] |
| DNA | CAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAACTGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGAA |
| | TGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 180] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 1 | AGOACTGTOTATCTGCAAATGAACAGCCTGAAAGCAGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 181] |
| DNA | GAGGTGCAGCTGGTGGAGTCTGGGGGAGGCTTGG |
| for | TACAGCCAGGGCGGTCCCTGAGACTCAGCTGTGCGGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACACACT |
| version | ACGCCGCGGCTGTGAGAGGCAGATTCACCATCTCGCGAGATGATTCTAAA |
| 2 | AGCACTGTCTATCTGCAAATGAACAGCCTGAAAGCAGAGGACACAGCCGT |
| | GTATTTCTGTGCTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 182] |

In certain embodiments, the VL of the variant of an anti-CSP antibody includes at least one, at least two, or at least three mutations, e.g., substitutions, in the CDR1 amino acid sequence set forth in SEQ ID NO: 183. In certain embodiments, the VL of the variant of an anti-CSP antibody includes at least one, at least two, or at least three mutations, e.g., substitutions, in the CDR2 amino acid sequence set forth in SEQ ID NO: 184. In certain embodiments, the VL of the variant of an anti-CSP antibody includes at least one, at least two, or at least three mutations, e.g., substitutions, in the CDR3 amino acid sequence set forth in SEQ ID NO: 185. In certain embodiments, the VH of the variant of an anti-CSP antibody includes at least one, at least two, or at least three mutations, e.g., substitutions, in the CDR1 amino acid sequence set forth in SEQ ID NO: 186. In certain embodiments, the VL of the variant of an anti-CSP antibody includes at least one, at least two, or at least three mutations, e.g., substitutions, in the CDR2 amino acid sequence set forth in SEQ ID NO: 187. In certain embodiments, the VL of the variant of an anti-CSP antibody includes at least one, at least two, or at least three mutations, e.g., substitutions, in the CDR3 amino acid sequence set forth in SEQ ID NO: 188.

In certain embodiments, the VL of the variant of an anti-CSP antibody includes at least one, at least two, or at least three mutations, e.g., substitutions, in the CDR1 amino acid sequence set forth in SEQ ID NO: 189. In certain embodiments, the VL of the variant of an anti-CSP antibody includes at least one, at least two, or at least three mutations, e.g., substitutions, in the CDR2 amino acid sequence set forth in SEQ ID NO: 190. In certain embodiments, the VL of the variant of an anti-CSP antibody includes at least one, at least two, or at least three mutations, e.g., substitutions, in the CDR3 amino acid sequence set forth in SEQ ID NO: 191. In certain embodiments, the VH of the variant of an anti-CSP antibody includes at least one, at least two, or at least three mutations, e.g., substitutions, in the CDR1 amino acid sequence set forth in SEQ ID NO: 192. In certain embodiments, the VL of the variant of an anti-CSP antibody includes at least one, at least two, or at least three mutations, e.g., substitutions, in the CDR2 amino acid sequence set forth in SEQ ID NO: 193. In certain embodiments, the VL of the variant of an anti-CSP antibody includes at least one, at least two, or at least three mutations, e.g., substitutions, in the CDR3 amino acid sequence set forth in SEQ ID NO: 194.

In certain embodiments, the anti-CSP antibody variant comprises a light chain variable region comprising a CDR1, a CDR2, and a CDR3 and having the amino acid sequence set forth in SEQ ID NO: 195, as shown in Table 22. In certain embodiments, the anti-CSP antibody variant comprises a heavy chain variable region comprising a CDR1, a CDR2, and a CDR3 and having the amino acid sequence set forth in SEQ ID NO: 196, as shown in Table 22. In certain embodiments, the anti-CSP antibody variant comprises a light chain having the amino acid sequence set forth in SEQ ID NO: 199, as shown in Table 22. In certain embodiments, the anti-CSP antibody variant comprises a heavy chain having the amino acid sequence set forth in SEQ ID NO: 200, as shown in Table 22. In certain embodiments, the anti-CSP antibody variant comprises a heavy chain having the amino acid sequence set forth in SEQ ID NO: 201, as shown in Table 22. Exemplary nucleic acid sequences of SEQ ID NOS: 195, 196, 199, 200, and 201 are provided in Table 22 below.

TABLE 22
Antibody ID: AB-000224.017

| | KABAT | ASN |
|---|---|---|
| CDR1- | TGMNSNIGAGYDVY [SEQ ID NO: | TGMNSNIGAGYDVY [SEQ ID NO: |
| VL | 183] | 189] |
| CDR2- | GNSNRPS [SEQ ID NO: 184] | GNSNRPS [SEQ ID NO: 190] |
| VL | | |
| CDR3- | QSYDTSLDGWA [SEQ ID NO: 185] | QSYDTSLDGWA [SEQ ID NO: 191] |
| VL | | |
| CDR1- | DHAMS [SEQ ID NO: 186] | DHAMS [SEQ ID NO: 192] |
| VH | | |
| CDR2- | FIRKTTYGATTKYAAAVKG [SEQ ID | FIRKTTYGATTKYAAAVKG [SEQ ID |
| VH | NO: 187] | NO: 193] |
| CDR3- | VQLDYGPGYQYYGMDV [SEQ ID NO: | VQLDYGPGYQYYGMDV [SEQ ID NO: |
| VH | 188] | 194] |

| | |
|---|---|
| VL | ESVLTQPPSVSGAPGQRVTISCTGMNSNIGA |
| | GYDVYWYQQLPGRAPKLLIYGNSNRPSGVPDRFSGSRSGTSASLAITGLQ |
| | AEDEADYYCQSYDTSLDGWAFGGGTKLTVLG |
| | [SEQ ID NO: 195] |
| VH | EVQLVESGGGLVQPGRSLRLPCTASGFS |
| | FGDHAMSWVRQAPGKGLEWVGFIRKTTYGATTKYAAAVKGRFTISRDDSK |
| | SIVYLQMNSLKTEDTAVYFCTRVQLDYGPGYQYYGMDVWGQGTTVTVSS |
| | [SEQ ID NO: 196] |
| DNA | GAGTCTGTGCTGACGCAGCCGCCCTCAGTGTCTGGGGCCCCAG |
| for VL | GGCAGAGGGTCACCATCTCCTGCACTGGGATGAACTCCAACATCGGGGCA |
| | GGTTATGATGTATACTGGTACCAACAACTTCCAGGAAGAGCCCCCAAACT |
| | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGGA |
| | CGGTTGGGCTTTCGGCGGAGGGACCAAGTTGACCGTCCTAGGC |
| | [SEQ ID NO: 197] |
| DNA | GAGGTGCAGCTGGTGGAGTCTGGGGGAGGCTTGG |
| forVH | TACAGCCAGGGCGGTCCCTGAGACTCCCCTGTACAGCCTCTGGGTTTAGT |
| | TTTGGTGATCATGCTATGAGCTGGGTCCGCCAGGCTCCAGGGAAGGGGCT |
| | GGAGTGGGTAGGTTTCATTAGAAAGACAACTTATGGTGCGACAACAAAGT |
| | ACGCCGCGGCTGTGAAGGGCAGATTCACCATCTCGCGAGATGATTCTAAA |
| | AGCATTGTCTATCTGCAAATGAACAGCCTGAAAACCGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCA |
| | [SEQ ID NO: 198] |
| Light | ESVLTQPPSVSGAPGQRVTISCTGMNSNIGA |
| Chain | GYDVYWYQQLPGRAPKLLIYGNSNRPSGVPDRFSGSRSGTSASLAITGLQ |
| | AEDEADYYCQSYDTSLDGWAFGGGTKLTVLGQPKAAPSVTLFPPSSEELQ |
| | ANKATLVCLVSDFYPGAVTVAWKADGSPVKVGVETTKPSKQSNNKYAASS |
| | YLSLTPEQWKSHRSYSCRVTHEGSTVEKTVAPAECS |
| | [SEQ ID NO: 199] |
| Heavy | EVQLVESGGGLVQPGRSLRLPCTASGFS |
| Chain | FGDHAMSWVRQAPGKGLEWVGFIRKTTYGATTKYAAAVKGRFTISRDDSK |
| version | SIVYLQMNSLKTEDTAVYFCTRVQLDYGPGYQYYGMDVWGQGTTVTVSSA |
| 1 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRWSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVMHEALHNHYTQKSLSLSPGK |
| | [SEQ ID NO: 200] |
| Heavy | EVQLVESGGGLVQPGRSLRLPCTASGFS |
| Chain | FGDHAMSWVRQAPGKGLEWVGFIRKTTYGATTKYAAAVKGRFTISRDDSK |
| version | SIVYLQMNSLKTEDTAVYFCTRVQLDYGPGYQYYGMDVWGQGTTVTVSSA |
| 2 | STKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPK |
| | SCDKTHTCPPCPAPELLGGPSVFLFPPKPKDTLMISRTPEVTCVVVDVSH |
| | EDPEVKFNWYVDGVEVHNAKTKPREEQYNSTYRWSVLTVLHQDWLNGKE |
| | YKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQ |
| | QGNVFSCSVLHEALHSHYTQKSLSLSPGK [SEQ ID NO: 201] |
| DNA | GAGTCTGTGCTGACGCAGCCGCCCTCAGTGTCTGGGGCCCCAG |
| for | GGCAGAGGGTCACCATCTCCTGCACTGGGATGAACTCCAACATCGGGGCA |
| Light | GGTTATGATGTATACTGGTACCAACAACTTCCAGGAAGAGCCCCCAAACT |
| Chain | CCTCATCTATGGTAACAGCAATCGGCCCTCAGGGGTCCCTGACCGATTCT |
| | CTGGCTCCAGGTCTGGCACCTCAGCCTCCCTGGCCATCACTGGGCTCCAG |
| | GCTGAGGATGAGGCTGATTATTACTGCCAGTCCTATGACACCAGCCTGGA |
| | CGGTTGGGCTTTCGGCGGAGGGACCAAGTTGACCGTCCTAGGCCAGCCCA |
| | AGGCTGCCCCCTCGGTCACTCTGTTCCCACCCTCCTCTGAGGAGCTTCAA |
| | GCCAACAAGGCCACACTGGTGTGTCTCGTAAGTGACTTCTACCCGGGAGC |
| | CGTGACAGTGGCCTGGAAGGCAGATGGCAGCCCCGTCAAGGTGGGAGTGG |
| | AGACCACCAAACCCTCCAAACAAAGCAACAACAAGTATGCGGCCAGCAGC |
| | TACCTGAGCCTGACGCCCGAGCAGTGGAAGTCCCACAGAAGCTACAGCTG |
| | CCGGGTCACGCATGAAGGGAGCACCGTGGAGAAGACAGTGGCCCCTGCAG |
| | AATGCTCT |
| | [SEQ ID NO: 202] |
| DNA | GAGGTGCAGCTGGTGGAGTCTGGGGGAGGCTTGG |
| for | TACAGCCAGGGCGGTCCCTGAGACTCCCCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACAAAGT |
| version | ACGCCGCGGCTGTGAAGGGCAGATTCACCATCTCGCGAGATGATTCTAAA |
| 1 | AGCATTGTCTATCTGCAAATGAACAGCCTGAAAACCGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGATGCATGAGGCTCTGCACAACCA |
| | CTACACACAGAAGAGCCTCTCCCTGTCTCCGGGTAAA [SEQ ID NO: 203] |
| DNA | GAGGTGCAGCTGGTGGAGTCTGGGGGAGGCTTGG |
| for | TACAGCCAGGGCGGTCCCTGAGACTCCCCTGTACAGCCTCTGGGTTTAGT |
| Heavy | TTTGGTGATCATGCTATGAGCTGGGTCCGCCAGGCTCCAGGGAAGGGGCT |
| Chain | GGAGTGGGTAGGTTTCATTAGAAAGACAACTTATGGTGCGACAACAAAGT |
| version | ACGCCGCGGCTGTGAAGGGCAGATTCACCATCTCGCGAGATGATTCTAAA |
| 2 | AGCATTGTCTATCTGCAAATGAACAGCCTGAAAACCGAGGACACAGCCGT |
| | GTATTTCTGTACTAGAGTGCAGCTTGACTATGGCCCGGGATACCAGTACT |
| | ACGGTATGGACGTCTGGGGCCAAGGGACCACGGTCACCGTCTCCTCAGCC |
| | TCCACCAAGGGCCCATCGGTCTTCCCCCTGGCACCCTCCTCCAAGAGCAC |
| | CTCTGGGGGCACAGCAGCCCTGGGCTGCCTGGTCAAGGACTACTTCCCCG |
| | AACCGGTGACGGTGTCGTGGAACTCAGGCGCCCTGACCAGCGGCGTGCAC |
| | ACCTTCCCGGCTGTCCTACAGTCCTCAGGACTCTACTCCCTCAGCAGCGT |
| | GGTGACCGTGCCCTCCAGCAGCTTGGGCACCCAGACCTACATCTGCAACG |
| | TGAATCACAAGCCCAGCAACACCAAGGTGGACAAGAAAGTTGAGCCCAAA |
| | TCTTGTGACAAAACTCACACATGCCCACCGTGCCCAGCACCTGAACTCCT |
| | GGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCAC |
| | GAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTG |
| | TGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAAC |
| | CATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTG |
| | GTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAG |
| | CAGGGGAACGTCTTCTCATGCTCCGTGCTGCATGAGGCTCTGCACTCCCA |
| | CTACACACAGAAGAGCCTCTCCCTGTCTCCGGGTAAA [SEQ ID NO: 204] |

### 3. Anti-CSP Antibody Variants of AB-007088

In certain embodiments, a variant of an anti-CSP antibody AB-007088 disclosed herein comprises modifications compared to AB-007088 that provide improved pharmacokinetic properties, increased serum stability, stronger binding, and/or improved in vivo protective effects compared to AB-007088. In certain embodiments, a variant of an anti-CSP antibody AB-007088 disclosed herein exhibits reduced immunogenicity and/or increased manufacturability as compared to AB-007088. In certain embodiments, a variant of an anti-CSP antibody AB-007088 disclosed herein has at least one modification, e.g., substitution, relative to the native AB-007088 variable heavy chain amino acid sequence (SEQ ID NO: 196) or variable light chain amino acid sequence (SEQ ID NO: 195), and has improved developability, e.g., decreased heterogeneity, increased yield, increased stability, improved net charges to improve pharmacokinetics, and/or reduced immunogenicity. In certain embodiments, a VH region or a VL region of such a variant of an anti-CSP antibody AB-007088 disclosed herein has at least two, three, four, five, or six, or more modifications, e.g., substitutions. In certain embodiments, a variant of the an anti-CSP antibody AB-007088 disclosed herein has a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications, e.g. substitutions, including both variable regions, compared to AB-007088.

In certain embodiments, a variant of an anti-CSP antibody AB-007088 disclosed herein exhibits increased serum half-life as compared to AB-007088. In certain embodiments, a variant of an anti-CSP antibody AB-007088 disclosed herein has at least one modification, e.g., substitution, relative to the native AB-007088 Fc region of the heavy chain herein, and has improved pharmacokinetics properties, e.g., half-life. In certain embodiments, an Fc region of the heavy chain of such a variant of an anti-CSP antibody AB-007088 disclosed herein has at least two, three, four, five, or six, or more modifications, e.g., substitutions. In certain embodiments, a variant of the an anti-CSP antibody AB-007088 disclosed herein has a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modifications, e.g. substitutions, including both heavy and light chains, compared to AB-007088. In certain non-limiting embodiments, an Fc region of the heavy chain of a variant of an anti-CSP antibody AB-007088 disclosed herein can include an isoleucine at position 250, a tyrosine at position 252, an isoleucine at position 259, a glutamine at position 307, a phenylalanine at position 308, a leucine at position 319, a leucine at position 428, a histidine at position 434, a phenylalanine at position 434, an alanine at position 434, a serine at position 434, a methionine at position 434, or a combination thereof, wherein the numbering is defined by EU index as in Kabat. In certain embodiments, an Fc region of the heavy chain of a variant of an anti-CSP antibody AB-007088 disclosed herein includes a leucine at position 428 and a serine at position 434, wherein the numbering is defined by EU index as in Kabat.

The light and heavy chain CDRs of AB-007088 as defined by Kabat numbering system are shown in Table 23 below:

TABLE 23
AB-007088 CDR sequences (Kabat)

| | CDR1 | CDR2 | CDR3 |
|---|---|---|---|
| VL | RASQSISSWLA | DASSLES | QQYNSYSFWT |
| region | (SEQ ID NO: | (SEQ ID | (SEQ ID |
| | 205) | NO: 206) | NO: 207) |
| VH | TYGMH | IIWYDGSQKYYAD | VRFSVGPHGSA |
| region | (SEQ ID NO: | SVQG (SEQ ID | FDL (SEQ ID |
| | 208) | NO: 209) | NO: 210) |

The light and heavy chain CDRs of AB-007088 as defined by ASN are shown in Table 24 below:

TABLE 24
AB-007088 CDR sequences (ASN)

| | CDR1 | CDR2 | CDR3 |
|---|---|---|---|
| VL | RASQSISSWLA | DASSLES | QQYNSYWT |
| region | (SEQ ID | (SEQ ID NO: 212) | (SEQ ID |
| | NO: 211) | | NO: 213) |
| VH | TYGMH | IIWYDGSQKYYADSVQG | SAFDL |
| region | (SEQ ID | (SEQ ID NO: 215) | (SEQ ID |
| | NO: 214) | | NO: 216) |

The heavy chain variable region (VH) and light chain variable region (VL) sequences and heavy and light chain sequences of AB-007088 are shown in Table 25 below:

TABLE 25
6AB-007088 variable region and full-length sequences

| | |
|---|---|
| VL region | GVQMTQSPSTLSASVGDRVTLTCRASQSISSWLAWYQQKPGKAPKLLIYDAS |
| | SLESGVPSRFSGSGSGTEFTLTISSLQPDDFATYYCQQYNSYSFWTFGQGTK |
| | VEIKR (SEQ ID NO: 217) |
| VH region | QVQLVESGGGWQPGRSLRLSCAASGFAFNTYGMHWVRQTPGKGLEWVAIIWY |
| | DGSQKYYADSVQGRFIISRDNHKNTLSLQMNGLRAEDTAVYFCVRVRFSVGP |
| | HGSAFDLWGQGTMVIVSS (SEQ ID NO: 218) |
| DNA for | GGCGTCCAGATGACCCAGTCTCCTTCCACCCTGT |
| VL region | CTGCATCTGTGGGAGACAGAGTCACCCTCACTTGCCGGGCCAGTCAGAGT |
| | ATTAGTAGTTGGTTGGCCTGGTATCAGCAGAAACCAGGGAAAGCCCCTAA |
| | ACTCCTGATCTATGATGCCTCCAGTTTGGAAAGTGGGGTCCCATCAAGGT |
| | TCAGCGGCAGTGGATCTGGGACAGAATTCACTCTCACCATCAGCAGCCTG |
| | CAACCTGATGATTTTGCAACTTATTACTGCCAACAGTATAATAGTTATTC |
| | TTTTTGGACGTTCGGCCAAGGGACCAAGGTGGAAATCAAACGC |
| | [SEQ ID NO: 219] |
| DNA for | CAGGTGCAACTGGTGGAGTCTGGGGGAGGCGTGG |
| VH region | TCCAGCCTGGGAGGTCCCTGAGACTCTCCTGTGCAGCGTCTGGGTTCGCT |
| | TTCAATACCTATGGCATGCACTGGGTCCGCCAGACTCCAGGCAAGGGGCT |
| | GGAGTGGGTGGCAATTATCTGGTATGATGGGAGCCAGAAATACTATGCAG |
| | ACTCCGTGCAGGGCCGATTCATCATCTCCAGAGACAATCACAAGAACACG |
| | TTGTCTCTGCAAATGAACGGCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCATCGTCTCTTCA |
| | [SEQ ID NO: 220] |
| Light Chain | GVQMTQSPSTLSASVGDRVTLTCRASQS |
| | ISSWLAWYQQKPGKAPKLLIYDASSLESGVPSRFSGSGSGTEFTLTISSL |
| | QPDDFATYYCQQYNSYSFWTFGQGTKVEIKRTVAAPSVFIFPPSDEQLKS |
| | GTASVVCLLNNFYPREAKVQWKVDNALQSGNSQESVTEQDSKDSTYSLSS |
| | TLTLSKADYEKHKVYACEVTHQGLSSPVTKSFNRGEC |
| | [SEQ ID NO: 221] |
| Heavy Chain | QVQLVESGGGVVQPGRSLRLSCAASGFA |
| version 1 | FNTYGMHWVRQTPGKGLEWVAIIWYDGSQKYYADSVQGRFIISRDNHKNT |
| | LSLQMNGLRAEDTAVYFCVRVRFSVGPHGSAFDLWGQGTMVIVSSASTKG |
| | PSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPE |
| | VKFNWYVDGVEVHNAKTKPREEQYNSTYRWSVLTVLHQDWLNGKEYKCK |
| | VSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNV |
| | FSCSVMHEALHNHYTQKSLSLSPGK [SEQ ID NO: 222] |
| Heavy Chain | QVQLVESGGGVVQPGRSLRLSCAASGFA |
| version 2 | FNTYGMHWVRQTPGKGLEWVAIIWYDGSQKYYADSVQGRFIISRDNHKNT |
| | LSLQMNGLRAEDTAVYFCVRVRFSVGPHGSAFDLWGQGTMVIVSSASTKG |
| | PSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPE |
| | VKFNWYVDGVEVHNAKTKPREEQYNSTYRWSVLTVLHQDWLNGKEYKCK |
| | VSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNV |
| | FSCSVLHEALHSHYTQKSLSLSPGK [SEQ ID NO: 223] |
| DNA for | GGCGTCCAGATGACCCAGTCTCCTTCCACCCTGT |
| Light Chain | CTGCATCTGTGGGAGACAGAGTCACCCTCACTTGCCGGGCCAGTCAGAGT |
| | ATTAGTAGTTGGTTGGCCTGGTATCAGCAGAAACCAGGGAAAGCCCCTAA |
| | ACTCCTGATCTATGATGCCTCCAGTTTGGAAAGTGGGGTCCCATCAAGGT |
| | TCAGCGGCAGTGGATCTGGGACAGAATTCACTCTCACCATCAGCAGCCTG |
| | CAACCTGATGATTTTGCAACTTATTACTGCCAACAGTATAATAGTTATTC |
| | TTTTTGGACGTTCGGCCAAGGGACCAAGGTGGAAATCAAACGCACTGTGG |
| | CTGCACCATCTGTCTTCATCTTCCCGCCATCTGATGAGCAGTTGAAATCT |
| | GGAACTGCCTCTGTTGTGTGCCTGCTGAATAACTTCTATCCCAGAGAGGC |
| | CAAAGTACAGTGGAAGGTGGATAACGCCCTCCAATCGGGTAACTCCCAGG |
| | AGAGTGTCACAGAGCAGGACAGCAAGGACAGCACCTACAGCCTCAGCAGC |
| | ACCCTGACGCTGAGCAAAGCAGACTACGAGAAACACAAAGTCTACGCCTG |
| | CGAAGTCACCCATCAGGGCCTGAGCTCGCCCGTCACAAAGAGCTTCAACA |
| | GGGGAGAGTGT |
| | [SEQ ID NO: 224] |
| DNA for | CAGGTGCAACTGGTGGAGTCTGGGGGAGGCGTGG |
| Heavy Chain | TCCAGCCTGGGAGGTCCCTGAGACTCTCCTGTGCAGCGTCTGGGTTCGCT |
| version 1 | TTCAATACCTATGGCATGCACTGGGTCCGCCAGACTCCAGGCAAGGGGCT |
| | GGAGTGGGTGGCAATTATCTGGTATGATGGGAGCCAGAAATACTATGCAG |
| | ACTCCGTGCAGGGCCGATTCATCATCTCCAGAGACAATCACAAGAACACG |
| | TTGTCTCTGCAAATGAACGGCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCATCGTCTCTTCAGCCTCCACCAAGGGC |
| | CCATCGGTCTTCCCCCTGGCACCCTCCTCCAAGAGCACCTCTGGGGGCAC |
| | AGCAGCCCTGGGCTGCCTGGTCAAGGACTACTTCCCCGAACCGGTGACGG |
| | TGTCGTGGAACTCAGGCGCCCTGACCAGCGGCGTGCACACCTTCCCGGCT |
| | GTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGAC |
| | AAAGCCGCGGGAGGAGCAGTACAACAGCACGTACCGTGTGGTCAGCGTCC |
| | TCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGC |
| | CAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCC |
| | TCTACAGCAAGCTCACCGTGGACAAGAGCAGGTGGCAGCAGGGGAACGTC |
| | TTCTCATGCTCCGTGATGCATGAGGCTCTGCACAACCACTACACACAGAA |
| | GAGCCTCTCCCTGTCTCCGGGTAAA [SEQ ID NO: 225] |
| DNA for | CAGGTGCAACTGGTGGAGTCTGGGGGAGGCGTGG |
| Heavy Chain | TCCAGCCTGGGAGGTCCCTGAGACTCTCCTGTGCAGCGTCTGGGTTCGCT |
| version 2 | TTCAATACCTATGGCATGCACTGGGTCCGCCAGACTCCAGGCAAGGGGCT |
| | GGAGTGGGTGGCAATTATCTGGTATGATGGGAGCCAGAAATACTATGCAG |
| | ACTCCGTGCAGGGCCGATTCATCATCTCCAGAGACAATCACAAGAACACG |
| | TTGTCTCTGCAAATGAACGGCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCATCGTCTCTTCAGCCTCCACCAAGGGC |
| | CCATCGGTCTTCCCCCTGGCACCCTCCTCCAAGAGCACCTCTGGGGGCAC |
| | AGCAGCCCTGGGCTGCCTGGTCAAGGACTACTTCCCCGAACCGGTGACGG |
| | TGTCGTGGAACTCAGGCGCCCTGACCAGCGGCGTGCACACCTTCCCGGCT |
| | GTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGAC |
| | AAAGCCGCGGGAGGAGCAGTACAACAGCACGTACCGTGTGGTCAGCGTCC |
| | TCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGC |
| | CAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCC |
| | TCTACAGCAAGCTCACCGTGGACAAGAGCAGGTGGCAGCAGGGGAACGTC |
| | TTCTCATGCTCCGTGCTGCATGAGGCTCTGCACTCCCACTACACACAGAA |
| | GAGCCTCTCCCTGTCTCCGGGTAAA [SEQ ID NO: 226] |

In certain embodiments, a variant of an anti-CSP antibody includes one, two, or three CDRs of a VL sequence of Table 25. In certain embodiments, a variant of an anti-CSP antibody includes at least one mutation, e.g., a substitution, and no more than 10, 20, 30, 40, or 50 mutations in the VL amino acid sequence set forth in SEQ ID NO: 217. In certain embodiments, the VL of the variant of an anti-CSP antibody includes at least one, at least two, or at least three mutations, e.g., substitutions, in the CDR1 amino acid sequence set forth in SEQ ID NO: 205. In certain embodiments, the VL of the variant of an anti-CSP antibody includes at least one, at least two, or at least three mutations, e.g., substitutions, in the CDR2 amino acid sequence set forth in SEQ ID NO: 206. In certain embodiments, the VL of the variant of an anti-CSP antibody includes at least one, at least two, or at least three mutations, e.g., substitutions, in the CDR3 amino acid sequence set forth in SEQ ID NO: 207. In certain embodiments, the VL of the variant of an anti-CSP antibody includes at least one, at least two, or at least three mutations, e.g., substitutions, in the CDR1 amino acid sequence set forth in SEQ ID NO: 211. In certain embodiments, the VL of the variant of an anti-CSP antibody includes at least one, at least two, or at least three mutations, e.g., substitutions, in the CDR2 amino acid sequence set forth in SEQ ID NO: 212. In certain embodiments, the VL of the variant of an anti-CSP antibody includes at least one, at least two, or at least three mutations, e.g., substitutions, in the CDR3 amino acid sequence set forth in SEQ ID NO: 213. In certain embodiments, the mutation is a conservative substitution. In certain embodiments, the VL of the variant of an anti-CSP antibody includes at least one, at least two, or at least three mutations, e.g., substitutions, in the framework of the amino acid sequence set forth in SEQ ID NO: 217. An exemplary nucleic acid sequence of SEQ ID NO: 217 is set forth in SEQ ID NO: 219.

In certain embodiments, a variant of an anti-CSP antibody includes one, two, or three CDRs of a VH sequence of Table 25. In certain embodiments, a variant of an anti-CSP antibody includes at least one mutation, e.g., a substitution, and no more than 10, 20, 30, 40, or 50 mutations in the VH amino acid sequence set forth in SEQ ID NO: 218. In certain embodiments, the VH of the variant of an anti-CSP antibody includes at least one, at least two, or at least three mutations, e.g., substitutions, in the CDR1 amino acid sequence set forth in SEQ ID NO: 208. In certain embodiments, the VH of the variant of an anti-CSP antibody includes at least one, at least two, or at least three mutations, e.g., substitutions, in the CDR2 amino acid sequence set forth in SEQ ID NO: 209. In certain embodiments, the VH of the variant of an anti-CSP antibody includes at least one, at least two, or at least three mutations, e.g., substitutions, in the CDR3 amino acid sequence set forth in SEQ ID NO: 210. In certain embodiments, the VH of the variant of an anti-CSP antibody includes at least one, at least two, or at least three mutations, e.g., substitutions, in the CDR1 amino acid sequence set forth in SEQ ID NO: 214. In certain embodiments, the VH of the variant of an anti-CSP antibody includes at least one, at least two, or at least three mutations, e.g., substitutions, in the CDR2 amino acid sequence set forth in SEQ ID NO: 215. In certain embodiments, the VH of the variant of an anti-CSP antibody includes at least one, at least two, or at least three mutations, e.g., substitutions, in the CDR3 amino acid sequence set forth in SEQ ID NO: 216. In certain embodiments, the mutation is a conservative substitution. In certain embodiments, the VH of the variant of an anti-CSP antibody includes at least one, at least two, at least three mutations, at least four mutations, at least five mutations, or at least six mutations, e.g., substitutions, in the framework of the amino acid sequence set forth in SEQ ID NO: 218. An exemplary nucleic acid sequence of SEQ ID NO: 218 is set forth in SEQ ID NO: 220. In certain embodiments, the VH of the variant of an anti-CSP antibody includes a substitution at position 28 of the SEQ ID NO: 218. In certain embodiments, the substitution is A28T. In certain embodiments, the VH of the variant of an anti-CSP antibody includes a substitution at position 40 of the SEQ ID NO: 218. In certain embodiments, the substitution is T40A. In certain embodiments, the VH of the variant of an anti-CSP antibody includes a substitution at position 69 of the SEQ ID NO: 218. In certain embodiments, the substitution is I69T. In certain embodiments, the VH of the variant of an anti-CSP antibody includes a substitution at position 80 of the SEQ ID NO: 218. In certain embodiments, the substitution is S80Y. In certain embodiments, the VH of the variant of an anti-CSP antibody includes a substitution at position 85 of the SEQ ID NO: 218. In certain embodiments, the substitution is G85S. In certain embodiments, the VH of the variant of an anti-CSP antibody includes a substitution at position 120 of the SEQ ID NO: 218. In certain embodiments, the substitution is I120T.

In certain embodiments, the light chain of the variant of an anti-CSP antibody has an amino acid sequence set forth in SEQ ID NO: 221. An exemplary nucleic acid sequence of SEQ ID NO: 221 is set forth in SEQ ID NO: 224. In certain embodiments, a variant of an anti-CSP antibody includes at least one mutation, e.g., substitution, and no more than 10, 20, 30, 40, or 50 mutations in the Fc region of the heavy chain amino acid sequence set forth in SEQ ID NO: 222. In certain embodiments, the heavy chain of the variant of an anti-CSP antibody has an amino acid sequence set forth in SEQ ID NO: 222. An exemplary nucleic acid sequence of SEQ ID NO: 222 is set forth in SEQ ID NO: 225. In certain embodiments, the heavy chain of the variant of an anti-CSP antibody includes a substitution at position 434 of the SEQ ID NO: 222. In certain embodiments, the substitution is M434L. In certain embodiments, the heavy chain of the variant of an anti-CSP antibody includes a substitution at position 440 of the SEQ ID NO: 222. In certain embodiments, the substitution is L440S. In certain embodiments, the heavy chain of the variant of an anti-CSP antibody includes a substitution at position 434 of the SEQ ID NO: 222 and a substitution at position 440 of the SEQ ID NO: 222. In certain embodiments, the substitutions are M434L and N440S. In certain embodiments, the heavy chain of the variant of an anti-CSP antibody has an amino acid sequence set forth in SEQ ID NO: 223. An exemplary nucleic acid sequence of SEQ ID NO: 223 is set forth in SEQ ID NO: 226.

In certain embodiments, the light chain of the anti-CSP antibody AB-007088 and variants thereof comprises a signal peptide. In certain embodiments, the signal peptide is an IGLV2-8 signal peptide. In certain embodiments, the signal peptide has an amino acid sequence set forth in SEQ ID NO: 277. In certain embodiments, the heavy chain of the anti-CSP antibody AB-007088 and variants thereof comprises a signal peptide. In certain embodiments, the signal peptide is an IGKV1-39 signal peptide. In certain embodiments, the signal peptide has an amino acid sequence set forth in SEQ ID NO: 278.

In certain embodiments, the anti-CSP antibody variant comprises a light chain variable region comprising a CDR1, a CDR2, and a CDR3 and having the amino acid sequence set forth in SEQ ID NO: 227, as shown in Table 26. In certain embodiments, the anti-CSP antibody variant comprises a heavy chain variable region comprising a CDR1, a CDR2, and a CDR3 and having the amino acid sequence set forth in SEQ ID NO: 228, as shown in Table 26. In certain embodiments, the anti-CSP antibody variant comprises a light chain having the amino acid sequence set forth in SEQ ID NO: 231, as shown in Table 26. In certain embodiments, the anti-CSP antibody variant comprises a heavy chain having the amino acid sequence set forth in SEQ ID NO: 232, as shown in Table 26. In certain embodiments, the anti-CSP antibody variant comprises a heavy chain having the amino acid sequence set forth in SEQ ID NO: 233, as shown in Table 26. Exemplary nucleic acid sequences of SEQ ID NOS: 227, 228, 231, 232, and 233 are provided in Table 26 below.

TABLE 26
Antibody ID: AB-007088.001

| | KABAT | ASN |
|---|---|---|
| CDR1- | RASQSISSWLA [SEQ ID NO: 205] | RASQSISSWLA [SEQ ID NO: 211] |
| VL | | |
| CDR2- | DASSLES [SEQ ID NO: 206] | DASSLES [SEQ ID NO: 212] |
| VL | | |
| CDR3- | QQYNSYSFWT [SEQ ID NO: 207] | QQYNSYSFWTF [SEQ ID NO: 213] |
| VL | | |
| CDR1- | TYGMH [SEQ ID NO: 208] | TYGMH [SEQ ID NO: 214] |
| VH | | |
| CDR2- | IIWYDGSQKYYADSVQG [SEQ ID | IIWYDGSQKYYADSVQG [SEQ ID NO: |
| VH | NO: 209] | 215] |
| CDR3- | VRFSVGPHGSAFDL [SEQ ID NO: | SAFDL [SEQ ID NO: 216] |
| VH | 210] | |

| | |
|---|---|
| VL | GVQMTQSPSTLSASVGDRVTLTCRASQS |
| | ISSWLAWYQQKPGKAPKLLIYDASSLESGVPSRFSGSGSGTEFTLTISSL |
| | QPDDFATYYCQQYNSYSFWTFGQGTKVEIKR |
| | [SEQ ID NO: 227] |
| VH | QVQLVESGGGVVQPGRSLRLSCAASGFA |
| | FNTYGMHWVRQAPGKGLEWVAIIWYDGSQKYYADSVQGRFIISRDNHKNT |
| | LSLQMNSLRAEDTAVYFCVRVRFSVGPHGSAFDLWGQGTMVTVSS |
| | [SEQ ID NO: 228] |
| DNA | GGCGTCCAGATGACCCAGTCTCCTTCCACCCTGT |
| for VL | CTGCATCTGTGGGAGACAGAGTCACCCTCACTTGCCGGGCCAGTCAGAGT |
| | ATTAGTAGTTGGTTGGCCTGGTATCAGCAGAAACCAGGGAAAGCCCCTAA |
| | ACTCCTGATCTATGATGCCTCCAGTTTGGAAAGTGGGGTCCCATCAAGGT |
| | TCAGCGGCAGTGGATCTGGGACAGAATTCACTCTCACCATCAGCAGCCTG |
| | CAACCTGATGATTTTGCAACTTATTACTGCCAACAGTATAATAGTTATTC |
| | TTTTTGGACGTTCGGCCAAGGGACCAAGGTGGAAATCAAACGC |
| | [SEQ ID NO: 229] |
| DNA | CAGGTGCAACTGGTGGAGTCTGGGGGAGGCGTGG |
| forVH | TCCAGCCTGGGAGGTCCCTGAGACTCTCCTGTGCAGCGTCTGGGTTCGCT |
| | TTCAATACCTATGGCATGCACTGGGTCCGCCAGGCACCAGGCAAGGGGCT |
| | GGAGTGGGTGGCAATTATCTGGTATGATGGGAGCCAGAAATACTATGCAG |
| | ACTCCGTGCAGGGCCGATTCATCATCTCCAGAGACAATCACAAGAACACG |
| | TTGTCTCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCA |
| | [SEQ ID NO: 230] |
| Light | GVQMTQSPSTLSASVGDRVTLTCRASQS |
| Chain | ISSWLAWYQQKPGKAPKLLIYDASSLESGVPSRFSGSGSGTEFTLTISSL |
| | QPDDFATYYCQQYNSYSFWTFGQGTKVEIKRTVAAPSVFIFPPSDEQLKS |
| | GTASVVCLLNNFYPREAKVQWKVDNALQSGNSQESVTEQDSKDSTYSLSS |
| | TLTLSKADYEKHKVYACEVTHQGLSSPVTKSFNRGEC |
| | [SEQ ID NO: 231] |
| Heavy | QVQLVESGGGVVQPGRSLRLSCAASGFA |
| Chain | FNTYGMHWVRQAPGKGLEWVAIIWYDGSQKYYADSVQGRFIISRDNHKNT |
| version | LSLQMNSLRAEDTAVYFCVRVRFSVGPHGSAFDLWGQGTMVTVSSASTKG |
| 1 | PSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNV |
| | FSCSVMHEALHNHYTQKSLSLSPGK |
| | [SEQ ID NO: 232] |
| Heavy | QVQLVESGGGVVQPGRSLRLSCAASGFA |
| Chain | FNTYGMHWVRQAPGKGLEWVAIIWYDGSQKYYADSVQGRFIISRDNHKNT |
| version | LSLQMNSLRAEDTAVYFCVRVRFSVGPHGSAFDLWGQGTMVTVSSASTKG |
| 2 | PSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNV |
| | FSCSVLHEALHSHYTQKSLSLSPGK [SEQ ID NO: 233] |
| DNA | GGCGTCCAGATGACCCAGTCTCCTTCCACCCTGT |
| for | CTGCATCTGTGGGAGACAGAGTCACCCTCACTTGCCGGGCCAGTCAGAGT |
| Light | ATTAGTAGTTGGTTGGCCTGGTATCAGCAGAAACCAGGGAAAGCCCCTAA |
| Chain | ACTCCTGATCTATGATGCCTCCAGTTTGGAAAGTGGGGTCCCATCAAGGT |
| | TCAGCGGCAGTGGATCTGGGACAGAATTCACTCTCACCATCAGCAGCCTG |
| | CAACCTGATGATTTTGCAACTTATTACTGCCAACAGTATAATAGTTATTC |
| | TTTTTGGACGTTCGGCCAAGGGACCAAGGTGGAAATCAAACGCACTGTGG |
| | CTGCACCATCTGTCTTCATCTTCCCGCCATCTGATGAGCAGTTGAAATCT |
| | GGAACTGCCTCTGTTGTGTGCCTGCTGAATAACTTCTATCCCAGAGAGGC |
| | CAAAGTACAGTGGAAGGTGGATAACGCCCTCCAATCGGGTAACTCCCAGG |
| | AGAGTGTCACAGAGCAGGACAGCAAGGACAGCACCTACAGCCTCAGCAGC |
| | ACCCTGACGCTGAGCAAAGCAGACTACGAGAAACACAAAGTCTACGCCTG |
| | CGAAGTCACCCATCAGGGCCTGAGCTCGCCCGTCACAAAGAGCTTCAACA |
| | GGGGAGAGTGT |
| | [SEQ ID NO: 234] |
| DNA | CAGGTGCAACTGGTGGAGTCTGGGGGAGGCGTGG |
| for | TCCAGCCTGGGAGGTCCCTGAGACTCTCCTGTGCAGCGTCTGGGTTCGCT |
| Heavy | TTCAATACCTATGGCATGCACTGGGTCCGCCAGGCACCAGGCAAGGGGCT |
| Chain | GGAGTGGGTGGCAATTATCTGGTATGATGGGAGCCAGAAATACTATGCAG |
| version | ACTCCGTGCAGGGCCGATTCATCATCTCCAGAGACAATCACAAGAACACG |
| 1 | TTGTCTCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCAGCCTCCACCAAGGGC |
| | CCATCGGTCTTCCCCCTGGCACCCTCCTCCAAGAGCACCTCTGGGGGCAC |
| | AGCAGCCCTGGGCTGCCTGGTCAAGGACTACTTCCCCGAACCGGTGACGG |
| | TGTCGTGGAACTCAGGCGCCCTGACCAGCGGCGTGCACACCTTCCCGGCT |
| | GTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGAC |
| | AAAGCCGCGGGAGGAGCAGTACAACAGCACGTACCGTGTGGTCAGCGTCC |
| | TCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGC |
| | CAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCC |
| | TCTACAGCAAGCTCACCGTGGACAAGAGCAGGTGGCAGCAGGGGAACGTC |
| | TTCTCATGCTCCGTGATGCATGAGGCTCTGCACAACCACTACACACAGAA |
| | GAGCCTCTCCCTGTCTCCGGGTAAA [SEQ ID NO: 235] |
| DNA | CAGGTGCAACTGGTGGAGTCTGGGGGAGGCGTGG |
| for | TCCAGCCTGGGAGGTCCCTGAGACTCTCCTGTGCAGCGTCTGGGTTCGCT |
| Heavy | TTCAATACCTATGGCATGCACTGGGTCCGCCAGGCACCAGGCAAGGGGCT |
| Chain | GGAGTGGGTGGCAATTATCTGGTATGATGGGAGCCAGAAATACTATGCAG |
| version | ACTCCGTGCAGGGCCGATTCATCATCTCCAGAGACAATCACAAGAACACG |
| 2 | TTGTCTCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCAGCCTCCACCAAGGGC |
| | CCATCGGTCTTCCCCCTGGCACCCTCCTCCAAGAGCACCTCTGGGGGCAC |
| | AGCAGCCCTGGGCTGCCTGGTCAAGGACTACTTCCCCGAACCGGTGACGG |
| | TGTCGTGGAACTCAGGCGCCCTGACCAGCGGCGTGCACACCTTCCCGGCT |
| | GTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGAC |
| | AAAGCCGCGGGAGGAGCAGTACAACAGCACGTACCGTGTGGTCAGCGTCC |
| | TCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGC |
| | CAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCC |
| | TCTACAGCAAGCTCACCGTGGACAAGAGCAGGTGGCAGCAGGGGAACGTC |
| | TTCTCATGCTCCGTGCTGCATGAGGCTCTGCACTCCCACTACACACAGAA |
| | GAGCCTCTCCCTGTCTCCGGGTAAA [SEQ ID NO: 236] |

In certain embodiments, the anti-CSP antibody variant comprises a light chain variable region comprising a CDR1, a CDR2, and a CDR3 and having the amino acid sequence set forth in SEQ ID NO: 237, as shown in Table 27. In certain embodiments, the anti-CSP antibody variant comprises a heavy chain variable region comprising a CDR1, a CDR2, and a CDR3 and having the amino acid sequence set forth in SEQ ID NO: 238, as shown in Table 27. In certain embodiments, the anti-CSP antibody variant comprises a light chain having the amino acid sequence set forth in SEQ ID NO: 241, as shown in Table 27. In certain embodiments, the anti-CSP antibody variant comprises a heavy chain having the amino acid sequence set forth in SEQ ID NO: 242, as shown in Table 27. In certain embodiments, the anti-CSP antibody variant comprises a heavy chain having the amino acid sequence set forth in SEQ ID NO: 243, as shown in Table 27. Exemplary nucleic acid sequences of SEQ ID NOS: 237, 238, 241, 242, and 243 are provided in Table 27 below.

TABLE 27
Antibody ID: AB-007088.002

| | KABAT | ASN |
|---|---|---|
| CDR1- | RASQSISSWLA [SEQ ID NO: 205] | RASQSISSWLA [SEQ ID NO: 211] |
| VL | | |
| CDR2- | DASSLES [SEQ ID NO: 206] | DASSLES [SEQ ID NO: 212] |
| VL | | |
| CDR3- | QQYNSYSFWT [SEQ ID NO: 207] | QQYNSYSFWTF [SEQ ID NO: 213] |
| VL | | |
| CDR1- | TYGMH [SEQ ID NO: 208] | TYGMH [SEQ ID NO: 214] |
| VH | | |
| CDR2- | IIWYDGSQKYYADSVQG [SEQ ID | IIWYDGSQKYYADSVQG [SEQ ID NO: |
| VH | NO: 209] | 215] |
| CDR3- | VRFSVGPHGSAFDL [SEQ ID NO: | SAFDL [SEQ ID NO: 216] |
| VH | 210] | |

| | |
|---|---|
| VL | GVQMTQSPSTLSASVGDRVTLTCRASQS |
| | ISSWLAWYQQKPGKAPKLLIYDASSLESGVPSRFSGSGSGTEFTLTISSL |
| | QPDDFATYYCQQYNSYSFWTFGQGTKVEIKR |
| | [SEQ ID NO: 237] |
| VH | QVQLVESGGGVVQPGRSLRLSCAASGFT |
| | FNTYGMHWVRQAPGKGLEWVAIIWYDGSQKYYADSVQGRFIISRDNHKNT |
| | LSLQMNSLRAEDTAVYFCVRVRFSVGPHGSAFDLWGQGTMVTVSS |
| | [SEQ ID NO: 238] |
| DNA | GGCGTCCAGATGACCCAGTCTCCTTCCACCCTGT |
| for VL | CTGCATCTGTGGGAGACAGAGTCACCCTCACTTGCCGGGCCAGTCAGAGT |
| | ATTAGTAGTTGGTTGGCCTGGTATCAGCAGAAACCAGGGAAAGCCCCTAA |
| | ACTCCTGATCTATGATGCCTCCAGTTTGGAAAGTGGGGTCCCATCAAGGT |
| | TCAGCGGCAGTGGATCTGGGACAGAATTCACTCTCACCATCAGCAGCCTG |
| | CAACCTGATGATTTTGCAACTTATTACTGCCAACAGTATAATAGTTATTC |
| | TTTTTGGACGTTCGGCCAAGGGACCAAGGTGGAAATCAAACGC |
| | [SEQ ID NO: 239] |
| DNA | CAGGTGCAACTGGTGGAGTCTGGGGGAGGCGTGG |
| forVH | TCCAGCCTGGGAGGTCCCTGAGACTCTCCTGTGCAGCGTCTGGGTTCACT |
| | TTCAATACCTATGGCATGCACTGGGTCCGCCAGGCACCAGGCAAGGGGCT |
| | GGAGTGGGTGGCAATTATCTGGTATGATGGGAGCCAGAAATACTATGCAG |
| | ACTCCGTGCAGGGCCGATTCATCATCTCCAGAGACAATCACAAGAACACG |
| | TTGTCTCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCA |
| | [SEQ ID NO: 240] |
| Light | GVQMTQSPSTLSASVGDRVTLTCRASQS |
| Chain | ISSWLAWYQQKPGKAPKLLIYDASSLESGVPSRFSGSGSGTEFTLTISSL |
| | QPDDFATYYCQQYNSYSFWTFGQGTKVEIKRTVAAPSVFIFPPSDEQLKS |
| | GTASVVCLLNNFYPREAKVQWKVDNALQSGNSQESVTEQDSKDSTYSLSS |
| | TLTLSKADYEKHKVYACEVTHQGLSSPVTKSFNRGEC |
| | [SEQ ID NO: 241] |
| Heavy | QVQLVESGGGVVQPGRSLRLSCAASGFT |
| Chain | FNTYGMHWVRQAPGKGLEWVAIIWYDGSQKYYADSVQGRFIISRDNHKNT |
| version | LSLQMNSLRAEDTAVYFCVRVRFSVGPHGSAFDLWGQGTMVTVSSASTKG |
| 1 | PSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNV |
| | FSCSVMHEALHNHYTQKSLSLSPGK |
| | [SEQ ID NO: 242] |
| Heavy | QVQLVESGGGVVQPGRSLRLSCAASGFT |
| Chain | FNTYGMHWVRQAPGKGLEWVAIIWYDGSQKYYADSVQGRFIISRDNHKNT |
| version | LSLQMNSLRAEDTAVYFCVRVRFSVGPHGSAFDLWGQGTMVTVSSASTKG |
| 2 | PSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNV |
| | FSCSVLHEALHSHYTQKSLSLSPGK [SEQ ID NO: 243] |
| DNA | GGCGTCCAGATGACCCAGTCTCCTTCCACCCTGT |
| for | CTGCATCTGTGGGAGACAGAGTCACCCTCACTTGCCGGGCCAGTCAGAGT |
| Light | ATTAGTAGTTGGTTGGCCTGGTATCAGCAGAAACCAGGGAAAGCCCCTAA |
| Chain | ACTCCTGATCTATGATGCCTCCAGTTTGGAAAGTGGGGTCCCATCAAGGT |
| | TCAGCGGCAGTGGATCTGGGACAGAATTCACTCTCACCATCAGCAGCCTG |
| | CAACCTGATGATTTTGCAACTTATTACTGCCAACAGTATAATAGTTATTC |
| | TTTTTGGACGTTCGGCCAAGGGACCAAGGTGGAAATCAAACGCACTGTGG |
| | CTGCACCATCTGTCTTCATCTTCCCGCCATCTGATGAGCAGTTGAAATCT |
| | GGAACTGCCTCTGTTGTGTGCCTGCTGAATAACTTCTATCCCAGAGAGGC |
| | CAAAGTACAGTGGAAGGTGGATAACGCCCTCCAATCGGGTAACTCCCAGG |
| | AGAGTGTCACAGAGCAGGACAGCAAGGACAGCACCTACAGCCTCAGCAGC |
| | ACCCTGACGCTGAGCAAAGCAGACTACGAGAAACACAAAGTCTACGCCTG |
| | CGAAGTCACCCATCAGGGCCTGAGCTCGCCCGTCACAAAGAGCTTCAACA |
| | GGGGAGAGTGT |
| | [SEQ ID NO: 244] |
| DNA | CAGGTGCAACTGGTGGAGTCTGGGGGAGGCGTGG |
| for | TCCAGCCTGGGAGGTCCCTGAGACTCTCCTGTGCAGCGTCTGGGTTCACT |
| Heavy | TTCAATACCTATGGCATGCACTGGGTCCGCCAGGCACCAGGCAAGGGGCT |
| Chain | GGAGTGGGTGGCAATTATCTGGTATGATGGGAGCCAGAAATACTATGCAG |
| version | ACTCCGTGCAGGGCCGATTCATCATCTCCAGAGACAATCACAAGAACACG |
| 1 | TTGTCTCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCAGCCTCCACCAAGGGC |
| | CCATCGGTCTTCCCCCTGGCACCCTCCTCCAAGAGCACCTCTGGGGGCAC |
| | AGCAGCCCTGGGCTGCCTGGTCAAGGACTACTTCCCCGAACCGGTGACGG |
| | TGTCGTGGAACTCAGGCGCCCTGACCAGCGGCGTGCACACCTTCCCGGCT |
| | GTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGAC |
| | AAAGCCGCGGGAGGAGCAGTACAACAGCACGTACCGTGTGGTCAGCGTCC |
| | TCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGC |
| | CAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCC |
| | TCTACAGCAAGCTCACCGTGGACAAGAGCAGGTGGCAGCAGGGGAACGTC |
| | TTCTCATGCTCCGTGATGCATGAGGCTCTGCACAACCACTACACACAGAA |
| | GAGCCTCTCCCTGTCTCCGGGTAAA [SEQ ID NO: 245] |
| DNA | CAGGTGCAACTGGTGGAGTCTGGGGGAGGCGTGG |
| for | TCCAGCCTGGGAGGTCCCTGAGACTCTCCTGTGCAGCGTCTGGGTTCACT |
| Heavy | TTCAATACCTATGGCATGCACTGGGTCCGCCAGGCACCAGGCAAGGGGCT |
| Chain | GGAGTGGGTGGCAATTATCTGGTATGATGGGAGCCAGAAATACTATGCAG |
| version | ACTCCGTGCAGGGCCGATTCATCATCTCCAGAGACAATCACAAGAACACG |
| 2 | TTGTCTCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCAGCCTCCACCAAGGGC |
| | CCATCGGTCTTCCCCCTGGCACCCTCCTCCAAGAGCACCTCTGGGGGCAC |
| | AGCAGCCCTGGGCTGCCTGGTCAAGGACTACTTCCCCGAACCGGTGACGG |
| | TGTCGTGGAACTCAGGCGCCCTGACCAGCGGCGTGCACACCTTCCCGGCT |
| | GTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGAC |
| | AAAGCCGCGGGAGGAGCAGTACAACAGCACGTACCGTGTGGTCAGCGTCC |
| | TCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGC |
| | CAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCC |
| | TCTACAGCAAGCTCACCGTGGACAAGAGCAGGTGGCAGCAGGGGAACGTC |
| | TTCTCATGCTCCGTGCTGCATGAGGCTCTGCACTCCCACTACACACAGAA |
| | GAGCCTCTCCCTGTCTCCGGGTAAA [SEQ ID NO: 246] |

In certain embodiments, the anti-CSP antibody variant comprises a light chain variable region comprising a CDR1, a CDR2, and a CDR3 and having the amino acid sequence set forth in SEQ ID NO: 247, as shown in Table 28. In certain embodiments, the anti-CSP antibody variant comprises a heavy chain variable region comprising a CDR1, a CDR2, and a CDR3 and having the amino acid sequence set forth in SEQ ID NO: 248, as shown in Table 28. In certain embodiments, the anti-CSP antibody variant comprises a light chain having the amino acid sequence set forth in SEQ ID NO: 251, as shown in Table 28. In certain embodiments, the anti-CSP antibody variant comprises a heavy chain having the amino acid sequence set forth in SEQ ID NO: 252, as shown in Table 28. In certain embodiments, the anti-CSP antibody variant comprises a heavy chain having the amino acid sequence set forth in SEQ ID NO: 253, as shown in Table 28. Exemplary nucleic acid sequences of SEQ ID NOS: 247, 248, 251, 252, and 253 are provided in Table 28 below.

TABLE 28
Antibody ID: AB-007088.003

| | KABAT | ASN |
|---|---|---|
| CDR1- | RASQSISSWLA [SEQ ID NO: 205] | RASQSISSWLA [SEQ ID NO: 211] |
| VL | | |
| CDR2- | DASSLES [SEQ ID NO: 206] | DASSLES [SEQ ID NO: 212] |
| VL | | |
| CDR3- | QQYNSYSFWT [SEQ ID NO: 207] | QQYNSYSFWTF [SEQ ID NO: 213] |
| VL | | |
| CDR1- | TYGMH [SEQ ID NO: 208] | TYGMH [SEQ ID NO: 214] |
| VH | | |
| CDR2- | IIWYDGSQKYYADSVQG [SEQ ID | IIWYDGSQKYYADSVQG [SEQ ID NO: |
| VH | NO: 209] | 215] |
| CDR3- | VRFSVGPHGSAFDL [SEQ ID NO: | SAFDL [SEQ ID NO: 216] |
| VH | 210] | |

| | |
|---|---|
| VL | GVQMTQSPSTLSASVGDRVTLTCRASQS |
| | ISSWLAWYQQKPGKAPKLLIYDASSLESGVPSRFSGSGSGTEFTLTISSL |
| | QPDDFATYYCQQYNSYSFWTFGQGTKVEIKR |
| | [SEQ ID NO: 247] |
| VH | QVQLVESGGGVVQPGRSLRLSCAASGFA |
| | FNTYGMHWVRQAPGKGLEWVAIIWYDGSQKYYADSVQGRFTISRDNHKNT |
| | LSLQMNSLRAEDTAVYFCVRVRFSVGPHGSAFDLWGQGTMVTVSS |
| | [SEQ ID NO: 248] |
| DNA | GGCGTCCAGATGACCCAGTCTCCTTCCACCCTGT |
| for VL | CTGCATCTGTGGGAGACAGAGTCACCCTCACTTGCCGGGCCAGTCAGAGT |
| | ATTAGTAGTTGGTTGGCCTGGTATCAGCAGAAACCAGGGAAAGCCCCTAA |
| | ACTCCTGATCTATGATGCCTCCAGTTTGGAAAGTGGGGTCCCATCAAGGT |
| | TCAGCGGCAGTGGATCTGGGACAGAATTCACTCTCACCATCAGCAGCCTG |
| | CAACCTGATGATTTTGCAACTTATTACTGCCAACAGTATAATAGTTATTC |
| | TTTTTGGACGTTCGGCCAAGGGACCAAGGTGGAAATCAAACGC |
| | [SEQ ID NO: 249] |
| DNA | CAGGTGCAACTGGTGGAGTCTGGGGGAGGCGTGG |
| forVH | TCCAGCCTGGGAGGTCCCTGAGACTCTCCTGTGCAGCGTCTGGGTTCGCT |
| | TTCAATACCTATGGCATGCACTGGGTCCGCCAGGCACCAGGCAAGGGGCT |
| | GGAGTGGGTGGCAATTATCTGGTATGATGGGAGCCAGAAATACTATGCAG |
| | ACTCCGTGCAGGGCCGATTCACTATCTCCAGAGACAATCACAAGAACACG |
| | TTGTCTCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCA |
| | [SEQ ID NO: 250] |
| Light | GVQMTQSPSTLSASVGDRVTLTCRASQS |
| Chain | ISSWLAWYQQKPGKAPKLLIYDASSLESGVPSRFSGSGSGTEFTLTISSL |
| | QPDDFATYYCQQYNSYSFWTFGQGTKVEIKRTVAAPSVFIFPPSDEQLKS |
| | GTASVVCLLNNFYPREAKVQWKVDNALQSGNSQESVTEQDSKDSTYSLSS |
| | TLTLSKADYEKHKVYACEVTHQGLSSPVTKSFNRGEC |
| | [SEQ ID NO: 251] |
| Heavy | QVQLVESGGGVVQPGRSLRLSCAASGFA |
| Chain | FNTYGMHWVRQAPGKGLEWVAIIWYDGSQKYYADSVQGRFTISRDNHKNT |
| version | LSLQMNSLRAEDTAVYFCVRVRFSVGPHGSAFDLWGQGTMVTVSSASTKG |
| 1 | PSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNV |
| | FSCSVMHEALHNHYTQKSLSLSPGK |
| | [SEQ ID NO: 252] |
| Heavy | QVQLVESGGGVVQPGRSLRLSCAASGFA |
| Chain | FNTYGMHWVRQAPGKGLEWVAIIWYDGSQKYYADSVQGRFTISRDNHKNT |
| version | LSLQMNSLRAEDTAVYFCVRVRFSVGPHGSAFDLWGQGTMVTVSSASTKG |
| 2 | PSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNV |
| | FSCSVLHEALHSHYTQKSLSLSPGK [SEQ ID NO: 253] |
| DNA | GGCGTCCAGATGACCCAGTCTCCTTCCACCCTGT |
| for | CTGCATCTGTGGGAGACAGAGTCACCCTCACTTGCCGGGCCAGTCAGAGT |
| Light | ATTAGTAGTTGGTTGGCCTGGTATCAGCAGAAACCAGGGAAAGCCCCTAA |
| Chain | ACTCCTGATCTATGATGCCTCCAGTTTGGAAAGTGGGGTCCCATCAAGGT |
| | TCAGCGGCAGTGGATCTGGGACAGAATTCACTCTCACCATCAGCAGCCTG |
| | CAACCTGATGATTTTGCAACTTATTACTGCCAACAGTATAATAGTTATTC |
| | TTTTTGGACGTTCGGCCAAGGGACCAAGGTGGAAATCAAACGCACTGTGG |
| | CTGCACCATCTGTCTTCATCTTCCCGCCATCTGATGAGCAGTTGAAATCT |
| | GGAACTGCCTCTGTTGTGTGCCTGCTGAATAACTTCTATCCCAGAGAGGC |
| | CAAAGTACAGTGGAAGGTGGATAACGCCCTCCAATCGGGTAACTCCCAGG |
| | AGAGTGTCACAGAGCAGGACAGCAAGGACAGCACCTACAGCCTCAGCAGC |
| | ACCCTGACGCTGAGCAAAGCAGACTACGAGAAACACAAAGTCTACGCCTG |
| | CGAAGTCACCCATCAGGGCCTGAGCTCGCCCGTCACAAAGAGCTTCAACA |
| | GGGGAGAGTGT |
| | [SEQ ID NO: 254] |
| DNA | CAGGTGCAACTGGTGGAGTCTGGGGGAGGCGTGG |
| for | TCCAGCCTGGGAGGTCCCTGAGACTCTCCTGTGCAGCGTCTGGGTTCGCT |
| Heavy | TTCAATACCTATGGCATGCACTGGGTCCGCCAGGCACCAGGCAAGGGGCT |
| Chain | GGAGTGGGTGGCAATTATCTGGTATGATGGGAGCCAGAAATACTATGCAG |
| version | ACTCCGTGCAGGGCCGATTCACTATCTCCAGAGACAATCACAAGAACACG |
| 1 | TTGTCTCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCAGCCTCCACCAAGGGC |
| | CCATCGGTCTTCCCCCTGGCACCCTCCTCCAAGAGCACCTCTGGGGGCAC |
| | AGCAGCCCTGGGCTGCCTGGTCAAGGACTACTTCCCCGAACCGGTGACGG |
| | TGTCGTGGAACTCAGGCGCCCTGACCAGCGGCGTGCACACCTTCCCGGCT |
| | GTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGAC |
| | AAAGCCGCGGGAGGAGCAGTACAACAGCACGTACCGTGTGGTCAGCGTCC |
| | TCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGC |
| | CAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCC |
| | TCTACAGCAAGCTCACCGTGGACAAGAGCAGGTGGCAGCAGGGGAACGTC |
| | TTCTCATGCTCCGTGATGCATGAGGCTCTGCACAACCACTACACACAGAA |
| | GAGCCTCTCCCTGTCTCCGGGTAAA [SEQ ID NO: 255] |
| DNA | CAGGTGCAACTGGTGGAGTCTGGGGGAGGCGTGG |
| for | TCCAGCCTGGGAGGTCCCTGAGACTCTCCTGTGCAGCGTCTGGGTTCGCT |
| Heavy | TTCAATACCTATGGCATGCACTGGGTCCGCCAGGCACCAGGCAAGGGGCT |
| Chain | GGAGTGGGTGGCAATTATCTGGTATGATGGGAGCCAGAAATACTATGCAG |
| version | ACTCCGTGCAGGGCCGATTCACTATCTCCAGAGACAATCACAAGAACACG |
| 2 | TTGTCTCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCAGCCTCCACCAAGGGC |
| | CCATCGGTCTTCCCCCTGGCACCCTCCTCCAAGAGCACCTCTGGGGGCAC |
| | AGCAGCCCTGGGCTGCCTGGTCAAGGACTACTTCCCCGAACCGGTGACGG |
| | TGTCGTGGAACTCAGGCGCCCTGACCAGCGGCGTGCACACCTTCCCGGCT |
| | GTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGAC |
| | AAAGCCGCGGGAGGAGCAGTACAACAGCACGTACCGTGTGGTCAGCGTCC |
| | TCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGC |
| | CAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCC |
| | TCTACAGCAAGCTCACCGTGGACAAGAGCAGGTGGCAGCAGGGGAACGTC |
| | TTCTCATGCTCCGTGCTGCATGAGGCTCTGCACTCCCACTACACACAGAA |
| | GAGCCTCTCCCTGTCTCCGGGTAAA [SEQ ID NO: 256] |

In certain embodiments, the anti-CSP antibody variant comprises a light chain variable region comprising a CDR1, a CDR2, and a CDR3 and having the amino acid sequence set forth in SEQ ID NO: 257, as shown in Table 29. In certain embodiments, the anti-CSP antibody variant comprises a heavy chain variable region comprising a CDR1, a CDR2, and a CDR3 and having the amino acid sequence set forth in SEQ ID NO: 258, as shown in Table 29. In certain embodiments, the anti-CSP antibody variant comprises a light chain having the amino acid sequence set forth in SEQ ID NO: 261, as shown in Table 29. In certain embodiments, the anti-CSP antibody variant comprises a heavy chain having the amino acid sequence set forth in SEQ ID NO: 262, as shown in Table 29. In certain embodiments, the anti-CSP antibody variant comprises a heavy chain having the amino acid sequence set forth in SEQ ID NO: 263, as shown in Table 29. Exemplary nucleic acid sequences of SEQ ID NOS: 257, 258, 261, 262, and 263 are provided in Table 29 below.

TABLE 29
Antibody ID: AB-007088.004

| | KABAT | ASN |
|---|---|---|
| CDR1- | RASQSISSWLA [SEQ ID NO: 205] | RASQSISSWLA [SEQ ID NO: 211] |
| VL | | |
| CDR2- | DASSLES [SEQ ID NO: 206] | DASSLES [SEQ ID NO: 212] |
| VL | | |
| CDR3- | QQYNSYSFWT [SEQ ID NO: 207] | QQYNSYSFWTF [SEQ ID NO: 213] |
| VL | | |
| CDR1- | TYGMH [SEQ ID NO: 208] | TYGMH [SEQ ID NO: 214] |
| VH | | |
| CDR2- | IIWYDGSQKYYADSVQG [SEQ ID | IIWYDGSQKYYADSVQG [SEQ ID NO: |
| VH | NO: 209] | 215] |
| CDR3- | VRFSVGPHGSAFDL [SEQ ID NO: | SAFDL [SEQ ID NO: 216] |
| VH | 210] | |

| | |
|---|---|
| VL | GVQMTQSPSTLSASVGDRVTLTCRASQS |
| | ISSWLAWYQQKPGKAPKLLIYDASSLESGVPSRFSGSGSGTEFTLTISSL |
| | QPDDFATYYCQQYNSYSFWTFGQGTKVEIKR |
| | [SEQ ID NO: 257] |
| VH | QVQLVESGGGVVQPGRSLRLSCAASGFT |
| | FNTYGMHWVRQAPGKGLEWVAIIWYDGSQKYYADSVQGRFTISRDNHKNT |
| | LSLQMNSLRAEDTAVYFCVRVRFSVGPHGSAFDLWGQGTMVTVSS |
| | [SEQ ID NO: 258] |
| DNA | GGCGTCCAGATGACCCAGTCTCCTTCCACCCTGT |
| for VL | CTGCATCTGTGGGAGACAGAGTCACCCTCACTTGCCGGGCCAGTCAGAGT |
| | ATTAGTAGTTGGTTGGCCTGGTATCAGCAGAAACCAGGGAAAGCCCCTAA |
| | ACTCCTGATCTATGATGCCTCCAGTTTGGAAAGTGGGGTCCCATCAAGGT |
| | TCAGCGGCAGTGGATCTGGGACAGAATTCACTCTCACCATCAGCAGCCTG |
| | CAACCTGATGATTTTGCAACTTATTACTGCCAACAGTATAATAGTTATTC |
| | TTTTTGGACGTTCGGCCAAGGGACCAAGGTGGAAATCAAACGC |
| | [SEQ ID NO: 259] |
| DNA | CAGGTGCAACTGGTGGAGTCTGGGGGAGGCGTGG |
| forVH | TCCAGCCTGGGAGGTCCCTGAGACTCTCCTGTGCAGCGTCTGGGTTCACT |
| | TTCAATACCTATGGCATGCACTGGGTCCGCCAGGCACCAGGCAAGGGGCT |
| | GGAGTGGGTGGCAATTATCTGGTATGATGGGAGCCAGAAATACTATGCAG |
| | ACTCCGTGCAGGGCCGATTCACTATCTCCAGAGACAATCACAAGAACACG |
| | TTGTCTCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCA |
| | [SEQ ID NO: 260] |
| Light | GVQMTQSPSTLSASVGDRVTLTCRASQS |
| Chain | ISSWLAWYQQKPGKAPKLLIYDASSLESGVPSRFSGSGSGTEFTLTISSL |
| | QPDDFATYYCQQYNSYSFWTFGQGTKVEIKRTVAAPSVFIFPPSDEQLKS |
| | GTASVVCLLNNFYPREAKVQWKVDNALQSGNSQESVTEQDSKDSTYSLSS |
| | TLTLSKADYEKHKVYACEVTHQGLSSPVTKSFNRGEC |
| | [SEQ ID NO: 261] |
| Heavy | QVQLVESGGGVVQPGRSLRLSCAASGFT |
| Chain | FNTYGMHWVRQAPGKGLEWVAIIWYDGSQKYYADSVQGRFTISRDNHKNT |
| version | LSLQMNSLRAEDTAVYFCVRVRFSVGPHGSAFDLWGQGTMVTVSSASTKG |
| 1 | PSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNV |
| | FSCSVMHEALHNHYTQKSLSLSPGK |
| | [SEQ ID NO: 262] |
| Heavy | QVQLVESGGGVVQPGRSLRLSCAASGFT |
| Chain | FNTYGMHWVRQAPGKGLEWVAIIWYDGSQKYYADSVQGRFTISRDNHKNT |
| version | LSLQMNSLRAEDTAVYFCVRVRFSVGPHGSAFDLWGQGTMVTVSSASTKG |
| 2 | PSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNV |
| | FSCSVLHEALHSHYTQKSLSLSPGK [SEQ ID NO: 263] |
| DNA | GGCGTCCAGATGACCCAGTCTCCTTCCACCCTGT |
| for | CTGCATCTGTGGGAGACAGAGTCACCCTCACTTGCCGGGCCAGTCAGAGT |
| Light | ATTAGTAGTTGGTTGGCCTGGTATCAGCAGAAACCAGGGAAAGCCCCTAA |
| Chain | ACTCCTGATCTATGATGCCTCCAGTTTGGAAAGTGGGGTCCCATCAAGGT |
| | TCAGCGGCAGTGGATCTGGGACAGAATTCACTCTCACCATCAGCAGCCTG |
| | CAACCTGATGATTTTGCAACTTATTACTGCCAACAGTATAATAGTTATTC |
| | TTTTTGGACGTTCGGCCAAGGGACCAAGGTGGAAATCAAACGCACTGTGG |
| | CTGCACCATCTGTCTTCATCTTCCCGCCATCTGATGAGCAGTTGAAATCT |
| | GGAACTGCCTCTGTTGTGTGCCTGCTGAATAACTTCTATCCCAGAGAGGC |
| | CAAAGTACAGTGGAAGGTGGATAACGCCCTCCAATCGGGTAACTCCCAGG |
| | AGAGTGTCACAGAGCAGGACAGCAAGGACAGCACCTACAGCCTCAGCAGC |
| | ACCCTGACGCTGAGCAAAGCAGACTACGAGAAACACAAAGTCTACGCCTG |
| | CGAAGTCACCCATCAGGGCCTGAGCTCGCCCGTCACAAAGAGCTTCAACA |
| | GGGGAGAGTGT |
| | [SEQ ID NO: 264] |
| DNA | CAGGTGCAACTGGTGGAGTCTGGGGGAGGCGTGG |
| for | TCCAGCCTGGGAGGTCCCTGAGACTCTCCTGTGCAGCGTCTGGGTTCACT |
| Heavy | TTCAATACCTATGGCATGCACTGGGTCCGCCAGGCACCAGGCAAGGGGCT |
| Chain | GGAGTGGGTGGCAATTATCTGGTATGATGGGAGCCAGAAATACTATGCAG |
| version | ACTCCGTGCAGGGCCGATTCACTATCTCCAGAGACAATCACAAGAACACG |
| 1 | TTGTCTCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCAGCCTCCACCAAGGGC |
| | CCATCGGTCTTCCCCCTGGCACCCTCCTCCAAGAGCACCTCTGGGGGCAC |
| | AGCAGCCCTGGGCTGCCTGGTCAAGGACTACTTCCCCGAACCGGTGACGG |
| | TGTCGTGGAACTCAGGCGCCCTGACCAGCGGCGTGCACACCTTCCCGGCT |
| | GTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGAC |
| | AAAGCCGCGGGAGGAGCAGTACAACAGCACGTACCGTGTGGTCAGCGTCC |
| | TCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGC |
| | CAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCC |
| | TCTACAGCAAGCTCACCGTGGACAAGAGCAGGTGGCAGCAGGGGAACGTC |
| | TTCTCATGCTCCGTGATGCATGAGGCTCTGCACAACCACTACACACAGAA |
| | GAGCCTCTCCCTGTCTCCGGGTAAA [SEQ ID NO: 265] |
| DNA | CAGGTGCAACTGGTGGAGTCTGGGGGAGGCGTGG |
| for | TCCAGCCTGGGAGGTCCCTGAGACTCTCCTGTGCAGCGTCTGGGTTCACT |
| Heavy | TTCAATACCTATGGCATGCACTGGGTCCGCCAGGCACCAGGCAAGGGGCT |
| Chain | GGAGTGGGTGGCAATTATCTGGTATGATGGGAGCCAGAAATACTATGCAG |
| version | ACTCCGTGCAGGGCCGATTCACTATCTCCAGAGACAATCACAAGAACACG |
| 2 | TTGTCTCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCAGCCTCCACCAAGGGC |
| | CCATCGGTCTTCCCCCTGGCACCCTCCTCCAAGAGCACCTCTGGGGGCAC |
| | AGCAGCCCTGGGCTGCCTGGTCAAGGACTACTTCCCCGAACCGGTGACGG |
| | TGTCGTGGAACTCAGGCGCCCTGACCAGCGGCGTGCACACCTTCCCGGCT |
| | GTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGAC |
| | AAAGCCGCGGGAGGAGCAGTACAACAGCACGTACCGTGTGGTCAGCGTCC |
| | TCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGC |
| | CAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCC |
| | TCTACAGCAAGCTCACCGTGGACAAGAGCAGGTGGCAGCAGGGGAACGTC |
| | TTCTCATGCTCCGTGCTGCATGAGGCTCTGCACTCCCACTACACACAGAA |
| | GAGCCTCTCCCTGTCTCCGGGTAAA [SEQ ID NO: 266] |

In certain embodiments, the anti-CSP antibody variant comprises a light chain variable region comprising a CDR1, a CDR2, and a CDR3 and having the amino acid sequence set forth in SEQ ID NO: 267, as shown in Table 30. In certain embodiments, the anti-CSP antibody variant comprises a heavy chain variable region comprising a CDR1, a CDR2, and a CDR3 and having the amino acid sequence set forth in SEQ ID NO: 268, as shown in Table 30. In certain embodiments, the anti-CSP antibody variant comprises a light chain having the amino acid sequence set forth in SEQ ID NO: 271, as shown in Table 30. In certain embodiments, the anti-CSP antibody variant comprises a heavy chain having the amino acid sequence set forth in SEQ ID NO: 272, as shown in Table 30. In certain embodiments, the anti-CSP antibody variant comprises a heavy chain having the amino acid sequence set forth in SEQ ID NO: 273, as shown in Table 30. Exemplary nucleic acid sequences of SEQ ID NOS: 267, 268, 271, 272, and 273 are provided in Table 30 below.

TABLE 30
Antibody ID: AB-007088.005

| | KABAT | ASN |
|---|---|---|
| CDR1- | RASQSISSWLA [SEQ ID NO: 205] | RASQSISSWLA [SEQ ID NO: 211] |
| VL | | |
| CDR2- | DASSLES [SEQ ID NO: 206] | DASSLES [SEQ ID NO: 212] |
| VL | | |
| CDR3- | QQYNSYSFWT [SEQ ID NO: 207] | QQYNSYSFWTF [SEQ ID NO: 213] |
| VL | | |
| CDR1- | TYGMH [SEQ ID NO: 208] | TYGMH [SEQ ID NO: 214] |
| VH | | |
| CDR2- | IIWYDGSQKYYADSVQG [SEQ ID | IIWYDGSQKYYADSVQG [SEQ ID NO: |
| VH | NO: 209] | 215] |
| CDR3- | VRFSVGPHGSAFDL [SEQ ID NO: | SAFDL [SEQ ID NO: 216] |
| VH | 210] | |

| | |
|---|---|
| VL | GVQMTQSPSTLSASVGDRVTLTCRASQS |
| | ISSWLAWYQQKPGKAPKLLIYDASSLESGVPSRFSGSGSGTEFTLTISSL |
| | QPDDFATYYCQQYNSYSFWTFGQGTKVEIKR |
| | [SEQ ID NO: 267] |
| VH | QVQLVESGGGVVQPGRSLRLSCAASGFT |
| | FNTYGMHWVRQAPGKGLEWVAIIWYDGSQKYYADSVQGRFTISRDNHKNT |
| | LYLQMNSLRAEDTAVYFCVRVRFSVGPHGSAFDLWGQGTMVTVSS |
| | [SEQ ID NO: 268] |
| DNA | GGCGTCCAGATGACCCAGTCTCCTTCCACCCTGT |
| for VL | CTGCATCTGTGGGAGACAGAGTCACCCTCACTTGCCGGGCCAGTCAGAGT |
| | ATTAGTAGTTGGTTGGCCTGGTATCAGCAGAAACCAGGGAAAGCCCCTAA |
| | ACTCCTGATCTATGATGCCTCCAGTTTGGAAAGTGGGGTCCCATCAAGGT |
| | TCAGCGGCAGTGGATCTGGGACAGAATTCACTCTCACCATCAGCAGCCTG |
| | CAACCTGATGATTTTGCAACTTATTACTGCCAACAGTATAATAGTTATTC |
| | TTTTTGGACGTTCGGCCAAGGGACCAAGGTGGAAATCAAACGC |
| | [SEQ ID NO: 269] |
| DNA | CAGGTGCAACTGGTGGAGTCTGGGGGAGGCGTGG |
| for VH | TCCAGCCTGGGAGGTCCCTGAGACTCTCCTGTGCAGCGTCTGGGTTCACT |
| | TTCAATACCTATGGCATGCACTGGGTCCGCCAGGCACCAGGCAAGGGGCT |
| | GGAGTGGGTGGCAATTATCTGGTATGATGGGAGCCAGAAATACTATGCAG |
| | ACTCCGTGCAGGGCCGATTCACTATCTCCAGAGACAATCACAAGAACACG |
| | TTGTACCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCA |
| | [SEQ ID NO: 270] |
| Light | GVQMTQSPSTLSASVGDRVTLTCRASQS |
| Chain | ISSWLAWYQQKPGKAPKLLIYDASSLESGVPSRFSGSGSGTEFTLTISSL |
| | QPDDFATYYCQQYNSYSFWTFGQGTKVEIKRTVAAPSVFIFPPSDEQLKS |
| | GTASVVCLLNNFYPREAKVQWKVDNALQSGNSQESVTEQDSKDSTYSLSS |
| | TLTLSKADYEKHKVYACEVTHQGLSSPVTKSFNRGEC |
| | [SEQ ID NO: 271] |
| Heavy | QVQLVESGGGVVQPGRSLRLSCAASGFT |
| Chain | FNTYGMHWVRQAPGKGLEWVAIIWYDGSQKYYADSVQGRFTISRDNHKNT |
| version | LYLQMNSLRAEDTAVYFCVRVRFSVGPHGSAFDLWGQGTMVTVSSASTKG |
| 1 | PSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNV |
| | FSCSVMHEALHNHYTQKSLSLSPGK |
| | [SEQ ID NO: 272] |
| Heavy | QVQLVESGGGVVQPGRSLRLSCAASGFT |
| Chain | FNTYGMHWVRQAPGKGLEWVAIIWYDGSQKYYADSVQGRFTISRDNHKNT |
| version | LYLQMNSLRAEDTAVYFCVRVRFSVGPHGSAFDLWGQGTMVTVSSASTKG |
| 2 | PSVFPLAPSSKSTSGGTAALGCLVKDYFPEPVTVSWNSGALTSGVHTFPA |
| | VLQSSGLYSLSSVVTVPSSSLGTQTYICNVNHKPSNTKVDKKVEPKSCDK |
| | THTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNV |
| | FSCSVLHEALHSHYTQKSLSLSPGK [SEQ ID NO: 273] |
| DNA | GGCGTCCAGATGACCCAGTCTCCTTCCACCCTGT |
| for | CTGCATCTGTGGGAGACAGAGTCACCCTCACTTGCCGGGCCAGTCAGAGT |
| Light | ATTAGTAGTTGGTTGGCCTGGTATCAGCAGAAACCAGGGAAAGCCCCTAA |
| Chain | ACTCCTGATCTATGATGCCTCCAGTTTGGAAAGTGGGGTCCCATCAAGGT |
| | TCAGCGGCAGTGGATCTGGGACAGAATTCACTCTCACCATCAGCAGCCTG |
| | CAACCTGATGATTTTGCAACTTATTACTGCCAACAGTATAATAGTTATTC |
| | TTTTTGGACGTTCGGCCAAGGGACCAAGGTGGAAATCAAACGCACTGTGG |
| | CTGCACCATCTGTCTTCATCTTCCCGCCATCTGATGAGCAGTTGAAATCT |
| | GGAACTGCCTCTGTTGTGTGCCTGCTGAATAACTTCTATCCCAGAGAGGC |
| | CAAAGTACAGTGGAAGGTGGATAACGCCCTCCAATCGGGTAACTCCCAGG |
| | AGAGTGTCACAGAGCAGGACAGCAAGGACAGCACCTACAGCCTCAGCAGC |
| | ACCCTGACGCTGAGCAAAGCAGACTACGAGAAACACAAAGTCTACGCCTG |
| | CGAAGTCACCCATCAGGGCCTGAGCTCGCCCGTCACAAAGAGCTTCAACA |
| | GGGGAGAGTGT |
| | [SEQ ID NO: 274] |
| DNA | CAGGTGCAACTGGTGGAGTCTGGGGGAGGCGTGG |
| for | TCCAGCCTGGGAGGTCCCTGAGACTCTCCTGTGCAGCGTCTGGGTTCACT |
| Heavy | TTCAATACCTATGGCATGCACTGGGTCCGCCAGGCACCAGGCAAGGGGCT |
| Chain | GGAGTGGGTGGCAATTATCTGGTATGATGGGAGCCAGAAATACTATGCAG |
| version | ACTCCGTGCAGGGCCGATTCACTATCTCCAGAGACAATCACAAGAACACG |
| 1 | TTGTACCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCAGCCTCCACCAAGGGC |
| | CCATCGGTCTTCCCCCTGGCACCCTCCTCCAAGAGCACCTCTGGGGGCAC |
| | AGCAGCCCTGGGCTGCCTGGTCAAGGACTACTTCCCCGAACCGGTGACGG |
| | TGTCGTGGAACTCAGGCGCCCTGACCAGCGGCGTGCACACCTTCCCGGCT |
| | GTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGAC |
| | AAAGCCGCGGGAGGAGCAGTACAACAGCACGTACCGTGTGGTCAGCGTCC |
| | TCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGC |
| | CAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCC |
| | TCTACAGCAAGCTCACCGTGGACAAGAGCAGGTGGCAGCAGGGGAACGTC |
| | TTCTCATGCTCCGTGATGCATGAGGCTCTGCACAACCACTACACACAGAA |
| | GAGCCTCTCCCTGTCTCCGGGTAAA [SEQ ID NO: 275] |
| DNA | CAGGTGCAACTGGTGGAGTCTGGGGGAGGCGTGG |
| for | TCCAGCCTGGGAGGTCCCTGAGACTCTCCTGTGCAGCGTCTGGGTTCACT |
| Heavy | TTCAATACCTATGGCATGCACTGGGTCCGCCAGGCACCAGGCAAGGGGCT |
| Chain | GGAGTGGGTGGCAATTATCTGGTATGATGGGAGCCAGAAATACTATGCAG |
| version | ACTCCGTGCAGGGCCGATTCACTATCTCCAGAGACAATCACAAGAACACG |
| 2 | TTGTACCTGCAAATGAACTCCCTGAGAGCCGAGGACACGGCTGTGTATTT |
| | CTGTGTGAGAGTCCGCTTTAGCGTTGGCCCCCACGGGAGTGCTTTTGATC |
| | TCTGGGGCCAGGGGACAATGGTCACAGTCTCTTCAGCCTCCACCAAGGGC |
| | CCATCGGTCTTCCCCCTGGCACCCTCCTCCAAGAGCACCTCTGGGGGCAC |
| | AGCAGCCCTGGGCTGCCTGGTCAAGGACTACTTCCCCGAACCGGTGACGG |
| | TGTCGTGGAACTCAGGCGCCCTGACCAGCGGCGTGCACACCTTCCCGGCT |
| | GTCCTACAGTCCTCAGGACTCTACTCCCTCAGCAGCGTGGTGACCGTGCC |
| | CTCCAGCAGCTTGGGCACCCAGACCTACATCTGCAACGTGAATCACAAGC |
| | CCAGCAACACCAAGGTGGACAAGAAAGTTGAGCCCAAATCTTGTGACAAA |
| | ACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGAC |
| | AAAGCCGCGGGAGGAGCAGTACAACAGCACGTACCGTGTGGTCAGCGTCC |
| | TCACCGTCCTGCACCAGGACTGGCTGAATGGCAAGGAGTACAAGTGCAAG |
| | GTCTCCAACAAAGCCCTCCCAGCCCCCATCGAGAAAACCATCTCCAAAGC |
| | CAAAGGGCAGCCCCGAGAACCACAGGTGTACACCCTGCCCCCATCCCGGG |
| | ATGAGCTGACCAAGAACCAGGTCAGCCTGACCTGCCTGGTCAAAGGCTTC |
| | TATCCCAGCGACATCGCCGTGGAGTGGGAGAGCAATGGGCAGCCGGAGAA |
| | CAACTACAAGACCACGCCTCCCGTGCTGGACTCCGACGGCTCCTTCTTCC |
| | TCTACAGCAAGCTCACCGTGGACAAGAGCAGGTGGCAGCAGGGGAACGTC |
| | TTCTCATGCTCCGTGCTGCATGAGGCTCTGCACTCCCACTACACACAGAA |
| | GAGCCTCTCCCTGTCTCCGGGTAAA [SEQ ID NO: 276] |

### 4. Glycosylation of Anti-CSP Antibodies and Variants Thereof

Glycosylation of antibodies and engineered antibodies has been previously disclosed (see, e.g., U.S. Pat. No. 6,602,684, the content of which is incorporated in its entirety). Antibody Fc regions are generally post-translationally modified via the addition of N-glycans at specific asparagine residues on the antibody heavy chain. IgG molecules bear a N-linked glycosylation asparagine of each heavy chain. It has been shown that a modified glycosylation profile can regulate the antibody functions. For example, without any limitation, altered glycosylation can improve the binding affinity or the half-life of the antibody as compared to the non-modified form.

In certain embodiments, the present disclosure provides anti-CSP antibodies and variants thereof with modified glycosylation. In certain embodiments, the antibodies disclosed herein include an Fc region with increased glycosylation. In certain non-limiting embodiments, the Fc region with increased glycosylation includes increased amounts of bisected oligosaccharides. In certain embodiments, the Fc region with increased glycosylation includes increased amounts of nonfucosylated oligosaccharides. In certain embodiments, the Fc region with increased glycosylation includes increased amounts of fucose-containing oligosaccharides.

In certain embodiments, the antibodies disclosed herein include an Fc region with decreased glycosylation. In certain non-limiting embodiments, the Fc region with decreased glycosylation includes reduced amounts of bisected oligosaccharides. In certain embodiments, the Fc region with decreased glycosylation includes reduced amounts of nonfucosylated oligosaccharides. In certain embodiments, the Fc region with increased glycosylation includes reduced amounts of fucose-containing oligosaccharides.

In certain embodiments, the antibodies disclosed herein include a V region with increased glycosylation. In certain non-limiting embodiments, the V region with increased glycosylation includes increased amounts of bisected oligosaccharides. In certain embodiments, the V region with increased glycosylation includes increased amounts of nonfucosylated oligosaccharides. In certain embodiments, the V region with increased glycosylation includes increased amounts of fucose-containing oligosaccharides.

In certain embodiments, the antibodies disclosed herein include a V region with decreased glycosylation. In certain non-limiting embodiments, the V region with decreased glycosylation includes reduced amounts of bisected oligosaccharides. In certain embodiments, the V region with decreased glycosylation includes reduced amounts of nonfucosylated oligosaccharides. In certain embodiments, the V region with increased glycosylation includes reduced amounts of fucose-containing oligosaccharides.

In certain embodiments, the modified glycosylation can be obtained by expressing any of the antibodies disclosed herein in a host cell with altered glycosylation machinery. For example, without any limitation, a host cell can include a functional disruption of the fucosyltransferase gene and antibodies expressed in this host cell with show reduced glycosylation, e.g., reduced fucosylation (see PCT Patent Publication No. WO 99/54342).

In certain embodiments, the present disclosure provides anti-CSP antibody variants disclosed herein including one or more amino acid substitution resulting in the alteration of a glycosylation acceptor site. In certain embodiments, the alteration includes the elimination of the glycosylation acceptor site. In certain embodiments, the alteration includes modification of a glycosylation acceptor site. In certain embodiments, the alteration includes insertion of a glycosylation acceptor site.

As used herein, “glycosylation acceptor site” refers to an amino acid residue of the light chain or heavy chain of the antibody which can be N- or O-glycosylated. In certain embodiments, the N-linked glycosylation acceptor site can be an asparagine residue. In certain embodiments, the O-linked glycosylation acceptor site can be a serine residue, a threonine residue, a tyrosine residue, a hydroxylysine residue, or a hydroxyproline residue.

In certain embodiments, the Fc region of the antibodies disclosed herein includes one or more glycosylation acceptor site. In certain embodiments, the V region of any of the antibodies disclosed herein includes one or more glycosylation acceptor site. In certain embodiments, the light chain of any of the antibodies disclosed herein includes one or more glycosylation acceptor site. In certain embodiments, the heavy chain of any one of the antibodies disclosed herein includes one or more glycosylation acceptor site. In certain embodiments, the light chain variable region of any of the antibodies disclosed herein includes one or more glycosylation acceptor site. In certain embodiments, the heavy chain variable region of any of the antibodies disclosed herein includes one or more glycosylation acceptor site.

### 5. PEGylation and Other Chemical Modifications of Anti-CSP Antibodies and Variants Thereof

The present disclosure provides anti-CSP antibodies and variants thereof including additional modifications. In certain embodiments, the modifications can improve pharmacological properties of the antibodies, e.g., half-life. In certain non-limiting embodiments, the modification includes PEGylation, deamination, derivatization with polymers, lipidation, removal and/or introduction of disulfide bonds, oxidation, and removal of C-terminal lysine

In certain embodiments, the modification is a PEGylation. PEGylation of antibodies and engineered antibodies includes attachment of one or more polyethylene glycol (PEG) to the antibody. In certain non-limitation embodiments, for example, the PEGylation can be carried out by an acylation reaction or an alkylation reaction with a reactive PEG molecule (or an analogous reactive water-soluble polymer). As used herein, the term “polyethylene glycol” refers to any of the forms of PEG that have been used to derivatize other proteins, such as mono (C1-C10) alkoxy- or aryloxy-polyethylene glycol or polyethylene glycol-maleimide.

In certain embodiments, the modification is the derivatization with a hydrophilic polymer. In certain non-limiting embodiments, for example, the hydrophilic polymer can be carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof.

In certain embodiments, the modification is a lipidation. Lipidation is the conjugation of a protein with a lipid. Lipidation of peptides improves metabolic stability, membrane permeability, bioavailability, and changes the pharmacokinetic and pharmacodynamic properties of the peptides. For example, a lipidated peptide has a high affinity with serum albumin resulting in increased half-life and stability. In certain non-limiting embodiments, for example, the lipid can be myristic acid, palmitic acid, stearic acid, lauric acid, cholesterol, and mixtures thereof.

In certain embodiments, the modification is a substitution of an amino acid residue to form a disulfide bond. In certain embodiments, the amino acid substitution introduces a cysteine. Under certain redox conditions, two cysteines can form a non-natural disulfide bond. In certain non-limiting embodiments, the disulfide bond improves the stability of the antibody, e.g., corrected pairing of the antibody chains. In certain embodiments, the cysteine is introduced in the V region. In certain embodiments, the cysteine is introduced in the Fc region. In certain embodiments, the modification is a substitution of an amino acid residue to remove a disulfide bond. In certain embodiments, the amino acid substitution removes a cysteine. In certain embodiments, the cysteine is substituted with a serine. In certain non-limiting embodiments, removing a cysteine improves the stability of the antibody, e.g., improved long-term stability. In certain embodiments, the cysteine is removed in the V region. In certain embodiments, the cysteine is removed in the Fc region.

### 6. Anti-CSP Antibody and Anti-CSP Antibody Variants Conjugates

In certain embodiments, the present disclosure provides an anti-CSP antibody or variant thereof conjugated or linked to therapeutic and/or imaging/detectable moieties. For example, without any limitation, the anti-CSP antibody or variant thereof can be conjugated to a detectable marker, a toxin, or a therapeutic agent. The moiety may be linked to the antibody covalently or by non-covalent linkages.

In certain embodiments, the antibody or variant thereof is conjugated to cytotoxic moiety or other moiety that inhibits cell proliferation. In certain embodiments, the antibody or variant thereof is conjugated to a cytotoxic agent including, but not limited to, a ricin A chain, doxorubicin, daunorubicin, a maytansinoid, taxol, ethidium bromide, mitomycin, etoposide, tenoposide, vincristine, vinblastine, colchicine, dihydroxy anthracin dione, actinomycin, a diphtheria toxin, exotoxin A from Pseudomonas, Pseudomonas exotoxin (PE) A, PE40, abrin, abrin A chain, modeccin A chain, alpha sarcin, gelonin, mitogellin, restrictocin, cobran venom factor, a ribonuclease, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc1065, or a cisplatin. In certain embodiments, the antibody or variant thereof can be linked to an agent such as an enzyme inhibitor, a proliferation inhibitor, a lytic agent, a DNA or RNA synthesis inhibitors, a membrane permeability modifier, a DNA metabolites, a dichloroethyl sulfide derivative, a protein production inhibitor, a ribosome inhibitor, or an inducer of apoptosis.

In certain embodiments, the antibody or variant thereof can be linked to a radionuclide, an iron-related compound, a dye, a fluorescent agent, or an imaging agent. In certain embodiments, an antibody may be linked to agents, such as, but not limited to, metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nano-compounds; magnetic resonance imaging contrast agents; light absorbing, reflecting and/or scattering agents; colloidal particles; fluorophores, such as near-infrared fluorophores.

In certain embodiments, the present disclosure provides bispecific molecules comprising an anti-CSP antibody, a variant thereof, or a fragment thereof, disclosed herein. The anti-CSP antibody, anti-CSP antibody variant or antigen-binding portions thereof can be derivatized or linked to another functional molecule, e.g., another peptide or protein (e.g., another antibody or ligand for a receptor) to generate a bispecific molecule that binds to at least two different binding sites or target molecules. The anti-CSP antibody or variant thereof disclosed herein can be derivatized or linked to more than one other functional molecule to generate multispecific molecules that bind to more than two different binding sites (e.g., two different epitopes on the CSP protein) and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association, or otherwise) to one or more other binding molecules, such as another antibody, antibody fragment, peptide or binding mimetic, such that a bispecific molecule results. In certain non-limiting embodiments, for example and without any limitation, the bispecific antibody can be created using the knobs-into-holes strategy. This strategy typically involves creation of a first half of a first antibody that recognizes a first antigen, e.g., CSP, and a second half of the antibody that recognizes a second antigen or binding site, and then joining the two halves to create the bispecific antibody. In certain embodiments, the first antigen and the second antigen are different epitopes of the CSP protein.

### 7. Activity

The activity of any of the anti-CSP antibodies disclosed herein can be assessed by using different endpoints. In certain embodiments, the activity is assessed for binding to CSP, either binding to a series of linear peptides with varying lengths representing the immunodominant regions of the CSP protein or to the entire CSP protein. In certain embodiments, the activity is assessed for the ability to protect against challenge with Plasmodium that comprises P. falciparum CSP, e.g., in in vivo animal models of malaria. In certain embodiments, effector function, e.g., ADCC, is also evaluated.

In certain embodiments, the binding activity of an anti-CSP antibody disclosed herein to P. falciparum CSP protein can be assessed by surface plasmon resonance (SPR) using a biosensor system. Systems suitable for use in SPR are, for example, and without any limitation, LSA™ (Carterra, Dublin, CA), Biacore™ (General Electric, Boston, MA), and OpenSPR (Nicoya, East Kitchener, ON, Canada). In an exemplary SPR assay, each antibody can be either directly immobilized to a Carterra CMD200M Chip or captured to the CMD200M Carterra Chip with a goat anti-human IgG Fc antibody. The uncoupled antibodies can be washed off and various concentration gradients of the targets can be flowed over the antibodies. In certain experimental conditions, the highest concentration of each target can be in the range of 0.5-8 μg/mL. For better accuracy, each antibody can be immobilized in different locations (e.g., at least 2) on the chip, and the affinity for each antibody-target combination can be determined using multiple (e.g., 4-5) target concentrations according to standard methods. If the variation between the two duplicates is >3-fold, the antibody-target measurement is repeated.

In certain embodiments, the binding activity of an anti-CSP antibody disclosed herein to P. falciparum CSP protein can be assessed by bio-layer interferometry (BLI). For BLI, each of the antigens (e.g., those disclosed in Table 35) can be immobilized on sensors according to the manufacturer's instructions. Systems suitable for use in BLI include, but are not limited to, Octet™ (ForteBio, Fremont, CA) and Gator™ (Probelife, Palo Alto, CA). In certain embodiments, for example and without any limitation, the antigen can be biotinylated and immobilized to streptavidin sensors. For better accuracy, each antibody can be evaluated in replicates at a suitable concentration (e.g., 5 μg/mL). If the variation between the two duplicates is >3-fold, the antibody-target measurement is repeated. The assays are typically performed under conditions according to the manufacturer's instructions. The assays can be performed under a temperature in the range of 20° C. to 37° C., for example, 20° C.-25° C. In certain embodiments, the assay is performed at 25° C. In certain embodiments, the assay is performed at 37° C.

In certain embodiments, binding to CSP protein is assessed in a competitive assay format with a reference antibody AB-000224 or a reference antibody having the variable regions of AB-000224. In certain embodiments, binding to CSP protein is assessed in a competitive assay format with a reference antibody AB-007088 or a reference antibody having the variable regions of AB-007088. In certain embodiments, a variant anti-CSP antibody disclosed herein can block binding of the reference antibody in a competition assay by about 50% or more.

Anti-CSP antibodies and anti-CSP antibody variants of the present disclosure may also be evaluated in various assays for their ability to mediate FcR-dependent activity. In certain embodiments, an antibody of the present disclosure has enhanced ADCC and/or serum stability compared to antibody AB-000224 when the antibodies are assayed in a human IgG1 isotype format. In certain embodiments, an antibody of the present disclosure has enhanced ADCC and/or serum stability compared to antibody AB-007088 when the antibodies are assayed in a human IgG1 isotype format.

In certain embodiments, the activity of an anti-CSP antibody can be evaluated in vivo in an animal model, e.g., as described in the Examples section. In certain non-limiting embodiments, for example, the mouse malaria liver burden assay can be used, as disclosed in, for example, Flores-Garcia Y, et al. Malar J. 2019; 18(1):426, doi:10.1186/s12936-019-3055-9, the content of which is herein incorporated by reference. Mice are administered antibody and infected with chimeric P. berghei expressing GFP-luciferase and P. falciparum CSP protein. Parasite liver load can be evaluated, e.g., by RT-qPCR or by measuring bioluminescence with an IVIS Spectrum imager. A reduction in parasite liver load reflects the prophylactic activity of an antibody.

In certain embodiments, the activity of an anti-CSP antibody can be determined by evaluating the in vivo protection and survival of animal models, e.g., mice. For example, but without any limitation, mice are administered antibody and challenged with chimeric P. berghei expressing P. falciparum CSP protein as disclosed in, for example, Espinosa, D., et al. npj Vaccines 2017; 2, 10 (2017); Espinosa, D., et al. Infect Immun. 2013 August; 81 (8): 2882-2887. The in vivo protection can be determined by detecting blood-stage parasitaemia in microscopy. The survival rate can be determined using the absence of parasitaemia during an observation period, e.g., two weeks, immediately following the challenge. An increased survival rate reflects the prophylactic and/or therapeutic activity of an antibody.

In certain embodiments, an anti-CSP antibody, e.g., AB-000224 or AB-007088, disclosed herein has at least 20%, or at least 30%, or at least 40%, or at least 50%, or at least 60%, or 70%, or greater, of the activity of antibody AB-000317 when evaluated under the same assay conditions. In certain embodiments, an anti-CSP antibody exhibits improved activity, i.e., greater than 100%, activity compared to antibody AB-000317. In certain non-limiting embodiments, an anti-CSP antibody disclosed herein exhibits at least 20%, or at least 30%, or at least 40%, or at least 50%, or at least 60%, or 70%, or greater reduction in parasite liver load as compared to antibody AB-000317. In certain non-limiting embodiments, an anti-CSP antibody disclosed herein exhibits at least 20%, or at least 30%, or at least 40%, or at least 50%, or at least 60%, or 70%, or greater increase in survival rate as compared to antibody AB-000317.

In certain embodiments, an anti-CSP antibody variant disclosed herein has at least 50%, or at least 60%, or 70%, or greater, of the activity of AB-000224 when evaluated under the same assay conditions. In certain embodiments, an anti-CSP antibody exhibits improved activity, i.e., greater than 100%, activity compared to AB-000224. In certain embodiments, the anti-CSP antibody variants disclosed herein have similar activity against malaria infection as compared to AB-000224. In certain embodiment, an anti-CSP antibody variant disclosed herein has at least 50%, or at least 60%, or 70%, or greater, of the activity of AB-007088 when evaluated under the same assay conditions. In certain embodiments, an anti-CSP antibody exhibits improved activity, i.e., greater than 100%, activity compared to AB-007088. In certain embodiments, the anti-CSP antibody variants disclosed herein have similar activity against malaria infection as compared to AB-007088. The term “similar activity,” when used to compare in vivo activity of antibodies, refers to that two measurements of the activity is no more than 30%, no more than 25%, no more than 20%, no more than 15% different, no more than 10%, no more than 8%, or no more than 5% different from each other.

In certain embodiments, the native anti-CSP antibody, AB-000224, is modified to have improved developability (i.e., reduced development liabilities), including but not limited to, decreased heterogeneity, increased yield, increased stability, improved net charges to improve pharmacokinetics, and or/reduced immunogenicity. In certain embodiments, antibodies having improved developability can be obtained by introducing mutations to reduce or eliminate potential development liabilities, as described in Table 1. In certain embodiments, antibodies having improved developability possess modifications as compared to AB-000224 in their amino acid sequence, as disclosed in Table 2.

In certain embodiments, the native anti-CSP antibody, AB-007088, is modified to have improved developability (i.e., reduced development liabilities), including but not limited to, decreased heterogeneity, increased yield, increased stability, improved net charges to improve pharmacokinetics, and or/reduced immunogenicity. In certain embodiments, antibodies having improved developability can be obtained by introducing mutations to reduce or eliminate potential development liabilities, as described in Table 1. In certain embodiments, antibodies having improved developability possess modifications as compared to AB-007088 in their amino acid sequence, as disclosed in Table 2.

In certain embodiments, the anti-CSP antibody variants disclosed herein have improved developability while maintaining comparable or improved binding affinity to the target as compared to AB-000224. Non-limiting examples of such anti-CSP antibody variants are disclosed herein. In certain embodiments, the anti-CSP antibody variants disclosed herein have improved developability while maintaining activities that are similar to AB-000224.

In certain embodiments, the anti-CSP antibody variants disclosed herein have improved developability while maintaining comparable or improved binding affinity to the target as compared to AB-007088. Non-limiting examples of such anti-CSP antibody variants are disclosed herein. In certain embodiments, the anti-CSP antibody variants disclosed herein have improved developability while maintaining activities that are similar to AB-007088.

### 8. Generation of Antibodies

CSP antibodies and variants thereof disclosed herein can be produced using vectors and recombinant methodology (see, e.g., Sambrook & Russell, Molecular Cloning: A Laboratory Manual, Cold Spring Harbor Laboratory Press; Ausubel, Current Protocols in Molecular Biology). Reagents, cloning vectors, and kits for genetic manipulation are available from commercial vendors.

The present disclosure provides isolated nucleic acids encoding a VH and/or VL region, or fragment thereof, of any of the anti-CSP antibodies and anti-CSP antibody variants disclosed herein. In certain embodiments, the present disclosure provides vectors comprising said nucleic acids and host cells into which the nucleic acids are introduced that are used to replicate the antibody-encoding nucleic acids and/or to express the antibodies. These nucleic acids can encode an amino acid sequence containing the VL, and/or an amino acid sequence containing the VH of the anti-CSP antibody or variant thereof (e.g., the light and/or heavy chains of the antibody). In certain embodiments, the host cell contains (1) a vector containing a polynucleotide that encodes the VL amino acid sequence and a polynucleotide that encodes the VH amino acid sequence, or (2) a first vector containing a polynucleotide that encodes the VL amino acid sequence and a second vector containing a polynucleotide that encodes the VH amino acid sequence.

In certain embodiments, the present disclosure provides a method of making an anti-CSP antibody disclosed herein. In certain embodiments, the method includes culturing a host cell previously described under conditions suitable for expression of the antibody. In certain embodiments, the antibody is subsequently recovered from the host cell (or host cell culture medium).

Suitable vectors containing polynucleotides encoding antibodies of the present disclosure, or fragments thereof, include cloning vectors and expression vectors. While the cloning vector selected can vary according to the host cell intended to be used, useful cloning vectors generally can self-replicate, can possess a single target for a particular restriction endonuclease, and/or may carry genes for a marker that can be used in selecting clones containing the vector. Non-limiting examples include plasmids and bacterial viruses, e.g., pUC18, pUC19, Bluescript (e.g., pBS SK+) and its derivatives, mp18, mp19, pBR322, pMB9, ColE1 plasmids, pCR1, RP4, phage DNAs, and shuttle vectors.

Expression vectors generally are replicable polynucleotide constructs that contain a nucleic acid of the present disclosure. The expression vector can replicate in the host cells either as episomes or as an integral part of the chromosomal DNA. Suitable expression vectors include, but are not limited to, plasmids and viral vectors, including adenoviruses, adeno-associated viruses, retroviruses, and any other vector.

Suitable host cells for expressing an anti-CSP antibody or anti-CSP antibody variant disclosed herein include both prokaryotic or eukaryotic cells. For example, but without any limitation, anti-CSP antibodies can be produced in bacteria, in particular when glycosylation and Fc effector function are not needed. After expression, the antibody can be isolated from the bacterial cell lysate in a soluble fraction and can be further purified. Alternatively, the host cell can be a eukaryotic host cell, including, without limitation, eukaryotic microorganisms, such as filamentous fungi or yeast, fungi and yeast strains whose glycosylation pathways have been “humanized,” resulting in the production of an antibody with a partially or fully human glycosylation pattern, vertebrate, invertebrate, and plant cells. Non-limiting examples of invertebrate cells include insect cells. Numerous baculoviral strains have been identified which may be used in conjunction with insect cells. Plant cell cultures can also be utilized as host cells.

In certain embodiments, vertebrate host cells are used for producing anti-CSP antibodies of the present disclosure. For example, without any limitation, mammalian cell lines that can be used to express anti-CSP antibodies include monkey kidney CV1 line transformed by SV40 (COS-7); human embryonic kidney line (293 or 293 cells); baby hamster kidney cells (BHK); mouse Sertoli cells (TM4 cells); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells; MRC 5 cells; and FS4 cells. In certain embodiments, the mammalian cell line used to express anti-CSP antibodies can be Chinese hamster ovary (CHO) cell line; DHFR-CHO cell line (Urlaub et al., Proc. Natl. Acad. Sci. USA 77:4216, 1980); and myeloma cell lines such as YO, NSO, and Sp2/0. Host cells of the present disclosure also include, without limitation, isolated cells, in vitro cultured cells, and ex vivo cultured cells.

A host cell transfected with an expression vector encoding an anti-CSP antibody of the present disclosure, or fragment thereof, can be cultured under appropriate conditions to allow expression of the polypeptide to occur. The polypeptides can be secreted and isolated from a mixture of cells and medium containing the polypeptides. Alternatively, the polypeptide can be retained in the cytoplasm or a membrane fraction and the cells harvested, lysed, and the polypeptide isolated using a desired method.

In certain embodiments, the present disclosure provides a method of generating variants of an anti-CSP antibody disclosed herein. In certain non-limiting embodiments, for example and without any limitation, a construct encoding a variant of VH CDR2 as described in the “anti-CSP Antibody Variant” section can be additionally modified and the VH region encoded by the additionally modified construct can be tested for binding activity to CSP and/or in vivo protective efficacy in the context of a VH region comprising the native AB-000224 CDR1 and CDR3, or a variant CDR1 or CDR3 as described herein, that is paired with a native AB-000224 VL region or variant region as described herein. Similarly, a construct encoding a variant VL CDR3 as described in the “anti-CSP Antibody Variant” section can be additionally modified and the VL region encoded by the additionally modified construct can be tested for binding activity to CSP and/or protective efficacy. Such an analysis can also be performed with other CDRs or framework regions and an antibody having the desired activity can then be selected.

### Pharmaceutical Compositions and Methods of Treatment

In certain embodiments, the present disclosure provides pharmaceutical compositions for the administration of an anti-CSP antibody and variants thereof. In certain embodiments, the pharmaceutical compositions can be administered to a mammalian subject, e.g., a human, who has malaria or is at risk for malaria, in a therapeutically effective amount and according to a schedule sufficient to prevent Plasmodium infection, e.g., infection with Plasmodium falciparum or a Plasmodium sp. having a cross-reactive CSP protein, or to reduce a symptom of malaria in the subject. In certain embodiments, the pharmaceutical compositions can include any of the anti-CSP antibodies and variants thereof disclosed herein, or a polynucleotide encoding the same, and a pharmaceutically acceptable diluent or carrier. In certain embodiments, a polynucleotide encoding the antibody can be contained in a plasmid vector for delivery, or a viral vector. In certain embodiments, the pharmaceutical composition comprises a therapeutically effective amount of the antibody. As used herein, a “therapeutically effective dose” or a “therapeutically effective amount” refers to an amount sufficient to prevent, cure, or at least partially arrest malaria or symptoms of malaria. A therapeutically effective dose can be determined by monitoring a patient's response to therapy. Typical benchmarks indicative of a therapeutically effective dose include amelioration or prevention of symptoms of malaria in the patient, including, for example, and without limitation, reduction in the number of parasites. Amounts effective for this use will depend upon the severity of the disease and the general state of the patient's health, including other factors such as age, weight, gender, administration route, etc. Single or multiple administrations of the antibody will be dependent on the dosage and frequency as required and tolerated by the patient.

In certain embodiments, the antibody is administered at a pre-erythrocyte stage of infection, i.e., the antibody is administered in a time frame to prevent or reduce hepatocyte infection.

Various pharmaceutically acceptable diluents, carriers, and excipients, and techniques for the preparation and use of pharmaceutical compositions are also disclosed herein. Illustrative pharmaceutical compositions and pharmaceutically acceptable diluents, carriers, and excipients are also described in Remington: The Science and Practice of Pharmacy 20th Ed. (Lippincott, Williams & Wilkins 2012). In certain embodiments, each carrier, diluent, or excipient is “acceptable” in the sense of being compatible with the other ingredients of the pharmaceutical composition and not injurious to the subject. Often, the pharmaceutically acceptable carrier is an aqueous pH-buffered solution. In certain non-limiting embodiments, for example, pharmaceutically-acceptable carriers, diluents or excipients include water; buffers, e.g., phosphate-buffered saline; sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

In certain embodiments, the pharmaceutical composition can be formulated for any suitable route of administration, including for example, parenteral, intrapulmonary, intranasal, or local administration. Parenteral administration can include intramuscular, intravenous, intraarterial, intraperitoneal, oral, or subcutaneous administration. In certain embodiments, the pharmaceutical composition is formulated for intravenous administration and has a concentration of antibody of 10-100 mg/ml, 10-50 mg/ml, 20 to 40 mg/ml, or about 30 mg/ml. In certain embodiments, the pharmaceutical composition is formulated for subcutaneous injection and has a concentration of antibody of 50-500 mg/ml, 50-250 mg/ml, or 100 to 150 mg/ml, and a viscosity less than 50 cP, less than 30 cP, less than 20 cP, or about 10 cP. In certain embodiments, the pharmaceutical compositions are liquids or solids. In certain embodiments, the pharmaceutical compositions are formulated for parenteral, e.g., intravenous, subcutaneous, intraperitoneal, or intramuscular administration.

In certain embodiments, the formulation of and delivery methods of pharmaceutical compositions are adapted according to the site and the disease to be treated. For example, without any limitation, formulations include those in which the antibody is encapsulated in micelles, liposomes, or drug-release capsules (active agents incorporated within a biocompatible coating designed for slow-release); ingestible formulations; formulations for topical use, such as creams, ointments, and gels; and other formulations such as inhalants, aerosols, and sprays.

In certain non-limiting embodiments, for example for parenteral administration, the antibodies or antigen-binding fragments thereof are formulated in a unit dosage injectable form (solution, suspension, emulsion) in association with a pharmaceutically acceptable, parenteral vehicle. Non-limiting examples of vehicles include water, saline, Ringer's solution, dextrose solution, and 5% human serum albumin. Nonaqueous vehicles such as fixed oils and ethyl oleate may also be used.

The dose and dosage regimen depend upon a variety of factors readily determined by a physician, such as the nature of the infection, the characteristics of the subject, and the subject's history. In certain embodiments, the amount of antibody or antigen-binding fragment thereof administered or provided to the subject is in the range of about 0.1 mg/kg to about 50 mg/kg of the subject's body weight. Depending on the type and severity of the infection, in certain embodiments, about 0.1 mg/kg to about 50 mg/kg body weight (e.g., about 0.1-15 mg/kg/dose) of antibody or antigen-binding fragment thereof may be provided as an initial candidate dosage to the patient, whether, for example, by one or more separate administrations, or by continuous infusion. The progress of the therapy is readily monitored by conventional methods and assays and based on criteria known to the physician or other persons of skill in the art.

An antibody or variant thereof of the present disclosure can be administered to a subject using any route of administration, e.g., systemic, parenterally, locally, in accordance with known methods. Such routes include, but are not limited to, intravenous administration, e.g., as a bolus or by continuous infusion over a period of time, by intramuscular, intraperitoneal, intracerobrospinal, subcutaneous, intraarticular, intrasynovial, intrathecal, oral, topical, or inhalation routes. A subject can be administered an antibody of the present invention one or more times; and can be administered before, after, or concurrently with another therapeutic agent as further described below.

In certain embodiments, the antibodies or variants thereof of the present disclosure can be administered to prevent malaria. In certain embodiments, the antibodies disclosed herein can inhibit or reduce the risk of Plasmodium infection. In certain embodiments, the antibodies disclosed herein can inhibit or reduce the pre-erythrocytic or sporozoite stage of infection. In certain embodiments, the antibodies disclosed herein can prevent malaria by targeting the Plasmodium at an early stage of entry to the vertebrate of a subject, to thereby arrest the infection from taking place.

In certain embodiments, antibody or variant thereof of the present disclosure can be administered to treat malaria. In certain embodiments, the antibodies disclosed herein can inhibit or reduce the progression of Plasmodium infection in the blood stream. In certain embodiments, the antibodies disclosed herein can inhibit or reduce the risk of transmission of Plasmodium from a subject to another via insect feeding, e.g., mosquito bite or via contact with infected blood.

In certain embodiments, the pharmaceutical compositions disclosed herein can be administered to a pediatric patient. As used herein, the term “pediatric patient” refers to a patient up to the age of 18 years old. In certain embodiments, the pediatric patient is a patient from age 3 months to less than 12 years old. In certain non-limiting embodiments, the pediatric patient can be a patient between from about 1 year old to about 2 years old, from about 2 years old to about 3 years old, from about 3 years old to about 4 years old, from about 4 years old to about 5 years old, from about 5 years old to about 6 years old, from about 6 years old to about 7 years old, from about 7 years old to about 8 years old, from about 8 years old to about 9 years old, from about 9 years old to about 10 years old, or from about 11 years old to about 12 years old. In certain embodiments, the pediatric patient is not responsive or poorly responsive to another treatment to malaria. In certain embodiments, the pediatric patient is human.

In certain embodiments, the dose of the pharmaceutical compositions disclosed herein is administered based on the weight of the pediatric patient. In certain non-limiting embodiments, the dose of the pharmaceutical compositions is about 5 mg/kg, about 10 mg/kg, about 15 mg/kg, about 20 mg/kg about 25 mg/kg, about 50 mg/kg, about 75 mg/kg, about 100 mg/kg, about 150 mg/kg, about 200 mg/kg, about 250 mg/kg, about 300 mg/kg, or about 350 mg/kg. In certain embodiments, the pediatric patient has a weight of from about 2.5 kg to about 5 kg, from about 5 kg to about 10 kg, from about 10 kg to about 15 kg, from about 15 kg to about 20 kg, from about 20 kg to about 30 kg, or from about 30 kg to about 40 kg.

In certain embodiments, the antibody is provided to the subject in combination with one or more additional therapeutic agents used to treat or prevent malaria or a related disease or disorder. In certain embodiments, a method for treating or preventing malaria is provided, comprising administering to the human a therapeutically effective amount of an antibody as disclosed herein, or a pharmaceutically acceptable salt thereof, in combination with a therapeutically effective amount of one or more additional therapeutic agents. In certain embodiments, a method for treating malaria in a human having or at risk of having the infection is provided, comprising administering to the human a therapeutically effective amount of an antibody as disclosed herein, or a pharmaceutically acceptable salt thereof, in combination with a therapeutically effective amount of one or more additional therapeutic agents.

In certain embodiments, when an antibody of the present disclosure as described herein is combined with one or more additional therapeutic agents as described above, the components of the composition are administered as a simultaneous or sequential regimen. When administered sequentially, the combination may be administered in two or more administrations.

In certain embodiments, an antibody as disclosed herein is combined with one or more additional therapeutic agents in a unitary dosage form for simultaneous administration to a patient.

A “patient” refers to any subject receiving the antibody regardless of whether they have malaria. In certain embodiments, a “patient” is a non-human subject, e.g., an animal that is used as a model for evaluating the effects of antibody administration.

“Co-administration” of an antibody disclosed herein with one or more additional therapeutic agents generally refers to simultaneous or sequential administration of an antibody or fragment thereof disclosed herein and one or more additional therapeutic agents, such that therapeutically effective amounts of the antibody or fragment thereof disclosed herein and one or more additional therapeutic agents are both present in the body of the patient. Co-administration includes administration of unit dosages of the antibody disclosed herein before or after administration of unit dosages of one or more additional therapeutic agents, for example, and without limitation, administration of the antibody within seconds, minutes, or hours of the administration of one or more additional therapeutic agents. In certain non-limiting embodiments, for example, a unit dose of an antibody disclosed herein is administered first, followed within seconds or minutes by administration of a unit dose of one or more additional therapeutic agents. In certain non-limiting embodiments, a unit dose of one or more additional therapeutic agents is administered first, followed by administration of a unit dose of an antibody within seconds or minutes. In certain embodiments, a unit dose of an antibody disclosed herein is administered first, followed, after a period of hours (e.g., 1-12 hours), by administration of a unit dose of one or more additional therapeutic agents. In certain embodiments, a unit dose of one or more additional therapeutic agents is administered first, followed, after a period of hours (e.g., 1-12 hours), by administration of a unit dose of the antibody.

The combined administration may be co-administration, using separate pharmaceutical compositions or a single pharmaceutical composition, or consecutive administration in either order, wherein there is optionally a time period while both (or all) therapeutic agents simultaneously exert their biological activities. Such combined therapy may result in a synergistic therapeutic effect. In certain embodiments, it is desirable to combine administration of an antibody of the invention with another antibody directed against another Plasmodium falciparum antigen, or against a different CSP target epitope.

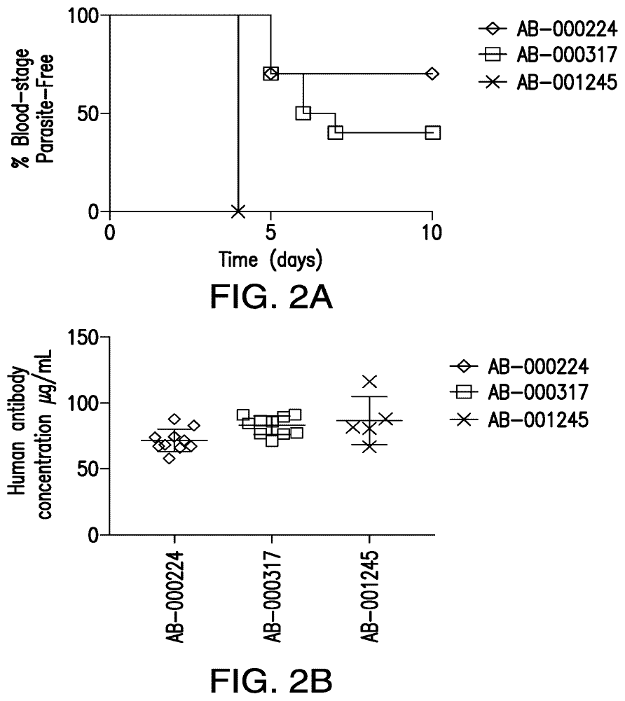

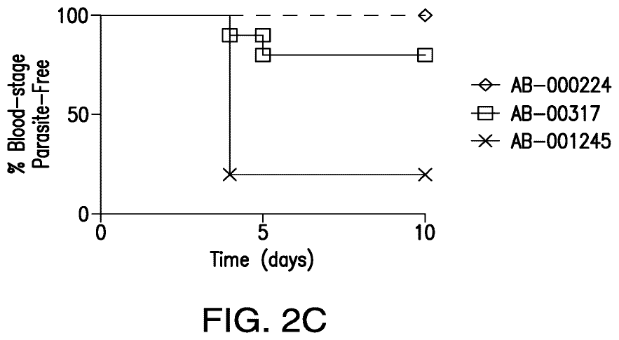

CSP has multiple domains and regions that include the N-terminal domain, the immunogenic central NANP repeat region, and the C-terminal (ctCSP) domain or α-thrombospondin repeat (αTSR) domain. Between the N-terminal domain and the central repeat region is the junctional region that contains an NPDP sequence and a minor repeat region that contains three NVDP motifs that are both related to the dominant NANP motif. (Pholcharee, T. et al., J. Mol. Bio. 432:1048-1063 (2020). In certain embodiments, an antibody disclosed herein is co-administered with an antibody that binds to ctCSP. In certain embodiments, an antibody disclosed herein is co-administered with an antibody that binds to the alpha epitope (α-ctCSP) domain of ctCSP. The α-ctCSP consists of an α-helix that includes the T-cell epitope Th2R (region III), and the CS flap, which contains another T-cell epitope Th3R (Beutler N, PLoS Pathog 18(3):e1010409 (2022), FIG. 2, incorporated by reference herein). In certain embodiments, an antibody disclosed herein is co-administered with an antibody that binds to the beta epitope (β-ctCSP) domain of ctCSP (Beutler N, PLoS Pathog 18(3):e1010409 (2022), FIG. 2).

In certain embodiments, the antibody can be administered by gene therapy via a nucleic acid comprising one or more polynucleotides encoding the antibody. In certain embodiments, the polynucleotide encodes an scFv. In certain embodiments, the polynucleotide comprises DNA, cDNA or RNA. In certain embodiments, the polynucleotide is present in a vector, e.g., a viral vector.

### EXEMPLARY EMBODIMENTS OF THE PRESENTLY DISCLOSED SUBJECT MATTER

The present disclosure provides to antibodies targeting Plasmodium falciparum. In certain non-limiting embodiments, the antibody is a recombinant anti-circumsporozoite (CSP) antibody. In certain embodiments, the recombinant antibody comprises a light chain variable region (VL) and a heavy chain variable region (VH), wherein the light chain variable region comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and the heavy chain variable region comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the VL of the recombinant antibodies disclosed herein comprises the amino acid sequence set forth in SEQ ID NO: 163; the amino acid sequence set forth in SEQ ID NO: 23; the amino acid sequence set forth in SEQ ID NO: 33; the amino acid sequence set forth in SEQ ID NO: 43; the amino acid sequence set forth in SEQ ID NO: 53; the amino acid sequence set forth in SEQ ID NO: 63; the amino acid sequence set forth in SEQ ID NO: 73; the amino acid sequence set forth in SEQ ID NO: 83; the amino acid sequence set forth in SEQ ID NO: 93; the amino acid sequence set forth in SEQ ID NO: 103; the amino acid sequence set forth in SEQ ID NO: 113; the amino acid sequence set forth in SEQ ID NO: 123; the amino acid sequence set forth in SEQ ID NO: 133; the amino acid sequence set forth in SEQ ID NO: 143; the amino acid sequence set forth in SEQ ID NO: 153; or the amino acid sequence set forth in SEQ ID NO: 173. In certain embodiments, the VH of the recombinant antibodies disclosed herein comprises the amino acid sequence set forth in SEQ ID NO: 164; the amino acid sequence set forth in SEQ ID NO: 24; the amino acid sequence set forth in SEQ ID NO: 34; the amino acid sequence set forth in SEQ ID NO: 44; the amino acid sequence set forth in SEQ ID NO: 54; the amino acid sequence set forth in SEQ ID NO: 64; the amino acid sequence set forth in SEQ ID NO: 74; the amino acid sequence set forth in SEQ ID NO: 84; the amino acid sequence set forth in SEQ ID NO: 94; the amino acid sequence set forth in SEQ ID NO: 104; the amino acid sequence set forth in SEQ ID NO: 114; the amino acid sequence set forth in SEQ ID NO: 124; the amino acid sequence set forth in SEQ ID NO: 134; the amino acid sequence set forth in SEQ ID NO: 144; the amino acid sequence set forth in SEQ ID NO: 154; or the amino acid sequence set forth in SEQ ID NO: 174.

In certain embodiments of the recombinant antibodies disclosed herein, the VL comprises the amino acid sequence set forth in SEQ ID NO: 163, and the VH comprises the amino acid sequence set forth in SEQ ID NO: 164; the VL comprises the amino acid sequence set forth in SEQ ID NO: 33, and the VH comprises the amino acid sequence set forth in SEQ ID NO: 34; the VL comprises the amino acid sequence set forth in SEQ ID NO: 43, and the VH comprises the amino acid sequence set forth in SEQ ID NO: 44; the VL comprises the amino acid sequence set forth in SEQ ID NO: 53, and the VH comprises the amino acid sequence set forth in SEQ ID NO: 54; the VL comprises the amino acid sequence set forth in SEQ ID NO: 63, and the VH comprises the amino acid sequence set forth in SEQ ID NO: 64; the VL comprises the amino acid sequence set forth in SEQ ID NO: 73, and the VH comprises the amino acid sequence set forth in SEQ ID NO: 74; the VL comprises the amino acid sequence set forth in SEQ ID NO: 83, and the VH comprises the amino acid sequence set forth in SEQ ID NO: 84; the VL comprises the amino acid sequence set forth in SEQ ID NO: 93, and the VH comprises the amino acid sequence set forth in SEQ ID NO: 94; the VL comprises the amino acid sequence set forth in SEQ ID NO: 103, and the VH comprises the amino acid sequence set forth in SEQ ID NO: 104; the VL comprises the amino acid sequence set forth in SEQ ID NO: 113, and the VH comprises the amino acid sequence set forth in SEQ ID NO: 114; the VL comprises the amino acid sequence set forth in SEQ ID NO: 123, and the VH comprises the amino acid sequence set forth in SEQ ID NO: 124; the VL comprises the amino acid sequence set forth in SEQ ID NO: 133, and the VH comprises the amino acid sequence set forth in SEQ ID NO: 134; the VL comprises the amino acid sequence set forth in SEQ ID NO: 143, and the VH comprises the amino acid sequence set forth in SEQ ID NO: 144; the VL comprises the amino acid sequence set forth in SEQ ID NO: 153, and the VH comprises the amino acid sequence set forth in SEQ ID NO: 154; or the VL comprises the amino acid sequence set forth in SEQ ID NO: 173, and the VH comprises the amino acid sequence set forth in SEQ ID NO: 174. In certain embodiments of the recombinant antibodies disclosed herein, the VL comprises the amino acid sequence set forth in SEQ ID NO: 63, and the VH comprises the amino acid sequence set forth in SEQ ID NO: 64. In certain embodiments of the recombinant antibodies disclosed herein, the VL comprises the amino acid sequence set forth in SEQ ID NO: 133, and the VH comprises the amino acid sequence set forth in SEQ ID NO: 134. In certain embodiments of the recombinant antibodies disclosed herein, the VL comprises the amino acid sequence set forth in SEQ ID NO: 163, and the VH comprises the amino acid sequence set forth in SEQ ID NO: 164.

In certain embodiments, the recombinant antibodies disclosed herein comprise a light chain (LC) and a heavy chain (HC). In certain embodiments, the LC of the recombinant antibodies disclosed herein comprises the amino acid sequence set forth in SEQ ID NO: 27; the amino acid sequence set forth in SEQ ID NO: 37; the amino acid sequence set forth in SEQ ID NO: 47; the amino acid sequence set forth in SEQ ID NO: 57; the amino acid sequence set forth in SEQ ID NO: 67; the amino acid sequence set forth in SEQ ID NO: 77; the amino acid sequence set forth in SEQ ID NO: 87; the amino acid sequence set forth in SEQ ID NO: 97; the amino acid sequence set forth in SEQ ID NO: 107; the amino acid sequence set forth in SEQ ID NO: 117; the amino acid sequence set forth in SEQ ID NO: 127; the amino acid sequence set forth in SEQ ID NO: 137; the amino acid sequence set forth in SEQ ID NO: 147; the amino acid sequence set forth in SEQ ID NO: 157; the amino acid sequence set forth in SEQ ID NO: 167; or the amino acid sequence set forth in SEQ ID NO: 177. In certain embodiments, the HC of the recombinant antibodies disclosed herein comprises the amino acid sequence set forth in SEQ ID NO: 28 or SEQ ID NO: 29; the amino acid sequence set forth in SEQ ID NO: 38 or SEQ ID NO: 39; the amino acid sequence set forth in SEQ ID NO: 48 or SEQ ID NO: 49; the amino acid sequence set forth in SEQ ID NO: 58 or SEQ ID NO: 59; the amino acid sequence set forth in SEQ ID NO: 68 or SEQ ID NO: 69; the amino acid sequence set forth in SEQ ID NO: 78 or SEQ ID NO: 79; the amino acid sequence set forth in SEQ ID NO: 88 or SEQ ID NO: 89; the amino acid sequence set forth in SEQ ID NO: 98 or SEQ ID NO: 99; the amino acid sequence set forth in SEQ ID NO: 108 or SEQ ID NO: 109; the amino acid sequence set forth in SEQ ID NO: 118 or SEQ ID NO: 119; the amino acid sequence set forth in SEQ ID NO: 128 or SEQ ID NO: 129; the amino acid sequence set forth in SEQ ID NO: 138 or SEQ ID NO: 139; the amino acid sequence set forth in SEQ ID NO: 148 or SEQ ID NO: 149; the amino acid sequence set forth in SEQ ID NO: 158 or SEQ ID NO: 159; the amino acid sequence set forth in SEQ ID NO: 168 or SEQ ID NO: 169; or the amino acid sequence set forth in SEQ ID NO: 178 or SEQ ID NO: 179.

In certain embodiments of the recombinant antibodies disclosed herein, the LC comprises the amino acid sequence set forth in SEQ ID NO: 27, and the HC comprises the amino acid sequence set forth in SEQ ID NO: 28 or SEQ ID NO: 29; the LC comprises the amino acid sequence set forth in SEQ ID NO: 37, and the HC comprises the amino acid sequence set forth in SEQ ID NO: 38 or SEQ ID NO: 39; the LC comprises the amino acid sequence set forth in SEQ ID NO: 47, and the HC comprises the amino acid sequence set forth in SEQ ID NO: 48 or SEQ ID NO: 49; the LC comprises the amino acid sequence set forth in SEQ ID NO: 57, and the HC comprises the amino acid sequence set forth in SEQ ID NO: 58 or SEQ ID NO: 59; the LC comprises the amino acid sequence set forth in SEQ ID NO: 67, and the HC comprises the amino acid sequence set forth in SEQ ID NO: 68 or SEQ ID NO: 69; the LC comprises the amino acid sequence set forth in SEQ ID NO: 77, and the HC comprises the amino acid sequence set forth in SEQ ID NO: 78 or SEQ ID NO: 79; the LC comprises the amino acid sequence set forth in SEQ ID NO: 87, and the HC comprises the amino acid sequence set forth in SEQ ID NO: 88 or SEQ ID NO: 89; the LC comprises the amino acid sequence set forth in SEQ ID NO: 97, and the HC comprises the amino acid sequence set forth in SEQ ID NO: 98 or SEQ ID NO: 99; the LC comprises the amino acid sequence set forth in SEQ ID NO: 107, and the HC comprises the amino acid sequence set forth in SEQ ID NO: 108 or SEQ ID NO: 109; the LC comprises the amino acid sequence set forth in SEQ ID NO: 117, and the HC comprises the amino acid sequence set forth in SEQ ID NO: 118 or SEQ ID NO: 119; the LC comprises the amino acid sequence set forth in SEQ ID NO: 127, and the HC comprises the amino acid sequence set forth in SEQ ID NO: 128 or SEQ ID NO: 129; the LC comprises the amino acid sequence set forth in SEQ ID NO: 137, and the HC comprises the amino acid sequence set forth in SEQ ID NO: 138 or SEQ ID NO: 139; the LC comprises the amino acid sequence set forth in SEQ ID NO: 147, and the HC comprises the amino acid sequence set forth in SEQ ID NO: 148 or SEQ ID NO: 149; the LC comprises the amino acid sequence set forth in SEQ ID NO: 157, and the HC comprises the amino acid sequence set forth in SEQ ID NO: 158 or SEQ ID NO: 159; the LC comprises the amino acid sequence set forth in SEQ ID NO: 167, and the HC comprises the amino acid sequence set forth in SEQ ID NO: 168 or SEQ ID NO: 169; or the LC comprises the amino acid sequence set forth in SEQ ID NO: 177, and the HC comprises the amino acid sequence set forth in SEQ ID NO: 178 or SEQ ID NO: 179. In certain embodiments of the recombinant antibodies disclosed herein, the LC comprises the amino acid sequence set forth in SEQ ID NO: 67, and the HC comprises the amino acid sequence set forth in SEQ ID NO: 69. In certain embodiments, the LC comprises the amino acid sequence set forth in SEQ ID NO: 137, and the HC comprises the amino acid sequence set forth in SEQ ID NO: 139. In certain embodiments, the LC comprises the amino acid sequence set forth in SEQ ID NO: 167, and the HC comprises the amino acid sequence set forth in SEQ ID NO: 169.

In certain embodiments, the VL of the recombinant antibodies disclosed herein comprises the amino acid sequence set forth in SEQ ID NO: 13. In certain embodiments of the recombinant antibodies disclosed herein, the VL comprises at least one amino acid substitution. In certain embodiments of the recombinant antibodies disclosed herein, the at least one amino acid substitution is at position 1 and/or at position 44. In certain embodiments of the recombinant antibodies disclosed herein, the amino acid substitution at position 1 is E1Q. In certain embodiments of the recombinant antibodies disclosed herein, the amino acid substitution at position 44 is R44T.

In certain embodiments, the VH of the recombinant antibodies disclosed herein comprises the amino acid sequence set forth in SEQ ID NO: 14. In certain embodiments of the recombinant antibodies disclosed herein, the VH comprises at least one amino acid substitution. In certain embodiments of the recombinant antibodies disclosed herein, the at least one amino acid substitution is at position 21, position 23, position 88, position 98, or a combination thereof. In certain embodiments of the recombinant antibodies disclosed herein, the amino acid substitution at position 1 is E1Q. In certain embodiments of the recombinant antibodies disclosed herein, the amino acid substitution at position 44 is R44T. In certain embodiments of the recombinant antibodies disclosed herein, the amino acid substitution at position 21 is P21S. In certain embodiments of the recombinant antibodies disclosed herein, the amino acid substitution at position 23 is T23A. In certain embodiments of the recombinant antibodies disclosed herein, the amino acid substitution at position 80 is I80T. In certain embodiments, the amino acid substitution at position 90 is T90A.

In certain embodiments, the HC of the recombinant antibodies disclosed herein comprises the amino acid sequence set forth in SEQ ID NO: 18. In certain embodiments of the recombinant antibodies disclosed herein, the HC comprises at least one amino acid substitution. In certain embodiments of the recombinant antibodies disclosed herein, the at least one amino acid substitution is at position 438 and/or or at position 444. In certain embodiments, the amino acid substitution at position 438 is M438L. In certain embodiments of the recombinant antibodies disclosed herein, the amino acid substitution at position 444 is N444S.

In certain non-limiting embodiments, the present disclosure provides a recombinant anti-circumsporozoite (CSP) antibody comprising a light chain variable region (VL) and a heavy chain variable region (VH), wherein the VL comprises the amino acid sequence set forth in SEQ ID NO: 63 and the VH comprises the amino acid sequence set forth in SEQ ID NO: 64. In certain embodiments, the recombinant antibody comprises a light chain (LC) and a heavy chain (HC), wherein the LC comprises the amino acid sequence set forth in SEQ ID NO: 67 and the HC comprises the amino acid sequence set forth in SEQ ID NO: 69.

In certain non-limiting embodiments, the present disclosure further provides a recombinant anti-circumsporozoite (CSP) antibody comprising a light chain variable region (VL) and a heavy chain variable region (VH), wherein the VL comprises the amino acid sequence set forth in SEQ ID NO: 133 and the VH comprises the amino acid sequence set forth in SEQ ID NO: 134. In certain embodiments, the recombinant antibody comprises a light chain (LC) and a heavy chain (HC), wherein the LC comprises the amino acid sequence set forth in SEQ ID NO: 137 and the HC comprises the amino acid sequence set forth in SEQ ID NO: 139.

In certain non-limiting embodiments, the present disclosure also provides a recombinant anti-circumsporozoite (CSP) antibody comprising a light chain variable region (VL) and a heavy chain variable region (VH), wherein the VL comprises the amino acid sequence set forth in SEQ ID NO: 163 and the VH comprises the amino acid sequence set forth in SEQ ID NO: 164. In certain embodiments, the recombinant antibody comprises a light chain (LC) and a heavy chain (HC), wherein the LC comprises the amino acid sequence set forth in SEQ ID NO: 167 and the HC comprises the amino acid sequence set forth in SEQ ID NO: 169.

In certain non-limiting embodiments, the present disclosure provides a recombinant anti-circumsporozoite (CSP) antibody comprising a light chain variable region (VL) and a heavy chain variable region (VH), wherein the light chain variable region comprises a CDR1 comprising the amino acid sequence set forth in SEQ ID NO: 183, a CDR2 comprising the amino acid sequence set forth in SEQ ID NO: 184, and a CDR3 comprising the amino acid sequence set forth in SEQ ID NO: 185; and the heavy chain variable region comprises a CDR1 comprising the amino acid sequence set forth in SEQ ID NO: 186, a CDR2 comprising the amino acid sequence set forth in SEQ ID NO: 187, and a CDR3 comprising the amino acid sequence set forth in SEQ ID NO: 188. In certain embodiments, the VL comprises the amino acid sequence set forth in SEQ ID NO: 195, and the VH comprises the amino acid sequence set forth in SEQ ID NO: 196. In certain embodiments, the recombinant antibody comprises comprising a LC and a HC. In certain embodiments of the recombinant antibodies disclosed herein, the LC comprises the amino acid sequence set forth in SEQ ID NO: 199, and the HC comprises the amino acid sequence set forth in SEQ ID NO: 200 or SEQ ID NO: 201.

In certain non-limiting embodiments, the present disclosure provides a recombinant anti-circumsporozoite (CSP) antibody comprising a light chain variable region (VL) and a heavy chain variable region (VH), wherein the light chain variable region comprises a CDR1 comprising the amino acid sequence set forth in SEQ ID NO: 205, a CDR2 comprising the amino acid sequence set forth in SEQ ID NO: 206, and a CDR3 comprising the amino acid sequence set forth in SEQ ID NO: 207; and the heavy chain variable region comprises a CDR1 comprising the amino acid sequence set forth in SEQ ID NO: 208, a CDR2 comprising the amino acid sequence set forth in SEQ ID NO: 209, and a CDR3 comprising the amino acid sequence set forth in SEQ ID NO: 210.

In certain embodiments, the VL of the recombinant antibodies disclosed herein comprises the amino acid sequence set forth in SEQ ID NO: 227; the amino acid sequence set forth in SEQ ID NO: 237; the amino acid sequence set forth in SEQ ID NO: 247; the amino acid sequence set forth in SEQ ID NO: 257; or the amino acid sequence set forth in SEQ ID NO: 267. In certain embodiments, the VH of the recombinant antibodies disclosed herein comprises the amino acid sequence set forth in SEQ ID NO: 228; the amino acid sequence set forth in SEQ ID NO: 238; the amino acid sequence set forth in SEQ ID NO: 248; the amino acid sequence set forth in SEQ ID NO: 258; or the amino acid sequence set forth in SEQ ID NO: 268. In certain embodiments of the recombinant antibodies disclosed herein, the VL comprises the amino acid sequence set forth in SEQ ID NO: 227, and the VH comprises the amino acid sequence set forth in SEQ ID NO: 228; the VL comprises the amino acid sequence set forth in SEQ ID NO: 237, and the VH comprises the amino acid sequence set forth in SEQ ID NO: 238; the VL comprises the amino acid sequence set forth in SEQ ID NO: 247, and the VH comprises the amino acid sequence set forth in SEQ ID NO: 248; the VL comprises the amino acid sequence set forth in SEQ ID NO: 257, and the VH comprises the amino acid sequence set forth in SEQ ID NO: 258; or the VL comprises the amino acid sequence set forth in SEQ ID NO: 267, and the VH comprises the amino acid sequence set forth in SEQ ID NO: 268.

In certain embodiments, the recombinant antibodies disclosed herein comprise a LC and a HC. In certain embodiments, the LC of the recombinant antibodies disclosed herein comprises the amino acid sequence set forth in SEQ ID NO: 231; the amino acid sequence set forth in SEQ ID NO: 241; the amino acid sequence set forth in SEQ ID NO: 251; the amino acid sequence set forth in SEQ ID NO: 261; or the amino acid sequence set forth in SEQ ID NO: 271. In certain embodiments, the HC of the recombinant antibodies disclosed herein comprises the amino acid sequence set forth in SEQ ID NO: 232 or SEQ ID NO: 233; the amino acid sequence set forth in SEQ ID NO: 242 or SEQ ID NO: 243; the amino acid sequence set forth in SEQ ID NO: 252 or SEQ ID NO: 253; the amino acid sequence set forth in SEQ ID NO: 262 or SEQ ID NO: 263; or the amino acid sequence set forth in SEQ ID NO: 272 or SEQ ID NO: 273. In certain embodiments of the recombinant antibodies disclosed herein, the LC comprises the amino acid sequence set forth in SEQ ID NO: 231, and the HC comprises the amino acid sequence set forth in SEQ ID NO: 232 or SEQ ID NO: 233; the LC comprises the amino acid sequence set forth in SEQ ID NO: 241, and the HC comprises the amino acid sequence set forth in SEQ ID NO: 242 or SEQ ID NO: 243; the LC comprises the amino acid sequence set forth in SEQ ID NO: 251, and the HC comprises the amino acid sequence set forth in SEQ ID NO: 252 or SEQ ID NO: 253; the LC comprises the amino acid sequence set forth in SEQ ID NO: 261, and the HC comprises the amino acid sequence set forth in SEQ ID NO: 262 or SEQ ID NO: 263; or the LC comprises the amino acid sequence set forth in SEQ ID NO: 271, and the HC comprises the amino acid sequence set forth in SEQ ID NO: 272 or SEQ ID NO: 273.

In certain embodiments, the VH of the recombinant antibodies disclosed herein comprises the amino acid sequence set forth in SEQ ID NO: 218. In certain embodiments of the recombinant antibodies disclosed herein, the VH comprises at least one amino acid substitution. In certain embodiments of the recombinant antibodies disclosed herein, the at least one amino acid substitution is at position 40, position 69, position 80, position 85, position 120, or a combination thereof. In certain embodiments of the recombinant antibodies disclosed herein, the amino acid substitution at position 40 is T40A. In certain embodiments of the recombinant antibodies disclosed herein, the amino acid substitution at position 69 is I69T. In certain embodiments of the recombinant antibodies disclosed herein, the amino acid substitution at position 80 is S80Y. In certain embodiments of the recombinant antibodies disclosed herein, the amino acid substitution at position 85 is G85S. In certain embodiments of the recombinant antibodies disclosed herein, the amino acid substitution at position 120 is I120T. In certain embodiment of the recombinant antibodies disclosed herein s, the HC comprises the amino acid sequence set forth in SEQ ID NO: 222. In certain embodiments of the recombinant antibodies disclosed herein, the HC comprises at least one amino acid substitution. In certain embodiments of the recombinant antibodies disclosed herein, the at least one amino acid substitution is at position 434 and/or at position 440. In certain embodiments of the recombinant antibodies disclosed herein, the amino acid substitution at position 434 is M434L. In certain embodiments of the recombinant antibodies disclosed herein, the amino acid substitution at position 440 is N440S.

In certain embodiments, the recombinant antibodies disclosed herein exhibit at least 20% reduction in parasite liver load as compared to a reference antibody. In certain embodiments, the recombinant antibodies disclosed herein exhibit at least 20% increase in survival rate as compared to a reference antibody. In certain embodiments, the recombinant antibodies disclosed herein exhibit increased conformational stability as compared to a reference antibody. In certain embodiments, the recombinant antibodies disclosed herein exhibit increased colloidal stability as compared to a reference antibody. In certain embodiments, the reference antibody is AB-000317. In certain embodiments, the reference antibody is AB-000224. In certain embodiments, the reference antibody is AB-007088.

In certain embodiments, the recombinant antibodies disclosed herein binds to a NANP repeat region. In certain embodiments, the recombinant antibodies disclosed herein binds to a polypeptide comprising or consisting of the amino acid sequence set forth in SEQ ID NO: 280.

In certain embodiments, the recombinant antibodies disclosed herein comprise at least one modification relative to the native AB-000224 variable heavy chain amino acid sequence set forth in SEQ ID NO: 14. In certain embodiments, the recombinant antibodies disclosed herein comprise at least one modification relative to the native AB-000224 variable light chain amino acid sequence set forth in SEQ ID NO: 13. In certain embodiments, the recombinant antibodies disclosed herein comprise at least one modification relative to the native AB-000224 variable heavy chain amino acid sequence set forth in SEQ ID NO: 14 and at least one modification relative to the native AB-000224 variable light chain amino acid sequence set forth in SEQ ID NO: 13.

In certain embodiments, the recombinant antibodies disclosed herein comprise at least one modification relative to the native AB-007088 variable heavy chain amino acid sequence set forth in SEQ ID NO: 218. In certain embodiments, the recombinant antibodies disclosed herein comprise at least one modification relative to the native AB-007088 variable light chain amino acid sequence set forth in SEQ ID NO: 217. In certain embodiments, the recombinant antibodies disclosed herein comprise at least one modification relative to the native AB-007088 variable heavy chain amino acid sequence set forth in SEQ ID NO: 218 and at least one modification relative to the native AB-000224 variable light chain amino acid sequence set forth in SEQ ID NO: 217. In certain non-limiting embodiments, the present disclosure provides a polynucleotide encoding a recombinant antibody disclosed herein. In certain non-limiting embodiments, the present disclosure provides an expression vector comprising the polynucleotide disclosed herein. In certain non-limiting embodiments, the present disclosure provides a host cell comprising the expression vector or the polynucleotide disclosed herein.

In certain non-limiting embodiments, the present disclosure provides a composition comprising the recombinant antibody disclosed herein. In certain embodiments, the composition further comprises a pharmaceutically acceptable carrier.

In certain non-limiting embodiments, the present disclosure provides a method of preventing and/or treating malaria in a subject in need thereof, comprising administering an effective amount of the recombinant antibody disclosed herein or of the composition disclosed herein. In certain embodiments, the subject is a pediatric patient.

In certain non-limiting embodiments, the present disclosure provides the recombinant antibodies or compositions disclosed herein for use in the prevention and/or treatment and/or prevention of malaria in a subject in need thereof. Additionally, in certain non-limiting embodiments, the present disclosure provides the recombinant antibodies or compositions disclosed herein for the manufacture of a medicament for the prevention and/or treatment and/or prevention of malaria in a subject in need thereof. Furthermore, the present disclosure provides use of the recombinant antibodies or compositions disclosed herein for the prevention and/or treatment and/or prevention of malaria in a subject in need thereof. In certain embodiments, the subject is a pediatric patient.

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

The foregoing written description is considered to be sufficient to enable one skilled in the art to practice the methods and/or obtain the compositions described herein. The following examples and detailed description are offered by way of illustration and not by way of limitation.

### EXAMPLES

The Examples are offered for illustrative purposes only, and are not intended to limit the scope of the present invention in any way. Indeed, various modifications in addition to those shown and described herein will become apparent to those skilled in the art from the foregoing description and fall within the scope of the appended claims.

It is understood that various other embodiments may be practiced, given the general description provided above.

### Example 1. Identification of Functionally Active Anti-CSP Antibodies

AB-000224 and AB-007088 were discovered in antibody repertoires generated by Immune Repertoire Capture® (IRC®) technology from plasmablast B cells isolated from two donors enrolled in a Phase 2a study evaluating the efficacy of the RTS,S vaccine in preventing malaria infection. The IRC® technology and its use in antibody discovery is well known and disclosed in, e.g., WO 2012148497A2, the entire content of which is herein incorporated by reference. The RTS,S vaccine is a pseudo-viral particle vaccine that combines the hepatitis B surface antigen and the central repeat and C-terminal regions of the CSP protein. RTS,S consists of two polypeptides; RTS is a single polypeptide chain corresponding to amino acids 207 to 395 of P. falciparum (3D7) that is fused to HBsAg and S is a polypeptide of 226 amino acids that corresponds to HBsAg. Stoute, et. al., N Engl J Med; 336:86-91 (1997); RTS,S Clinical Trials Partnership, PLoS Med. 11(7):e1001685, (2014), WO1993/10152. The RTS,S vaccine was administered with the adjuvant AS01B to increase efficacy. AS01B is a liposome-based formulation that contains the immunostimulants monophosphoryl lipid A (MPL) and QS21 and was shown to be more immunogenic than another adjuvant, AS02A, used in initial studies. Kester, et al., J Infect Dis 200:337-346 (2009). All study participants were vaccinated with one of two vaccine schedules (standard full-dose: 0, 1, 2 M or fractional-third dose: 0, 1, 7 M), or placebo and subsequently challenged with a controlled human malaria parasite infection. The donors from whom AB-000224 and AB-007088 were identified and protected following challenge. Heavy and light chain AB-000224 sequences were expressed as a human IgG1 monoclonal antibody. Heavy and light chain AB-007088 sequences were expressed as a human IgG1 monoclonal antibody. Compared to other antibodies obtained from the same or different donors, AB-000224 and AB-007088 demonstrated strong binding and affinity to CSP protein in vitro, no binding to Hepatitis B protein, and exceptional functional activity when tested in vivo.

The present example provides the design of improved variants of AB-000224 or AB-007088. In certain embodiments, the variants generated have improved developability, e.g., as identified through various in vitro assays, such as aggregation assessment by HPLC or UPLC, hydrophobic interaction chromatography (HIC), polyspecificity assays (e.g., baculovirus particle binding), self-interaction nanoparticle spectroscopy (SINS), or mass spec analysis after incubation in an accelerated degradation condition such as high temperature, low pH, high pH, or oxidative H₂O₂. Mutations are successful if the activity is maintained (or enhanced) while removing or reducing the severity of the liability.

### Example 2. Generation of Anti-CSP Antibody Variants

### Assessment of AB-000224

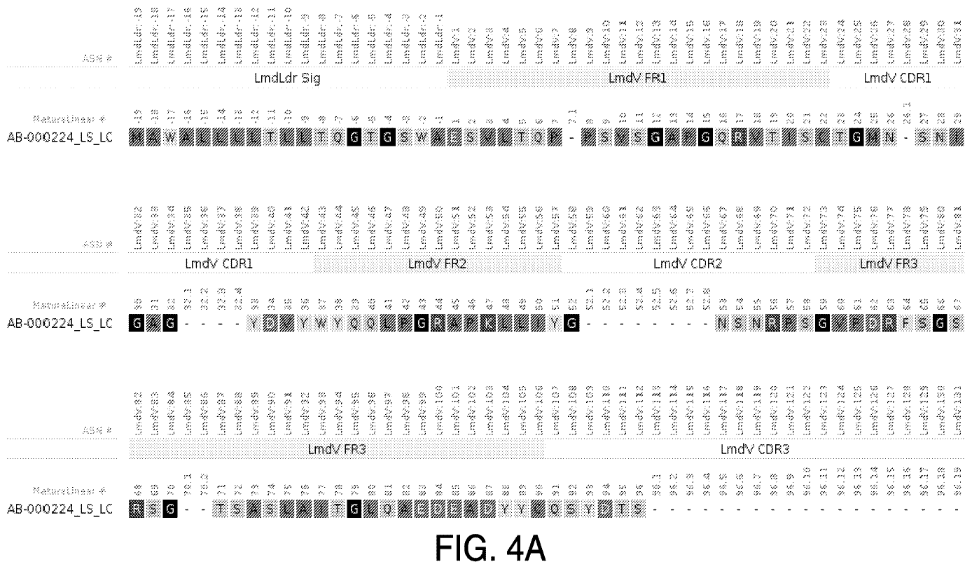

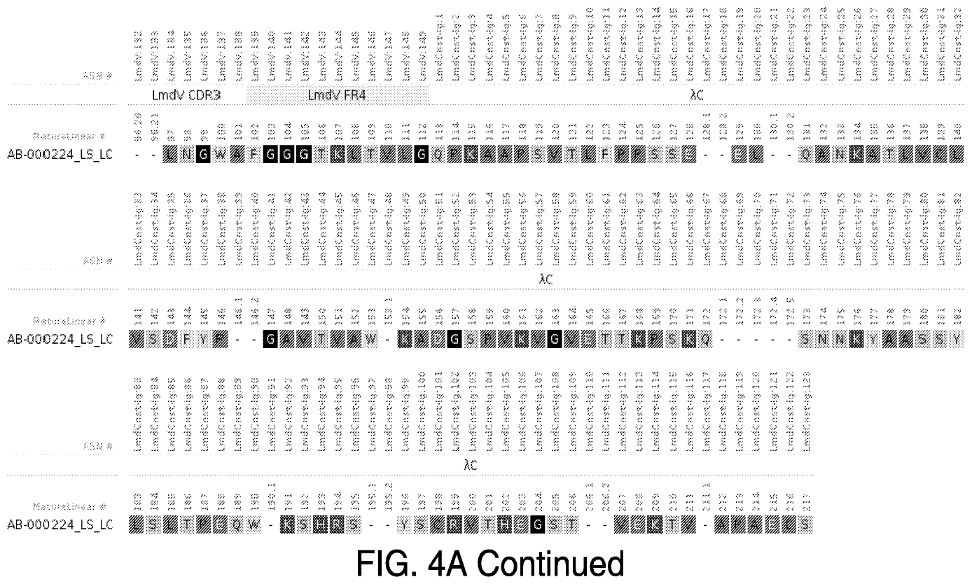

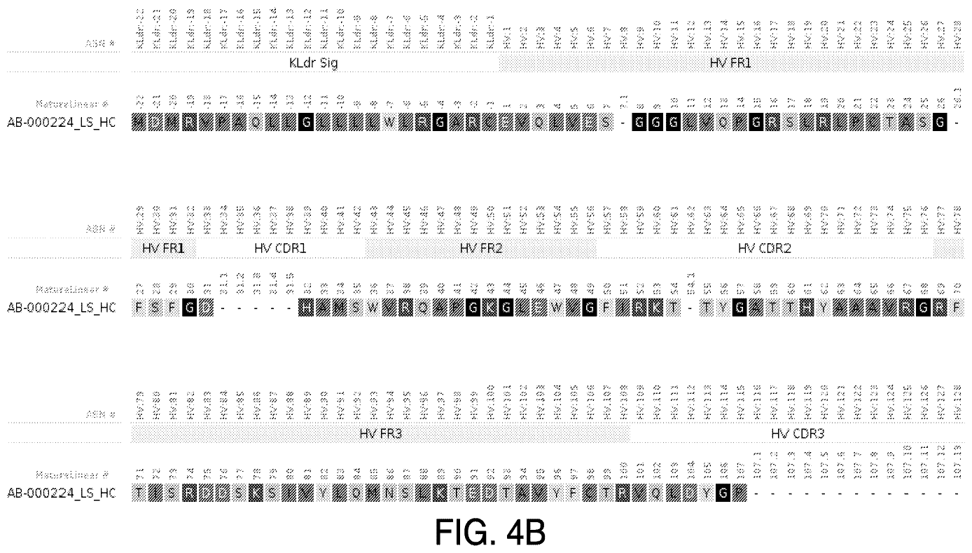

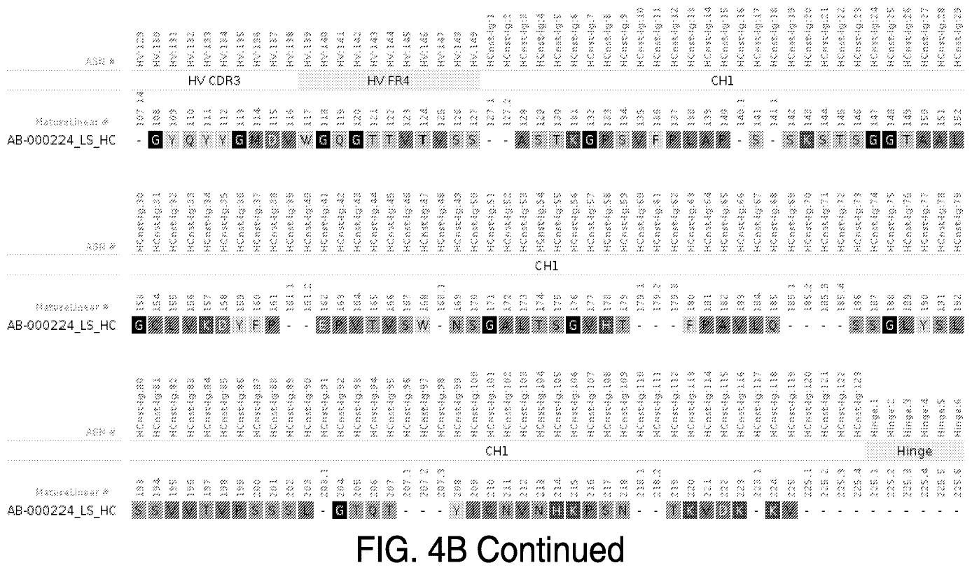

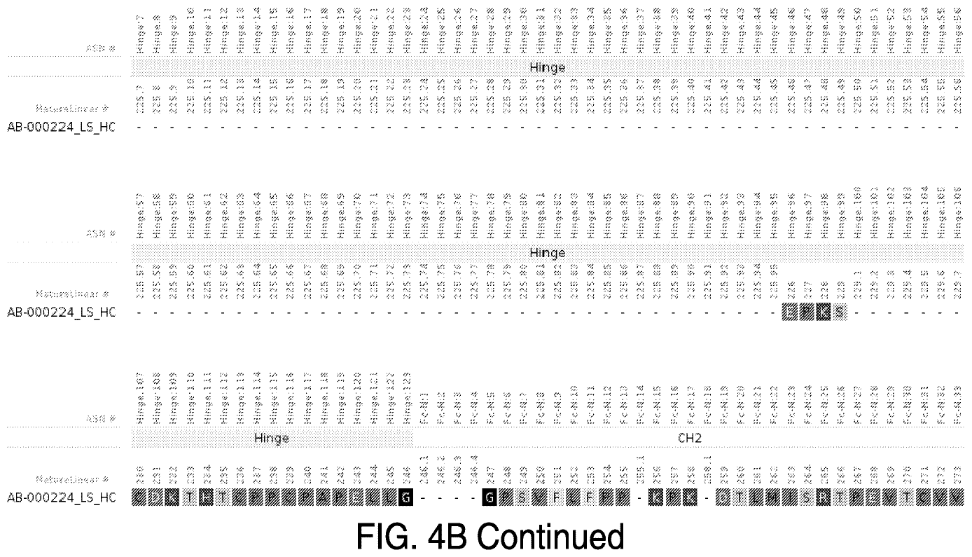

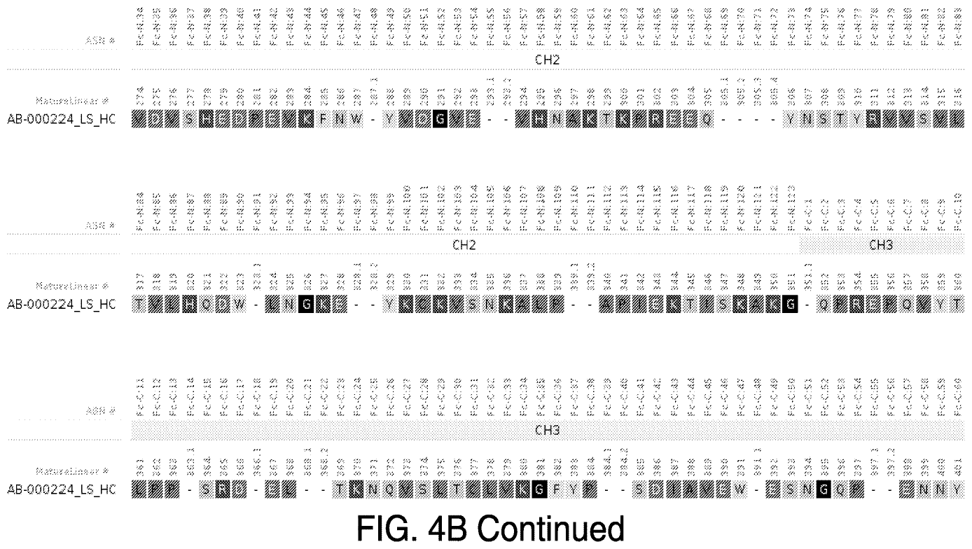

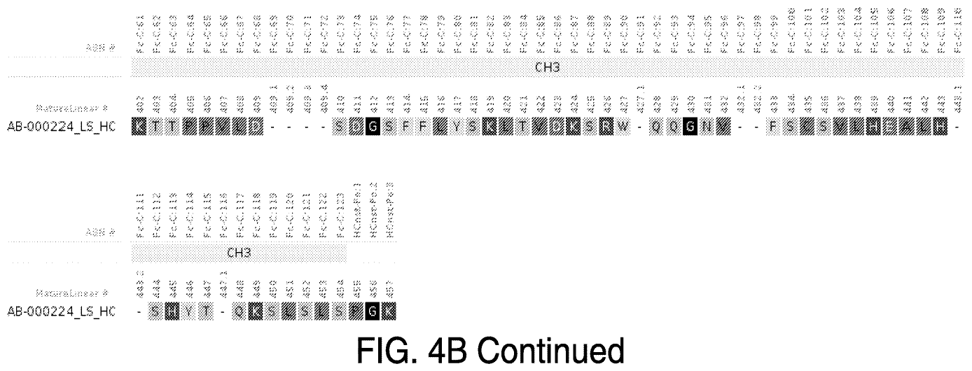

In vivo generated antibodies undergo genomic recombination followed by somatic hypermutation. Germline information was obtained from the AB-000224 antibody and used for the optimization of variant design. FIGS. 4A and 4B show the alignment of the AB-0002224 to the putative V and J germlines genes. CDRs, germline deviations, and potential liabilities were identified. N-linked glycosylation sites, non-typical cysteine residues, and other potential liability motifs were identified across the VH and VL.

The close siblings to AB-000224 are AB-007110, AB-007111, and AB-007112. These were evaluated at the sequence and structure level to find any possible beneficial modifications to AB-000224. AB-007110 in particular has been shown in biophysical characterization to have improved thermal stability over AB-000224. The positions evaluated below differ from AB-000224 and are either consistent amongst the siblings or of structural interest.

### Design of Variants of AB-000224

Framework and complementary-determining region (CDR) germline deviations in AB-000224 were analyzed for their potential to be mutated, individually or in combination, to germline sequence, without negatively impacting binding to the (NANP)3 region of the CSP protein or potency. For each of the candidate mutations from AB-000224 sequence to germline sequence, the risk of making the mutation was assessed based on: (1) the change in charge, if any, since change in charge is intrinsically risky, and a change to more positive charge is particularly risky given the already net positive charge of AB-000224 Fv; (2) conservation of the native AB-000224 residue in the lineage versus the presence of the germline residue or other mutations at that position in the lineage and (3) the structural location of the position with respect to the NANP motif. Some mutations were noted to be coupled to at least one other mutation, meaning that the risk prediction is based on making the mutation in conjunction with the other mutation(s). Proposed AB-000224 Residue Modifications according to the ASN numbering system are shown in Table 31 below:

TABLE 31

| Site | Mutation | Design Group |
|---|---|---|
| LmdV:E1 | Q | Standard |
| LmdV:R50 | T | Standard |
| LmdV:N135 | NS | Siblings |
| HV:P22 | S | Standard |
| HV:T24 | TA | Standard |
| HV:H69 | HK | Siblings |
| HV:R75 | RK | Siblings |
| HV:D84 | DN | Do not repair |
| HV:I88 | IT | Standard |
| HV:T98 | TA | Standard |
| HV:F105 | FY | Do not repair |
| HV:T107 | TA | Standard |

Mutations were built by grouping the “Standard” Design Group in all combinations. This results in 16 variants. Add a single variant using the “Sibling” Design Group with all three sites together. This totals 17 variants. Adding the parent results in 18 antibodies required for production. Mutation site positions in the AB-000224 variants according to the ASN numbering system are specified in the table below:

TABLE 32

| Variant | LC-LmdV:E1 | LC-LmdV:R50 | LC-LmdV:N135 | HV:P22 | HV:T24 | HV:H69 | HV:R75 | HV:I88 | HV:T98 | HV:T107 |
|---|---|---|---|---|---|---|---|---|---|---|
| AB-000224_LS | | | | | | | | | | |
| AB-000224.001 | Q | T | | S | | | | | | |
| AB-000224.002 | Q | T | | s | A | | | | | |
| AB-000224.003 | Q | T | | s | | | | T | | |
| AB-000224.004 | Q | T | | s | | | | | A | |
| AB-000224.005 | Q | T | | s | | | | | | A |
| AB-000224.006 | Q | T | | S | A | | | T | | |
| AB-000224.007 | Q | T | | S | A | | | | A | |
| AB-000224.008 | Q | T | | S | A | | | | | A |
| AB-000224.009 | Q | T | | S | | | | T | A | |
| AB-000224.010 | Q | T | | S | | | | T | | A |
| AB-000224.011 | Q | T | | S | | | | | A | A |
| AB-000224.012 | Q | T | | S | A | | | T | A | |
| AB-000224.013 | Q | T | | S | A | | | T | | A |
| AB-000224.014 | Q | T | | S | A | | | | A | A |
| AB-000224.015 | Q | T | | S | | | | T | A | A |
| AB-000224.016 | Q | T | | S | A | | | T | A | A |
| AB-000224.017 | | | D | | | K | K | | | |

### Assessment of AB-007088

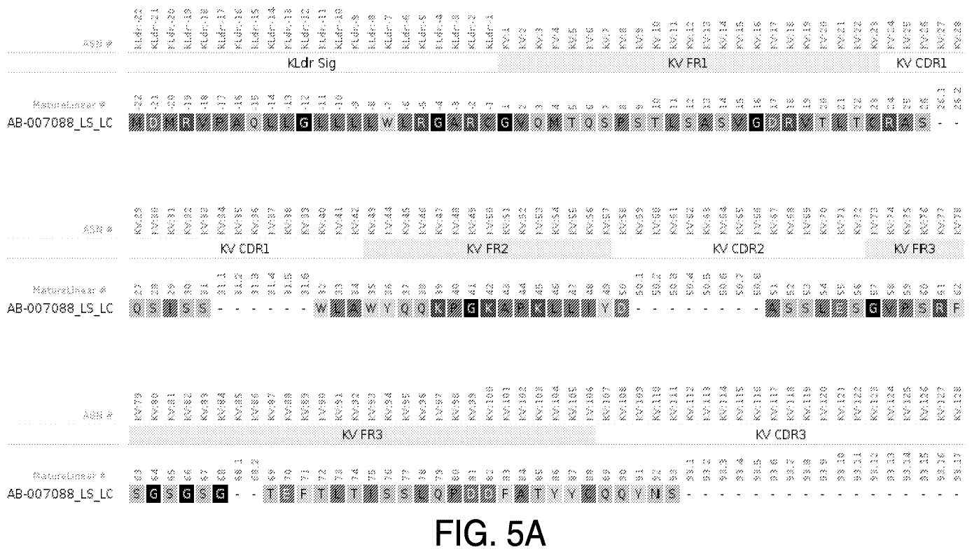

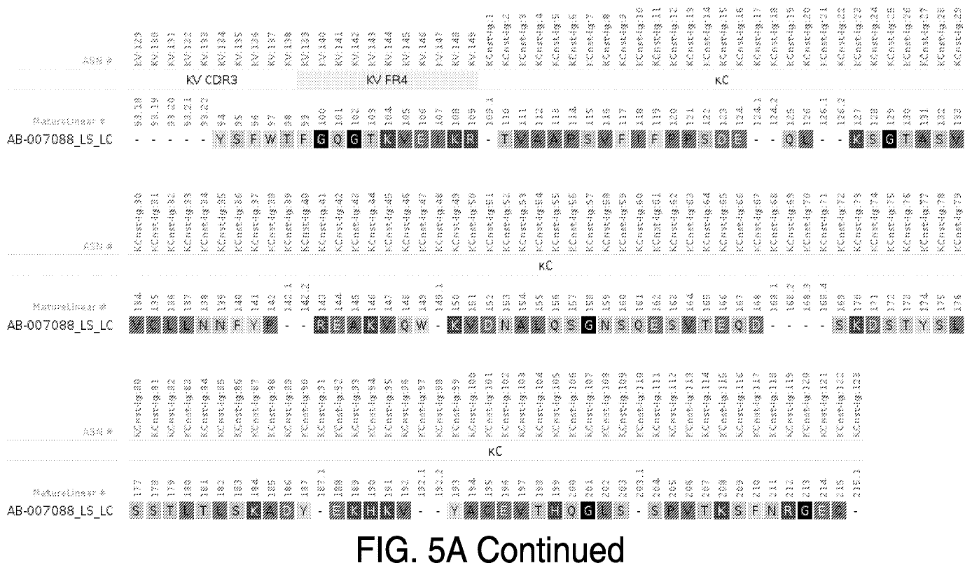

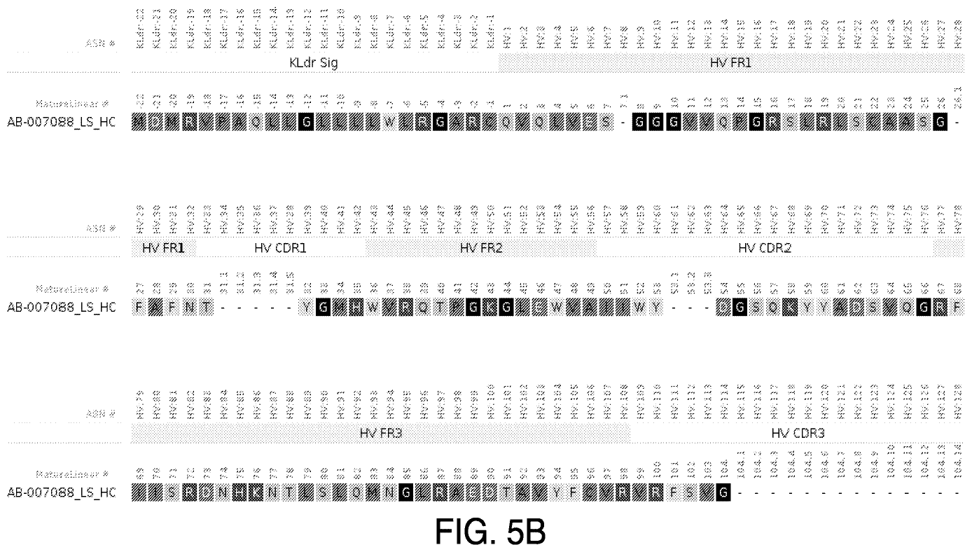

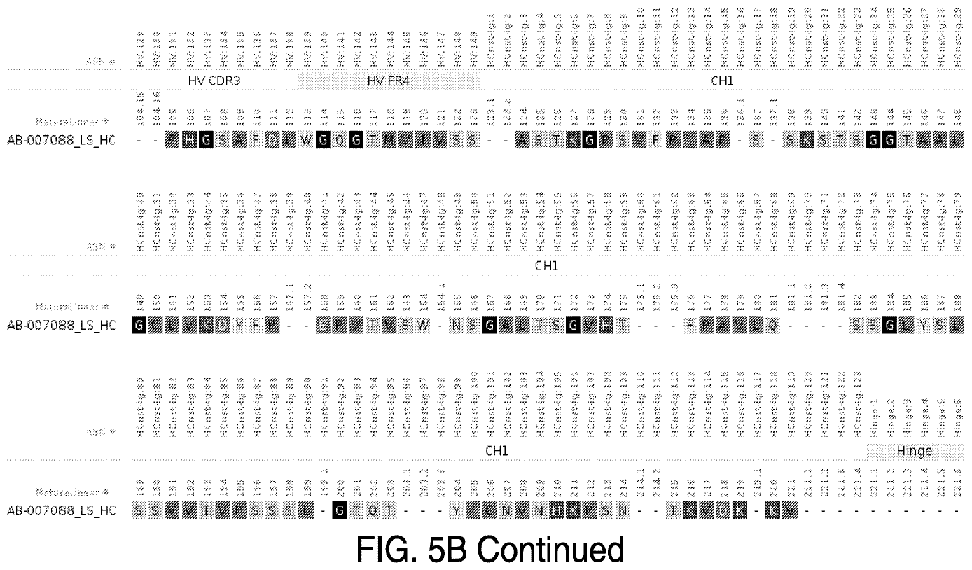

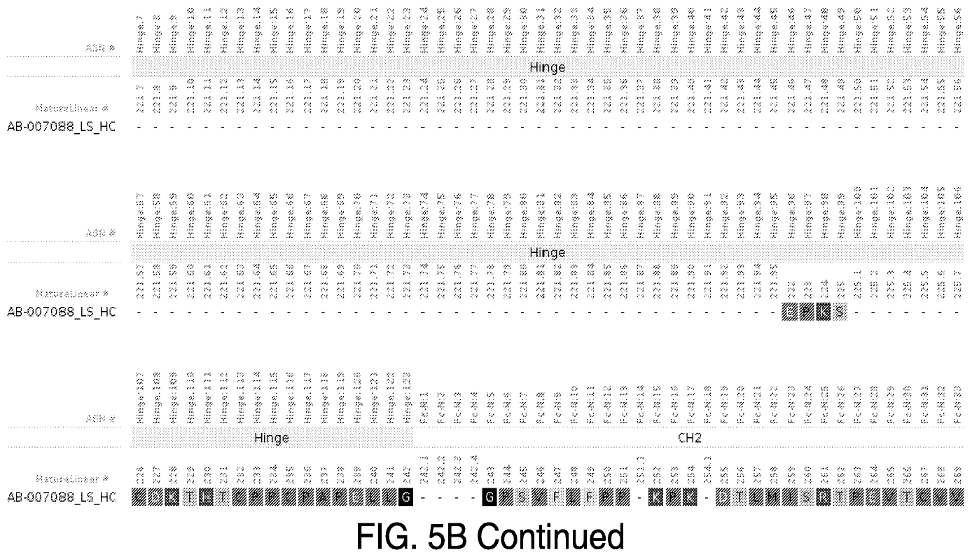

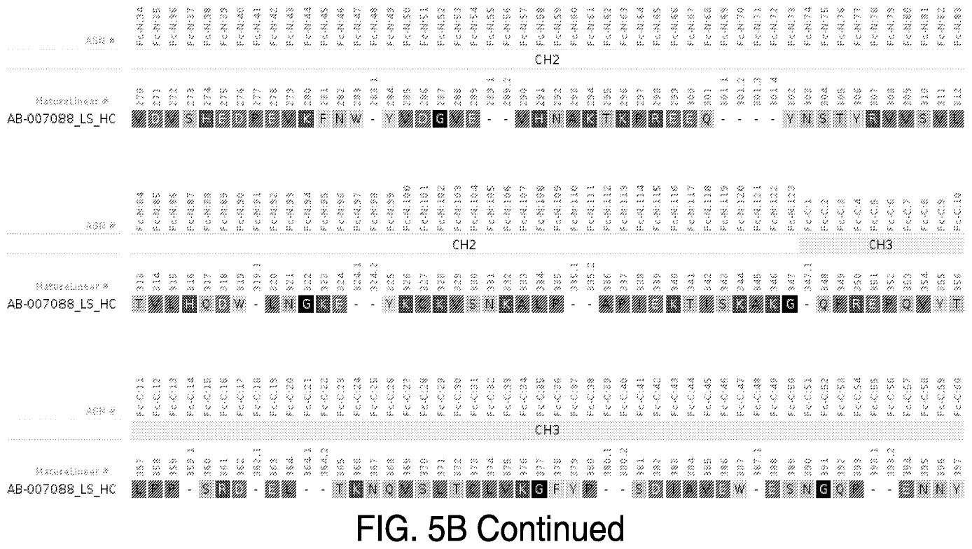

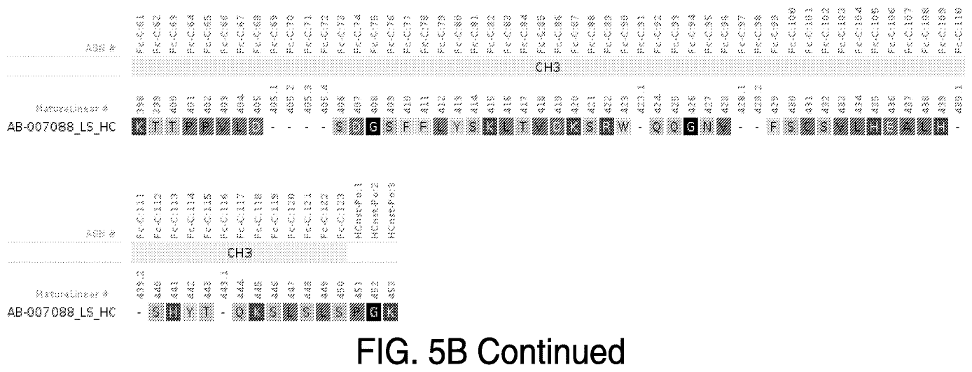

Germline information was obtained from the AB-007088 antibody and used for the optimization of variant design. FIGS. 5A and 5B show the alignment of the AB-0070884 to the putative V and J germlines genes. CDRs, germline deviations, and potential liabilities were identified. N-linked glycosylation sites, non-typical cysteine residues, and other potential liability motifs were identified across the VH and VL.

### Design of Variants of AB-007088

Framework and complementary-determining region (CDR) germline deviations in AB-007088 were analyzed for their potential to be mutated, individually or in combination, to germline sequence, without negatively impacting binding to the (NANP) 3 region of the CSP protein or potency. For each of the candidate mutations from AB-007088 sequence to germline sequence, the risk of making the mutation was assessed based on: (1) the change in charge, if any, since change in charge is intrinsically risky, and a change to more positive charge is particularly risky given the already net positive charge of AB-007088 Fv; (2) conservation of the native AB-007088 residue in the lineage versus the presence of the germline residue or other mutations at that position in the lineage and (3) the structural location of the position with respect to the NANP motif. Some mutations were noted to be coupled to at least one other mutation, meaning that the risk prediction is based on making the mutation in conjunction with the other mutation(s). Proposed AB-007088 Residue Modifications according to the ASN numbering system are shown in Table 33 below:

TABLE 33

| Site | Mutation | Design Group |
|---|---|---|
| HV:A30 | AT | Group 1 |
| HV:T47 | A | Group 1 |
| HV:I79 | IT | Group 1 |
| HV:S90 | SY | Group 2 |
| HV:G95 | S | Group 1 |
| HV:I146 | T | Group 1 |

Mutations were built by grouping the “Group 1” Design Group in all combinations. This resulted in 4 variants. By adding a single variant using HV: S90Y to the design with all mutations, a total of five designs were obtained. Mutation site positions in the AB-007088 variants according to the ASN numbering system are specified in the table below:

TABLE 34

| Variant | HV:A30 | HV:T47 | HV:I79 | HV:S90 | HV:G95 | HV:I146 |
|---|---|---|---|---|---|---|
| AB-007088_LS | | | | | | |
| AB-007088.001 | | A | | | S | T |
| AB-007088.002 | T | A | | | S | T |
| AB-007088.003 | | A | T | | S | T |
| AB-007088.004 | T | A | T | | S | T |
| AB-007088.005 | T | A | T | Y | S | T |

### Example 3. Binding Assays

AB-000224 and AB-007088 were evaluated for binding to the complete CSP protein and a series of linear peptides representing the immunodominant NANP repeat region. Two assay platforms, bio-layer interferometry (BLI) and surface plasmon resonance (SPR), were used to quantify antibody-target binding strength. Five binding targets were evaluated in the SPR assay and six targets were evaluated in the BLI platform and described in Table 35 below:

TABLE 35

| | | Used in |
|---|---|---|
| | | BLI, SPR, |
| Target | | or both |
| Name | Peptide Sequence | assays |
| (NANP)6 | NANPNANPNANPNANPNANPNANP (SEQ ID NO: 279) | Both |
| (NPNA)3 | NPNANPNANPNA (SEQ ID NO: 280) | Both |
| (NVDP)3 | NVDPNANPNVDPNANPNVDP (SEQ ID NO: 281) | Both |
| (NANP)2 | | |
| NANPNV | NPDPNANPNVDPNANP (SEQ ID NO: 282) | Both |
| DPNANP | | |
| NANPNV | DPNANPNVDPNA (SEQ ID NO: 283) | BLI only |
| DP | | |
| N- | KQPADGNPDPNANPN (SEQ ID NO: 284) | BLI only |
| Interface | | |
| CSP | MMRKLAILSVSSFLFVEALFQEYQCYGSSSNTRVLNELNYDNAGIN | SPR only |
| Protein | LYNELEMNYYGKQENWYSLKKNSRSLGENDDGNNNNGDNGREGKDE | |
| | DKRDGNNEDNEKLRKPKHKKLKQPGDGNPDPNANPNVDPNANPNVD | |
| | PNANPNVDPNANPNANPNANPNANPNANPNANPNANPNANPNANPN | |
| | ANPNANPNANPNANPNANPNANPNANPNANPNANPNANPNANPNAN | |
| | PNANPNANPNANPNANPNANPNANPNANPNANPNANPNANPNANPN | |
| | ANPNANPNANPNANPNANPNANPNKNNQGNGQGHNMPNDPNRNVDE | |
| | NANANNAVKNNNNEEPSDKHIEQYLKKIQNSLSTEWSPCSVTCGNG | |
| | IQVRIKPGSANKPKDELDYENDIEKKICKMEKCSSVFNVVNSSIGL | |
| | IMVLSFLFLN (SEQ ID NO: 285) | |

For BLI, each of the targets specified in Table 35 was biotinylated and immobilized to streptavidin sensors. Each antibody was evaluated in duplicate at 5 μg/mL. If the variation between the two duplicates was >3-fold, the antibody-target measurement was repeated.

For SPR, each antibody was either directly coupled to a Carterra Chip or coupled using a goat anti-human Fc antibody. The uncoupled antibodies were washed off and various concentration gradients of the targets were flowed over the antibodies, where the highest concentration of each target was in the range 0.5-8 μg/mL. Each antibody was immobilized in two different locations on the chip to allow for duplicate measurements. The affinity for each antibody-target combination was determined using 4-5 target concentrations in Mathematica software. If the variation between the two duplicates was >3-fold, the antibody-target measurement was repeated.

While the data generated by the BLI and SPR assays are similar, the assays were designed with opposite orientations of the target and antibody. Specifically, the target was immobilized while the antibody flowed over it in the BLI assay, while the SPR assay was designed so that the antibody was immobilized and the target flowed over it. Given these orientations, an antibody, when evaluated in the BLI assay, would be more likely to engage in binding interactions that involve multiple target molecules. As such, the binding of antibodies to targets in the BLI assay may exhibit more similarities to binding the complete CSP protein, which coats the surface of the malaria sporozoite. In contrast, the activity measured in the SPR assay would more accurately represent an interaction between an antibody F (ab) and a single target molecule. The data generated for the antibodies AB-000224 and AB-007088 are summarized in Table 36 below:

TABLE 36

| Antibody ID | AB-000224 | AB-007088 |
|---|---|---|
| NANP6 K_{ON} BLI | 5.44 | 5.22 |
| NANP6 K_{OFF} BLI | −4.75 | −4.32 |
| NANP6 K_{D} BLI | −10.19 | −9.55 |
| NANP6 K_{ON} SPR | 5.01 | 5.52 |
| NANP6 K_{OFF} SPR | −5.00 | −4.14 |
| NANP6 K_{D} SPR | −10.00 | −9.63 |
| NPNA3 K_{ON} BLI | 5.35 | 5.23 |
| NPNA3 K_{OFF} BLI | −4.32 | −6.00 |
| NPNA3 K_{D} BLI | −9.67 | −11.23 |
| NPNA3 K_{ON} SPR | 4.87 | 5.08 |
| NPNA3 K_{OFF} SPR | −3.47 | −3.25 |
| NPNA3 K_{D} SPR | −8.34 | −8.33 |
| NVDP3NANP2 K_{ON} BLI | 5.53 | 4.94 |
| NVDP3NANP2 K_{OFF} BLI | −5.12 | −6.00 |
| NVDP3NANP2 K_{D} BLI | −10.65 | −10.94 |
| NVDP3NANP2 K_{ON} SPR | 4.81 | 4.29 |
| NVDP3NANP2 K_{OFF} SPR | −4.02 | −4.40 |
| NVDP3NANP2 K_{D} SPR | −8.77 | −8.71 |
| NANPNVDPNANP K_{ON} BLI | 5.59 | 5.03 |
| NANPNVDPNANP K_{OFF} BLI | −2.68 | −6.00 |
| NANPNVDPNANP K_{D} BLI | −8.27 | −11.03 |
| NANPNVDPNANP K_{ON} SPR | 4.08 | <LLOQ |
| NANPNVDPNANP K_{OFF} SPR | −1.77 | <LLOQ |
| NANPNVDPNANP K_{D} SPR | −5.85 | <LLOQ |
| NANPNVDP K_{ON} BLI | <LLOQ | 4.89 |
| NANPNVDP K_{OFF} BLI | <LLOQ | −6.00 |
| NANPNVDP K_{D} BLI | <LLOQ | −10.89 |
| NVDPNANP K_{ON} BLI | 5.35 | 5.02 |
| NVDPNANP K_{OFF} BLI | −4.50 | −6.00 |
| NVDPNANP K_{D} BLI | −9.85 | −11.02 |
| N-Interface K_{ON} BLI | 6.42 | 5.13 |
| N-Interface K_{OFF} BLI | −1.92 | −6.00 |
| N-Interface K_{D} BLI | −8.34 | −11.13 |
| N-Interface K_{ON} SPR | <LLOQ | 4.17 |
| N-Interface K_{OFF} SPR | <LLOQ | −2.42 |
| N-Interface K_{D} SPR | <LLOQ | −6.60 |
| CSP K_{ON} SPR | 5.47 | 5.08 |
| CSP K_{OFF} SPR | −5.00 | −4.59 |
| CSP K_{D} SPR | −10.44 | −9.67 |

### Summary of AB-000224 Antibody Variants

Seventeen (17) variants (AB-000224.001, AB-000224.002, AB-000224.003, AB-000224.004, AB-000224.005, AB-000224.006, AB-000224.007, AB-000224.008, AB-000224.009, AB-000224.010, AB-000224.011, AB-000224.012, AB-000224.013, AB-000224.014, AB-000224.015, AB-000224.016, AB-000224.017)) were designed to germline antibodies by mutating residues in either the framework regions or CDRs to reduce the risk of antibody-directed immunogenicity.

### Summary of AB-007088 Antibody Variants

Five (5) variants (AB-007088.001, AB-007088.002, AB-007088.003, AB-007088.004, AB-007088.005) were designed to germline antibodies by mutating residues in either the framework regions or CDRs to reduce the risk of antibody-directed immunogenicity.

### Example 4. In Vivo Performance of the Antibodies

### Liver Burden Assay

AB-000224 and AB-007088 and variants thereof (with those in format comprising Heavy Chain version 2 indicated with the “LS”) were evaluated for in vivo activity in a mouse malaria liver burden assay, as described in Flores-Garcia Y, et al. Malar J. 2019; 18(1):426, doi:10.1186/s12936-019-3055-9. Experimental antibodies were compared to both positive (AB-000317) and negative (AB-001245) antibody controls. AB-000317 is an anti-CSP antibody described in WO2020/172220. AB-001245 is non-CSP isotype control.

For each antibody, five C57Bl/6 mice per experimental or control arm were administered 100 μg of antibody 16 hours prior to intravenous infection with fluorescent chimeric P. berghei sporozoites expressing P. falciparum CSP protein. Forty-two (42) hours following parasite challenge, the sporozoite liver load was quantified by bioluminescence. For each experimental mouse, the percent liver burden was calculated by subtracting the average background luminescence measured from two untreated, naive mice and calculating the percent reduction as compared to the average luminescence measured in five untreated, infected mice. The average percent reduction was reported for each of the experimental antibody groups.

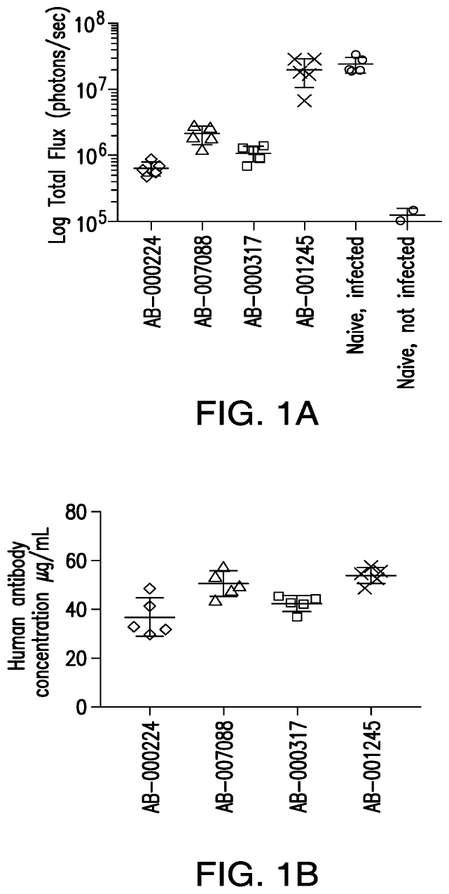

Mice administered AB-000224, AB-007088 or positive control antibody AB-000317 all exhibited a similar reduction in liver burden load as compared to naïve infected mice and mice treated with the negative control, AB-001245 (FIG. 1A). The level of experimental and control human antibodies circulating in the mice at the time of infection was quantified via an ELISA assay (FIG. 1B) and was similar between the experimental and control antibodies.

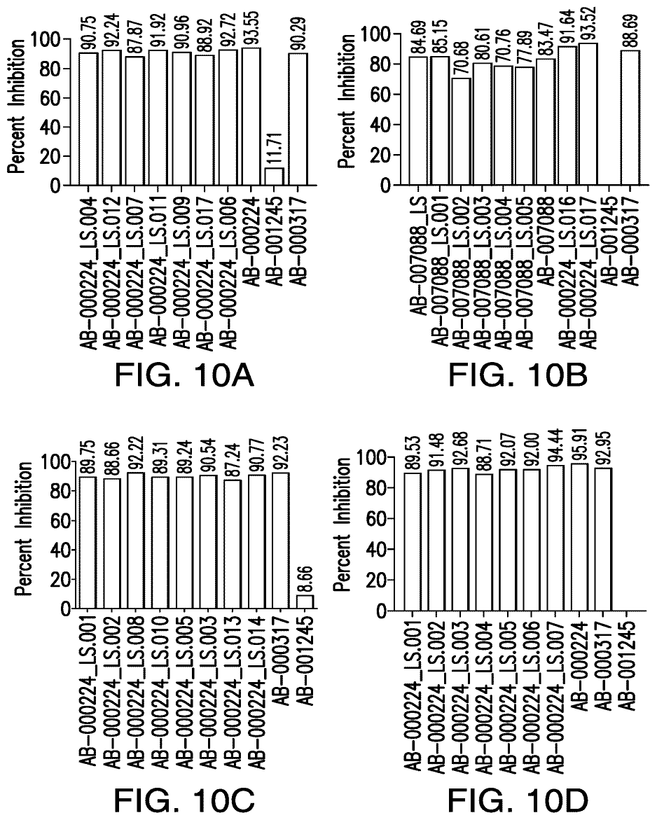

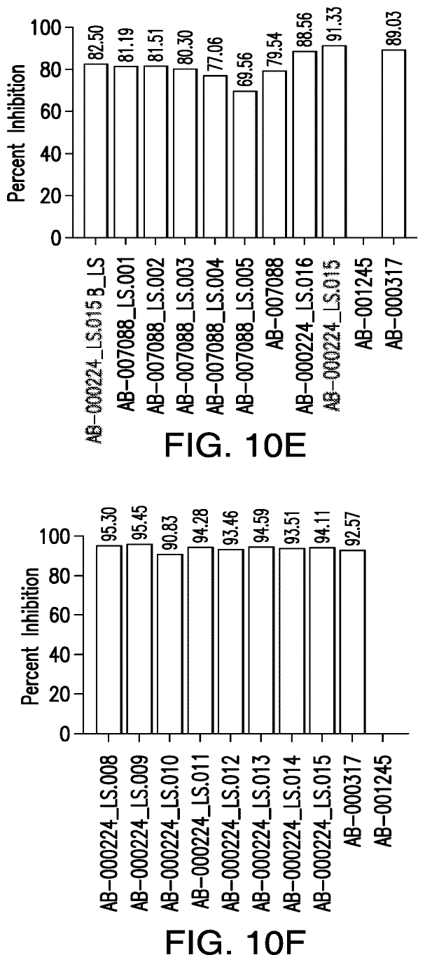

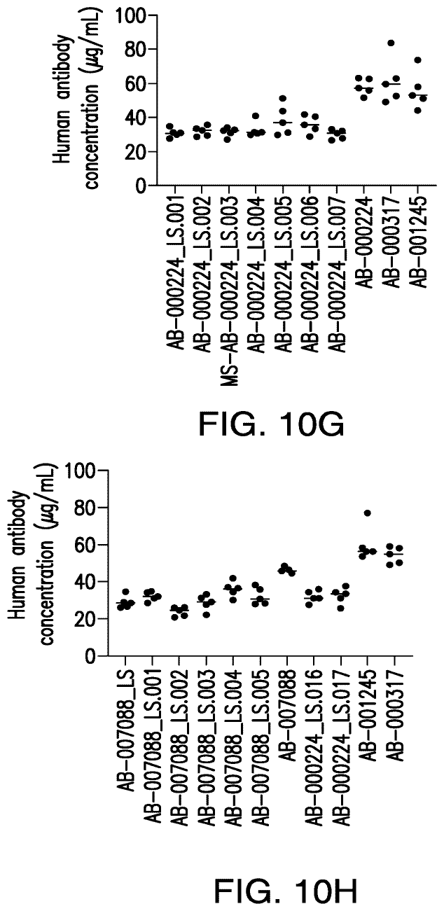

Variants of AB-000224 and AB-007088 drove reduction in liver burden load in treated mice that was comparable to the respective parent antibody AB-000224 or AB-007088 and the positive control antibody AB-000317. Liver burden levels measured in animals administered either the variants, the parent molecules or positive control antibody AB-000317 was significantly lower than liver burden levels in either the naïve infected mice or mice treated with the negative control, AB-001245 (FIGS. 10A-10F). Biological replicates of each antibody variant are shown in FIGS. 10A-10F. The concentration of human antibodies that were circulating in the mice at the time of infection was determined by ELISA and is shown in FIGS. 10G-10I.

### Bite Parasitemia Assay

AB-000224 and AB-007088 and variants thereof were also evaluated for their ability to protect against infection (with those in format comprising Heavy Chain version 2 indicated with the “LS”). In this experiment, animals were exposed to mosquitoes infected with chimeric P. berghei expressing P. falciparum CSP protein, as described in Espinosa, D., et al. npj Vaccines 2017; 2, 10 (2017); Espinosa, D., et al. Infect Immun. 2013 August; 81(8): 2882-2887.

C57Bl/6 Mice were administered 150 μg of antibody and 16 hours later were exposed to six or seven chimeric P. berghei-infected mosquitoes. At least 70%-80% of the mosquito population are infected with chimeric P. berghei expressing P. falciparum CSP protein, resulting in exposure to at least one infectious event. Each mouse was subsequently evaluated for blood stage parasitemia from days 4-10 following infection by microscopy. Parasitemia data were recorded as positive or negative and the data used to generate a survival curve. Experimental antibodies were compared to both the positive (AB-000317) and negative (AB-001245) controls.

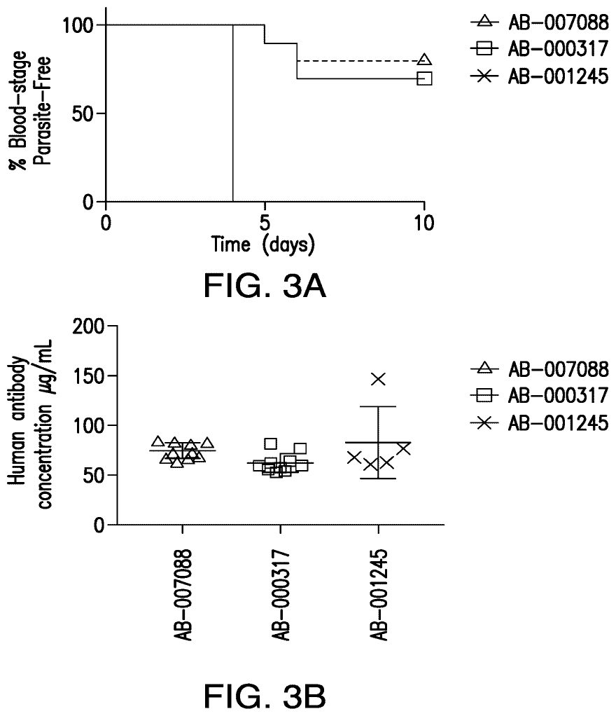

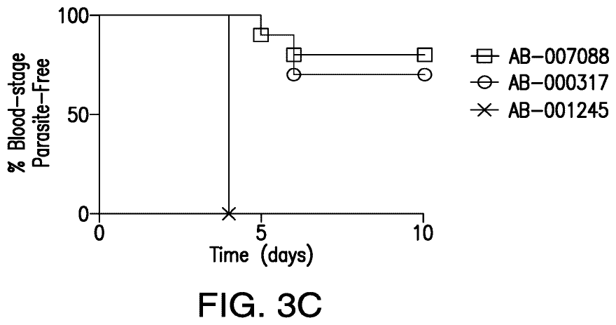

Mice treated with AB-000224, AB-007088 or the positive control AB-000317 were less likely to develop parasitemia as compared to mice treated with the negative control AB-001245. (FIGS. 2A and 2C show AB-000224 experimental data and FIGS. 3A and 3C show AB-007088 experimental data). The level of human antibodies circulating in the mice at the time of infection was quantified via an ELISA assay (FIGS. 2B and 3B) and was similar between the experimental and control antibodies.

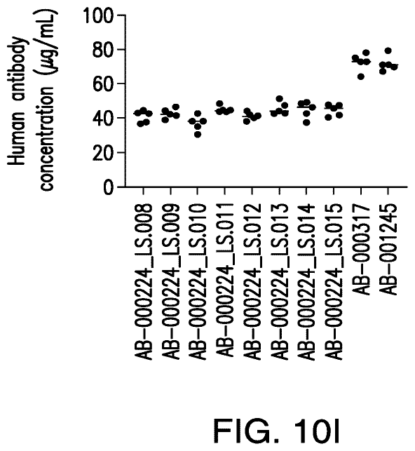

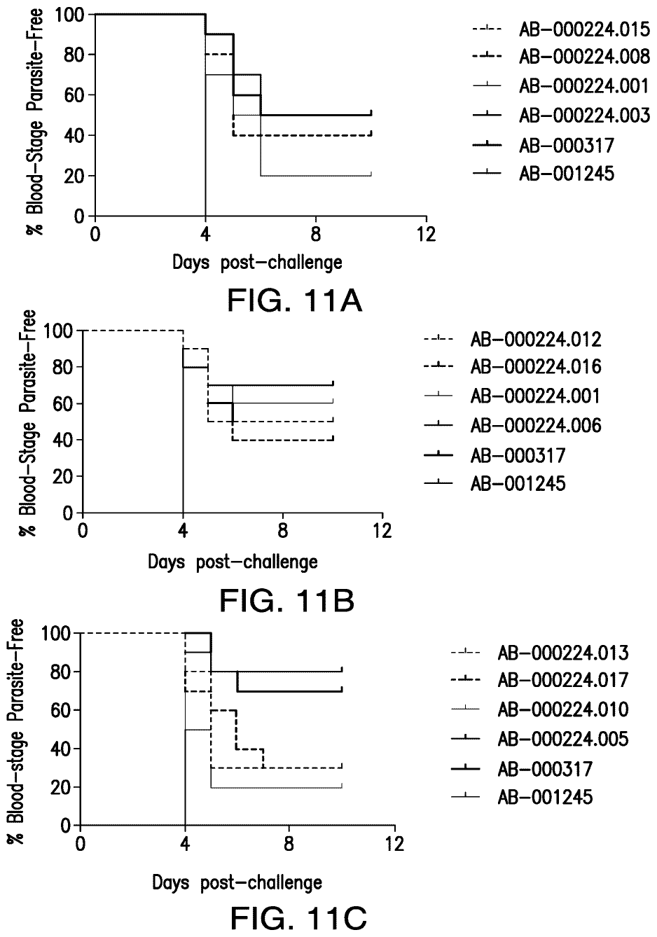

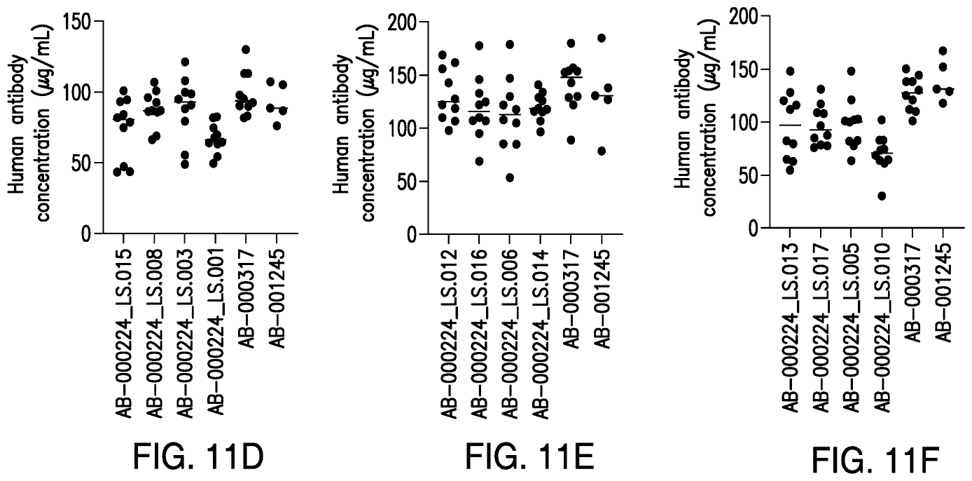

Finally, mice administered variants of AB-000224 or AB-007088 were comparably likely to remain parasite-free as mice treated with either of the parent antibodies, or the positive control antibody AB-000317 (FIGS. 11A-11C). The concentration of human antibodies that were circulating in the mice at the time of infection was determined by ELISA and is shown in FIGS. 11D-11F.

### Example 5. Biophysical Characterization

The present example describes the biophysical hallmarks of the anti-CSP antibody variants. AB-000224 and AB-007088 and variants thereof (all in the format comprising Heavy Chain version 2) were evaluated for their conformational stability and colloidal stability. Several methods were used to analyze these endpoints and are summarized in the Tables 37 and 38 below:

TABLE 37
Conformational Stability Methods

| | | | | Values that indicate |
|---|---|---|---|---|
| Method Name | Attribute Measured | Characterization | Desired Outcome | instability |
| Differential | Melting temperature | Lower Tm(app) is | Increase in Tm or formation of | Weighted Shoulder |
| scanning | | indicative of decreased | additional Tm's indicating | Score (WSS) Below 20 |
| fluorimetry (DSF) | | conformational stability | increased domain stability | |
| Thermal hold | Temperature induced | Define conditions for | Increase in thermal | Absorbance at |
| | aggregation | precipitation relating | stability indicated by | 350 nm is above 0.5 |
| | | to potential destabilization | the absence of | |
| | | during room temperature | precipitation | |
| | | incubation | | |
| Low pH | Low pH stability by | Molecules with an increase in | No significant | More than 10% |
| Aggregation | high molecular | high molecule weight following | increase in high | aggregation after |
| | weight | neutralization after low pH | molecular weight | low pH exposure |
| | | exposure may show increased | species following | |
| | | aggregation during low pH | low pH exposure and | |
| | | viral inactivation | neutralization | |
| Chemical | Conformational | Molecules with an increased | Increased inflection | Less than 2.1M |
| Unfolding | stability | inflection point may show | point compared to | inflection point |
| | | lower rates of aggregation | the parent molecule | |
| | | during storage and are more | | |
| | | conformationally stable | | |

TABLE 38
Colloidal Stability Methods

| | | | | Values that indicate |
|---|---|---|---|---|
| Method Name | Attribute Measured | Characterization | Desired Outcome | instability |
| Self-interaction | Protein-protein | Define relative protein | Decrease in parameters | Wavelength |
| nanoparticle | interaction | self-association to help | indicating self-association | Maximum Above |
| spectroscopy | | identify potential for | of the molecule | 550 nm |
| (SINS) | | higher viscosity during | | |
| | | concentration and problems | | |
| | | with filterability | | |
| Standup Monolayer | Colloidal stability | Molecules with reduced | Decreased main peak | Longer retention times, |
| Affinity | | retention times may show | retention time | there is no established |
| Chromatography | | increase solubility and | compared to parent | values correlating to |
| (Zenix Column) | | lower rates of aggregation | molecule | instability at this time |
| | | during storage | | |
| Relative Solubility | Polyethylene glycol | Molecules with higher relative | Increased solubility | 50% Loss of protein |
| Analysis (RSA) | based solubility | solubility may show lower rates | compared to parent | occurring less than |
| | analysis | of aggregation during storage | molecule | 7% PEG |
| Polyreactivity | Non-specific binding | Molecules that have the | Decreased non- | Absorbance at 405 nm |
| | | potential to non-specifically | specific binding | is above 1.5-2.0 |
| | | bind to an array of different | compared to the | |
| | | antigens can potentially have a | parent molecule | |
| | | higher clearance rate. | | |

### AB-000224 Variants Showed Improved Stability

AB-000224 and variants thereof showed comparable harvest yield and titer results. Thus, additional end-points were assessed. AB-000224 and variants thereof were evaluated for their thermal stability by differential scanning fluorimetry (DSF). Thermal unfolding was monitored by measuring the intensity of an extrinsic dye (sypro orange) as the sample was heated from 20° C. to 90° C. The data was reported as Tm1 and Tm2 with the first transition correlating with the CH2 domain and the 2nd transition correlating with the unfolding of the Fab and CH3 domain regions. 10 Higher unfolding temperatures are desirable and have been linked with an increase in a product's conformational stability. Lack of a Tm2 is indicative of the Fab unfolding at the same or similar temperature to the CH2 domain, reported as Tm1. Additional information is also obtained from a proprietary parameter termed the weighted shoulder score which accounts for multiple pieces of information from the unfolding curve. Again, higher values are indicative of greater conformational stability. The DSF analysis was conducted in PBS buffer, with all samples being diluted down to a final antibody concentration of 0.15 mg/mL.

The propensity to aggregate at elevated temperatures was assessed using the thermal aggregation method. Samples were place in a 96 well Biorad PCR plate and heated to various temperatures for 5 minutes using a Biorad Thermal Cycler. After heating, protein precipitation was determined by reading the absorbance at 350 nm (A350) using a Spectrostar nano plate reader. Almost all AB-000224 variants showed higher WSS as compared to the parent antibody AB-000224, a T2 that approached 80° C., and no precipitation during thermal hold assays. However, AB-000224.017 did not have a T2 and showed precipitation during thermal hold assay.

Further, AB-000224 and variants thereof were also evaluated for their chemical stability. Sensitivity to low pH was assessed by titrating the samples to pH 3.3 using acetic acid, holding the samples for 30 minutes, neutralizing the samples to pH 5 with tris base, and measuring aggregation by size exclusion HPLC. Samples that were diluted with PBS using the same volume of acetic acid and tris base as used in the test samples were used as a control. AB-000224 and all variants thereof did not show low pH instability.

AB-000224 and variants thereof were tested for stability against chemical unfolding as assayed by denaturation with guanidine. Chemical unfolding curves were produced by exposing the antibodies to increasing concentrations of guanidine hydrochloride. After 24 hours, the intrinsic fluorescence of the samples is measured using a SUPR-UV plate reader. The collected raw data was then processed and the chemical unfolding curve and its inflection point calculated from the processed data as a function of denaturant condition. Antibodies with denaturation inflection points higher than 2.1 M guanidine are considered to be conformationally stable by this method. AB-000224 and all variants showed inflection points above 2 M guanidine (Gdn). Notably, AB-000224.005, AB-000224.008, AB-000224.010, AB-000224.011, AB-000224.013, and AB-000224.015 showed improved stability against chemical unfolding as compared to AB-000224, each having an inflection point above 2.3 M guanidine.

AB-000224 and variants thereof were also evaluated for their colloidal stability by self-interaction nanoparticle spectroscopy (SINS) which monitors protein-protein interactions by capture on the surface of a gold colloid and measuring shifts of the wavelength of maximum absorption. Maximum absorption values of wavelengths higher than 550 nm are consider interacting and could manifest in increase viscosity and filterability issues. All variants had maximum absorption values less than 550 nm and showed a slight improvement in the SINS values as compared to AB-000224.

AB-000224 and variants thereof were tested for potential hydrophobic interactions that might results in manufacturability challenges by monitoring retention time on a Zenix HPLC column. Undiluted samples were loaded onto the Zenix column and eluted isocratically with a 100 mM sodium phosphate, pH 7.0 running buffer and monitored at 220 nm. Longer retention times indicates hydrophobic interactions. Most antibodies have a retention time of 8.5-9.0 minutes under conditions tested. AB-00224 and all variants thereof showed comparable retention times of approximately 10 minutes.

Solubility of AB-000224 and variants thereof was assayed by precipitating the antibody samples with increasing amounts of PEG 10,000, filtering the samples, and measuring the soluble protein concentration. For each parent molecule, an initial experiment was performed to determine the ideal PEG concentration to precipitate the parent by approximately 50%, subsequent experiments used this PEG concentration on all variants to assess if a variant is more or less soluble than the parent molecule. Antibodies that precipitate above 8-10% PEG are considered highly soluble while poorly soluble antibodies precipitate at 4-5% PEG. AB-000224 and all variants thereof showed high solubility.

Polyreactivity of AB-000224 and variants thereof was determined by testing the antibodies for binding to KLH, insulin and dsDNA by Elisa. Samples were diluted to 1 μg/mL and a secondary anti-human antibody was used to detect the amount of protein that has bound to the different antigens. After substrate addition, absorbance was measured at 405 nm. A polyreactive antibody was used as a positive control. Non-specific binding to such common physiological components can cause an increase clearance rate, negatively impacting pK. Absorbance values above 1.5 for KLH and Insulin and 2.0 for dsDNA could indicate non-specific binding issues for a molecule. While most of the AB-000224 variants did not show any polyreactivity, some variants showed polyreactive signal against insulin (AB-000224.006, AB-000224.007, AB-000224.008, AB-000224.009, AB-000224.010).

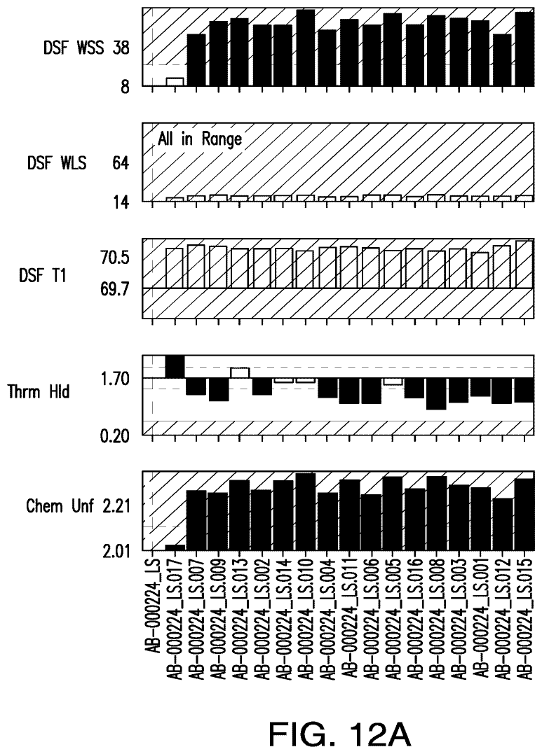

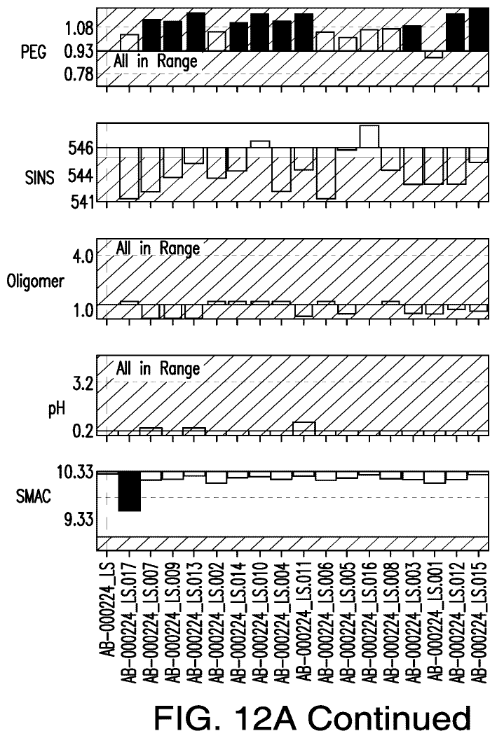

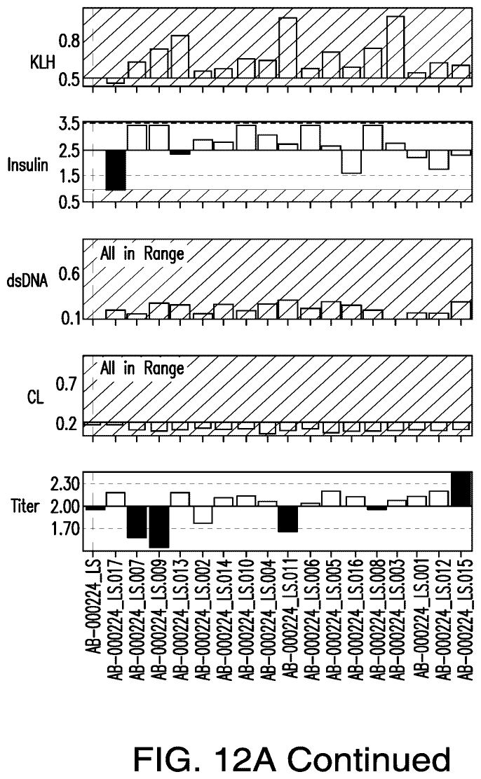

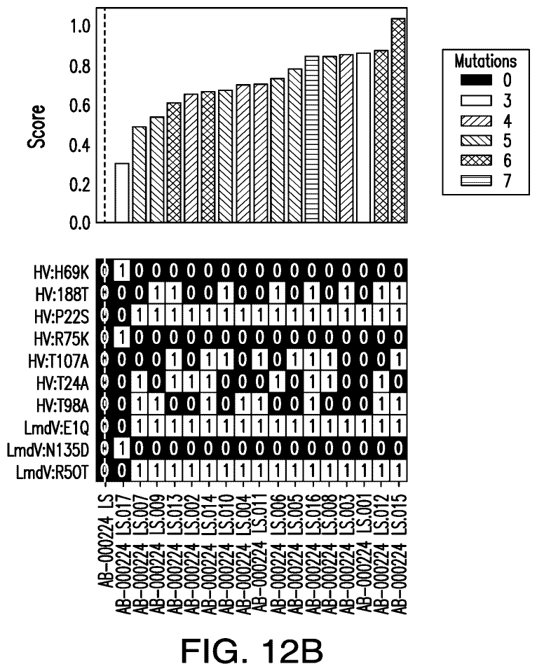

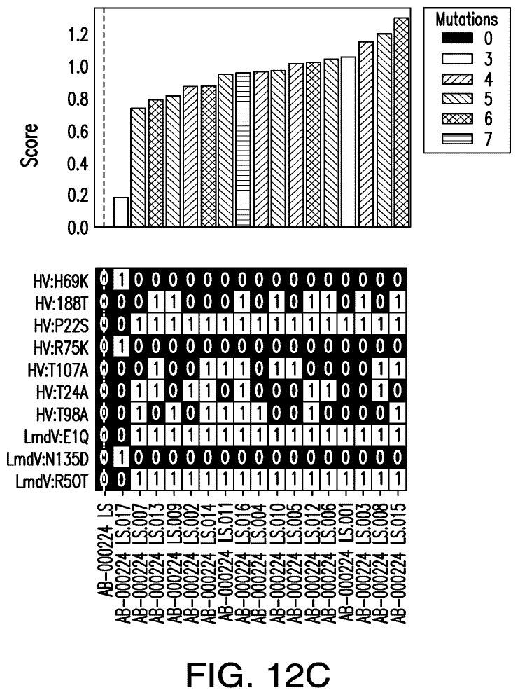

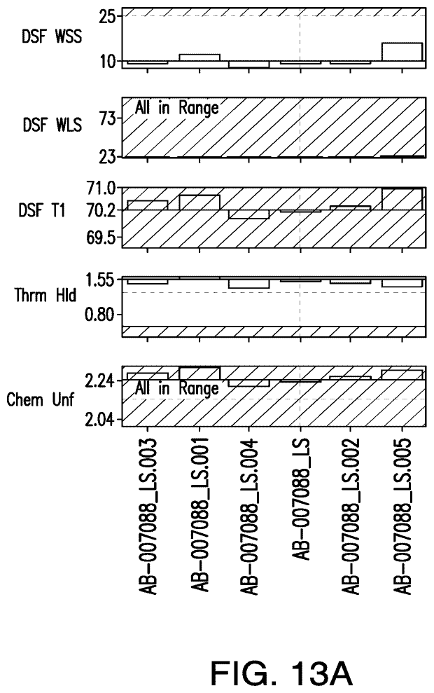

The AB-000224 and variants thereof were ranked based on the different assays and titer results and ranked as described in FIGS. 12B and 12C.

### AB-007088 Variants Showed Improved Stability

The harvest yield and titer of the AB-007088 and variants thereof were initially evaluated. The AB-007088 variants showed comparable harvest yield and titer results. AB-007088 and variants thereof were evaluated to determine conformational and colloidal stability as described above for AB-000224 and variants thereof.

The AB-007088.005 variant showed improved thermal stability by the DFS method and a T2 that approached 75° C., while the other variants were similar to the parent AB-007088 with Tl values of approximately 70° C. and no measurable T2. In addition, AB-007088.005 showed a slightly reduced precipitation as compared to the parent AB-007088 and the other variants. When chemical stability was evaluated, AB-007088 and all variants thereof did not show low pH instability (at pH 3.3). Further, AB-007088 and all variants thereof showed stability against chemical unfolding with an inflection point above 2 M guanidine (Gdn). Notably, AB-007088.001 showed improved stability as compared to AB-007088 with an inflection point above 2.3 M guanidine.

AB-007088 and variants thereof were also evaluated for their colloidal stability. When protein-protein interactions were monitored (SINS), all variants showed very low SINS values, indicating absence of protein-protein interactions. In addition, AB-007088 and variants thereof showed comparable retention times on Zenix column. Notably, when PEG solubility was determined, AB-007088 and all variants thereof showed high solubility. Further, while most of the AB-007088 variants did not show any polyreactivity, some variants showed polyreactive signal against insulin (AB-007088.001, AB-007088.003, AB-007088.004).

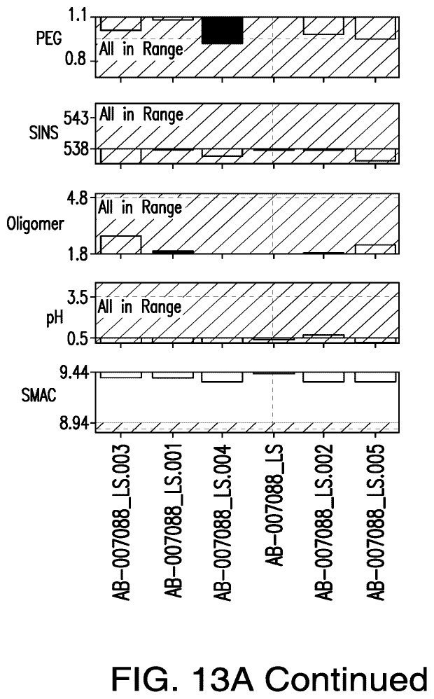

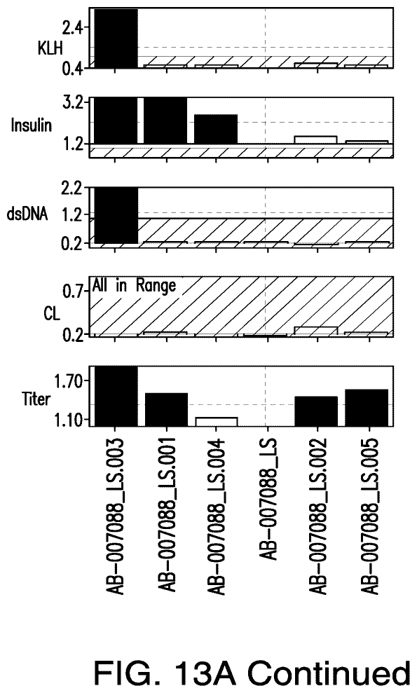

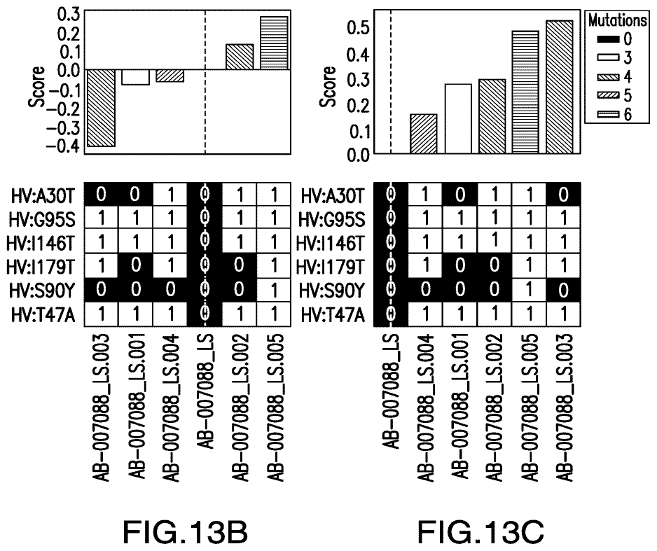

The AB-007088 and variants thereof were ranked based on the different assays and titer results, and ranked as described in FIGS. 13B and 13C.

1-28. (canceled)

29. A light chain polypeptide comprising a light chain variable region (VL), wherein the VL comprises a set of complementarity determining regions (CDRs) selected from the group consisting of:
(a) a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3;
(b) a CDR1 comprising the amino acid sequence set forth in SEQ ID NO: 183, a CDR2 comprising the amino acid sequence set forth in SEQ ID NO: 184, and a CDR3 comprising the amino acid sequence set forth in SEQ ID NO: 185; and
(c) a CDR1 comprising the amino acid sequence set forth in SEQ ID NO: 205, a CDR2 comprising the amino acid sequence set forth in SEQ ID NO: 206, and a CDR3 comprising the amino acid sequence set forth in SEQ ID NO: 207.

30. A heavy chain polypeptide comprising a heavy chain variable region (VH), wherein the VH comprises a set of complementarity determining regions (CDRs) selected from the group consisting of:
(a) a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
(b) a CDR1 comprising the amino acid sequence set forth in SEQ ID NO: 186, a CDR2 comprising the amino acid sequence set forth in SEQ ID NO: 187, and a CDR3 comprising the amino acid sequence set forth in SEQ ID NO: 188; and
(c) a CDR1 comprising the amino acid sequence set forth in SEQ ID NO: 208, a CDR2 comprising the amino acid sequence set forth in SEQ ID NO: 209, and a CDR3 comprising the amino acid sequence set forth in SEQ ID NO: 210.

31. The light chain polypeptide of claim 29, wherein the VL comprises an amino acid sequence selected from the group consisting of SEQ ID NO: 163, 23, 33, 43, 53, 63, 73, 83, 93, 103, 113, 123, 133, 143, 153, 173, 195, 227, 237, 247, 257, and 267.

32. The heavy chain polypeptide of claim 30, wherein the VH comprises an amino acid sequence selected from the group consisting of SEQ ID NO: 164, 24, 34, 44, 54, 64, 74, 84, 94, 104, 114, 124, 134, 144, 154, 174, 196, 228, 238, 248, 258, and 268.

33. The light chain polypeptide of claim 29, wherein the light chain comprises an amino acid sequence selected from the group consisting of SEQ ID NO: 167, 27, 37, 47, 57, 67, 77, 87, 97, 107, 117, 127, 137, 147, 157, 177, 199, 231, 241, 251, 261, and 271.

34. The heavy chain polypeptide of claim 30, wherein the heavy chain comprises an amino acid sequence selected from the group consisting of SEQ ID NO: 168, 169, 28, 29, 38, 39, 48, 49, 58, 59, 68, 69, 78, 79, 88, 89, 98, 99, 108, 109, 118, 119, 128, 129, 138, 139, 148, 149, 158, 159, 178, 179, 200, 201, 232, 233, 242, 243, 252, 253, 262, 263, 272, and 273.

35. The light chain polypeptide of claim 29, wherein the VL comprises the amino acid sequence set forth in SEQ ID NO: 13 and at least one amino acid substitution at position 1 and/or position 44.

36. The light chain polypeptide of claim 35, wherein the amino acid substitution at position 1 is E1Q and the amino acid substitution at position 44 is R44T.

37. The heavy chain polypeptide of claim 30, wherein the VH comprises the amino acid sequence set forth in SEQ ID NO: 14 and at least one amino acid substitution at position 21, position 23, position 80, position 90, or a combination thereof.

38. The heavy chain polypeptide of claim 37, wherein:
(a) the amino acid substitution at position 21 is P21S;
(b) the amino acid substitution at position 23 is T23A;
(c) the amino acid substitution at position 80 is I80T; and
(d) the amino acid substitution at position 90 is T90A.

39. The heavy chain polypeptide of claim 30, wherein the heavy chain comprises the amino acid sequence set forth in SEQ ID NO: 18 and at least one amino acid substitution at position 438 and/or position 444.

40. The heavy chain polypeptide of claim 39, wherein the amino acid substitution at position 438 is M438L and the amino acid substitution at position 444 is N444S.

41. The heavy chain polypeptide of claim 30, wherein the VH comprises the amino acid sequence set forth in SEQ ID NO: 218 and at least one amino acid substitution at position 40, position 69, position 80, position 85, position 120, or a combination thereof.

42. The heavy chain polypeptide of claim 41, wherein:
(a) the amino acid substitution at position 40 is T40A;
(b) the amino acid substitution at position 69 is I69T;
(c) the amino acid substitution at position 80 is S80Y;
(d) the amino acid substitution at position 85 is G85S; and
(e) the amino acid substitution at position 120 is I120T.

43. The heavy chain polypeptide of claim 30, wherein the heavy chain comprises the amino acid sequence set forth in SEQ ID NO: 222 and at least one amino acid substitution at position 434 and/or position 440.

44. The heavy chain polypeptide of claim 43, wherein the amino acid substitution at position 434 is M434L and the amino acid substitution at position 440 is N440S.

45. A polynucleotide encoding a light chain polypeptide, wherein the polynucleotide comprises a nucleotide sequence selected from the group consisting of SEQ ID NO: 165, 25, 35, 45, 55, 65, 75, 85, 95, 105, 115, 125, 135, 145, 155, 175, 197, 229, 239, 249, 259, and 269 or encoding a heavy chain polypeptide, wherein the polynucleotide comprises a nucleotide sequence selected from the group consisting of SEQ ID NO: 166, 26, 36, 46, 56, 66, 76, 86, 96, 106, 116, 126, 136, 146, 156, 176, 198, 230, 240, 250, 260, and 270.

46. A vector comprising the polynucleotide of claim 45, optionally wherein the vector is a plasmid or a viral vector, and optionally wherein the viral vector is an adenovirus, an adeno-associated virus, or a retrovirus.


SEQUENCE LISTING (289 sequences)

SEQ ID NO: 1 - protein, 14 aa, synthetic construct
  REGION 1..14: note = Synthetic
TGMNSNIGAGYDVY

SEQ ID NO: 2 - protein, 7 aa, synthetic construct
  REGION 1..7: note = Synthetic
GNSNRPS

SEQ ID NO: 3 - protein, 11 aa, synthetic construct
  REGION 1..11: note = Synthetic
QSYDTSLNGWA

SEQ ID NO: 4 - protein, 5 aa, synthetic construct
  REGION 1..5: note = Synthetic
DHAMS

SEQ ID NO: 5 - protein, 19 aa, synthetic construct
  REGION 1..19: note = Synthetic
FIRKTTYGATTHYAAAVRG

SEQ ID NO: 6 - protein, 16 aa, synthetic construct
  REGION 1..16: note = Synthetic
VQLDYGPGYQYYGMDV

SEQ ID NO: 7 - protein, 14 aa, synthetic construct
  REGION 1..14: note = Synthetic
TGMNSNIGAGYDVY

SEQ ID NO: 8 - protein, 7 aa, synthetic construct
  REGION 1..7: note = Synthetic
GNSNRPS

SEQ ID NO: 9 - protein, 11 aa, synthetic construct
  REGION 1..11: note = Synthetic
QSYDTSLNGWA

SEQ ID NO: 10 - protein, 5 aa, synthetic construct
  REGION 1..5: note = Synthetic
DHAMS

SEQ ID NO: 11 - protein, 19 aa, synthetic construct
  REGION 1..19: note = Synthetic
FIRKTTYGATTHYAAAVRG

SEQ ID NO: 12 - protein, 16 aa, synthetic construct
  REGION 1..16: note = Synthetic
VQLDYGPGYQYYGMDV

SEQ ID NO: 13 - protein, 112 aa, synthetic construct
  REGION 1..112: note = Synthetic
ESVLTQPPSVSGAPGQRVTISCTGMNSNIGAGYDVYWYQQLPGRAPKLLIYGNSNRPSGVPDRFSGSRSGTSASLAITGLQAEDEADYYCQSYDTSLNGWAFGGGTKLTVLG

SEQ ID NO: 14 - protein, 127 aa, synthetic construct
  REGION 1..127: note = Synthetic
EVQLVESGGGLVQPGRSLRLPCTASGFSFGDHAMSWVRQAPGKGLEWVGFIRKTTYGATTHYAAAVRGRFTISRDDSKSIVYLQMNSLKTEDTAVYFCTRVQLDYGPGYQYYGMDVWGQGTTVTVSS

SEQ ID NO: 15 - DNA, 336 bp, synthetic construct
  misc_feature 1..336: note = Synthetic
  source 1..336: mol_type = other DNA
gagtctgtgctgacgcagccgccctcagtgtctggggccccagggcagagggtcaccatctcctgcactgggatgaactccaacatcggggcaggttatgatgtatactggtaccaacaacttccaggaagagcccccaaactcctcatctatggtaacagcaatcggccctcaggggtccctgaccgattctctggctccaggtctggcacctcagcctccctggccatcactgggctccaggctgaggatgaggctgattattactgccagtcctatgacaccagcctgaatggttgggctttcggcggagggaccaagttgaccgtcctaggc

SEQ ID NO: 16 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcccctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtactagagtgcagcttgactatggcccgggataccagtactacggtatggacgtctggggccaagggaccacggtcaccgtctcctca

SEQ ID NO: 17 - protein, 217 aa, synthetic construct
  REGION 1..217: note = Synthetic
ESVLTQPPSVSGAPGQRVTISCTGMNSNIGAGYDVYWYQQLPGRAPKLLIYGNSNRPSGVPDRFSGSRSGTSASLAITGLQAEDEADYYCQSYDTSLNGWAFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 18 - protein, 457 aa, synthetic construct
  REGION 1..457: note = Synthetic
EVQLVESGGGLVQPGRSLRLPCTASGFSFGDHAMSWVRQAPGKGLEWVGFIRKTTYGATTHYAAAVRGRFTISRDDSKSIVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 457 aa, synthetic construct
  REGION 1..457: note = Synthetic
EVQLVESGGGLVQPGRSLRLPCTASGFSFGDHAMSWVRQAPGKGLEWVGFIRKTTYGATTHYAAAVRGRFTISRDDSKSIVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 20 - DNA, 651 bp, synthetic construct
  misc_feature 1..651: note = Synthetic
  source 1..651: mol_type = other DNA
gagtctgtgctgacgcagccgccctcagtgtctggggccccagggcagagggtcaccatctcctgcactgggatgaactccaacatcggggcaggttatgatgtatactggtaccaacaacttccaggaagagcccccaaactcctcatctatggtaacagcaatcggccctcaggggtccctgaccgattctctggctccaggtctggcacctcagcctccctggccatcactgggctccaggctgaggatgaggctgattattactgccagtcctatgacaccagcctgaatggttgggctttcggcggagggaccaagttgaccgtcctaggccagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 21 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcccctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 22 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcccctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 23 - protein, 112 aa, synthetic construct
  REGION 1..112: note = Synthetic
QSVLTQPPSVSGAPGQRVTISCTGMNSNIGAGYDVYWYQQLPGTAPKLLIYGNSNRPSGVPDRFSGSRSGTSASLAITGLQAEDEADYYCQSYDTSLNGWAFGGGTKLTVLG

SEQ ID NO: 24 - protein, 127 aa, synthetic construct
  REGION 1..127: note = Synthetic
EVQLVESGGGLVQPGRSLRLSCTASGFSFGDHAMSWVRQAPGKGLEWVGFIRKTTYGATTHYAAAVRGRFTISRDDSKSIVYLQMNSLKTEDTAVYFCTRVQLDYGPGYQYYGMDVWGQGTTVTVSS

SEQ ID NO: 25 - DNA, 336 bp, synthetic construct
  misc_feature 1..336: note = Synthetic
  source 1..336: mol_type = other DNA
cagtctgtgctgacgcagccgccctcagtgtctggggccccagggcagagggtcaccatctcctgcactgggatgaactccaacatcggggcaggttatgatgtatactggtaccaacaacttccaggaactgcccccaaactcctcatctatggtaacagcaatcggccctcaggggtccctgaccgattctctggctccaggtctggcacctcagcctccctggccatcactgggctccaggctgaggatgaggctgattattactgccagtcctatgacaccagcctgaatggttgggctttcggcggagggaccaagttgaccgtcctaggc

SEQ ID NO: 26 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtactagagtgcagcttgactatggcccgggataccagtactacggtatggacgtctggggccaagggaccacggtcaccgtctcctca

SEQ ID NO: 27 - protein, 217 aa, synthetic construct
  REGION 1..217: note = Synthetic
QSVLTQPPSVSGAPGQRVTISCTGMNSNIGAGYDVYWYQQLPGTAPKLLIYGNSNRPSGVPDRFSGSRSGTSASLAITGLQAEDEADYYCQSYDTSLNGWAFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 28 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSIVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSIVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 30 - DNA, 651 bp, synthetic construct
  misc_feature 1..651: note = Synthetic
  source 1..651: mol_type = other DNA
cagtctgtgctgacgcagccgccctcagtgtctggggccccagggcagagggtcaccatctcctgcactgggatgaactccaacatcggggcaggttatgatgtatactggtaccaacaacttccaggaactgcccccaaactcctcatctatggtaacagcaatcggccctcaggggtccctgaccgattctctggctccaggtctggcacctcagcctccctggccatcactgggctccaggctgaggatgaggctgattattactgccagtcctatgacaccagcctgaatggttgggctttcggcggagggaccaagttgaccgtcctaggccagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 31 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 32 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 33 - protein, 112 aa, synthetic construct; identical to SEQ ID NO: 23
  REGION 1..112: note = Synthetic

SEQ ID NO: 34 - protein, 127 aa, synthetic construct
  REGION 1..127: note = Synthetic
EVQLVESGGGLVQPGRSLRLSCAASGFSFGDHAMSWVRQAPGKGLEWVGFIRKTTYGATTHYAAAVRGRFTISRDDSKSIVYLQMNSLKTEDTAVYFCTRVQLDYGPGYQYYGMDVWGQGTTVTVSS

SEQ ID NO: 35 - DNA, 336 bp, synthetic construct; identical to SEQ ID NO: 25
  misc_feature 1..336: note = Synthetic
  source 1..336: mol_type = other DNA

SEQ ID NO: 36 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtactagagtgcagcttgactatggcccgggataccagtactacggtatggacgtctggggccaagggaccacggtcaccgtctcctca

SEQ ID NO: 37 - protein, 217 aa, synthetic construct; identical to SEQ ID NO: 27
  REGION 1..217: note = Synthetic

SEQ ID NO: 38 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSIVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSIVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 40 - DNA, 651 bp, synthetic construct; identical to SEQ ID NO: 30
  misc_feature 1..651: note = Synthetic
  source 1..651: mol_type = other DNA

SEQ ID NO: 41 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 42 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 43 - protein, 112 aa, synthetic construct; identical to SEQ ID NO: 23
  REGION 1..112: note = Synthetic

SEQ ID NO: 44 - protein, 127 aa, synthetic construct
  REGION 1..127: note = Synthetic
EVQLVESGGGLVQPGRSLRLSCTASGFSFGDHAMSWVRQAPGKGLEWVGFIRKTTYGATTHYAAAVRGRFTISRDDSKSTVYLQMNSLKTEDTAVYFCTRVQLDYGPGYQYYGMDVWGQGTTVTVSS

SEQ ID NO: 45 - DNA, 336 bp, synthetic construct; identical to SEQ ID NO: 25
  misc_feature 1..336: note = Synthetic
  source 1..336: mol_type = other DNA

SEQ ID NO: 46 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaaaccgaggacacagccgtgtatttctgtactagagtgcagcttgactatggcccgggataccagtactacggtatggacgtctggggccaagggaccacggtcaccgtctcctca

SEQ ID NO: 47 - protein, 217 aa, synthetic construct; identical to SEQ ID NO: 27
  REGION 1..217: note = Synthetic

SEQ ID NO: 48 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSTVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSTVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 50 - DNA, 651 bp, synthetic construct; identical to SEQ ID NO: 30
  misc_feature 1..651: note = Synthetic
  source 1..651: mol_type = other DNA

SEQ ID NO: 51 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaaaccgaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 52 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaaaccgaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 53 - protein, 112 aa, synthetic construct; identical to SEQ ID NO: 23
  REGION 1..112: note = Synthetic

SEQ ID NO: 54 - protein, 127 aa, synthetic construct
  REGION 1..127: note = Synthetic
EVQLVESGGGLVQPGRSLRLSCTASGFSFGDHAMSWVRQAPGKGLEWVGFIRKTTYGATTHYAAAVRGRFTISRDDSKSIVYLQMNSLKAEDTAVYFCTRVQLDYGPGYQYYGMDVWGQGTTVTVSS

SEQ ID NO: 55 - DNA, 336 bp, synthetic construct; identical to SEQ ID NO: 25
  misc_feature 1..336: note = Synthetic
  source 1..336: mol_type = other DNA

SEQ ID NO: 56 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaagcagaggacacagccgtgtatttctgtactagagtgcagcttgactatggcccgggataccagtactacggtatggacgtctggggccaagggaccacggtcaccgtctcctca

SEQ ID NO: 57 - protein, 217 aa, synthetic construct; identical to SEQ ID NO: 27
  REGION 1..217: note = Synthetic

SEQ ID NO: 58 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSIVYLQMNSLKAEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSIVYLQMNSLKAEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 60 - DNA, 651 bp, synthetic construct; identical to SEQ ID NO: 30
  misc_feature 1..651: note = Synthetic
  source 1..651: mol_type = other DNA

SEQ ID NO: 61 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaagcagaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 62 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaagcagaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 63 - protein, 112 aa, synthetic construct; identical to SEQ ID NO: 23
  REGION 1..112: note = Synthetic

SEQ ID NO: 64 - protein, 127 aa, synthetic construct
  REGION 1..127: note = Synthetic
EVQLVESGGGLVQPGRSLRLSCTASGFSFGDHAMSWVRQAPGKGLEWVGFIRKTTYGATTHYAAAVRGRFTISRDDSKSIVYLQMNSLKTEDTAVYFCARVQLDYGPGYQYYGMDVWGQGTTVTVSS

SEQ ID NO: 65 - DNA, 336 bp, synthetic construct; identical to SEQ ID NO: 25
  misc_feature 1..336: note = Synthetic
  source 1..336: mol_type = other DNA

SEQ ID NO: 66 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtgctagagtgcagcttgactatggcccgggataccagtactacggtatggacgtctggggccaagggaccacggtcaccgtctcctca

SEQ ID NO: 67 - protein, 217 aa, synthetic construct; identical to SEQ ID NO: 27
  REGION 1..217: note = Synthetic

SEQ ID NO: 68 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSIVYLQMNSLKTEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSIVYLQMNSLKTEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 70 - DNA, 651 bp, synthetic construct; identical to SEQ ID NO: 30
  misc_feature 1..651: note = Synthetic
  source 1..651: mol_type = other DNA

SEQ ID NO: 71 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 72 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 73 - protein, 112 aa, synthetic construct; identical to SEQ ID NO: 23
  REGION 1..112: note = Synthetic

SEQ ID NO: 74 - protein, 127 aa, synthetic construct
  REGION 1..127: note = Synthetic
EVQLVESGGGLVQPGRSLRLSCAASGFSFGDHAMSWVRQAPGKGLEWVGFIRKTTYGATTHYAAAVRGRFTISRDDSKSTVYLQMNSLKTEDTAVYFCTRVQLDYGPGYQYYGMDVWGQGTTVTVSS

SEQ ID NO: 75 - DNA, 336 bp, synthetic construct; identical to SEQ ID NO: 25
  misc_feature 1..336: note = Synthetic
  source 1..336: mol_type = other DNA

SEQ ID NO: 76 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaaaccgaggacacagccgtgtatttctgtactagagtgcagcttgactatggcccgggataccagtactacggtatggacgtctggggccaagggaccacggtcaccgtctcctca

SEQ ID NO: 77 - protein, 217 aa, synthetic construct; identical to SEQ ID NO: 27
  REGION 1..217: note = Synthetic

SEQ ID NO: 78 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSTVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSTVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 80 - DNA, 651 bp, synthetic construct; identical to SEQ ID NO: 30
  misc_feature 1..651: note = Synthetic
  source 1..651: mol_type = other DNA

SEQ ID NO: 81 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaaaccgaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 82 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaaaccgaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 83 - protein, 112 aa, synthetic construct; identical to SEQ ID NO: 23
  REGION 1..112: note = Synthetic

SEQ ID NO: 84 - protein, 127 aa, synthetic construct
  REGION 1..127: note = Synthetic
EVQLVESGGGLVQPGRSLRLSCAASGFSFGDHAMSWVRQAPGKGLEWVGFIRKTTYGATTHYAAAVRGRFTISRDDSKSIVYLQMNSLKAEDTAVYFCTRVQLDYGPGYQYYGMDVWGQGTTVTVSS

SEQ ID NO: 85 - DNA, 336 bp, synthetic construct; identical to SEQ ID NO: 25
  misc_feature 1..336: note = Synthetic
  source 1..336: mol_type = other DNA

SEQ ID NO: 86 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaagcagaggacacagccgtgtatttctgtactagagtgcagcttgactatggcccgggataccagtactacggtatggacgtctggggccaagggaccacggtcaccgtctcctca

SEQ ID NO: 87 - protein, 217 aa, synthetic construct; identical to SEQ ID NO: 27
  REGION 1..217: note = Synthetic

SEQ ID NO: 88 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSIVYLQMNSLKAEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSIVYLQMNSLKAEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 90 - DNA, 651 bp, synthetic construct; identical to SEQ ID NO: 30
  misc_feature 1..651: note = Synthetic
  source 1..651: mol_type = other DNA

SEQ ID NO: 91 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaagcagaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 92 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaagcagaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 93 - protein, 112 aa, synthetic construct; identical to SEQ ID NO: 23
  REGION 1..112: note = Synthetic

SEQ ID NO: 94 - protein, 127 aa, synthetic construct
  REGION 1..127: note = Synthetic
EVQLVESGGGLVQPGRSLRLSCAASGFSFGDHAMSWVRQAPGKGLEWVGFIRKTTYGATTHYAAAVRGRFTISRDDSKSIVYLQMNSLKTEDTAVYFCARVQLDYGPGYQYYGMDVWGQGTTVTVSS

SEQ ID NO: 95 - DNA, 336 bp, synthetic construct; identical to SEQ ID NO: 25
  misc_feature 1..336: note = Synthetic
  source 1..336: mol_type = other DNA

SEQ ID NO: 96 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtgctagagtgcagcttgactatggcccgggataccagtactacggtatggacgtctggggccaagggaccacggtcaccgtctcctca

SEQ ID NO: 97 - protein, 217 aa, synthetic construct; identical to SEQ ID NO: 27
  REGION 1..217: note = Synthetic

SEQ ID NO: 98 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSIVYLQMNSLKTEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSIVYLQMNSLKTEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 100 - DNA, 651 bp, synthetic construct; identical to SEQ ID NO: 30
  misc_feature 1..651: note = Synthetic
  source 1..651: mol_type = other DNA

SEQ ID NO: 101 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 102 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaaaccgaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 103 - protein, 112 aa, synthetic construct; identical to SEQ ID NO: 23
  REGION 1..112: note = Synthetic

SEQ ID NO: 104 - protein, 127 aa, synthetic construct
  REGION 1..127: note = Synthetic
EVQLVESGGGLVQPGRSLRLSCTASGFSFGDHAMSWVRQAPGKGLEWVGFIRKTTYGATTHYAAAVRGRFTISRDDSKSTVYLQMNSLKAEDTAVYFCTRVQLDYGPGYQYYGMDVWGQGTTVTVSS

SEQ ID NO: 105 - DNA, 336 bp, synthetic construct; identical to SEQ ID NO: 25
  misc_feature 1..336: note = Synthetic
  source 1..336: mol_type = other DNA

SEQ ID NO: 106 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaagcagaggacacagccgtgtatttctgtactagagtgcagcttgactatggcccgggataccagtactacggtatggacgtctggggccaagggaccacggtcaccgtctcctca

SEQ ID NO: 107 - protein, 217 aa, synthetic construct; identical to SEQ ID NO: 27
  REGION 1..217: note = Synthetic

SEQ ID NO: 108 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSTVYLQMNSLKAEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSTVYLQMNSLKAEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 110 - DNA, 651 bp, synthetic construct; identical to SEQ ID NO: 30
  misc_feature 1..651: note = Synthetic
  source 1..651: mol_type = other DNA

SEQ ID NO: 111 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaagcagaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 112 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaagcagaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 113 - protein, 112 aa, synthetic construct; identical to SEQ ID NO: 23
  REGION 1..112: note = Synthetic

SEQ ID NO: 114 - protein, 127 aa, synthetic construct
  REGION 1..127: note = Synthetic
EVQLVESGGGLVQPGRSLRLSCTASGFSFGDHAMSWVRQAPGKGLEWVGFIRKTTYGATTHYAAAVRGRFTISRDDSKSTVYLQMNSLKTEDTAVYFCARVQLDYGPGYQYYGMDVWGQGTTVTVSS

SEQ ID NO: 115 - DNA, 336 bp, synthetic construct; identical to SEQ ID NO: 25
  misc_feature 1..336: note = Synthetic
  source 1..336: mol_type = other DNA

SEQ ID NO: 116 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaaaccgaggacacagccgtgtatttctgtgctagagtgcagcttgactatggcccgggataccagtactacggtatggacgtctggggccaagggaccacggtcaccgtctcctca

SEQ ID NO: 117 - protein, 217 aa, synthetic construct; identical to SEQ ID NO: 27
  REGION 1..217: note = Synthetic

SEQ ID NO: 118 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSTVYLQMNSLKTEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSTVYLQMNSLKTEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 120 - DNA, 651 bp, synthetic construct; identical to SEQ ID NO: 30
  misc_feature 1..651: note = Synthetic
  source 1..651: mol_type = other DNA

SEQ ID NO: 121 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaaaccgaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 122 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaaaccgaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 123 - protein, 112 aa, synthetic construct; identical to SEQ ID NO: 23
  REGION 1..112: note = Synthetic

SEQ ID NO: 124 - protein, 127 aa, synthetic construct
  REGION 1..127: note = Synthetic
EVQLVESGGGLVQPGRSLRLSCTASGFSFGDHAMSWVRQAPGKGLEWVGFIRKTTYGATTHYAAAVRGRFTISRDDSKSIVYLQMNSLKAEDTAVYFCARVQLDYGPGYQYYGMDVWGQGTTVTVSS

SEQ ID NO: 125 - DNA, 336 bp, synthetic construct; identical to SEQ ID NO: 25
  misc_feature 1..336: note = Synthetic
  source 1..336: mol_type = other DNA

SEQ ID NO: 126 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaagcagaggacacagccgtgtatttctgtgctagagtgcagcttgactatggcccgggataccagtactacggtatggacgtctggggccaagggaccacggtcaccgtctcctca

SEQ ID NO: 127 - protein, 217 aa, synthetic construct; identical to SEQ ID NO: 27
  REGION 1..217: note = Synthetic

SEQ ID NO: 128 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSIVYLQMNSLKAEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSIVYLQMNSLKAEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 130 - DNA, 651 bp, synthetic construct; identical to SEQ ID NO: 30
  misc_feature 1..651: note = Synthetic
  source 1..651: mol_type = other DNA

SEQ ID NO: 131 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaagcagaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 132 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaagcagaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 133 - protein, 112 aa, synthetic construct; identical to SEQ ID NO: 23
  REGION 1..112: note = Synthetic

SEQ ID NO: 134 - protein, 127 aa, synthetic construct
  REGION 1..127: note = Synthetic
EVQLVESGGGLVQPGRSLRLSCAASGFSFGDHAMSWVRQAPGKGLEWVGFIRKTTYGATTHYAAAVRGRFTISRDDSKSTVYLQMNSLKAEDTAVYFCTRVQLDYGPGYQYYGMDVWGQGTTVTVSS

SEQ ID NO: 135 - DNA, 336 bp, synthetic construct; identical to SEQ ID NO: 25
  misc_feature 1..336: note = Synthetic
  source 1..336: mol_type = other DNA

SEQ ID NO: 136 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaagcagaggacacagccgtgtatttctgtactagagtgcagcttgactatggcccgggataccagtactacggtatggacgtctggggccaagggaccacggtcaccgtctcctca

SEQ ID NO: 137 - protein, 217 aa, synthetic construct; identical to SEQ ID NO: 27
  REGION 1..217: note = Synthetic

SEQ ID NO: 138 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSTVYLQMNSLKAEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSTVYLQMNSLKAEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 140 - DNA, 651 bp, synthetic construct; identical to SEQ ID NO: 30
  misc_feature 1..651: note = Synthetic
  source 1..651: mol_type = other DNA

SEQ ID NO: 141 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaagcagaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 142 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaagcagaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 143 - protein, 112 aa, synthetic construct; identical to SEQ ID NO: 23
  REGION 1..112: note = Synthetic

SEQ ID NO: 144 - protein, 127 aa, synthetic construct
  REGION 1..127: note = Synthetic
EVQLVESGGGLVQPGRSLRLSCAASGFSFGDHAMSWVRQAPGKGLEWVGFIRKTTYGATTHYAAAVRGRFTISRDDSKSTVYLQMNSLKTEDTAVYFCARVQLDYGPGYQYYGMDVWGQGTTVTVSS

SEQ ID NO: 145 - DNA, 336 bp, synthetic construct; identical to SEQ ID NO: 25
  misc_feature 1..336: note = Synthetic
  source 1..336: mol_type = other DNA

SEQ ID NO: 146 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaaaccgaggacacagccgtgtatttctgtgctagagtgcagcttgactatggcccgggataccagtactacggtatggacgtctggggccaagggaccacggtcaccgtctcctca

SEQ ID NO: 147 - protein, 217 aa, synthetic construct; identical to SEQ ID NO: 27
  REGION 1..217: note = Synthetic

SEQ ID NO: 148 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSTVYLQMNSLKTEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSTVYLQMNSLKTEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 150 - DNA, 651 bp, synthetic construct; identical to SEQ ID NO: 30
  misc_feature 1..651: note = Synthetic
  source 1..651: mol_type = other DNA

SEQ ID NO: 151 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaaaccgaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 152 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaaaccgaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 153 - protein, 112 aa, synthetic construct; identical to SEQ ID NO: 23
  REGION 1..112: note = Synthetic

SEQ ID NO: 154 - protein, 127 aa, synthetic construct
  REGION 1..127: note = Synthetic
EVQLVESGGGLVQPGRSLRLSCAASGFSFGDHAMSWVRQAPGKGLEWVGFIRKTTYGATTHYAAAVRGRFTISRDDSKSIVYLQMNSLKAEDTAVYFCARVQLDYGPGYQYYGMDVWGQGTTVTVSS

SEQ ID NO: 155 - DNA, 336 bp, synthetic construct; identical to SEQ ID NO: 25
  misc_feature 1..336: note = Synthetic
  source 1..336: mol_type = other DNA

SEQ ID NO: 156 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaagcagaggacacagccgtgtatttctgtgctagagtgcagcttgactatggcccgggataccagtactacggtatggacgtctggggccaagggaccacggtcaccgtctcctca

SEQ ID NO: 157 - protein, 217 aa, synthetic construct; identical to SEQ ID NO: 27
  REGION 1..217: note = Synthetic

SEQ ID NO: 158 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSIVYLQMNSLKAEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSIVYLQMNSLKAEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 160 - DNA, 651 bp, synthetic construct; identical to SEQ ID NO: 30
  misc_feature 1..651: note = Synthetic
  source 1..651: mol_type = other DNA

SEQ ID NO: 161 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaagcagaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 162 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcattgtctatctgcaaatgaacagcctgaaagcagaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 163 - protein, 112 aa, synthetic construct; identical to SEQ ID NO: 23
  REGION 1..112: note = Synthetic

SEQ ID NO: 164 - protein, 127 aa, synthetic construct
  REGION 1..127: note = Synthetic
EVQLVESGGGLVQPGRSLRLSCTASGFSFGDHAMSWVRQAPGKGLEWVGFIRKTTYGATTHYAAAVRGRFTISRDDSKSTVYLQMNSLKAEDTAVYFCARVQLDYGPGYQYYGMDVWGQGTTVTVSS

SEQ ID NO: 165 - DNA, 336 bp, synthetic construct; identical to SEQ ID NO: 25
  misc_feature 1..336: note = Synthetic
  source 1..336: mol_type = other DNA

SEQ ID NO: 166 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaagcagaggacacagccgtgtatttctgtgctagagtgcagcttgactatggcccgggataccagtactacggtatggacgtctggggccaagggaccacggtcaccgtctcctca

SEQ ID NO: 167 - protein, 217 aa, synthetic construct; identical to SEQ ID NO: 27
  REGION 1..217: note = Synthetic

SEQ ID NO: 168 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSTVYLQMNSLKAEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 457 aa, synthetic construct
  REGION 1..457: note = Synthetic
EVQLVESGGGLVQPGRSLRLSCTASGFSFGDHAMSWVRQAPGKGLEWVGFIRKTTYGATTHYAAAVRGRFTISRDDSKSTVYLQMNSLKAEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 170 - DNA, 651 bp, synthetic construct; identical to SEQ ID NO: 30
  misc_feature 1..651: note = Synthetic
  source 1..651: mol_type = other DNA

SEQ ID NO: 171 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaagcagaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 172 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtacagcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaagcagaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 173 - protein, 112 aa, synthetic construct; identical to SEQ ID NO: 23
  REGION 1..112: note = Synthetic

SEQ ID NO: 174 - protein, 127 aa, synthetic construct
  REGION 1..127: note = Synthetic
EVQLVESGGGLVQPGRSLRLSCAASGFSFGDHAMSWVRQAPGKGLEWVGFIRKTTYGATTHYAAAVRGRFTISRDDSKSTVYLQMNSLKAEDTAVYFCARVQLDYGPGYQYYGMDVWGQGTTVTVSS

SEQ ID NO: 175 - DNA, 336 bp, synthetic construct; identical to SEQ ID NO: 25
  misc_feature 1..336: note = Synthetic
  source 1..336: mol_type = other DNA

SEQ ID NO: 176 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaagcagaggacacagccgtgtatttctgtgctagagtgcagcttgactatggcccgggataccagtactacggtatggacgtctggggccaagggaccacggtcaccgtctcctca

SEQ ID NO: 177 - protein, 217 aa, synthetic construct; identical to SEQ ID NO: 27
  REGION 1..217: note = Synthetic

SEQ ID NO: 178 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSTVYLQMNSLKAEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 457 aa, synthetic construct
  REGION 1..457: note = Synthetic
EVQLVESGGGLVQPGRSLRLSCAASGFSFGDHAMSWVRQAPGKGLEWVGFIRKTTYGATTHYAAAVRGRFTISRDDSKSTVYLQMNSLKAEDTAVYFCARVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 180 - DNA, 651 bp, synthetic construct; identical to SEQ ID NO: 30
  misc_feature 1..651: note = Synthetic
  source 1..651: mol_type = other DNA

SEQ ID NO: 181 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaagcagaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 182 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcagctgtgcggcctctgggtttagttttggtgatcatgctatgagctgggtccgccaggctccagggaaggggctggagtgggtaggtttcattagaaagacaacttatggtgcgacaacacactacgccgcggctgtgagaggcagattcaccatctcgcgagatgattctaaaagcactgtctatctgcaaatgaacagcctgaaagcagaggacacagccgtgtatttctgtgctagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 183 - protein, 14 aa, synthetic construct
  REGION 1..14: note = Synthetic
TGMNSNIGAGYDVY

SEQ ID NO: 184 - protein, 7 aa, synthetic construct
  REGION 1..7: note = Synthetic
GNSNRPS

SEQ ID NO: 185 - protein, 11 aa, synthetic construct
  REGION 1..11: note = Synthetic
QSYDTSLDGWA

SEQ ID NO: 186 - protein, 5 aa, synthetic construct
  REGION 1..5: note = Synthetic
DHAMS

SEQ ID NO: 187 - protein, 19 aa, synthetic construct
  REGION 1..19: note = Synthetic
FIRKTTYGATTKYAAAVKG

SEQ ID NO: 188 - protein, 16 aa, synthetic construct
  REGION 1..16: note = Synthetic
VQLDYGPGYQYYGMDV

SEQ ID NO: 189 - protein, 14 aa, synthetic construct
  REGION 1..14: note = Synthetic
TGMNSNIGAGYDVY

SEQ ID NO: 190 - protein, 7 aa, synthetic construct
  REGION 1..7: note = Synthetic
GNSNRPS

SEQ ID NO: 191 - protein, 11 aa, synthetic construct
  REGION 1..11: note = Synthetic
QSYDTSLDGWA

SEQ ID NO: 192 - protein, 5 aa, synthetic construct
  REGION 1..5: note = Synthetic
DHAMS

SEQ ID NO: 193 - protein, 19 aa, synthetic construct
  REGION 1..19: note = Synthetic
FIRKTTYGATTKYAAAVKG

SEQ ID NO: 194 - protein, 16 aa, synthetic construct
  REGION 1..16: note = Synthetic
VQLDYGPGYQYYGMDV

SEQ ID NO: 195 - protein, 112 aa, synthetic construct
  REGION 1..112: note = Synthetic
ESVLTQPPSVSGAPGQRVTISCTGMNSNIGAGYDVYWYQQLPGRAPKLLIYGNSNRPSGVPDRFSGSRSGTSASLAITGLQAEDEADYYCQSYDTSLDGWAFGGGTKLTVLG

SEQ ID NO: 196 - protein, 127 aa, synthetic construct
  REGION 1..127: note = Synthetic
EVQLVESGGGLVQPGRSLRLPCTASGFSFGDHAMSWVRQAPGKGLEWVGFIRKTTYGATTKYAAAVKGRFTISRDDSKSIVYLQMNSLKTEDTAVYFCTRVQLDYGPGYQYYGMDVWGQGTTVTVSS

SEQ ID NO: 197 - DNA, 336 bp, synthetic construct
  misc_feature 1..336: note = Synthetic
  source 1..336: mol_type = other DNA
gagtctgtgctgacgcagccgccctcagtgtctggggccccagggcagagggtcaccatctcctgcactgggatgaactccaacatcggggcaggttatgatgtatactggtaccaacaacttccaggaagagcccccaaactcctcatctatggtaacagcaatcggccctcaggggtccctgaccgattctctggctccaggtctggcacctcagcctccctggccatcactgggctccaggctgaggatgaggctgattattactgccagtcctatgacaccagcctggacggttgggctttcggcggagggaccaagttgaccgtcctaggc

SEQ ID NO: 198 - DNA, 381 bp, synthetic construct
  misc_feature 1..381: note = Synthetic
  source 1..381: mol_type = other DNA
gaggtgcagctggtggagtctgggggaggcttggtacagccagggcggtccctgagactcccctgtacagcctctgggtttagttttggtgatcatgctatgagctgggtccgccaggctccagggaaggggctggagtgggtaggtttcattagaaagacaacttatggtgcgacaacaaagtacgccgcggctgtgaagggcagattcaccatctcgcgagatgattctaaaagcattgtctatctgcaaatgaacagcctgaaaaccgaggacacagccgtgtatttctgtactagagtgcagcttgactatggcccgggataccagtactacggtatggacgtctggggccaagggaccacggtcaccgtctcctca

SEQ ID NO: 199 - protein, 217 aa, synthetic construct
  REGION 1..217: note = Synthetic
ESVLTQPPSVSGAPGQRVTISCTGMNSNIGAGYDVYWYQQLPGRAPKLLIYGNSNRPSGVPDRFSGSRSGTSASLAITGLQAEDEADYYCQSYDTSLDGWAFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 200 - protein, 457 aa, synthetic construct
  REGION 1..457: note = Synthetic
EVQLVESGGGLVQPGRSLRLPCTASGFSFGDHAMSWVRQAPGKGLEWVGFIRKTTYGATTKYAAAVKGRFTISRDDSKSIVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 457 aa, synthetic construct
  REGION 1..457: note = Synthetic
EVQLVESGGGLVQPGRSLRLPCTASGFSFGDHAMSWVRQAPGKGLEWVGFIRKTTYGATTKYAAAVKGRFTISRDDSKSIVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 202 - DNA, 651 bp, synthetic construct
  misc_feature 1..651: note = Synthetic
  source 1..651: mol_type = other DNA
gagtctgtgctgacgcagccgccctcagtgtctggggccccagggcagagggtcaccatctcctgcactgggatgaactccaacatcggggcaggttatgatgtatactggtaccaacaacttccaggaagagcccccaaactcctcatctatggtaacagcaatcggccctcaggggtccctgaccgattctctggctccaggtctggcacctcagcctccctggccatcactgggctccaggctgaggatgaggctgattattactgccagtcctatgacaccagcctggacggttgggctttcggcggagggaccaagttgaccgtcctaggccagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 203 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcccctgtacagcctctgggtttagttttggtgatcatgctatgagctgggtccgccaggctccagggaaggggctggagtgggtaggtttcattagaaagacaacttatggtgcgacaacaaagtacgccgcggctgtgaagggcagattcaccatctcgcgagatgattctaaaagcattgtctatctgcaaatgaacagcctgaaaaccgaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 204 - DNA, 1371 bp, synthetic construct
  misc_feature 1..1371: note = Synthetic
  source 1..1371: mol_type = other DNA
gaggtgcagctggtggagtctgggggaggcttggtacagccagggcggtccctgagactcccctgtacagcctctgggtttagttttggtgatcatgctatgagctgggtccgccaggctccagggaaggggctggagtgggtaggtttcattagaaagacaacttatggtgcgacaacaaagtacgccgcggctgtgaagggcagattcaccatctcgcgagatgattctaaaagcattgtctatctgcaaatgaacagcctgaaaaccgaggacacagccgtgtatttctgtactagagtgcagcttgactatggcccgggataccagtactacggtatggacgtctggggccaagggaccacggtcaccgtctcctcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 205 - protein, 11 aa, synthetic construct
  REGION 1..11: note = Synthetic
RASQSISSWLA

SEQ ID NO: 206 - protein, 7 aa, synthetic construct
  REGION 1..7: note = Synthetic
DASSLES

SEQ ID NO: 207 - protein, 10 aa, synthetic construct
  REGION 1..10: note = Synthetic
QQYNSYSFWT

SEQ ID NO: 208 - protein, 5 aa, synthetic construct
  REGION 1..5: note = Synthetic
TYGMH

SEQ ID NO: 209 - protein, 17 aa, synthetic construct
  REGION 1..17: note = Synthetic
IIWYDGSQKYYADSVQG

SEQ ID NO: 210 - protein, 14 aa, synthetic construct
  REGION 1..14: note = Synthetic
VRFSVGPHGSAFDL

SEQ ID NO: 211 - protein, 11 aa, synthetic construct
  REGION 1..11: note = Synthetic
RASQSISSWLA

SEQ ID NO: 212 - protein, 7 aa, synthetic construct
  REGION 1..7: note = Synthetic
DASSLES

SEQ ID NO: 213 - protein, 11 aa, synthetic construct
  REGION 1..11: note = Synthetic
QQYNSYSFWTF

SEQ ID NO: 214 - protein, 5 aa, synthetic construct
  REGION 1..5: note = Synthetic
TYGMH

SEQ ID NO: 215 - protein, 17 aa, synthetic construct
  REGION 1..17: note = Synthetic
IIWYDGSQKYYADSVQG

SEQ ID NO: 216 - protein, 5 aa, synthetic construct
  REGION 1..5: note = Synthetic
SAFDL

SEQ ID NO: 217 - protein, 109 aa, synthetic construct
  REGION 1..109: note = Synthetic
GVQMTQSPSTLSASVGDRVTLTCRASQSISSWLAWYQQKPGKAPKLLIYDASSLESGVPSRFSGSGSGTEFTLTISSLQPDDFATYYCQQYNSYSFWTFGQGTKVEIKR

SEQ ID NO: 218 - protein, 123 aa, synthetic construct
  REGION 1..123: note = Synthetic
QVQLVESGGGVVQPGRSLRLSCAASGFAFNTYGMHWVRQTPGKGLEWVAIIWYDGSQKYYADSVQGRFIISRDNHKNTLSLQMNGLRAEDTAVYFCVRVRFSVGPHGSAFDLWGQGTMVIVSS

SEQ ID NO: 219 - DNA, 327 bp, synthetic construct
  misc_feature 1..327: note = Synthetic
  source 1..327: mol_type = other DNA
ggcgtccagatgacccagtctccttccaccctgtctgcatctgtgggagacagagtcaccctcacttgccgggccagtcagagtattagtagttggttggcctggtatcagcagaaaccagggaaagcccctaaactcctgatctatgatgcctccagtttggaaagtggggtcccatcaaggttcagcggcagtggatctgggacagaattcactctcaccatcagcagcctgcaacctgatgattttgcaacttattactgccaacagtataatagttattctttttggacgttcggccaagggaccaaggtggaaatcaaacgc

SEQ ID NO: 220 - DNA, 369 bp, synthetic construct
  misc_feature 1..369: note = Synthetic
  source 1..369: mol_type = other DNA
caggtgcaactggtggagtctgggggaggcgtggtccagcctgggaggtccctgagactctcctgtgcagcgtctgggttcgctttcaatacctatggcatgcactgggtccgccagactccaggcaaggggctggagtgggtggcaattatctggtatgatgggagccagaaatactatgcagactccgtgcagggccgattcatcatctccagagacaatcacaagaacacgttgtctctgcaaatgaacggcctgagagccgaggacacggctgtgtatttctgtgtgagagtccgctttagcgttggcccccacgggagtgcttttgatctctggggccaggggacaatggtcatcgtctcttca

SEQ ID NO: 221 - protein, 215 aa, synthetic construct
  REGION 1..215: note = Synthetic
GVQMTQSPSTLSASVGDRVTLTCRASQSISSWLAWYQQKPGKAPKLLIYDASSLESGVPSRFSGSGSGTEFTLTISSLQPDDFATYYCQQYNSYSFWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 453 aa, synthetic construct
  REGION 1..453: note = Synthetic
QVQLVESGGGVVQPGRSLRLSCAASGFAFNTYGMHWVRQTPGKGLEWVAIIWYDGSQKYYADSVQGRFIISRDNHKNTLSLQMNGLRAEDTAVYFCVRVRFSVGPHGSAFDLWGQGTMVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 453 aa, synthetic construct
  REGION 1..453: note = Synthetic
QVQLVESGGGVVQPGRSLRLSCAASGFAFNTYGMHWVRQTPGKGLEWVAIIWYDGSQKYYADSVQGRFIISRDNHKNTLSLQMNGLRAEDTAVYFCVRVRFSVGPHGSAFDLWGQGTMVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 224 - DNA, 645 bp, synthetic construct
  misc_feature 1..645: note = Synthetic
  source 1..645: mol_type = other DNA
ggcgtccagatgacccagtctccttccaccctgtctgcatctgtgggagacagagtcaccctcacttgccgggccagtcagagtattagtagttggttggcctggtatcagcagaaaccagggaaagcccctaaactcctgatctatgatgcctccagtttggaaagtggggtcccatcaaggttcagcggcagtggatctgggacagaattcactctcaccatcagcagcctgcaacctgatgattttgcaacttattactgccaacagtataatagttattctttttggacgttcggccaagggaccaaggtggaaatcaaacgcactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 225 - DNA, 1359 bp, synthetic construct
  misc_feature 1..1359: note = Synthetic
  source 1..1359: mol_type = other DNA
caggtgcaactggtggagtctgggggaggcgtggtccagcctgggaggtccctgagactctcctgtgcagcgtctgggttcgctttcaatacctatggcatgcactgggtccgccagactccaggcaaggggctggagtgggtggcaattatctggtatgatgggagccagaaatactatgcagactccgtgcagggccgattcatcatctccagagacaatcacaagaacacgttgtctctgcaaatgaacggcctgagagccgaggacacggctgtgtatttctgtgtgagagtccgctttagcgttggcccccacgggagtgcttttgatctctggggccaggggacaatggtcatcgtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 226 - DNA, 1359 bp, synthetic construct
  misc_feature 1..1359: note = Synthetic
  source 1..1359: mol_type = other DNA
caggtgcaactggtggagtctgggggaggcgtggtccagcctgggaggtccctgagactctcctgtgcagcgtctgggttcgctttcaatacctatggcatgcactgggtccgccagactccaggcaaggggctggagtgggtggcaattatctggtatgatgggagccagaaatactatgcagactccgtgcagggccgattcatcatctccagagacaatcacaagaacacgttgtctctgcaaatgaacggcctgagagccgaggacacggctgtgtatttctgtgtgagagtccgctttagcgttggcccccacgggagtgcttttgatctctggggccaggggacaatggtcatcgtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 227 - protein, 109 aa, synthetic construct; identical to SEQ ID NO: 217
  REGION 1..109: note = Synthetic

SEQ ID NO: 228 - protein, 123 aa, synthetic construct
  REGION 1..123: note = Synthetic
QVQLVESGGGVVQPGRSLRLSCAASGFAFNTYGMHWVRQAPGKGLEWVAIIWYDGSQKYYADSVQGRFIISRDNHKNTLSLQMNSLRAEDTAVYFCVRVRFSVGPHGSAFDLWGQGTMVTVSS

SEQ ID NO: 229 - DNA, 327 bp, synthetic construct; identical to SEQ ID NO: 219
  misc_feature 1..327: note = Synthetic
  source 1..327: mol_type = other DNA

SEQ ID NO: 230 - DNA, 369 bp, synthetic construct
  misc_feature 1..369: note = Synthetic
  source 1..369: mol_type = other DNA
caggtgcaactggtggagtctgggggaggcgtggtccagcctgggaggtccctgagactctcctgtgcagcgtctgggttcgctttcaatacctatggcatgcactgggtccgccaggcaccaggcaaggggctggagtgggtggcaattatctggtatgatgggagccagaaatactatgcagactccgtgcagggccgattcatcatctccagagacaatcacaagaacacgttgtctctgcaaatgaactccctgagagccgaggacacggctgtgtatttctgtgtgagagtccgctttagcgttggcccccacgggagtgcttttgatctctggggccaggggacaatggtcacagtctcttca

SEQ ID NO: 231 - protein, 215 aa, synthetic construct; identical to SEQ ID NO: 221
  REGION 1..215: note = Synthetic

SEQ ID NO: 232 - protein, 453 aa, synthetic construct
  REGION 1..453: note = Synthetic
QVQLVESGGGVVQPGRSLRLSCAASGFAFNTYGMHWVRQAPGKGLEWVAIIWYDGSQKYYADSVQGRFIISRDNHKNTLSLQMNSLRAEDTAVYFCVRVRFSVGPHGSAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 453 aa, synthetic construct
  REGION 1..453: note = Synthetic
QVQLVESGGGVVQPGRSLRLSCAASGFAFNTYGMHWVRQAPGKGLEWVAIIWYDGSQKYYADSVQGRFIISRDNHKNTLSLQMNSLRAEDTAVYFCVRVRFSVGPHGSAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 234 - DNA, 645 bp, synthetic construct; identical to SEQ ID NO: 224
  misc_feature 1..645: note = Synthetic
  source 1..645: mol_type = other DNA

SEQ ID NO: 235 - DNA, 1359 bp, synthetic construct
  misc_feature 1..1359: note = Synthetic
  source 1..1359: mol_type = other DNA
caggtgcaactggtggagtctgggggaggcgtggtccagcctgggaggtccctgagactctcctgtgcagcgtctgggttcgctttcaatacctatggcatgcactgggtccgccaggcaccaggcaaggggctggagtgggtggcaattatctggtatgatgggagccagaaatactatgcagactccgtgcagggccgattcatcatctccagagacaatcacaagaacacgttgtctctgcaaatgaactccctgagagccgaggacacggctgtgtatttctgtgtgagagtccgctttagcgttggcccccacgggagtgcttttgatctctggggccaggggacaatggtcacagtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 236 - DNA, 1359 bp, synthetic construct
  misc_feature 1..1359: note = Synthetic
  source 1..1359: mol_type = other DNA
caggtgcaactggtggagtctgggggaggcgtggtccagcctgggaggtccctgagactctcctgtgcagcgtctgggttcgctttcaatacctatggcatgcactgggtccgccaggcaccaggcaaggggctggagtgggtggcaattatctggtatgatgggagccagaaatactatgcagactccgtgcagggccgattcatcatctccagagacaatcacaagaacacgttgtctctgcaaatgaactccctgagagccgaggacacggctgtgtatttctgtgtgagagtccgctttagcgttggcccccacgggagtgcttttgatctctggggccaggggacaatggtcacagtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 237 - protein, 109 aa, synthetic construct; identical to SEQ ID NO: 217
  REGION 1..109: note = Synthetic

SEQ ID NO: 238 - protein, 123 aa, synthetic construct
  REGION 1..123: note = Synthetic
QVQLVESGGGVVQPGRSLRLSCAASGFTFNTYGMHWVRQAPGKGLEWVAIIWYDGSQKYYADSVQGRFIISRDNHKNTLSLQMNSLRAEDTAVYFCVRVRFSVGPHGSAFDLWGQGTMVTVSS

SEQ ID NO: 239 - DNA, 327 bp, synthetic construct; identical to SEQ ID NO: 219
  misc_feature 1..327: note = Synthetic
  source 1..327: mol_type = other DNA

SEQ ID NO: 240 - DNA, 369 bp, synthetic construct
  misc_feature 1..369: note = Synthetic
  source 1..369: mol_type = other DNA
caggtgcaactggtggagtctgggggaggcgtggtccagcctgggaggtccctgagactctcctgtgcagcgtctgggttcactttcaatacctatggcatgcactgggtccgccaggcaccaggcaaggggctggagtgggtggcaattatctggtatgatgggagccagaaatactatgcagactccgtgcagggccgattcatcatctccagagacaatcacaagaacacgttgtctctgcaaatgaactccctgagagccgaggacacggctgtgtatttctgtgtgagagtccgctttagcgttggcccccacgggagtgcttttgatctctggggccaggggacaatggtcacagtctcttca

SEQ ID NO: 241 - protein, 215 aa, synthetic construct; identical to SEQ ID NO: 221
  REGION 1..215: note = Synthetic

SEQ ID NO: 242 - protein, 453 aa, synthetic construct
  REGION 1..453: note = Synthetic
QVQLVESGGGVVQPGRSLRLSCAASGFTFNTYGMHWVRQAPGKGLEWVAIIWYDGSQKYYADSVQGRFIISRDNHKNTLSLQMNSLRAEDTAVYFCVRVRFSVGPHGSAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 453 aa, synthetic construct
  REGION 1..453: note = Synthetic
QVQLVESGGGVVQPGRSLRLSCAASGFTFNTYGMHWVRQAPGKGLEWVAIIWYDGSQKYYADSVQGRFIISRDNHKNTLSLQMNSLRAEDTAVYFCVRVRFSVGPHGSAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 244 - DNA, 645 bp, synthetic construct; identical to SEQ ID NO: 224
  misc_feature 1..645: note = Synthetic
  source 1..645: mol_type = other DNA

SEQ ID NO: 245 - DNA, 1359 bp, synthetic construct
  misc_feature 1..1359: note = Synthetic
  source 1..1359: mol_type = other DNA
caggtgcaactggtggagtctgggggaggcgtggtccagcctgggaggtccctgagactctcctgtgcagcgtctgggttcactttcaatacctatggcatgcactgggtccgccaggcaccaggcaaggggctggagtgggtggcaattatctggtatgatgggagccagaaatactatgcagactccgtgcagggccgattcatcatctccagagacaatcacaagaacacgttgtctctgcaaatgaactccctgagagccgaggacacggctgtgtatttctgtgtgagagtccgctttagcgttggcccccacgggagtgcttttgatctctggggccaggggacaatggtcacagtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 246 - DNA, 1359 bp, synthetic construct
  misc_feature 1..1359: note = Synthetic
  source 1..1359: mol_type = other DNA
caggtgcaactggtggagtctgggggaggcgtggtccagcctgggaggtccctgagactctcctgtgcagcgtctgggttcactttcaatacctatggcatgcactgggtccgccaggcaccaggcaaggggctggagtgggtggcaattatctggtatgatgggagccagaaatactatgcagactccgtgcagggccgattcatcatctccagagacaatcacaagaacacgttgtctctgcaaatgaactccctgagagccgaggacacggctgtgtatttctgtgtgagagtccgctttagcgttggcccccacgggagtgcttttgatctctggggccaggggacaatggtcacagtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 247 - protein, 109 aa, synthetic construct; identical to SEQ ID NO: 217
  REGION 1..109: note = Synthetic

SEQ ID NO: 248 - protein, 123 aa, synthetic construct
  REGION 1..123: note = Synthetic
QVQLVESGGGVVQPGRSLRLSCAASGFAFNTYGMHWVRQAPGKGLEWVAIIWYDGSQKYYADSVQGRFTISRDNHKNTLSLQMNSLRAEDTAVYFCVRVRFSVGPHGSAFDLWGQGTMVTVSS

SEQ ID NO: 249 - DNA, 327 bp, synthetic construct; identical to SEQ ID NO: 219
  misc_feature 1..327: note = Synthetic
  source 1..327: mol_type = other DNA

SEQ ID NO: 250 - DNA, 369 bp, synthetic construct
  misc_feature 1..369: note = Synthetic
  source 1..369: mol_type = other DNA
caggtgcaactggtggagtctgggggaggcgtggtccagcctgggaggtccctgagactctcctgtgcagcgtctgggttcgctttcaatacctatggcatgcactgggtccgccaggcaccaggcaaggggctggagtgggtggcaattatctggtatgatgggagccagaaatactatgcagactccgtgcagggccgattcactatctccagagacaatcacaagaacacgttgtctctgcaaatgaactccctgagagccgaggacacggctgtgtatttctgtgtgagagtccgctttagcgttggcccccacgggagtgcttttgatctctggggccaggggacaatggtcacagtctcttca

SEQ ID NO: 251 - protein, 215 aa, synthetic construct; identical to SEQ ID NO: 221
  REGION 1..215: note = Synthetic

SEQ ID NO: 252 - protein, 453 aa, synthetic construct
  REGION 1..453: note = Synthetic
QVQLVESGGGVVQPGRSLRLSCAASGFAFNTYGMHWVRQAPGKGLEWVAIIWYDGSQKYYADSVQGRFTISRDNHKNTLSLQMNSLRAEDTAVYFCVRVRFSVGPHGSAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - protein, 453 aa, synthetic construct
  REGION 1..453: note = Synthetic
QVQLVESGGGVVQPGRSLRLSCAASGFAFNTYGMHWVRQAPGKGLEWVAIIWYDGSQKYYADSVQGRFTISRDNHKNTLSLQMNSLRAEDTAVYFCVRVRFSVGPHGSAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 254 - DNA, 645 bp, synthetic construct; identical to SEQ ID NO: 224
  misc_feature 1..645: note = Synthetic
  source 1..645: mol_type = other DNA

SEQ ID NO: 255 - DNA, 1359 bp, synthetic construct
  misc_feature 1..1359: note = Synthetic
  source 1..1359: mol_type = other DNA
caggtgcaactggtggagtctgggggaggcgtggtccagcctgggaggtccctgagactctcctgtgcagcgtctgggttcgctttcaatacctatggcatgcactgggtccgccaggcaccaggcaaggggctggagtgggtggcaattatctggtatgatgggagccagaaatactatgcagactccgtgcagggccgattcactatctccagagacaatcacaagaacacgttgtctctgcaaatgaactccctgagagccgaggacacggctgtgtatttctgtgtgagagtccgctttagcgttggcccccacgggagtgcttttgatctctggggccaggggacaatggtcacagtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 256 - DNA, 1359 bp, synthetic construct
  misc_feature 1..1359: note = Synthetic
  source 1..1359: mol_type = other DNA
caggtgcaactggtggagtctgggggaggcgtggtccagcctgggaggtccctgagactctcctgtgcagcgtctgggttcgctttcaatacctatggcatgcactgggtccgccaggcaccaggcaaggggctggagtgggtggcaattatctggtatgatgggagccagaaatactatgcagactccgtgcagggccgattcactatctccagagacaatcacaagaacacgttgtctctgcaaatgaactccctgagagccgaggacacggctgtgtatttctgtgtgagagtccgctttagcgttggcccccacgggagtgcttttgatctctggggccaggggacaatggtcacagtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 257 - protein, 109 aa, synthetic construct; identical to SEQ ID NO: 217
  REGION 1..109: note = Synthetic

SEQ ID NO: 258 - protein, 123 aa, synthetic construct
  REGION 1..123: note = Synthetic
QVQLVESGGGVVQPGRSLRLSCAASGFTFNTYGMHWVRQAPGKGLEWVAIIWYDGSQKYYADSVQGRFTISRDNHKNTLSLQMNSLRAEDTAVYFCVRVRFSVGPHGSAFDLWGQGTMVTVSS

SEQ ID NO: 259 - DNA, 327 bp, synthetic construct; identical to SEQ ID NO: 219
  misc_feature 1..327: note = Synthetic
  source 1..327: mol_type = other DNA

SEQ ID NO: 260 - DNA, 369 bp, synthetic construct
  misc_feature 1..369: note = Synthetic
  source 1..369: mol_type = other DNA
caggtgcaactggtggagtctgggggaggcgtggtccagcctgggaggtccctgagactctcctgtgcagcgtctgggttcactttcaatacctatggcatgcactgggtccgccaggcaccaggcaaggggctggagtgggtggcaattatctggtatgatgggagccagaaatactatgcagactccgtgcagggccgattcactatctccagagacaatcacaagaacacgttgtctctgcaaatgaactccctgagagccgaggacacggctgtgtatttctgtgtgagagtccgctttagcgttggcccccacgggagtgcttttgatctctggggccaggggacaatggtcacagtctcttca

SEQ ID NO: 261 - protein, 215 aa, synthetic construct; identical to SEQ ID NO: 221
  REGION 1..215: note = Synthetic

SEQ ID NO: 262 - protein, 453 aa, synthetic construct
  REGION 1..453: note = Synthetic
QVQLVESGGGVVQPGRSLRLSCAASGFTFNTYGMHWVRQAPGKGLEWVAIIWYDGSQKYYADSVQGRFTISRDNHKNTLSLQMNSLRAEDTAVYFCVRVRFSVGPHGSAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - protein, 453 aa, synthetic construct
  REGION 1..453: note = Synthetic
QVQLVESGGGVVQPGRSLRLSCAASGFTFNTYGMHWVRQAPGKGLEWVAIIWYDGSQKYYADSVQGRFTISRDNHKNTLSLQMNSLRAEDTAVYFCVRVRFSVGPHGSAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 264 - DNA, 645 bp, synthetic construct; identical to SEQ ID NO: 224
  misc_feature 1..645: note = Synthetic
  source 1..645: mol_type = other DNA

SEQ ID NO: 265 - DNA, 1359 bp, synthetic construct
  misc_feature 1..1359: note = Synthetic
  source 1..1359: mol_type = other DNA
caggtgcaactggtggagtctgggggaggcgtggtccagcctgggaggtccctgagactctcctgtgcagcgtctgggttcactttcaatacctatggcatgcactgggtccgccaggcaccaggcaaggggctggagtgggtggcaattatctggtatgatgggagccagaaatactatgcagactccgtgcagggccgattcactatctccagagacaatcacaagaacacgttgtctctgcaaatgaactccctgagagccgaggacacggctgtgtatttctgtgtgagagtccgctttagcgttggcccccacgggagtgcttttgatctctggggccaggggacaatggtcacagtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 266 - DNA, 1359 bp, synthetic construct
  misc_feature 1..1359: note = Synthetic
  source 1..1359: mol_type = other DNA
caggtgcaactggtggagtctgggggaggcgtggtccagcctgggaggtccctgagactctcctgtgcagcgtctgggttcactttcaatacctatggcatgcactgggtccgccaggcaccaggcaaggggctggagtgggtggcaattatctggtatgatgggagccagaaatactatgcagactccgtgcagggccgattcactatctccagagacaatcacaagaacacgttgtctctgcaaatgaactccctgagagccgaggacacggctgtgtatttctgtgtgagagtccgctttagcgttggcccccacgggagtgcttttgatctctggggccaggggacaatggtcacagtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 267 - protein, 109 aa, synthetic construct; identical to SEQ ID NO: 217
  REGION 1..109: note = Synthetic

SEQ ID NO: 268 - protein, 123 aa, synthetic construct
  REGION 1..123: note = Synthetic
QVQLVESGGGVVQPGRSLRLSCAASGFTFNTYGMHWVRQAPGKGLEWVAIIWYDGSQKYYADSVQGRFTISRDNHKNTLYLQMNSLRAEDTAVYFCVRVRFSVGPHGSAFDLWGQGTMVTVSS

SEQ ID NO: 269 - DNA, 327 bp, synthetic construct; identical to SEQ ID NO: 219
  misc_feature 1..327: note = Synthetic
  source 1..327: mol_type = other DNA

SEQ ID NO: 270 - DNA, 369 bp, synthetic construct
  misc_feature 1..369: note = Synthetic
  source 1..369: mol_type = other DNA
caggtgcaactggtggagtctgggggaggcgtggtccagcctgggaggtccctgagactctcctgtgcagcgtctgggttcactttcaatacctatggcatgcactgggtccgccaggcaccaggcaaggggctggagtgggtggcaattatctggtatgatgggagccagaaatactatgcagactccgtgcagggccgattcactatctccagagacaatcacaagaacacgttgtacctgcaaatgaactccctgagagccgaggacacggctgtgtatttctgtgtgagagtccgctttagcgttggcccccacgggagtgcttttgatctctggggccaggggacaatggtcacagtctcttca

SEQ ID NO: 271 - protein, 215 aa, synthetic construct; identical to SEQ ID NO: 221
  REGION 1..215: note = Synthetic

SEQ ID NO: 272 - protein, 453 aa, synthetic construct
  REGION 1..453: note = Synthetic
QVQLVESGGGVVQPGRSLRLSCAASGFTFNTYGMHWVRQAPGKGLEWVAIIWYDGSQKYYADSVQGRFTISRDNHKNTLYLQMNSLRAEDTAVYFCVRVRFSVGPHGSAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 273 - protein, 453 aa, synthetic construct
  REGION 1..453: note = Synthetic
QVQLVESGGGVVQPGRSLRLSCAASGFTFNTYGMHWVRQAPGKGLEWVAIIWYDGSQKYYADSVQGRFTISRDNHKNTLYLQMNSLRAEDTAVYFCVRVRFSVGPHGSAFDLWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 274 - DNA, 645 bp, synthetic construct; identical to SEQ ID NO: 224
  misc_feature 1..645: note = Synthetic
  source 1..645: mol_type = other DNA

SEQ ID NO: 275 - DNA, 1359 bp, synthetic construct
  misc_feature 1..1359: note = Synthetic
  source 1..1359: mol_type = other DNA
caggtgcaactggtggagtctgggggaggcgtggtccagcctgggaggtccctgagactctcctgtgcagcgtctgggttcactttcaatacctatggcatgcactgggtccgccaggcaccaggcaaggggctggagtgggtggcaattatctggtatgatgggagccagaaatactatgcagactccgtgcagggccgattcactatctccagagacaatcacaagaacacgttgtacctgcaaatgaactccctgagagccgaggacacggctgtgtatttctgtgtgagagtccgctttagcgttggcccccacgggagtgcttttgatctctggggccaggggacaatggtcacagtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 276 - DNA, 1359 bp, synthetic construct
  misc_feature 1..1359: note = Synthetic
  source 1..1359: mol_type = other DNA
caggtgcaactggtggagtctgggggaggcgtggtccagcctgggaggtccctgagactctcctgtgcagcgtctgggttcactttcaatacctatggcatgcactgggtccgccaggcaccaggcaaggggctggagtgggtggcaattatctggtatgatgggagccagaaatactatgcagactccgtgcagggccgattcactatctccagagacaatcacaagaacacgttgtacctgcaaatgaactccctgagagccgaggacacggctgtgtatttctgtgtgagagtccgctttagcgttggcccccacgggagtgcttttgatctctggggccaggggacaatggtcacagtctcttcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcactcccactacacacagaagagcctctccctgtctccgggtaaa

SEQ ID NO: 277 - protein, 19 aa, synthetic construct
  REGION 1..19: note = Synthetic
MAWALLLLTLLTQGTGSWA

SEQ ID NO: 278 - protein, 22 aa, synthetic construct
  REGION 1..22: note = Synthetic
MDMRVPAQLLGLLLLWLRGARC

SEQ ID NO: 279 - protein, 24 aa, synthetic construct
  REGION 1..24: note = Synthetic
NANPNANPNANPNANPNANPNANP

SEQ ID NO: 280 - protein, 12 aa, synthetic construct
  REGION 1..12: note = Synthetic
NPNANPNANPNA

SEQ ID NO: 281 - protein, 20 aa, synthetic construct
  REGION 1..20: note = Synthetic
NVDPNANPNVDPNANPNVDP

SEQ ID NO: 282 - protein, 16 aa, synthetic construct
  REGION 1..16: note = Synthetic
NPDPNANPNVDPNANP

SEQ ID NO: 283 - protein, 12 aa, synthetic construct
  REGION 1..12: note = Synthetic
DPNANPNVDPNA

SEQ ID NO: 284 - protein, 15 aa, synthetic construct
  REGION 1..15: note = Synthetic
KQPADGNPDPNANPN

SEQ ID NO: 285 - protein, 424 aa, synthetic construct
  REGION 1..424: note = Synthetic
MMRKLAILSVSSFLFVEALFQEYQCYGSSSNTRVLNELNYDNAGINLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIQNSLSTEWSPCSVTCGNGIQVRIKPGSANKPKDELDYENDIEKKICKMEKCSSVFNVVNSSIGLIMVLSFLFLN

SEQ ID NO: 286 - protein, 236 aa, synthetic construct
  REGION 1..236: note = Synthetic
MAWALLLLTLLTQGTGSWAESVLTQPPSVSGAPGQRVTISCTGMNSNIGAGYDVYWYQQLPGRAPKLLIYGNSNRPSGVPDRFSGSRSGTSASLAITGLQAEDEADYYCQSYDTSLNGWAFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 287 - protein, 479 aa, synthetic construct
  REGION 1..479: note = Synthetic
MDMRVPAQLLGLLLLWLRGARCEVQLVESGGGLVQPGRSLRLPCTASGFSFGDHAMSWVRQAPGKGLEWVGFIRKTTYGATTHYAAAVRGRFTISRDDSKSIVYLQMNSLKTEDTAVYFCTRVQLDYGPGYQYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 288 - protein, 237 aa, synthetic construct
  REGION 1..237: note = Synthetic
MDMRVPAQLLGLLLLWLRGARCGVQMTQSPSTLSASVGDRVTLTCRASQSISSWLAWYQQKPGKAPKLLIYDASSLESGVPSRFSGSGSGTEFTLTISSLQPDDFATYYCQQYNSYSFWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 289 - protein, 475 aa, synthetic construct
  REGION 1..475: note = Synthetic
MDMRVPAQLLGLLLLWLRGARCQVQLVESGGGVVQPGRSLRLSCAASGFAFNTYGMHWVRQTPGKGLEWVAIIWYDGSQKYYADSVQGRFIISRDNHKNTLSLQMNGLRAEDTAVYFCVRVRFSVGPHGSAFDLWGQGTMVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK